SEQUENCE LISTING (54 sequences)

SEQ ID NO: 1 - protein, 12 aa, synthetic construct
QNHFVDEYDPTI

SEQ ID NO: 2 - protein, 10 aa, synthetic construct
EYDPTIEDSY

SEQ ID NO: 3 - protein, 13 aa, synthetic construct
FVDEYDPTIEDSY

SEQ ID NO: 4 - protein, 9 aa, synthetic construct
QNHFVDEYD

SEQ ID NO: 5 - protein, 16 aa, synthetic construct
QNHFVDEYDPTIEDSY

SEQ ID NO: 6 - protein, 17 aa, synthetic construct
QNHFVDEYDPTIEDSYR

SEQ ID NO: 7 - protein, 9 aa, synthetic construct
GGSGGSGGT

SEQ ID NO: 8 - skipped

SEQ ID NO: 9 - protein, 8 aa, synthetic construct
GGSGGSGG

SEQ ID NO: 10 - protein, 5 aa, synthetic construct
GGSGG

SEQ ID NO: 11 - protein, 73 aa, synthetic construct
YKLVVVGAGGVGKSALTIQLIQNHFVDEYDPTIEDSYRKQVVIDGETCLLDILDTAGQEEYSAMRDQYMRTGE

SEQ ID NO: 12 - protein, 188 aa, synthetic construct [sequence omitted]

SEQ ID NO: 13 - protein, 57 aa, synthetic construct
MTEYKLVVVGAGGVGKSALTIQLIQNHFVDEYDPTIEDSYRKQVVIDGETCLLDILD

SEQ ID NO: 14 - protein, 9 aa, synthetic construct
LPPLERLTL

SEQ ID NO: 15 - protein, 129 aa, synthetic construct
EPQQSFSEAQQQLCNTRQEVNELRKLLEEERDQRVAAENALSVAEEQIRRLEHSEWDSSRTPIIGSCGTQEQALLIDLTSNSCRRTRSGVGWKRVLRSLCHSRTRVPLLAAIYFLMIHVLLILCFTGHL

SEQ ID NO: 16 - protein, 11 aa, synthetic construct
MGCIKSKRKDK

SEQ ID NO: 17 - protein, 81 aa, synthetic construct
PSKTSNTIRVFLPNKQRTVVNVRNGMSLHDCLMKALKVRGLQPECCAVFRLLHEHKGKKARLDWNTDAASLIGEELQVDFL

SEQ ID NO: 18 - protein, 81 aa, synthetic construct
PSKTSNTIRVLLPNHERTVVKVRNGMSLHDSLMKALKRHGLQPESSAVFRLLHEHKGKKARLDWNTDAASLIGEELHVDFL

SEQ ID NO: 19 - protein, 81 aa, synthetic construct
PSKTSNTIRVLLPNQEWTVVKVRNGMSLHDSLMKALKRHGLQPESSAVFRLLHEHKGKKARLDWNTDAASLIGEELHVDFL

SEQ ID NO: 20 - protein, 81 aa, synthetic construct
PSKTSNTIRVFLPNKESTVVNVRNGMSLHDSLMKALKRHGLQPESSAVFRLLHEHKGKKARLDWNTDAASLIGEELHVDFL

SEQ ID NO: 21 - protein, 81 aa, synthetic construct
PSKTSNTIRVLLPNNERTVVNVRNGMSLHDSLMKALKRHGLQPESSAVFRLLHEHKGKKARLDWNTDAASLIGEELHVDFL

SEQ ID NO: 22 - DNA, 39 bp, synthetic construct
  source 1..39: mol_type = other DNA
gtttaaacttaagcttgccaccatggtgagcaagggcga

SEQ ID NO: 23 - DNA, 28 bp, synthetic construct
  source 1..28: mol_type = other DNA
gcatgcgcttgtacagctcgtccatgcc

SEQ ID NO: 24 - DNA, 43 bp, synthetic construct
  source 1..43: mol_type = other DNA
ctgtacaagcgcatgcatccttctaagacaagcaacactatcc

SEQ ID NO: 25 - DNA, 45 bp, synthetic construct
  source 1..45: mol_type = other DNA
acctcctatagtggggtcgtattcgtccacaaaatgattctgact

SEQ ID NO: 26 - DNA, 50 bp, synthetic construct
  source 1..50: mol_type = other DNA
cctgtacctccactacctcccaggaaatctacttgaagttcttctccaat

SEQ ID NO: 27 - DNA, 46 bp, synthetic construct
  source 1..46: mol_type = other DNA
tccacaaaatgattctgactacctcctgtacctccactacctccca

SEQ ID NO: 28 - DNA, 49 bp, synthetic construct
  source 1..49: mol_type = other DNA
acgaccccactataggaggtacatctaaaggtgaagaattattcactgg

SEQ ID NO: 29 - DNA, 39 bp, synthetic construct
  source 1..39: mol_type = other DNA
gcagaccacttcctttgtacaattcattcataccctcgg

SEQ ID NO: 30 - DNA, 36 bp, synthetic construct
  source 1..36: mol_type = other DNA
gctccagggggggcagaccacttcctttgtacaatt

SEQ ID NO: 31 - DNA, 35 bp, synthetic construct
  source 1..35: mol_type = other DNA
gcctgaccctgtaatctagagggcccgtttaaacc

SEQ ID NO: 32 - DNA, 33 bp, synthetic construct
  source 1..33: mol_type = other DNA
cccctggagcgcctgaccctgtaatctagaggg

SEQ ID NO: 33 - DNA, 23 bp, synthetic construct
  source 1..23: mol_type = other DNA
gtctgccccccctggagcgcctg

SEQ ID NO: 34 - DNA, 29 bp, synthetic construct
  source 1..29: mol_type = other DNA
gcgagcccctgatgctcttcgtccagatc

SEQ ID NO: 35 - DNA, 39 bp, synthetic construct; identical to SEQ ID NO: 22
  source 1..39: mol_type = other DNA

SEQ ID NO: 36 - DNA, 60 bp, synthetic construct
  source 1..60: mol_type = other DNA
tcccactacctttgtacaattcattcataccctcggtaataccagcagcagtcaaaaatt

SEQ ID NO: 37 - DNA, 55 bp, synthetic construct
  source 1..55: mol_type = other DNA
attaccgagggtatgaatgaattgtacaaaggtagtgggactgcctccggaacgg

SEQ ID NO: 38 - DNA, 29 bp, synthetic construct
  source 1..29: mol_type = other DNA
gcgagcccctgatgctcttcgtccagatc

SEQ ID NO: 39 - DNA, 29 bp, synthetic construct
  source 1..29: mol_type = other DNA
gatctggacgaagagcatcaggggctcgc

SEQ ID NO: 40 - DNA, 37 bp, synthetic construct
  source 1..37: mol_type = other DNA
catggtggcaagcttaagtttaaacgctagccagctt

SEQ ID NO: 41 - DNA, 35 bp, synthetic construct
  source 1..35: mol_type = other DNA
cttgctcttgatgcagcccatggtggcaagcttaa

SEQ ID NO: 42 - DNA, 31 bp, synthetic construct
  source 1..31: mol_type = other DNA
ttgtccttgcgcttgctcttgatgcagccca

SEQ ID NO: 43 - DNA, 36 bp, synthetic construct
  source 1..36: mol_type = other DNA
ttccaccacttccacccttgtccttgcgcttgctct

SEQ ID NO: 44 - DNA, 28 bp, synthetic construct
  source 1..28: mol_type = other DNA
caggtggtagcgtgagcaagggcgagga

SEQ ID NO: 45 - DNA, 33 bp, synthetic construct
  source 1..33: mol_type = other DNA
ggtggaagtggtggaacaggtggtagcgtgagc

SEQ ID NO: 46 - DNA, 29 bp, synthetic construct
  source 1..29: mol_type = other DNA
gcgagcccctgatgctcttcgtccagatc

SEQ ID NO: 47 - DNA, 60 bp, synthetic construct
  source 1..60: mol_type = other DNA
ggctagcgtttaaacttaagcttgccaccatggtgagcaagggcgaggaggataacatgg

SEQ ID NO: 48 - DNA, 52 bp, synthetic construct
  source 1..52: mol_type = other DNA
agagcctcctgtgccaccagagccacccttgtacagctcgtccatgccgccg

SEQ ID NO: 49 - DNA, 29 bp, synthetic construct
  source 1..29: mol_type = other DNA
gatctggacgaagagcatcaggggctcgc

SEQ ID NO: 50 - DNA, 29 bp, synthetic construct
  source 1..29: mol_type = other DNA
gatctggacgaagagcatcaggggctcgc

SEQ ID NO: 51 - DNA, 31 bp, synthetic construct
  source 1..31: mol_type = other DNA
ttgatgagtttggacaaaccacaactagaat

SEQ ID NO: 52 - DNA, 31 bp, synthetic construct
  source 1..31: mol_type = other DNA
tctagagggcccgtttaaacccgctgatcag

SEQ ID NO: 53 - DNA, 29 bp, synthetic construct
  source 1..29: mol_type = other DNA
gatctggacgaagagcatcaggggctcgc

SEQ ID NO: 54 - DNA, 37 bp, synthetic construct; identical to SEQ ID NO: 40
  source 1..37: mol_type = other DNA

(19) United States
(12) Patent Application Publication
(10) Pub. No.: US 2026/0072030 A1
(43) Pub. Date: Mar. 12, 2026

(54) RAS BIOSENSORS

(71) Applicant: The Regents of the University of California, Oakland, CA (US)

(72) Inventors: Jin Zhang, La Jolla, CA (US); Ryan Weeks, La Jolla, CA (US)

(21) Appl. No.: 19/107,453

(22) PCT Filed: Aug. 29, 2023
(86) PCT No.: PCT/US2023/073116

§ 371 Date: Feb. 27, 2025

Related U.S. Application Data
(60) Provisional application No. 63/373,843, filed on Aug. 29, 2022.

(51) Int. Cl. G01N 33/573; C07K 14/47; G01N 33/542; G01N 33/58

(52) U.S. Cl. CPC G01N 33/573; C07K 14/47; G01N 33/542; G01N 33/582; C07K 2319/60; G01N 2333/914

(57) ABSTRACT

Provided herein are, inter alia, biosensor recombinant proteins (e.g. ratiometric Ras activity reporter (RasAR)), which provide quantitative measurement of Ras activity in living cells with high spatiotemporal resolution. The biosensor recombinant proteins provided herein include, for example, a novel pseudoligand and are, inter alia, useful for detecting a Ras protein and activity of a Ras protein in a cell.

### RELATED APPLICATION DATA

This application claims the benefit of priority under 35 U.S.C. § 119(e) of the U.S. Patent Application No. 63/373,843, filed on Aug. 29, 2022, which is hereby incorporated by reference in its entirety and for all purposes.

### GOVERNMENT SUPPORT CLAUSE

This invention was made with government support under R35 CA197622 awarded by the National Institutes of Health. The government has certain rights in the invention.

### SEQUENCE LISTING

The material in the accompanying Sequence Listing is hereby incorporated by reference in its entirety. The accompanying file, named “037866-718001WO_SL_ST26.xml” was created on Aug. 28, 2023 and is 48,731 bytes in size.

### BACKGROUND

The small GTPase Ras is a critical regulator of cell growth and proliferation. Its activity is frequently dysregulated in cancers, prompting decades of work to pharmacologically target Ras. Understanding Ras biology and developing effective Ras therapeutics both require probing Ras activity in its native context, yet tools to measure its activities in cellulo are limited. The compositions and methods provided herein, inter alia, address these and other problems in the art.

### BRIEF SUMMARY

In an aspect is provided a recombinant protein including a first fluorophore, a ligand binding domain, a pseudoligand domain, and a second fluorophore.

In another aspect is provided a complex including the recombinant protein described herein including embodiments thereof non-covalently bound to a Ras protein.

In another aspect is provided an isolated nucleic acid encoding the recombinant protein described herein including embodiments thereof.

In another aspect is provided an expression vector including the isolated nucleic acid described herein including embodiments thereof.

In an aspect is provided a method for detecting a Ras protein in a cell, the method including: (a) contacting a cell with an expression vector including an isolated nucleic acid, wherein the isolated nucleic acid encodes a recombinant protein including: a first fluorophore, a ligand binding domain, a pseudoligand domain, and a second fluorophore; (b) transducing the cell with the expression vector; (c) allowing the cell to express the recombinant protein and bind to the Ras protein within the cell; and (d) detecting a fluorescent signal form the recombinant protein thereby detecting the Ras protein in the cell.

### BRIEF DESCRIPTION OF THE DRAWINGS

FIGS. 1A-1F show that RasAR provides quantitative measurement of live-cell activities of all the primary isoforms of Ras. Design (FIG. 1A) and domain structure (FIG. 1B) of RasAR, which contains the Ras binding domain (RBD) derived from Raf1 and a pseudoligand designed from the effector binding region of Ras (SEQ ID NO:1) sandwiched between the FRET pair mCerulean and YPet. Ras binding to RasAR displaces the pseudoligand and leads to a decrease in FRET between mCerulean and YPet. (FIG. 1C) Representative images of the cyan-over-yellow (C/Y) emission ratios from COS-7 cells co-expressing RasAR along with different variants of mCherry-KRas. Co-expression with KRasWT or the dominant negative S17N mutant shows lower activity and cytosolic localization in comparison to the constitutively active Q61L or G12C mutants. Scale bars, 25 μm. Images are representative of 3 independent experiments. (FIGS. 1D-1F) C/Y emission ratios of COS-7 cells expressing RasAR or RasAR(R89L) along with mCherry-labelled variants of KRas (FIG. 1D), HRas (FIG. 1E), or NRas (FIG. 1F). Data are combined from 3 independent experiments, n>800 cells in each group. For KRas (FIG. 1D), from left to right, n=1078, 1077, 952, 873, 954, 1039, 1059, 1140 cells. For HRas (FIG. 1E), from left to right, n=1158, 959, 1201, 950, 937, 957 cells. For NRas (FIG. 1F), from left to right, n=1449, 1422, 1138, 1203, 1131, 1472, 1393, 1135 cells. All groups significantly different from each other as assessed by one-way ANOVA, p<0.0001 for both RasAR and RasAR(R89L) groups. Cohen's f values show effect sizes by one-way ANOVA and are indicated above each group. All violin plots show median and quartiles.

FIGS. 2A-2C show that RasAR detects EGF-stimulated Ras activity in living cells. (FIGS. 2A-2C) Left, representative images of the C/Y emission ratios from COS-7 cells co-expressing RasAR along with mCherry tagged KRasWT, HRasWT, or NRasWT before (top) and 12.5 min after (bottom) EGF stimulation. Scale bars, 25 μm. Right, average time courses showing the C/Y emission ratio changes from RasAR (black curves) or the RasAR (R89L) negative-control mutant in cells co-expressing mCherry-RasWT and treated with 100 ng/ml EGF followed by 200 nM AG1478 as indicated. Combined results of three independent experiments, n=90 cells per group. Solid lines indicate the mean, and shaded areas represent s.e.m.

FIGS. 3A-3F demonstrate probing Src regulation of HRas using RasAR. (FIG. 3A) Domain structures of GA-RasAR and μm-RasAR. (FIG. 3B) Representative images showing localization of targeted sensors. Scale bars, 25 μm. (FIG. 3C) Average time courses of the C/Y emission ratio change from GA-RasAR (left) and violin plots of the ΔR/R0 at t=24 min (right) in COS-7 cells co-expressing mCherry-HRasWT and pretreated with the Src inhibitor PP2 (black, n=300 cells) or DMSO control (n=300 cells). Mann-Whitney pairwise test, p<0.0001. (FIG. 3D) Average time courses of the C/Y emission ratio change from μm-RasAR (left) and violin plots of the ΔR/R0 at t=5.5 min (right) in COS-7 cells co-expressing mCherry-HRasWT and pretreated with the Src inhibitor PP2 (black, n=300 cells) or DMSO control (n=300 cells). ns, not significant. (FIG. 3E) Average time courses of the C/Y emission ratio change from RasAR (left) and violin plots of the ΔR/R0 at t=5.5 min (right) in COS-7 cells co-expressing mCherry-HRasWT and pretreated with the Src inhibitor PP2 (black, n=180 cells) or DMSO control (n=180 cells). p<0.0001. (FIG. 3F) Average time courses of the C/Y emission ratio change from RasAR (left) and violin plots of the ΔR/R0 at t=5.5 min (right) in COS-7 cells co-expressing mCherry-HRasY32F/Y64F and pretreated with the Src inhibitor PP2 (black, n=180 cells) or DMSO control (n=180 cells). n=180 cells, p<0.0001. Pooled results of 5 (FIGS. 3C-3D) or 3 (FIGS. 3E-3F) independent experiments. Solid lines in FIGS. 3C-3F indicate the mean, and shaded areas represent s.e.m. Violin plots in FIGS. 3C-3F display median and quartiles, and error bars represent s.e.m.

FIGS. 4A-4G show that RasAR measures Ras inhibition in living cells. (FIG. 4A) General schematic of our Ras inhibition assay. RasAR is introduced to cells expressing mutant Ras and treated with Ras-pathway inhibitors. Spatiotemporal differences in the activity of Ras as well as the entire signaling pathway caused by inhibitor treatment can be visualized in living cells to provide information about target engagement. (FIG. 4B) C/Y emission ratios of COS-7 cells co-expressing mCherry-KRasG12C or KRasWT plus RasAR treated with either ARS1620 (10 μM) or DMSO for 24 hr. (FIG. 4C) C/Y emission ratios of COS-7 cells co-expressing mCherry-KRasG12C and RasAR-treated with the indicated concentrations of ARS1620 for 24 hr. (FIG. 4D) Y/C emission ratios of COS-7 cells co-expressed mCherry-KRasG12C and EKAR treated with ARS1620 (10 μM) for 3 hr. EGF control included to verify EKAR response. (FIGS. 4E-4G) C/Y emission ratios recorded from COS-7 cells co-expressing RasAR and mCherry-KRasG12C after incubation with (FIG. 4E) ARS1620 (10 μM), (FIG. 4F) Sotorasib (10 μM), or (FIG. 4G) Adagrasib (10 μM) for the indicated times. Each data set is pooled from 3 independent experiments with n>800 cells per treatment group. For (FIG. 4B), from left to right, n=1082, 1116, 1038, 1153 cells. For (FIG. 4C), from left to right, n=1156, 1037, 1076, 1029, 962, 866 cells. For (FIG. 4D), from left to right, n=4668, 3902, 4056, 3162, 2033, 2454 cells. For (FIG. 4E), from left to right, n=2170, 2094, 2226, 2172, 2203, 2833 cells. For (FIG. 4F), from left to right, n=2447, 2610, 2398, 2777, 2851, 4287 cells. For (FIG. 4G), from left to right, n=1932, 2055, 1986, 1770, 1952, 2161 cells. Violin plots show median and quartiles. Cohen's d-statistic from pairwise t-test displayed for (FIG. 4B) and (FIG. 4D). For (FIG. 4C) and (FIGS. 4E-4F), one way ANOVA is performed with Cohen's f-statistic displayed. All groups significantly different from each other by one-way ANOVA (FIGS. 4C, 4E-4G), or by t-test (FIGS. 4B and 4D), p<0.0001.

FIGS. 5A-5F show the design of RasAR and its emission ratios across different expression levels. (FIG. 5A) Crystal structure PDB: 4GON, Ras bound to the RBD of Raf1. The residues chosen for the pseudoligand of RasAR (SEQ ID NO:13) are underlined (SEQ ID NO:1). Dotted lines indicate where the N- and C-termini of the respective protein domains attach to either fluorescent proteins or connecting flexible linker (FIG. 5B-5F) RasAR C/Y emission ratio values are plotted against RFP intensity values for each variant of mCherry-Ras. n-numbers identical to the corresponding groups of FIGS. 1C-1F.

FIGS. 6A-6C shows a colocalization analysis of RasAR and Ras. (FIGS. 6A-6C) Colocalization of RasAR with mCherry-KRas (FIG. 6A), HRas (FIG. 6B), and NRas (FIG. 6C) in representative cells, quantified by Pearson correlation coefficient. Cells expressing CA mutants of Ras display high colocalization. Comparison of Pearson correlation coefficients (n=30). ****, p<0.0001, by Mann-Whitney test. Scale bars, 25 μm.

FIGS. 7A-7H show further analysis of acutely stimulated Ras activity. (FIG. 7A) Kinetic comparison of C/Y emission ratio changes obtained from ROIs drawn selectively around the Golgi apparatus (GA) or in the cytosolic Golgi-excluded regions in COS-7 cells expressing HRasWT and RasAR (n=180 cells). Mann-Whitney pairwise test performed; ***, p<0.001. (FIG. 7B) Comparison of time to half-maximal response (t½) for RasAR response in COS-7 cells co-expressing KRas, HRas, or NRas (n=180 cells each). Mann-Whitney pairwise test performed; **, p<0.01; *, p<0.05. (FIG. 7C) RFP intensity of COS-7 cells expressing mCherry labeled wildtype Ras isoforms. RasAR-NES or RasAR-NES-R89L is co-expressed with mCherry-HRasWT in (FIG. 7D) HEK293T or (FIG. 7E) HeLa cells and stimulated with EGF (100 ng/ml). Combined results of 3 independent experiments, (n=180 cells). (FIG. 7F) Ras-RAICHU-EV expressed in COS-7 cells and stimulated with EGF (100 ng/ml). Combined results of 3 independent experiments. (n=65 cells). (FIG. 7G) Comparison of time to half-maximal response (t½) of Ras-RAICHU-EV vs. RasAR-NES co-expressed with mCherry-KRasWT. Mann-Whitney pairwise test performed; ****, p<0.0001. (FIG. 7H) Active Ras (Ras-GTP) pulldown assays in serum-starved COS-7 cells treated with EGF (100 ng/ml) (left) and quantification of active GTP-bound Ras normalized to total Ras (right). Data are representative of three independent experiments. One-way ANOVA followed by Dunnett's statistical test for (FIG. 7H), ****, p<0.0001

FIG. 8 shows Ras activity at the Golgi. COS-7 cells were co-transfected with RasAR. mScarlet-Giantin as a Golgi marker, and the indicated unlabeled Ras isoforms and stimulated with EGF. Merged images show the colocalization of RasAR and mScarlet-Giantin. Representative images (far right) show EGF-simulated Ras activity concentrated at the Golgi. Scale bars, 25 μm.

FIGS. 9A-9G show validation of targeted biosensors and Src inhibition experiments. (FIG. 9A) GA-RasAR and mScarlet-Giantin are co-expressed and merged to demonstrate colocalization. (FIG. 9B) Line scan shows the plasma membrane localization of μm-RasAR. (FIGS. 9C-9D) COS-7 cells co-expressing mCherry-HRasWT and either GA-RasAR (FIG. 9C) or μm-RasAR (FIG. 9D) were treated with 100 ng/ml EGF and then (10 μM) AG1478, compared with negative controls. Each experimental group contains n=60 cells, from 2 independent experiments. (FIG. 9E) t½ comparison for GA-RasAR and pm-RasAR. ****, p<0.0001; Mann-Whitney pairwise comparison. (FIG. 9F) Average time courses of normalized emission ratio (left) and violin plot showing the C/Y emission ratio at t=5.5 min (right) in COS-7 cells co-expressing RasAR with mCherry-HRasWT and pretreated with (1 μM) SrcI1 (grey curve, n=180) or DMSO control (n=180) and stimulated with 100 ng/ml EGF. ****, p<0.0001; Mann-Whitney pairwise comparison. (FIG. 9G) Average time courses of normalized emission ratio (left) and violin plot showing the C/Y emission ratio at t=5.5 min (right) in COS-7 cells co-expressing RasAR with mCherry-HRasY32F pretreated with PP2 (1 μM) grey curve, n 5=180) or DMSO (=180) were stimulated with 100 ng/ml EGF. **, p<0.01; Mann-Whitney pairwise comparison. Data are pooled from and representative of 3 independent experiments. Solid lines in FIGS. 9C, 9D, 9F, and 9G indicate the mean, and shaded areas represent s.e.m. Violin plots in FIGS. 9E-9G display median and quartiles, and error bars represent s.e.m.

FIGS. 10A-10I show measurements of Ras inhibition in living cells. (FIG. 10A) C/Y emission

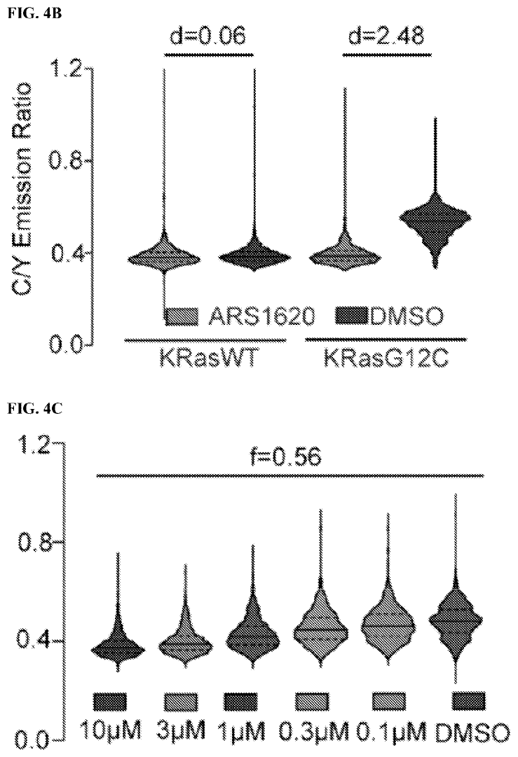

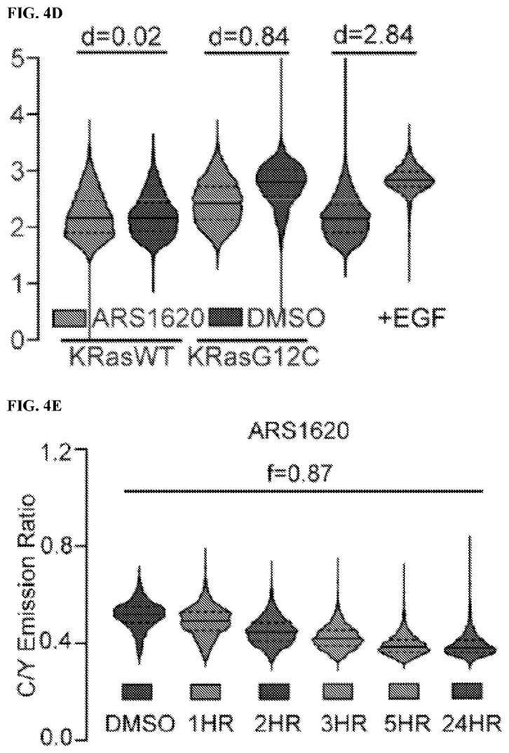

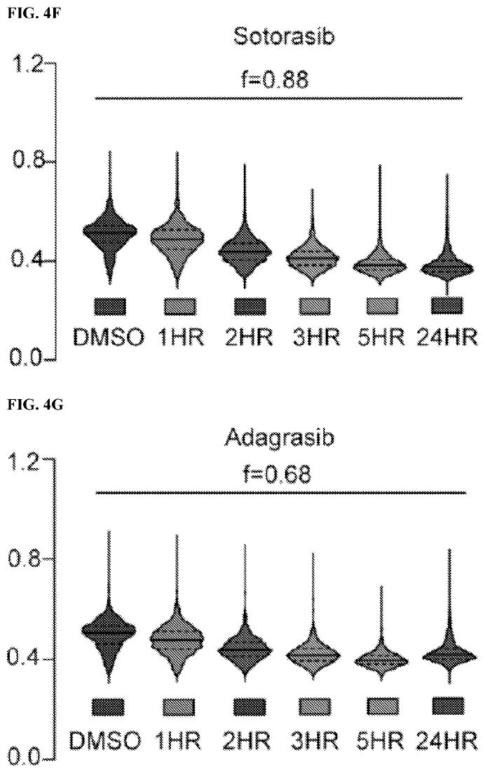

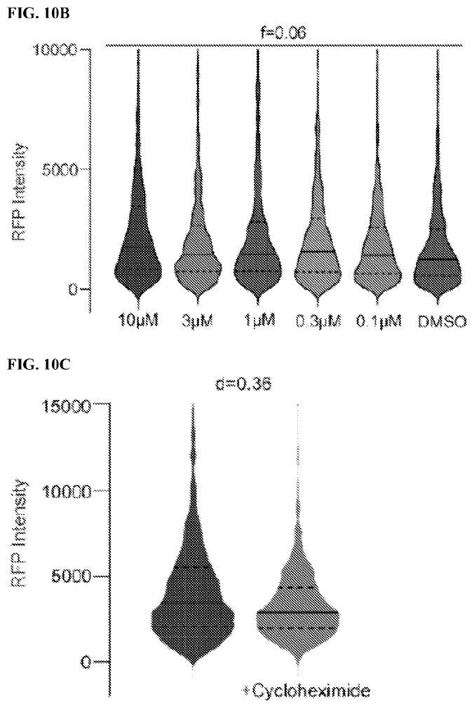

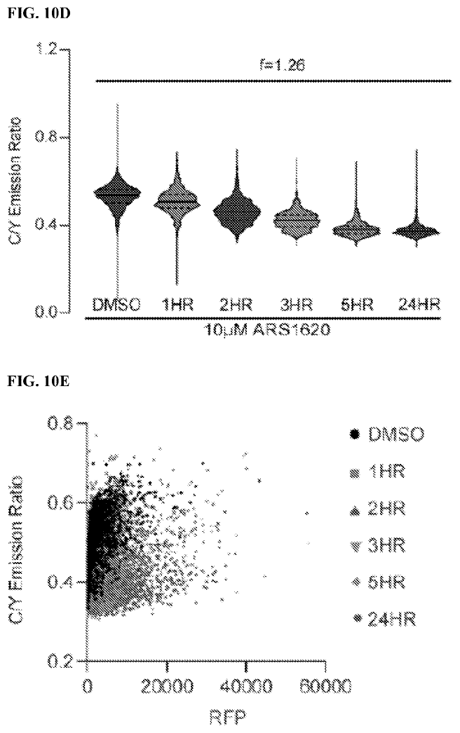

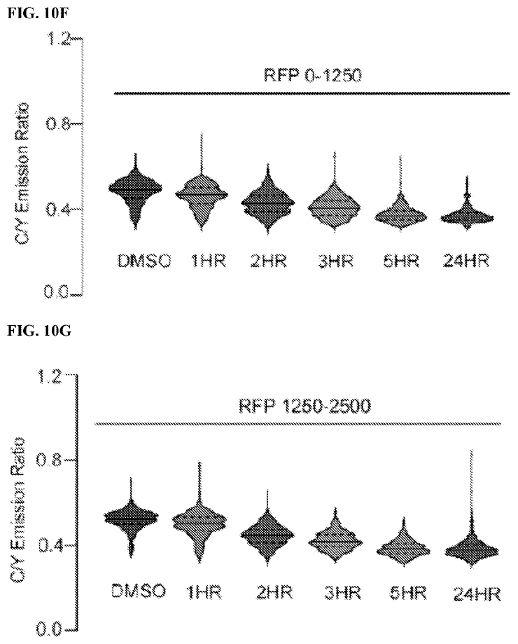

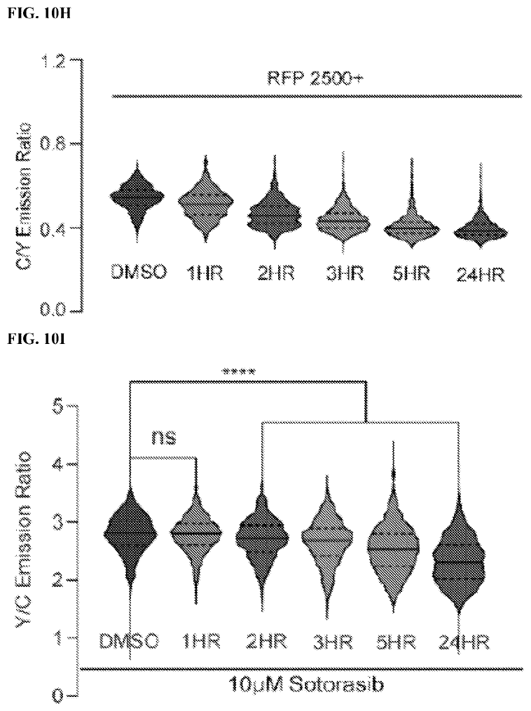

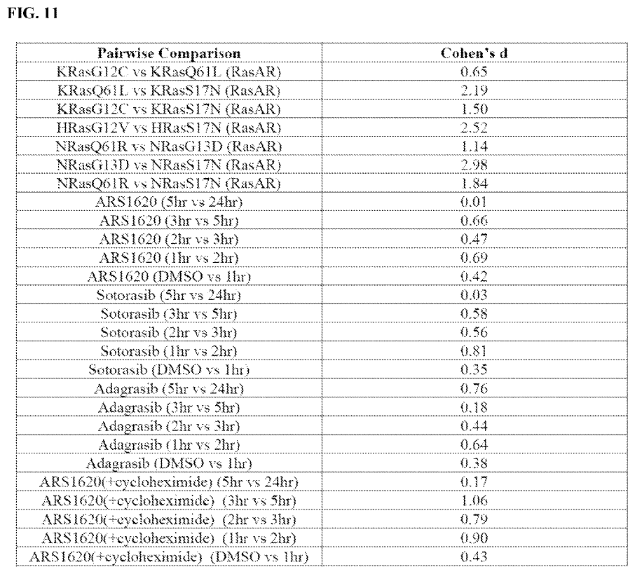

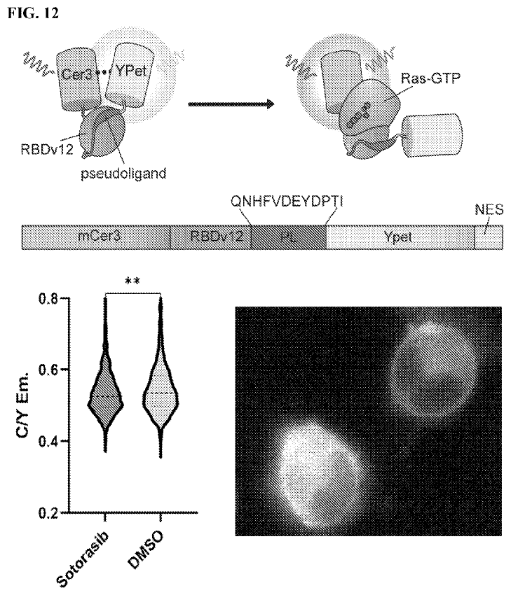

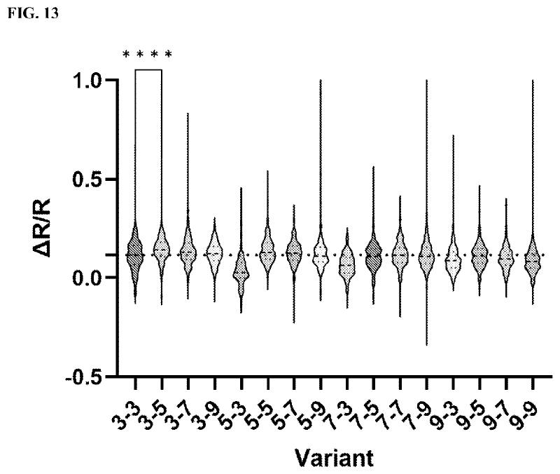

ratio versus RFP intensity for ARS1620 inhibition experiment in FIG. 4b. (FIG. 10B) RFP intensities from the ARS1620 titration experiment show little variation in Ras expression across treatment groups. Cohen's f=0.06. (FIG. 10C) RFP intensities from COS-7 cells co-expressing mCherry-KRasG12C and RasAR subjected to ARS1620 (10 μM) treatment with and without cycloheximide (100 μg/ml) treatment. Cohen's d=0.36. (FIG. 10D) Effect of cycloheximide treatment (24 hr) on mCherry-KRasG12C expression evaluated by RFP intensity. Cohen's f=1.26. From left to right, n=734, 896, 761, 813, 866, 632 cells. (FIG. 10E) C/Y emission ratio versus RFP intensity for the ARS1620 kinetics experiment from FIG. 4E. (FIGS. 10F-10H) Cells at each time point were binned into (FIG. 10F) low (0-1250), (FIG. 10G) medium (1250-2500), and (FIG. 10H) high 2500+ expression groups and replotted as shown. All groups significantly different by one-way ANOVA, p<0.0001. (FIG. 10I) COS-7 cells co-expressing EKAR-NES and KRasG12C were treated with Sotorasib (10 μM) for 24 hr. DMSO-treated vs 1-hr treatment shows no significant difference, but DMSO vs 2 hr-24 hr time points produce significant differences by Mann-Whitney pairwise test (p<0.0001).

FIG. 11 shows the Cohen's d values for pairwise comparison of experiments described in Example 1.

FIG. 12 shows an exemplary RasAR biosensor described herein and use thereof (Top) Mechanism and domain structure of RasARv12 including a pseudoligand domain (SEQ ID NO:1). (Bottom-left) C/Y emission ratio values of MIA PaCa-2 treated with Sotorasib or DMSO control. (Bottom-Right) CFP channel of RasARv12 showing localization of the sensor at the plasma membrane.

FIG. 13 shows experimental data from a screen of RasARv12 linker variants. ΔR/R is defined by HRasG12V normalized to median of HRasDN C/Y Emission values. X-X nomenclature refers to linker length between mCer3-RBDv12 (left) or PL-Ypet (right).

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., Ras) in other proteins with different numbering systems. For example, by performing a simple sequence alignment with a protein (e.g., Ras) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of: e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “specifically (or selectively) binds to” when referring to a protein or peptide, refers to a binding reaction that is determinative of the presence of the protein, often in a heterogeneous population of proteins and other biologics. Thus, under designated immunoassay conditions, the specified proteins bind to a particular protein at least two times the background and more typically more than 10 to 100 times background.

A “ligand” refers to an agent, e.g., a polypeptide or other molecule, capable of binding to a specific protein or fragment thereof.

The term “ligand binding domain” is used herein according to its plain ordinary meaning and refers to a polypeptide which binds a ligand. In embodiments, the ligand binding domain is a Ras binding domain (RBD). In embodiments, the RBD is derived from a Ras effector. In embodiments, the Ras effector is C-Raf. In embodiments, the RBD binds a Ras protein with a higher affinity than a Rap protein. In embodiments, the RBD comprises a charged lysine at a position corresponding to position 84 of RBD. In embodiments, the RBD is RBDwt, RBDv12, RBDv1, RBDv2, or RBDv13. In embodiments, the the ligand binding domain includes the amino acid sequence of SEQ ID NO: 17, SEQ ID NO:18, SEQ ID NO:19, SEQ ID NO:20, or SEQ ID NO:21.

The term “pseudoligand domain” is used herein according to its plain ordinary meaning and refers to a compound including a polypeptide which binds a ligand binding domain. In embodiments, the pseudoligand binds a Ras binding domain (RBD). In embodiments, the pseudoligand domain includes the amino acid sequence of SEQ ID NO: 1. In embodiments, the pseudoligand domain includes the amino acid sequence of SEQ ID NO:2. In embodiments, the pseudoligand domain includes the amino acid sequence of SEQ ID NO:3. In embodiments, the pseudoligand domain includes the amino acid sequence of SEQ ID NO:4 In embodiments, the pseudoligand domain includes the amino acid sequence of SEQ ID NO:5. In embodiments, the pseudoligand domain includes the amino acid sequence of SEQ ID NO:6. In embodiments, the pseudoligand domain includes the amino acid sequence of SEQ ID NO: 11.

The terms “FRET pair” and “FRET fluorophore pair” are used herein according to their plain ordinary meaning and refer a pair of light-sensitive chemical compounds that are capable of transferring energy through nonradiative dipole coupling with one member being a donor and the other member being an acceptor. In embodiments, the pair of light-sensitive chemical compounds includes a first light-sensitive chemical compound and a second light-sensitive chemical compound. In embodiments, the pair of light-sensitive chemical compounds are capable of emitting light upon light excitation. In embodiments, the pair of light-sensitive chemical compounds absorb light energy of a specific wavelength and emit the light at a specific longer wavelength. In embodiments, the pair of light-sensitive chemical compounds are a pair of fluorescent chemical compounds. In embodiments, the pair of fluorescent chemical compounds are a pair of fluorophores. In embodiments, upon excitation by light, the first fluorophore transfers energy to the second fluorophore. In embodiments, this energy transfer between the pair of fluorophores is Forster resonance energy transfer (FRET). In embodiments, the first fluorophore is a donor fluorophore. In embodiments, the second fluorophore is an acceptor fluorophore. In embodiments, the FRET occurs in when the pair of fluorophores are in close proximity. In embodiments, the FRET occurs when radius between the pair of fluorophores is smaller than the wavelength of light emitted. For the compositions and methods provided herein including embodiments thereof any one of the FRET fluorophore pairs described in Bajar et al., Sensors (Basel), 2016; 16(9):1488 which is incorporated herein by reference in its entirety and for all purposes, may be used. In embodiments, the FRET pair is a cyan-yellow or a red-green FRET pair. In embodiments, the FRET pair is a cyan-yellow FRET pair. In embodiments, the FRET pair is a red-green FRET pair. In embodiments, the FRET pair includes a mCerulean3 fluorophore and a YPet fluorophore. In embodiments, the first fluorophore or the second fluorophore is a mCerulean3 fluorophore or a YPet fluorophore.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The terms “Ras protein” and “Ras” as used herein include any of the recombinant or naturally-occurring forms of the Rat sarcoma virus, also known as Ras GTPase, or variants or homologs thereof that maintain Ras activity (e.g. within at least 50%, 80%, 90%, 95%, 96%, 97%, 98%, 99% or 100% activity compared to Ras). In some aspects, the variants or homologs have at least 90%, 95%, 96%, 97%, 98%, 99% or 100% amino acid sequence identity across the whole sequence or a portion of the sequence (e.g. a 25, 50, 100, 150 or continuous amino acid portion) compared to a naturally occurring Ras protein. In embodiments, the Ras protein is substantially identical to the protein identified by the UniProt reference number P01116 or a variant or homolog having substantial identity thereto. In embodiments, the Ras protein includes the amino acid sequence of SEQ ID NO:12. In embodiments, the Ras protein includes the amino acid sequence of SEQ ID NO: 13.

The term “GTPase” is used herein according to its plain ordinary meaning and refers to a hydrolase enzyme that catalyzes a hydrolysis reaction. In embodiments, the GTPase binds the nucleotide guanosine triphosphate (GTP) and hydrolyzes it to guanosine diphosphate (GDP).

The terms “nuclear export signal” and “NES” are used herein according to their plain ordinary meaning and refer to a amino acid sequence which can be attached to a polypeptide. In embodiments, the NES promotes cytosolic localization of the polypeptide. In embodiments, the NES includes the amino acid sequence of SEQ ID NO:14.

The term “Giantin tag” is used herein according to its plain ordinary meaning and refers to an amino acid sequence which can be attached to a polypeptide. In embodiments, the Giantin tag promotes localization of the polypeptide to the Golgi apparatus. In embodiments, the Giantin tag promotes Golgi apparatus localization through C-terminal fusion with a Giantin fragment. In embodiments, the Giantin tag includes the amino acid sequence of SEQ ID NO:15

The term “Lyn-targeting motif” is used herein according to its plain ordinary meaning and refers to an amino acid sequence which can be attached to a polypeptide. In embodiments, the Lyn-targeting motif promotes localization of the polypeptide to the plasma membrane. In embodiments, the Lyn-targeting motif includes a N-terminal tag from a Lyn kinase. In embodiments, the Lyn-targeting motif includes the amino acid sequence of SEQ ID NO: 16.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes, regulatory elements necessary for the expression of genes, proteins, and/or recombinant proteins (e.g. RasAR). Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide specifically reactive with a target peptide. Any appropriate method known in the art for conjugating a peptide to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated, however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to a protein-inhibitor interaction means negatively affecting (e.g. decreasing) the activity or function of the protein (e.g. Ras protein) relative to the activity or function of the protein in the absence of the inhibitor. In embodiments inhibition means negatively affecting (e.g. decreasing) the concentration or levels of Ras relative to the concentration or level of the protein in the absence of the inhibitor. In embodiments inhibition refers to reduction of a disease or symptoms of disease. In embodiments, inhibition refers to a reduction in the activity of Ras. Thus, inhibition includes, at least in part, partially or totally blocking stimulation, decreasing, preventing, or delaying activation, or inactivating, desensitizing, or down-regulating signal transduction or enzymatic activity or the amount of Ras. In embodiments, inhibition refers to a reduction of activity of Ras resulting from a direct interaction (e.g. an inhibitor binds to Ras). In embodiments, inhibition refers to a reduction of activity of Ras from an indirect interaction (e.g. an inhibitor binds to a protein that activates Ras, thereby preventing target protein activation).

Thus, the terms “inhibitor,” “repressor” or “antagonist” or “downregulator” interchangeably refer to a substance capable of detectably decreasing the expression or activity of a given gene or protein (e.g. Ras protein). The antagonist can decrease Ras expression or activity 10%, 20%, 30%, 40%, 50%, 60%, 70%, 80%, 90% or more in comparison to a control in the absence of the antagonist. In certain instances, Ras expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “Ras-ERK signaling pathway” or “Ras-ERK pathway” are used herein according to their plain ordinary meaning and refers to a series of interactions between cellular and optionally extracellular components that communicates a signal from a receptor on the surface of a cell to the DNA in the nucleus of the cell. The Ras-Extracellular signal-regulated kinase (ERK) pathway is also known as the Ras-mitogen-activated protein kinase (MAPK) pathway. In embodiments, the Ras-ERK pathway regulates cell cycle entry and cell proliferation.

In embodiments, the Ras-ERK pathway comprises a Ras protein. In embodiments, the Ras-ERK pathway includes a signaling receptor-associated protein. In embodiments the signaling receptor-associated protein is a receptor-linked tyrosine kinase. In embodiments, the Ras-ERK pathway includes a docking protein. In embodiments, the docking protein is GRB2. In embodiments, the GRB2 includes an SH2 domain. In embodiments, the docking protein binds a phosphorylated receptor-linked tyrosine kinase. In embodiments, the Ras-ERK pathway includes a guanine nucleotide exchange factor (GEF). In embodiments, the GEF is a Son of Sevenless (SOS) protein. In embodiments, the GEF binds the docking protein through the SH2 domain. In embodiments, the Ras-ERK pathway includes a GTPase. In embodiments, the GTPase is inactive. In embodiments, the inactive GTPase is bound to guanosine diphosphate (GDP). In embodiments, the receptor-linked tyrosine kinase-docking protein-GEF complex activates the GTPase. In embodiments, the GTPase is activated by exchanging the bound GDP for a guanosine triphosphate (GTP). In embodiments, the GTPase is a Ras protein.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

The terms “1-ethyl-3-(-3-dimethyoaminopropyl) carbodiimide”, “EDC”, “EDAC” and “EDCI” as used herein refer to a water-soluble carbodiimide usually handled as the hydrochloride. It is generally used as a carboxyl activating agent for the coupling of primary amines to yield amide bonds. Additionally, EDC can also be used to activate phosphate groups in order to form phosphomonoesters and phosphodiesters. Common uses for this carbodiimide include peptide synthesis, protein crosslinking to nucleic acids, but also in the preparation of immunoconjugates. EDC is often used in combination with N-hydroxysuccinimide (NHS) for the immobilisation of large biomolecules. Recent work has also used EDC to assess the structure state of uracil nucleobases in RNA.

The terms “N-hydroxysulfosuccinimide”, “sulfo-NHS” and “NHS” refer to a compound that enables control and modification of carbodiimide crosslinking reactions involving activation of carboxylates (—COOH) for conjugation with primary amines (—NH₂). Derivatives can be synthesized by mixing the NHS with a carboxyl-containing molecule and a dehydrating agent such as the carbodiimide EDC. The method is the basis for generating many types of protein labeling reagents, including amine-reactive fluorescent dyes, biotin affinity tags and pegylation compounds.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Biosensor Recombinant Proteins

Provided herein are, inter alia, biosensor recombinant proteins (e.g., ratiometric Ras activity reporter (RasAR)), which provide quantitative measurement of Ras activity in living cells with high spatiotemporal resolution. The biosensor recombinant proteins provided herein include, for example, a novel pseudoligand and are, inter alia, useful for detecting a Ras protein and activity of a Ras protein in a cell. Thus, in an aspect is provided recombinant protein including a first fluorophore, a ligand binding domain, a pseudoligand domain, and a second fluorophore.

In embodiments, the first fluorophore is bound to the ligand binding domain through a first peptidyl linker, the pseudoligand domain is bound to the ligand binding domain through a second peptidyl linker, and the second fluorophore is bound to the pseudoligand domain through a third peptidyl linker.

In embodiments, the first fluorophore and the second fluorophore are independently Förster Resonance Energy Transfer (FRET) fluorophores. In embodiments, the first fluorophore or the second fluorophore is a FRET fluorophore. In embodiments, the first fluorophore and the second fluorophore are FRET fluorophores. In embodiments, the first fluorophore is a FRET fluorophore. In embodiments, the second fluorophore is a FRET fluorophore.

In embodiments, the first FRET fluorophore or the second FRET fluorophore is a mCerulean3 fluorophore or a YPet fluorophore. In embodiments, the first FRET fluorophore is a mCerulean3 fluorophore or a YPet fluorophore. In embodiments, the first FRET fluorophore is a mCerulean3 fluorophore. In embodiments, the first FRET fluorophore is a YPet fluorophore. In embodiments, the second FRET fluorophore is a mCerulean3 fluorophore or a YPet fluorophore. In embodiments, the second FRET fluorophore is a mCerulean3 fluorophore. In embodiments, the second FRET fluorophore is a YPet fluorophore.

In embodiments, the pseudoligand domain includes the amino acid sequence of SEQ ID NO:1, SEQ ID NO:2, SEQ ID NO:3, SEQ ID NO:4, SEQ ID NO:5, or SEQ ID NO:6. In embodiments, the pseudoligand domain includes the amino acid sequence of SEQ ID NO: 1. In embodiments, the pseudoligand domain includes the amino acid sequence of SEQ ID NO:2. In embodiments, the pseudoligand domain includes the amino acid sequence of SEQ ID NO:3. In embodiments, the pseudoligand domain includes the amino acid sequence of SEQ ID NO:4 In embodiments, the pseudoligand domain includes the amino acid sequence of SEQ ID NO:5. In embodiments, the pseudoligand domain includes the amino acid sequence of SEQ ID NO:6 In embodiments, the pseudoligand domain is the amino acid sequence of SEQ ID NO: 1, SEQ ID NO:2, SEQ ID NO:3, SEQ ID NO:4, SEQ ID NO:5, or SEQ ID NO:6. In embodiments, the pseudoligand domain is the amino acid sequence of SEQ ID NO: 1. In embodiments, the pseudoligand domain is the amino acid sequence of SEQ ID NO:2. In embodiments, the pseudoligand domain is the amino acid sequence of SEQ ID NO: 3. In embodiments, the pseudoligand domain is the amino acid sequence of SEQ ID NO: 4. In embodiments, the pseudoligand domain is the amino acid sequence of SEQ ID NO: 5. In embodiments, the pseudoligand domain is the amino acid sequence of SEQ ID NO: 6.

In embodiments, the pseudoligand domain includes a sequence having 70%, 75%, 80%, 85%, 90%, 95%, 96%, 97%, 98%, 99% or 100% sequence identity to 5, 6, 7, 8, 9, 10, 11, or 12 contiguous amino acids of the sequence of SEQ ID NO:1. In embodiments, the pseudoligand domain includes a sequence having 70% sequence identity to 5, 6, 7, 8, 9, 10, 11, or 12 contiguous amino acids of the sequence of SEQ ID NO:1. In embodiments, the pseudoligand domain includes a sequence having 75% sequence identity to 5, 6, 7, 8, 9, 10, 11, or 12 contiguous amino acids of the sequence of SEQ ID NO:1. In embodiments, the pseudoligand domain includes a sequence having 80% sequence identity to 5, 6, 7, 8, 9, 10, 11, or 12 contiguous amino acids of the sequence of SEQ ID NO:1. In embodiments, the pseudoligand domain includes a sequence having 85% sequence identity to 5, 6, 7, 8, 9, 10, 11, or 12 contiguous amino acids of the sequence of SEQ ID NO:1. In embodiments, the pseudoligand domain includes a sequence having 90% sequence identity to 5, 6, 7, 8, 9, 10, 11, or 12 contiguous amino acids of the sequence of SEQ ID NO:1. In embodiments, the pseudoligand domain includes a sequence having 95% sequence identity to 5, 6, 7, 8, 9, 10, 11, or 12 contiguous amino acids of the sequence of SEQ ID NO:1. In embodiments, the pseudoligand domain includes a sequence having 96% sequence identity to 5, 6, 7, 8, 9, 10, 11, or 12 contiguous amino acids of the sequence of SEQ ID NO:1. In embodiments, the pseudoligand domain includes a sequence having 97% sequence identity to 5, 6, 7, 8, 9, 10, 11, or 12 contiguous amino acids of the sequence of SEQ ID NO:1. In embodiments, the pseudoligand domain includes a sequence having 98% sequence identity to 5, 6, 7, 8, 9, 10, 11, or 12 contiguous amino acids of the sequence of SEQ ID NO:1. In embodiments, the pseudoligand domain includes a sequence having 99% sequence identity to 5, 6, 7, 8, 9, 10, 11, or 12 contiguous amino acids of the sequence of SEQ ID NO:1. In embodiments, the pseudoligand domain includes a sequence having 100% sequence identity to 5, 6, 7, 8, 9, 10, 11, or 12 contiguous amino acids of the sequence of SEQ ID NO:1.

In embodiments, the pseudoligand domain includes a sequence having 70%, 75%, 80%, 85%, 90%, 95%, 96%, 97%, 98%, 99% or 100% sequence identity to 5, 6, 7, 8, 9, or 10 contiguous amino acids of the sequence of SEQ ID NO:2. In embodiments, the pseudoligand domain includes a sequence having 70% sequence identity to 5, 6, 7, 8, 9, or 10 contiguous amino acids of the sequence of SEQ ID NO:2. In embodiments, the pseudoligand domain includes a sequence having 75% sequence identity to 5, 6, 7, 8, 9, or 10 contiguous amino acids of the sequence of SEQ ID NO:2. In embodiments, the pseudoligand domain includes a sequence having 80% sequence identity to 5, 6, 7, 8, 9, or 10 contiguous amino acids of the sequence of SEQ ID NO:2. In embodiments, the pseudoligand domain includes a sequence having 85% sequence identity to 5, 6, 7, 8, 9, or 10 contiguous amino acids of the sequence of SEQ ID NO:2. In embodiments, the pseudoligand domain includes a sequence having 90% sequence identity to 5, 6, 7, 8, 9, or 10 contiguous amino acids of the sequence of SEQ ID NO:2. In embodiments, the pseudoligand domain includes a sequence having 95% sequence identity to 5, 6, 7, 8, 9, or 10 contiguous amino acids of the sequence of SEQ ID NO:2. In embodiments, the pseudoligand domain includes a sequence having 96% sequence identity to 5, 6, 7, 8, 9, or 10 contiguous amino acids of the sequence of SEQ ID NO:2. In embodiments, the pseudoligand domain includes a sequence having 97% sequence identity to 5, 6, 7, 8, 9, or 10 contiguous amino acids of the sequence of SEQ ID NO:2. In embodiments, the pseudoligand domain includes a sequence having 98% sequence identity to 5, 6, 7, 8, 9, or 10 contiguous amino acids of the sequence of SEQ ID NO:2. In embodiments, the pseudoligand domain includes a sequence having 99% sequence identity to 5, 6, 7, 8, 9, or 10 contiguous amino acids of the sequence of SEQ ID NO:2. In embodiments, the pseudoligand domain includes a sequence having 100% sequence identity to 5, 6, 7, 8, 9, or 10 contiguous amino acids of the sequence of SEQ ID NO:2.

In embodiments, the pseudoligand domain includes a sequence having 70%, 75%, 80%, 85%, 90%, 95%, 96%, 97%, 98%, 99% or 100% sequence identity to 5, 6, 7, 8, 9, 10, 11, 12, or 13 contiguous amino acids of the sequence of SEQ ID NO:3. In embodiments, the pseudoligand domain includes a sequence having 70% sequence identity to 5, 6, 7, 8, 9, 10, 11, 12, or 13 contiguous amino acids of the sequence of SEQ ID NO:3. In embodiments, the pseudoligand domain includes a sequence having 75% sequence identity to 5, 6, 7, 8, 9, 10, 11, 12, or 13 contiguous amino acids of the sequence of SEQ ID NO:3. In embodiments, the pseudoligand domain includes a sequence having 80% sequence identity to 5, 6, 7, 8, 9, 10, 11, 12, or 13 contiguous amino acids of the sequence of SEQ ID NO:3. In embodiments, the pseudoligand domain includes a sequence having 85% sequence identity to 5, 6, 7, 8, 9, 10, 11, 12, or 13 contiguous amino acids of the sequence of SEQ ID NO:3. In embodiments, the pseudoligand domain includes a sequence having 90% sequence identity to 5, 6, 7, 8, 9, 10, 11, 12, or 13 contiguous amino acids of the sequence of SEQ ID NO:3. In embodiments, the pseudoligand domain includes a sequence having 95% sequence identity to 5, 6, 7, 8, 9, 10, 11, 12, or 13 contiguous amino acids of the sequence of SEQ ID NO:3. In embodiments, the pseudoligand domain includes a sequence having 96% sequence identity to 5, 6, 7, 8, 9, 10, 11, 12, or 13 contiguous amino acids of the sequence of SEQ ID NO:3. In embodiments, the pseudoligand domain includes a sequence having 97% sequence identity to 5, 6, 7, 8, 9, 10, 11, 12, or 13 contiguous amino acids of the sequence of SEQ ID NO:3. In embodiments, the pseudoligand domain includes a sequence having 98% sequence identity to 5, 6, 7, 8, 9, 10, 11, 12, or 13 contiguous amino acids of the sequence of SEQ ID NO:3. In embodiments, the pseudoligand domain includes a sequence having 99% sequence identity to 5, 6, 7, 8, 9, 10, 11, 12, or 13 contiguous amino acids of the sequence of SEQ ID NO:3. In embodiments, the pseudoligand domain includes a sequence having 100% sequence identity to 5, 6, 7, 8, 9, 10, 11, 12, or 13 contiguous amino acids of the sequence of SEQ ID NO:3.

In embodiments, the pseudoligand domain includes a sequence having 70%, 75%, 80%, 85%, 90%, 95%, 96%, 97%, 98%, 99% or 100% sequence identity to 5, 6, 7, 8, or 9 contiguous amino acids of the sequence of SEQ ID NO:4. In embodiments, the pseudoligand domain includes a sequence having 70% sequence identity to 5, 6, 7, 8, or 9 contiguous amino acids of the sequence of SEQ ID NO:4. In embodiments, the pseudoligand domain includes a sequence having 75% sequence identity to 5, 6, 7, 8, or 9 contiguous amino acids of the sequence of SEQ ID NO:4. In embodiments, the pseudoligand domain includes a sequence having 80% sequence identity to 5, 6, 7, 8, or 9 contiguous amino acids of the sequence of SEQ ID NO:4. In embodiments, the pseudoligand domain includes a sequence having 85% sequence identity to 5, 6, 7, 8, or 9 contiguous amino acids of the sequence of SEQ ID NO:4. In embodiments, the pseudoligand domain includes a sequence having 90% sequence identity to 5, 6, 7, 8, or 9 contiguous amino acids of the sequence of SEQ ID NO:4. In embodiments, the pseudoligand domain includes a sequence having 95% sequence identity to 5, 6, 7, 8, or 9 contiguous amino acids of the sequence of SEQ ID NO:4. In embodiments, the pseudoligand domain includes a sequence having 96% sequence identity to 5, 6, 7, 8, or 9 contiguous amino acids of the sequence of SEQ ID NO:4. In embodiments, the pseudoligand domain includes a sequence having 97% sequence identity to 5, 6, 7, 8, or 9 contiguous amino acids of the sequence of SEQ ID NO:4. In embodiments, the pseudoligand domain includes a sequence having 98% sequence identity to 5, 6, 7, 8, or 9 contiguous amino acids of the sequence of SEQ ID NO:4. In embodiments, the pseudoligand domain includes a sequence having 99% sequence identity to 5, 6, 7, 8, or 9 contiguous amino acids of the sequence of SEQ ID NO:4. In embodiments, the pseudoligand domain includes a sequence having 100% sequence identity to 5, 6, 7, 8, or 9 contiguous amino acids of the sequence of SEQ ID NO:4.

In embodiments, the pseudoligand domain includes a sequence having 70%, 75%, 80%, 85%, 90%, 95%, 96%, 97%, 98%, 99% or 100% sequence identity to 5, 10, 11, 12, 13, 14, 15, or 16 contiguous amino acids of the sequence of SEQ ID NO:5. In embodiments, the pseudoligand domain includes a sequence having 70% sequence identity to 5, 10, 11, 12, 13, 14, 15, or 16 contiguous amino acids of the sequence of SEQ ID NO:5. In embodiments, the pseudoligand domain includes a sequence having 75% sequence identity to 5, 10, 11, 12, 13, 14, 15, or 16 contiguous amino acids of the sequence of SEQ ID NO:5. In embodiments, the pseudoligand domain includes a sequence having 80% sequence identity to 5, 10, 11, 12, 13, 14, 15, or 16 contiguous amino acids of the sequence of SEQ ID NO:5. In embodiments, the pseudoligand domain includes a sequence having 85% sequence identity to 5, 10, 11, 12, 13, 14, 15, or 16 contiguous amino acids of the sequence of SEQ ID NO:5. In embodiments, the pseudoligand domain includes a sequence having 90% sequence identity to 5, 10, 11, 12, 13, 14, 15, or 16 contiguous amino acids of the sequence of SEQ ID NO:5. In embodiments, the pseudoligand domain includes a sequence having 95% sequence identity to 5, 10, 11, 12, 13, 14, 15, or 16 contiguous amino acids of the sequence of SEQ ID NO:5. In embodiments, the pseudoligand domain includes a sequence having 96% sequence identity to 5, 10, 11, 12, 13, 14, 15, or 16 contiguous amino acids of the sequence of SEQ ID NO:5. In embodiments, the pseudoligand domain includes a sequence having 97% sequence identity to 5, 10, 11, 12, 13, 14, 15, or 16 contiguous amino acids of the sequence of SEQ ID NO:5. In embodiments, the pseudoligand domain includes a sequence having 98% sequence identity to 5, 10, 11, 12, 13, 14, 15, or 16 contiguous amino acids of the sequence of SEQ ID NO:5. In embodiments, the pseudoligand domain includes a sequence having 99% sequence identity to 5, 10, 11, 12, 13, 14, 15, or 16 contiguous amino acids of the sequence of SEQ ID NO:5. In embodiments, the pseudoligand domain includes a sequence having 100% sequence identity to 5, 10, 11, 12, 13, 14, 15, or 16 contiguous amino acids of the sequence of SEQ ID NO:5.

In embodiments, the pseudoligand domain includes a sequence having 70%, 75%, 80%, 85%, 90%, 95%, 96%, 97%, 98%, 99% or 100% sequence identity to 5, 10, 11, 12, 13, 14, 15, 16, or 17 contiguous amino acids of the sequence of SEQ ID NO:6. In embodiments, the pseudoligand domain includes a sequence having 70% sequence identity to 5, 10, 11, 12, 13, 14, 15, 16, or 17 contiguous amino acids of the sequence of SEQ ID NO:6. In embodiments, the pseudoligand domain includes a sequence having 75% sequence identity to 5, 10, 11, 12, 13, 14, 15, 16, or 17 contiguous amino acids of the sequence of SEQ ID NO:6. In embodiments, the pseudoligand domain includes a sequence having 80% sequence identity to 5, 10, 11, 12, 13, 14, 15, 16, or 17 contiguous amino acids of the sequence of SEQ ID NO:6. In embodiments, the pseudoligand domain includes a sequence having 85% sequence identity to 5, 10, 11, 12, 13, 14, 15, 16, or 17 contiguous amino acids of the sequence of SEQ ID NO:6. In embodiments, the pseudoligand domain includes a sequence having 90% sequence identity to 5, 10, 11, 12, 13, 14, 15, 16, or 17 contiguous amino acids of the sequence of SEQ ID NO:6. In embodiments, the pseudoligand domain includes a sequence having 95% sequence identity to 5, 10, 11, 12, 13, 14, 15, 16, or 17 contiguous amino acids of the sequence of SEQ ID NO:6. In embodiments, the pseudoligand domain includes a sequence having 96% sequence identity to 5, 10, 11, 12, 13, 14, 15, 16, or 17 contiguous amino acids of the sequence of SEQ ID NO:6. In embodiments, the pseudoligand domain includes a sequence having 97% sequence identity to 5, 10, 11, 12, 13, 14, 15, 16, or 17 contiguous amino acids of the sequence of SEQ ID NO:6. In embodiments, the pseudoligand domain includes a sequence having 98% sequence identity to 5, 10, 11, 12, 13, 14, 15, 16, or 17 contiguous amino acids of the sequence of SEQ ID NO:6. In embodiments, the pseudoligand domain includes a sequence having 99% sequence identity to 5, 10, 11, 12, 13, 14, 15, 16, or 17 contiguous amino acids of the sequence of SEQ ID NO:6. In embodiments, the pseudoligand domain includes a sequence having 100% sequence identity to 5, 10, 11, 12, 13, 14, 15, 16, or 17 contiguous amino acids of the sequence of SEQ ID NO:6.

In embodiments, the pseudoligand domain includes a sequence having 70%, 75%, 80%, 85%, 90%, 95%, 96%, 97%, 98%, 99% or 100% sequence identity to 5, 10, 15, 20, 30, 40, 50, 60, or 70 contiguous amino acids of the sequence of SEQ ID NO:11. In embodiments, the pseudoligand domain includes a sequence having 70% sequence identity to 5, 10, 15, 20, 30, 40, 50, 60, or 70 contiguous amino acids of the sequence of SEQ ID NO:11. In embodiments, the pseudoligand domain includes a sequence having 75% sequence identity to 5, 10, 15, 20, 30, 40, 50, 60, or 70 contiguous amino acids of the sequence of SEQ ID NO:11. In embodiments, the pseudoligand domain includes a sequence having 80% sequence identity to 5, 10, 15, 20, 30, 40, 50, 60, or 70 contiguous amino acids of the sequence of SEQ ID NO:11. In embodiments, the pseudoligand domain includes a sequence having 85% sequence identity to 5, 10, 15, 20, 30, 40, 50, 60, or 70 contiguous amino acids of the sequence of SEQ ID NO:11. In embodiments, the pseudoligand domain includes a sequence having 90% sequence identity to 5, 10, 15, 20, 30, 40, 50, 60, or 70 contiguous amino acids of the sequence of SEQ ID NO: 11. In embodiments, the pseudoligand domain includes a sequence having 95% sequence identity to 5, 10, 15, 20, 30, 40, 50, 60, or 70 contiguous amino acids of the sequence of SEQ ID NO:11. In embodiments, the pseudoligand domain includes a sequence having 96% sequence identity to 5, 10, 15, 20, 30, 40, 50, 60, or 70 contiguous amino acids of the sequence of SEQ ID NO:11. In embodiments, the pseudoligand domain includes a sequence having 97% sequence identity to 5, 10, 15, 20, 30, 40, 50, 60, or 70 contiguous amino acids of the sequence of SEQ ID NO:11. In embodiments, the pseudoligand domain includes a sequence having 98% sequence identity to 55, 10, 15, 20, 30, 40, 50, 60, or 70 contiguous amino acids of the sequence of SEQ ID NO:11. In embodiments, the pseudoligand domain includes a sequence having 99% sequence identity to 5, 10, 15, 20, 30, 40, 50, 60, or 70 contiguous amino acids of the sequence of SEQ ID NO:11. In embodiments, the pseudoligand domain includes a sequence having 100% sequence identity to 5, 10, 15, 20, 30, 40, 50, 60, or 70 contiguous amino acids of the sequence of SEQ ID NO:11.

In embodiments, the ligand binding domain includes a Ras binding domain (RBD). In embodiments, the ligand binding domain includes the amino acid sequence of SEQ ID NO:17, SEQ ID NO:18, SEQ ID NO:19, SEQ ID NO:20, or SEQ ID NO:21. In embodiments, the ligand binding domain includes the amino acid sequence of SEQ ID NO: 17. In embodiments, the ligand binding domain includes the amino acid sequence of SEQ ID NO: 18. In embodiments, the ligand binding domain includes the amino acid sequence of SEQ ID NO: 19. In embodiments, the ligand binding domain includes the amino acid sequence of SEQ ID NO:20. In embodiments, the ligand binding domain includes the amino acid sequence of SEQ ID NO:21. In embodiments, the ligand binding domain is the amino acid sequence of SEQ ID NO:17, SEQ ID NO:18, SEQ ID NO:19, SEQ ID NO:20, or SEQ ID NO:21. In embodiments, the ligand binding domain is the amino acid sequence of SEQ ID NO:17. In embodiments, the ligand binding domain is the amino acid sequence of SEQ ID NO: 18. In embodiments, the ligand binding domain is the amino acid sequence of SEQ ID NO: 19. In embodiments, the ligand binding domain is the amino acid sequence of SEQ ID NO:20. In embodiments, the ligand binding domain is the amino acid sequence of SEQ ID NO:21.

In embodiments, the C-terminus of the first fluorophore is attached to the N-terminus of the ligand binding domain through the first peptidyl linker. In embodiments, the C-terminus of the ligand binding domain is attached to the N-terminus of the pseudoligand domain through the second peptidyl linker. In embodiments, the C-terminus of the pseudoligand binding domain is attached to the N-terminus of the second fluorophore through the third peptidyl linker.

In embodiments, the recombinant protein further includes a targeting moiety. In embodiments, the targeting moiety is a cytosolic targeting moiety, a plasma membrane targeting moiety, a Golgi apparatus targeting moiety. In embodiments, the targeting moiety is a cytosolic targeting moiety. In embodiments, the targeting moiety is a plasma membrane targeting moiety. In embodiments, the targeting moiety is a Golgi apparatus targeting moiety. In embodiments, the targeting moiety is a nuclear export signal (NES), a Giantin tag, or a Lyn-targeting motif. In embodiments, the targeting moiety is a nuclear export signal (NES). In embodiments, the targeting moiety is a Giantin tag. In embodiments, the targeting moiety is a Lyn-targeting motif.

In embodiments, the targeting moiety includes the amino acid sequence of SEQ ID NO: 14, SEQ ID NO:15, or SEQ ID NO: 16. In embodiments, the targeting moiety includes the amino acid sequence of SEQ ID NO: 14. In embodiments, the targeting moiety includes the amino acid sequence of SEQ ID NO:15. In embodiments, the targeting moiety includes the amino acid sequence of SEQ ID NO: 16. In embodiments, the targeting moiety is the amino acid sequence of SEQ ID NO: 14. In embodiments, the targeting moiety is the amino acid sequence of SEQ ID NO:15. In embodiments, the targeting moiety is the amino acid sequence of SEQ ID NO:16.

In embodiments, the C-terminus of the targeting moiety is attached to the N-terminus of the first fluorophore through a fourth peptidyl linker. In embodiments, the C-terminus of the second fluorophore is attached to the N-terminus of the targeting moiety through a fourth peptidyl linker.

In embodiments, the first peptidyl linker, the second peptidyl linker, the third peptidyl linker, and/or the fourth peptidyl linker are independently an amino acid sequence of about 1 to about 1000 amino acids. In embodiments, the first peptidyl linker, the second peptidyl linker, the third peptidyl linker, and/or the fourth peptidyl linker are independently an amino acid sequence of about 2 to about 1000 amino acids. In embodiments, the first peptidyl linker, the second peptidyl linker, the third peptidyl linker, and/or the fourth peptidyl linker are independently an amino acid sequence of about 3 to about 1000 amino acids. In embodiments, the first peptidyl linker, the second peptidyl linker, the third peptidyl linker, and/or the fourth peptidyl linker are independently an amino acid sequence of about 4 to about 1000 amino acids. In embodiments, the first peptidyl linker, the second peptidyl linker, the third peptidyl linker, and/or the fourth peptidyl linker are independently an amino acid sequence of about 5 to about 1000 amino acids. In embodiments, the first peptidyl linker, the second peptidyl linker, the third peptidyl linker, and/or the fourth peptidyl linker are independently an amino acid sequence of about 6 to about 1000 amino acids. In embodiments, the first peptidyl linker, the second peptidyl linker, the third peptidyl linker, and/or the fourth peptidyl linker are independently an amino acid sequence of about 7 to about 1000 amino acids. In embodiments, the first peptidyl linker, the second peptidyl linker, the third peptidyl linker, and/or the fourth peptidyl linker are independently an amino acid sequence of about 8 to about 1000 amino acids. In embodiments, the first peptidyl linker, the second peptidyl linker, the third peptidyl linker, and/or the fourth peptidyl linker are independently an amino acid sequence of about 9 to about 1000 amino acids. In embodiments, the first peptidyl linker, the second peptidyl linker, the third peptidyl linker, and/or the fourth peptidyl linker are independently an amino acid sequence of about 10 to about 1000 amino acids. In embodiments, the first peptidyl linker, the second peptidyl linker, the third peptidyl linker, and/or the fourth peptidyl linker are independently an amino acid sequence of about 20 to about 1000 amino acids. In embodiments, the first peptidyl linker, the second peptidyl linker, the third peptidyl linker, and/or the fourth peptidyl linker are independently an amino acid sequence of about 30 to about 1000 amino acids. In embodiments, the first peptidyl linker, the second peptidyl linker, the third peptidyl linker, and/or the fourth peptidyl linker are independently an amino acid sequence of about 40 to about 1000 amino acids. In embodiments, the first peptidyl linker, the second peptidyl linker, the third peptidyl linker, and/or the fourth peptidyl linker are independently an amino acid sequence of about 50 to about 1000 amino acids. In embodiments, the first peptidyl linker, the second peptidyl linker, the third peptidyl linker, and/or the fourth peptidyl linker are independently an amino acid sequence of about 100 to about 1000 amino acids. In embodiments, the first peptidyl linker, the second peptidyl linker, the third peptidyl linker, and/or the fourth peptidyl linker are independently an amino acid sequence of about 200 to about 1000 amino acids. In embodiments, the first peptidyl linker, the second peptidyl linker, the third peptidyl linker, and/or the fourth peptidyl linker are independently an amino acid sequence of about 300 to about 1000 amino acids. In embodiments, the first peptidyl linker, the second peptidyl linker, the third peptidyl linker, and/or the fourth peptidyl linker are independently an amino acid sequence of about 400 to about 1000 amino acids. In embodiments, the first peptidyl linker, the second peptidyl linker, the third peptidyl linker, and/or the fourth peptidyl linker are independently an amino acid sequence of about 500 to about 1000 amino acids. In embodiments, the first peptidyl linker, the second peptidyl linker, the third peptidyl linker, and/or the fourth peptidyl linker are independently an amino acid sequence of about 600 to about 1000 amino acids. In embodiments, the first peptidyl linker, the second peptidyl linker, the third peptidyl linker, and/or the fourth peptidyl linker are independently an amino acid sequence of about 700 to about 1000 amino acids. In embodiments, the first peptidyl linker, the second peptidyl linker, the third peptidyl linker, and/or the fourth peptidyl linker are independently an amino acid sequence of about 800 to about 1000 amino acids. In embodiments, the first peptidyl linker, the second peptidyl linker, the third peptidyl linker, and/or the fourth peptidyl linker are independently an amino acid sequence of about 900 to about 1000 amino acids.

In embodiments, the first peptidyl linker, the second peptidyl linker, the third peptidyl linker, and/or the fourth peptidyl linker are independently an amino acid sequence of about 1 to about 900 amino acids. In embodiments, the first peptidyl linker, the second peptidyl linker, the third peptidyl linker, and/or the fourth peptidyl linker are independently an amino acid sequence of about 1 to about 800 amino acids. In embodiments, the first peptidyl linker, the second peptidyl linker, the third peptidyl linker, and/or the fourth peptidyl linker are independently an amino acid sequence of about 1 to about 700 amino acids. In embodiments, the first peptidyl linker, the second peptidyl linker, the third peptidyl linker, and/or the fourth peptidyl linker are independently an amino acid sequence of about 1 to about 600 amino acids. In embodiments, the first peptidyl linker, the second peptidyl linker, the third peptidyl linker, and/or the fourth peptidyl linker are independently an amino acid sequence of about 1 to about 500 amino acids. In embodiments, the first peptidyl linker, the second peptidyl linker, the third peptidyl linker, and/or the fourth peptidyl linker are independently an amino acid sequence of about 1 to about 400 amino acids. In embodiments, the first peptidyl linker, the second peptidyl linker, the third peptidyl linker, and/or the fourth peptidyl linker are independently an amino acid sequence of about 1 to about 300 amino acids. In embodiments, the first peptidyl linker, the second peptidyl linker, the third peptidyl linker, and/or the fourth peptidyl linker are independently an amino acid sequence of about 1 to about 200 amino acids. In embodiments, the first peptidyl linker, the second peptidyl linker, the third peptidyl linker, and/or the fourth peptidyl linker are independently an amino acid sequence of about 1 to about 100 amino acids. In embodiments, the first peptidyl linker, the second peptidyl linker, the third peptidyl linker, and/or the fourth peptidyl linker are independently an amino acid sequence of about 1 to about 50 amino acids. In embodiments, the first peptidyl linker, the second peptidyl linker, the third peptidyl linker, and/or the fourth peptidyl linker are independently an amino acid sequence of about 1 to about 40 amino acids. In embodiments, the first peptidyl linker, the second peptidyl linker, the third peptidyl linker, and/or the fourth peptidyl linker are independently an amino acid sequence of about 1 to about 30 amino acids. In embodiments, the first peptidyl linker, the second peptidyl linker, the third peptidyl linker, and/or the fourth peptidyl linker are independently an amino acid sequence of about 1 to about 20 amino acids. In embodiments, the first peptidyl linker, the second peptidyl linker, the third peptidyl linker, and/or the fourth peptidyl linker are independently an amino acid sequence of about 1 to about 10 amino acids. In embodiments, the first peptidyl linker, the second peptidyl linker, the third peptidyl linker, and/or the fourth peptidyl linker are independently an amino acid sequence of about 1 to about 9 amino acids. In embodiments, the first peptidyl linker, the second peptidyl linker, the third peptidyl linker, and/or the fourth peptidyl linker are independently an amino acid sequence of about 1 to about 8 amino acids. In embodiments, the first peptidyl linker, the second peptidyl linker, the third peptidyl linker, and/or the fourth peptidyl linker are independently an amino acid sequence of about 1 to about 7 amino acids. In embodiments, the first peptidyl linker, the second peptidyl linker, the third peptidyl linker, and/or the fourth peptidyl linker are independently an amino acid sequence of about 1 to about 6 amino acids. In embodiments, the first peptidyl linker, the second peptidyl linker, the third peptidyl linker, and/or the fourth peptidyl linker are independently an amino acid sequence of about 1 to about 5 amino acids. In embodiments, the first peptidyl linker, the second peptidyl linker, the third peptidyl linker, and/or the fourth peptidyl linker are independently an amino acid sequence of about 1 to about 4 amino acids. In embodiments, the first peptidyl linker, the second peptidyl linker, the third peptidyl linker, and/or the fourth peptidyl linker are independently an amino acid sequence of about 1 to about 3 amino acids. In embodiments, the first peptidyl linker, the second peptidyl linker, the third peptidyl linker, and/or the fourth peptidyl linker are independently an amino acid sequence of about 1 to about 2 amino acids.

In embodiments, the first peptidyl linker, the second peptidyl linker, the third peptidyl linker, and/or the fourth peptidyl linker are independently an amino acid sequence of 1 to 1000 amino acids. In embodiments, the first peptidyl linker, the second peptidyl linker, the third peptidyl linker, and/or the fourth peptidyl linker are independently an amino acid sequence of 2 to 1000 amino acids. In embodiments, the first peptidyl linker, the second peptidyl linker, the third peptidyl linker, and/or the fourth peptidyl linker are independently an amino acid sequence of 3 to 1000 amino acids. In embodiments, the first peptidyl linker, the second peptidyl linker, the third peptidyl linker, and/or the fourth peptidyl linker are independently an amino acid sequence of 4 to 1000 amino acids. In embodiments, the first peptidyl linker, the second peptidyl linker, the third peptidyl linker, and/or the fourth peptidyl linker are independently an amino acid sequence of 5 to 1000 amino acids. In embodiments, the first peptidyl linker, the second peptidyl linker, the third peptidyl linker, and/or the fourth peptidyl linker are independently an amino acid sequence of 6 to 1000 amino acids. In embodiments, the first peptidyl linker, the second peptidyl linker, the third peptidyl linker, and/or the fourth peptidyl linker are independently an amino acid sequence of 7 to 1000 amino acids. In embodiments, the first peptidyl linker, the second peptidyl linker, the third peptidyl linker, and/or the fourth peptidyl linker are independently an amino acid sequence of 8 to 1000 amino acids. In embodiments, the first peptidyl linker, the second peptidyl linker, the third peptidyl linker, and/or the fourth peptidyl linker are independently an amino acid sequence of 9 to 1000 amino acids. In embodiments, the first peptidyl linker, the second peptidyl linker, the third peptidyl linker, and/or the fourth peptidyl linker are independently an amino acid sequence of 10 to 1000 amino acids. In embodiments, the first peptidyl linker, the second peptidyl linker, the third peptidyl linker, and/or the fourth peptidyl linker are independently an amino acid sequence of 20 to 1000 amino acids. In embodiments, the first peptidyl linker, the second peptidyl linker, the third peptidyl linker, and/or the fourth peptidyl linker are independently an amino acid sequence of 30 to 1000 amino acids. In embodiments, the first peptidyl linker, the second peptidyl linker, the third peptidyl linker, and/or the fourth peptidyl linker are independently an amino acid sequence of 40 to 1000 amino acids. In embodiments, the first peptidyl linker, the second peptidyl linker, the third peptidyl linker, and/or the fourth peptidyl linker are independently an amino acid sequence of 50 to 1000 amino acids. In embodiments, the first peptidyl linker, the second peptidyl linker, the third peptidyl linker, and/or the fourth peptidyl linker are independently an amino acid sequence of 100 to 1000 amino acids. In embodiments, the first peptidyl linker, the second peptidyl linker, the third peptidyl linker, and/or the fourth peptidyl linker are independently an amino acid sequence of 200 to 1000 amino acids. In embodiments, the first peptidyl linker, the second peptidyl linker, the third peptidyl linker, and/or the fourth peptidyl linker are independently an amino acid sequence of 300 to 1000 amino acids. In embodiments, the first peptidyl linker, the second peptidyl linker, the third peptidyl linker, and/or the fourth peptidyl linker are independently an amino acid sequence of 400 to 1000 amino acids. In embodiments, the first peptidyl linker, the second peptidyl linker, the third peptidyl linker, and/or the fourth peptidyl linker are independently an amino acid sequence of 500 to 1000 amino acids. In embodiments, the first peptidyl linker, the second peptidyl linker, the third peptidyl linker, and/or the fourth peptidyl linker are independently an amino acid sequence of 600 to 1000 amino acids. In embodiments, the first peptidyl linker, the second peptidyl linker, the third peptidyl linker, and/or the fourth peptidyl linker are independently an amino acid sequence of 700 to 1000 amino acids. In embodiments, the first peptidyl linker, the second peptidyl linker, the third peptidyl linker, and/or the fourth peptidyl linker are independently an amino acid sequence of 800 to 1000 amino acids. In embodiments, the first peptidyl linker, the second peptidyl linker, the third peptidyl linker, and/or the fourth peptidyl linker are independently an amino acid sequence of 900 to 1000 amino acids.

In embodiments, the first peptidyl linker, the second peptidyl linker, the third peptidyl linker, and/or the fourth peptidyl linker are independently an amino acid sequence of 1 to 900 amino acids. In embodiments, the first peptidyl linker, the second peptidyl linker, the third peptidyl linker, and/or the fourth peptidyl linker are independently an amino acid sequence of 1 to 800 amino acids. In embodiments, the first peptidyl linker, the second peptidyl linker, the third peptidyl linker, and/or the fourth peptidyl linker are independently an amino acid sequence of 1 to 700 amino acids. In embodiments, the first peptidyl linker, the second peptidyl linker, the third peptidyl linker, and/or the fourth peptidyl linker are independently an amino acid sequence of 1 to 600 amino acids. In embodiments, the first peptidyl linker, the second peptidyl linker, the third peptidyl linker, and/or the fourth peptidyl linker are independently an amino acid sequence of 1 to 500 amino acids. In embodiments, the first peptidyl linker, the second peptidyl linker, the third peptidyl linker, and/or the fourth peptidyl linker are independently an amino acid sequence of 1 to 400 amino acids. In embodiments, the first peptidyl linker, the second peptidyl linker, the third peptidyl linker, and/or the fourth peptidyl linker are independently an amino acid sequence of 1 to 300 amino acids. In embodiments, the first peptidyl linker, the second peptidyl linker, the third peptidyl linker, and/or the fourth peptidyl linker are independently an amino acid sequence of 1 to 200 amino acids. In embodiments, the first peptidyl linker, the second peptidyl linker, the third peptidyl linker, and/or the fourth peptidyl linker are independently an amino acid sequence of 1 to 100 amino acids. In embodiments, the first peptidyl linker, the second peptidyl linker, the third peptidyl linker, and/or the fourth peptidyl linker are independently an amino acid sequence of 1 to 50 amino acids. In embodiments, the first peptidyl linker, the second peptidyl linker, the third peptidyl linker, and/or the fourth peptidyl linker are independently an amino acid sequence of 1 to 40 amino acids. In embodiments, the first peptidyl linker, the second peptidyl linker, the third peptidyl linker, and/or the fourth peptidyl linker are independently an amino acid sequence of 1 to 30 amino acids. In embodiments, the first peptidyl linker, the second peptidyl linker, the third peptidyl linker, and/or the fourth peptidyl linker are independently an amino acid sequence of 1 to 20 amino acids. In embodiments, the first peptidyl linker, the second peptidyl linker, the third peptidyl linker, and/or the fourth peptidyl linker are independently an amino acid sequence of 1 to 10 amino acids. In embodiments, the first peptidyl linker, the second peptidyl linker, the third peptidyl linker, and/or the fourth peptidyl linker are independently an amino acid sequence of 1 to 9 amino acids. In embodiments, the first peptidyl linker, the second peptidyl linker, the third peptidyl linker, and/or the fourth peptidyl linker are independently an amino acid sequence of 1 to 8 amino acids. In embodiments, the first peptidyl linker, the second peptidyl linker, the third peptidyl linker, and/or the fourth peptidyl linker are independently an amino acid sequence of 1 to 7 amino acids. In embodiments, the first peptidyl linker, the second peptidyl linker, the third peptidyl linker, and/or the fourth peptidyl linker are independently an amino acid sequence of 1 to 6 amino acids. In embodiments, the first peptidyl linker, the second peptidyl linker, the third peptidyl linker, and/or the fourth peptidyl linker are independently an amino acid sequence of 1 to 5 amino acids. In embodiments, the first peptidyl linker, the second peptidyl linker, the third peptidyl linker, and/or the fourth peptidyl linker are independently an amino acid sequence of 1 to 4 amino acids. In embodiments, the first peptidyl linker, the second peptidyl linker, the third peptidyl linker, and/or the fourth peptidyl linker are independently an amino acid sequence of 1 to 3 amino acids. In embodiments, the first peptidyl linker, the second peptidyl linker, the third peptidyl linker, and/or the fourth peptidyl linker are independently an amino acid sequence of 1 to 2 amino acids.

In embodiments, the first peptidyl linker, the second peptidyl linker, the third peptidyl linker, or the fourth peptidyl linker is an amino acid sequence of about 1 to about 1000 amino acids. In embodiments, the first peptidyl linker, the second peptidyl linker, the third peptidyl linker, and the fourth peptidyl linker are an amino acid sequence of about 1 to about 1000 amino acids.

In embodiments, the first peptidyl linker is an amino acid sequence of about 1 to about 1000 amino acids. In embodiments, the first peptidyl linker is an amino acid sequence of about 2 to about 1000 amino acids. In embodiments, the first peptidyl linker is an amino acid sequence of about 3 to about 1000 amino acids. In embodiments, the first peptidyl linker is an amino acid sequence of about 4 to about 1000 amino acids. In embodiments, the first peptidyl linker is an amino acid sequence of about 5 to about 1000 amino acids. In embodiments, the first peptidyl linker is an amino acid sequence of about 6 to about 1000 amino acids. In embodiments, the first peptidyl linker is an amino acid sequence of about 7 to about 1000 amino acids. In embodiments, the first peptidyl linker is an amino acid sequence of about 8 to about 1000 amino acids. In embodiments, the first peptidyl linker is an amino acid sequence of about 9 to about 1000 amino acids. In embodiments, the first peptidyl linker is an amino acid sequence of about 10 to about 1000 amino acids. In embodiments, the first peptidyl linker is an amino acid sequence of about 20 to about 1000 amino acids. In embodiments, the first peptidyl linker is an amino acid sequence of about 30 to about 1000 amino acids. In embodiments, the first peptidyl linker is an amino acid sequence of about 40 to about 1000 amino acids. In embodiments, the first peptidyl linker is an amino acid sequence of about 50 to about 1000 amino acids. In embodiments, the first peptidyl linker is an amino acid sequence of about 100 to about 1000 amino acids. In embodiments, the first peptidyl linker is an amino acid sequence of about 200 to about 1000 amino acids. In embodiments, the first peptidyl linker is an amino acid sequence of about 300 to about 1000 amino acids. In embodiments, the first peptidyl linker is an amino acid sequence of about 400 to about 1000 amino acids. In embodiments, the first peptidyl linker is an amino acid sequence of about 500 to about 1000 amino acids. In embodiments, the first peptidyl linker is an amino acid sequence of about 600 to about 1000 amino acids. In embodiments, the first peptidyl linker is an amino acid sequence of about 700 to about 1000 amino acids. In embodiments, the first peptidyl linker is an amino acid sequence of about 800 to about 1000 amino acids. In embodiments, the first peptidyl linker is an amino acid sequence of about 900 to about 1000 amino acids.

In embodiments, the first peptidyl linker is an amino acid sequence of about 1 to about 900 amino acids. In embodiments, the first peptidyl linker is an amino acid sequence of about 1 to about 800 amino acids. In embodiments, the first peptidyl linker is an amino acid sequence of about 1 to about 700 amino acids. In embodiments, the first peptidyl linker is an amino acid sequence of about 1 to about 600 amino acids. In embodiments, the first peptidyl linker is an amino acid sequence of about 1 to about 500 amino acids. In embodiments, the first peptidyl linker is an amino acid sequence of about 1 to about 400 amino acids. In embodiments, the first peptidyl linker is an amino acid sequence of about 1 to about 300 amino acids. In embodiments, the first peptidyl linker is an amino acid sequence of about 1 to about 200 amino acids. In embodiments, the first peptidyl linker is an amino acid sequence of about 1 to about 100 amino acids. In embodiments, the first peptidyl linker is an amino acid sequence of about 1 to about 50 amino acids. In embodiments, the first peptidyl linker is an amino acid sequence of about 1 to about 40 amino acids. In embodiments, the first peptidyl linker is an amino acid sequence of about 1 to about 30 amino acids. In embodiments, the first peptidyl linker is an amino acid sequence of about 1 to about 20 amino acids. In embodiments, the first peptidyl linker is an amino acid sequence of about 1 to about 10 amino acids. In embodiments, the first peptidyl linker is an amino acid sequence of about 1 to about 9 amino acids. In embodiments, the first peptidyl linker is an amino acid sequence of about 1 to about 8 amino acids. In embodiments, the first peptidyl linker is an amino acid sequence of about 1 to about 7 amino acids. In embodiments, the first peptidyl linker is an amino acid sequence of about 1 to about 6 amino acids. In embodiments, the first peptidyl linker is an amino acid sequence of about 1 to about 5 amino acids. In embodiments, the first peptidyl linker is an amino acid sequence of about 1 to about 4 amino acids. In embodiments, the first peptidyl linker is an amino acid sequence of about 1 to about 3 amino acids. In embodiments, the first peptidyl linker is an amino acid sequence of about 1 to about 2 amino acids.

In embodiments, the first peptidyl linker is an amino acid sequence of 1 to 1000 amino acids. In embodiments, the first peptidyl linker is an amino acid sequence of 2 to 1000 amino acids. In embodiments, the first peptidyl linker is an amino acid sequence of 3 to 1000 amino acids. In embodiments, the first peptidyl linker is an amino acid sequence of 4 to 1000 amino acids. In embodiments, the first peptidyl linker is an amino acid sequence of 5 to 1000 amino acids. In embodiments, the first peptidyl linker is an amino acid sequence of 6 to 1000 amino acids. In embodiments, the first peptidyl linker is an amino acid sequence of 7 to 1000 amino acids. In embodiments, the first peptidyl linker is an amino acid sequence of 8 to 1000 amino acids. In embodiments, the first peptidyl linker is an amino acid sequence of 9 to 1000 amino acids. In embodiments, the first peptidyl linker is an amino acid sequence of 10 to 1000 amino acids. In embodiments, the first peptidyl linker is an amino acid sequence of 20 to 1000 amino acids. In embodiments, the first peptidyl linker is an amino acid sequence of 30 to 1000 amino acids. In embodiments, the first peptidyl linker is an amino acid sequence of 40 to 1000 amino acids. In embodiments, the first peptidyl linker is an amino acid sequence of 50 to 1000 amino acids. In embodiments, the first peptidyl linker is an amino acid sequence of 100 to 1000 amino acids. In embodiments, the first peptidyl linker is an amino acid sequence of 200 to 1000 amino acids. In embodiments, the first peptidyl linker is an amino acid sequence of 300 to 1000 amino acids. In embodiments, the first peptidyl linker is an amino acid sequence of 400 to 1000 amino acids. In embodiments, the first peptidyl linker is an amino acid sequence of 500 to 1000 amino acids. In embodiments, the first peptidyl linker is an amino acid sequence of 600 to 1000 amino acids. In embodiments, the first peptidyl linker is an amino acid sequence of 700 to 1000 amino acids. In embodiments, the first peptidyl linker is an amino acid sequence of 800 to 1000 amino acids. In embodiments, the first peptidyl linker is an amino acid sequence of 900 to 1000 amino acids.

In embodiments, the first peptidyl linker is an amino acid sequence of 1 to 900 amino acids. In embodiments, the first peptidyl linker is an amino acid sequence of 1 to 800 amino acids. In embodiments, the first peptidyl linker is an amino acid sequence of 1 to 700 amino acids. In embodiments, the first peptidyl linker is an amino acid sequence of 1 to 600 amino acids. In embodiments, the first peptidyl linker is an amino acid sequence of 1 to 500 amino acids. In embodiments, the first peptidyl linker is an amino acid sequence of 1 to 400 amino acids. In embodiments, the first peptidyl linker is an amino acid sequence of 1 to 300 amino acids. In embodiments, the first peptidyl linker is an amino acid sequence of 1 to 200 amino acids. In embodiments, the first peptidyl linker is an amino acid sequence of 1 to 100 amino acids. In embodiments, the first peptidyl linker is an amino acid sequence of 1 to 50 amino acids. In embodiments, the first peptidyl linker is an amino acid sequence of 1 to 40 amino acids. In embodiments, the first peptidyl linker is an amino acid sequence of 1 to 30 amino acids. In embodiments, the first peptidyl linker is an amino acid sequence of 1 to 20 amino acids. In embodiments, the first peptidyl linker is an amino acid sequence of 1 to 10 amino acids. In embodiments, the first peptidyl linker is an amino acid sequence of 1 to 9 amino acids. In embodiments, the first peptidyl linker is an amino acid sequence of 1 to 8 amino acids. In embodiments, the first peptidyl linker is an amino acid sequence of 1 to 7 amino acids. In embodiments, the first peptidyl linker is an amino acid sequence of 1 to 6 amino acids. In embodiments, the first peptidyl linker is an amino acid sequence of 1 to 5 amino acids. In embodiments, the first peptidyl linker is an amino acid sequence of 1 to 4 amino acids. In embodiments, the first peptidyl linker is an amino acid sequence of 1 to 3 amino acids. In embodiments, the first peptidyl linker is an amino acid sequence of 1 to 2 amino acids.

In embodiments, the second peptidyl linker is an amino acid sequence of about 1 to about 1000 amino acids. In embodiments, the second peptidyl linker is an amino acid sequence of about 2 to about 1000 amino acids. In embodiments, the second peptidyl linker is an amino acid sequence of about 3 to about 1000 amino acids. In embodiments, the second peptidyl linker is an amino acid sequence of about 4 to about 1000 amino acids. In embodiments, the second peptidyl linker is an amino acid sequence of about 5 to about 1000 amino acids. In embodiments, the second peptidyl linker is an amino acid sequence of about 6 to about 1000 amino acids. In embodiments, the second peptidyl linker is an amino acid sequence of about 7 to about 1000 amino acids. In embodiments, the second peptidyl linker is an amino acid sequence of about 8 to about 1000 amino acids. In embodiments, the second peptidyl linker is an amino acid sequence of about 9 to about 1000 amino acids. In embodiments, the second peptidyl linker is an amino acid sequence of about 10 to about 1000 amino acids. In embodiments, the second peptidyl linker is an amino acid sequence of about 20 to about 1000 amino acids. In embodiments, the second peptidyl linker is an amino acid sequence of about 30 to about 1000 amino acids. In embodiments, the second peptidyl linker is an amino acid sequence of about 40 to about 1000 amino acids. In embodiments, the second peptidyl linker is an amino acid sequence of about 50 to about 1000 amino acids. In embodiments, the second peptidyl linker is an amino acid sequence of about 100 to about 1000 amino acids. In embodiments, the second peptidyl linker is an amino acid sequence of about 200 to about 1000 amino acids. In embodiments, the second peptidyl linker is an amino acid sequence of about 300 to about 1000 amino acids. In embodiments, the second peptidyl linker is an amino acid sequence of about 400 to about 1000 amino acids. In embodiments, the second peptidyl linker is an amino acid sequence of about 500 to about 1000 amino acids. In embodiments, the second peptidyl linker is an amino acid sequence of about 600 to about 1000 amino acids. In embodiments, the second peptidyl linker is an amino acid sequence of about 700 to about 1000 amino acids. In embodiments, the second peptidyl linker is an amino acid sequence of about 800 to about 1000 amino acids. In embodiments, the second peptidyl linker is an amino acid sequence of about 900 to about 1000 amino acids.

In embodiments, the second peptidyl linker is an amino acid sequence of about 1 to about 900 amino acids. In embodiments, the second peptidyl linker is an amino acid sequence of about 1 to about 800 amino acids. In embodiments, the second peptidyl linker is an amino acid sequence of about 1 to about 700 amino acids. In embodiments, the second peptidyl linker is an amino acid sequence of about 1 to about 600 amino acids. In embodiments, the second peptidyl linker is an amino acid sequence of about 1 to about 500 amino acids. In embodiments, the second peptidyl linker is an amino acid sequence of about 1 to about 400 amino acids. In embodiments, the second peptidyl linker is an amino acid sequence of about 1 to about 300 amino acids. In embodiments, the second peptidyl linker is an amino acid sequence of about 1 to about 200 amino acids. In embodiments, the second peptidyl linker is an amino acid sequence of about 1 to about 100 amino acids. In embodiments, the second peptidyl linker is an amino acid sequence of about 1 to about 50 amino acids. In embodiments, the second peptidyl linker is an amino acid sequence of about 1 to about 40 amino acids. In embodiments, the second peptidyl linker is an amino acid sequence of about 1 to about 30 amino acids. In embodiments, the second peptidyl linker is an amino acid sequence of about 1 to about 20 amino acids. In embodiments, the second peptidyl linker is an amino acid sequence of about 1 to about 10 amino acids. In embodiments, the second peptidyl linker is an amino acid sequence of about 1 to about 9 amino acids. In embodiments, the second peptidyl linker is an amino acid sequence of about 1 to about 8 amino acids. In embodiments, the second peptidyl linker is an amino acid sequence of about 1 to about 7 amino acids. In embodiments, the second peptidyl linker is an amino acid sequence of about 1 to about 6 amino acids. In embodiments, the second peptidyl linker is an amino acid sequence of about 1 to about 5 amino acids. In embodiments, the second peptidyl linker is an amino acid sequence of about 1 to about 4 amino acids. In embodiments, the second peptidyl linker is an amino acid sequence of about 1 to about 3 amino acids. In embodiments, the second peptidyl linker is an amino acid sequence of about 1 to about 2 amino acids.

In embodiments, the second peptidyl linker is an amino acid sequence of 1 to 1000 amino acids. In embodiments, the second peptidyl linker is an amino acid sequence of 2 to 1000 amino acids. In embodiments, the second peptidyl linker is an amino acid sequence of 3 to 1000 amino acids. In embodiments, the second peptidyl linker is an amino acid sequence of 4 to 1000 amino acids. In embodiments, the second peptidyl linker is an amino acid sequence of 5 to 1000 amino acids. In embodiments, the second peptidyl linker is an amino acid sequence of 6 to 1000 amino acids. In embodiments, the second peptidyl linker is an amino acid sequence of 7 to 1000 amino acids. In embodiments, the second peptidyl linker is an amino acid sequence of 8 to 1000 amino acids. In embodiments, the second peptidyl linker is an amino acid sequence of 9 to 1000 amino acids. In embodiments, the second peptidyl linker is an amino acid sequence of 10 to 1000 amino acids. In embodiments, the second peptidyl linker is an amino acid sequence of 20 to 1000 amino acids. In embodiments, the second peptidyl linker is an amino acid sequence of 30 to 1000 amino acids. In embodiments, the second peptidyl linker is an amino acid sequence of 40 to 1000 amino acids. In embodiments, the second peptidyl linker is an amino acid sequence of 50 to 1000 amino acids. In embodiments, the second peptidyl linker is an amino acid sequence of 100 to 1000 amino acids. In embodiments, the second peptidyl linker is an amino acid sequence of 200 to 1000 amino acids. In embodiments, the second peptidyl linker is an amino acid sequence of 300 to 1000 amino acids. In embodiments, the second peptidyl linker is an amino acid sequence of 400 to 1000 amino acids. In embodiments, the second peptidyl linker is an amino acid sequence of 500 to 1000 amino acids. In embodiments, the second peptidyl linker is an amino acid sequence of 600 to 1000 amino acids. In embodiments, the second peptidyl linker is an amino acid sequence of 700 to 1000 amino acids. In embodiments, the second peptidyl linker is an amino acid sequence of 800 to 1000 amino acids. In embodiments, the second peptidyl linker is an amino acid sequence of 900 to 1000 amino acids.

In embodiments, the second peptidyl linker is an amino acid sequence of 1 to 900 amino acids. In embodiments, the second peptidyl linker is an amino acid sequence of 1 to 800 amino acids. In embodiments, the second peptidyl linker is an amino acid sequence of 1 to 700 amino acids. In embodiments, the second peptidyl linker is an amino acid sequence of 1 to 600 amino acids. In embodiments, the second peptidyl linker is an amino acid sequence of 1 to 500 amino acids. In embodiments, the second peptidyl linker is an amino acid sequence of 1 to 400 amino acids. In embodiments, the second peptidyl linker is an amino acid sequence of 1 to 300 amino acids. In embodiments, the second peptidyl linker is an amino acid sequence of 1 to 200 amino acids. In embodiments, the second peptidyl linker is an amino acid sequence of 1 to 100 amino acids. In embodiments, the second peptidyl linker is an amino acid sequence of 1 to 50 amino acids. In embodiments, the second peptidyl linker is an amino acid sequence of 1 to 40 amino acids. In embodiments, the second peptidyl linker is an amino acid sequence of 1 to 30 amino acids. In embodiments, the second peptidyl linker is an amino acid sequence of 1 to 20 amino acids. In embodiments, the second peptidyl linker is an amino acid sequence of 1 to 10 amino acids. In embodiments, the second peptidyl linker is an amino acid sequence of 1 to 9 amino acids. In embodiments, the second peptidyl linker is an amino acid sequence of 1 to 8 amino acids. In embodiments, the second peptidyl linker is an amino acid sequence of 1 to 7 amino acids. In embodiments, the second peptidyl linker is an amino acid sequence of 1 to 6 amino acids. In embodiments, the second peptidyl linker is an amino acid sequence of 1 to 5 amino acids. In embodiments, the second peptidyl linker is an amino acid sequence of 1 to 4 amino acids. In embodiments, the second peptidyl linker is an amino acid sequence of 1 to 3 amino acids. In embodiments, the second peptidyl linker is an amino acid sequence of 1 to 2 amino acids.

In embodiments, the third peptidyl linker is an amino acid sequence of about 1 to about 1000 amino acids. In embodiments, the third peptidyl linker is an amino acid sequence of about 2 to about 1000 amino acids. In embodiments, the third peptidyl linker is an amino acid sequence of about 3 to about 1000 amino acids. In embodiments, the third peptidyl linker is an amino acid sequence of about 4 to about 1000 amino acids. In embodiments, the third peptidyl linker is an amino acid sequence of about 5 to about 1000 amino acids. In embodiments, the third peptidyl linker is an amino acid sequence of about 6 to about 1000 amino acids. In embodiments, the third peptidyl linker is an amino acid sequence of about 7 to about 1000 amino acids. In embodiments, the third peptidyl linker is an amino acid sequence of about 8 to about 1000 amino acids. In embodiments, the third peptidyl linker is an amino acid sequence of about 9 to about 1000 amino acids. In embodiments, the third peptidyl linker is an amino acid sequence of about 10 to about 1000 amino acids. In embodiments, the third peptidyl linker is an amino acid sequence of about 20 to about 1000 amino acids. In embodiments, the third peptidyl linker is an amino acid sequence of about 30 to about 1000 amino acids. In embodiments, the third peptidyl linker is an amino acid sequence of about 40 to about 1000 amino acids. In embodiments, the third peptidyl linker is an amino acid sequence of about 50 to about 1000 amino acids. In embodiments, the third peptidyl linker is an amino acid sequence of about 100 to about 1000 amino acids. In embodiments, the third peptidyl linker is an amino acid sequence of about 200 to about 1000 amino acids. In embodiments, the third peptidyl linker is an amino acid sequence of about 300 to about 1000 amino acids. In embodiments, the third peptidyl linker is an amino acid sequence of about 400 to about 1000 amino acids. In embodiments, the third peptidyl linker is an amino acid sequence of about 500 to about 1000 amino acids. In embodiments, the third peptidyl linker is an amino acid sequence of about 600 to about 1000 amino acids. In embodiments, the third peptidyl linker is an amino acid sequence of about 700 to about 1000 amino acids. In embodiments, the third peptidyl linker is an amino acid sequence of about 800 to about 1000 amino acids. In embodiments, the third peptidyl linker is an amino acid sequence of about 900 to about 1000 amino acids.

In embodiments, the third peptidyl linker is an amino acid sequence of about 1 to about 900 amino acids. In embodiments, the third peptidyl linker is an amino acid sequence of about 1 to about 800 amino acids. In embodiments, the third peptidyl linker is an amino acid sequence of about 1 to about 700 amino acids. In embodiments, the third peptidyl linker is an amino acid sequence of about 1 to about 600 amino acids. In embodiments, the third peptidyl linker is an amino acid sequence of about 1 to about 500 amino acids. In embodiments, the third peptidyl linker is an amino acid sequence of about 1 to about 400 amino acids. In embodiments, the third peptidyl linker is an amino acid sequence of about 1 to about 300 amino acids. In embodiments, the third peptidyl linker is an amino acid sequence of about 1 to about 200 amino acids. In embodiments, the third peptidyl linker is an amino acid sequence of about 1 to about 100 amino acids. In embodiments, the third peptidyl linker is an amino acid sequence of about 1 to about 50 amino acids. In embodiments, the third peptidyl linker is an amino acid sequence of about 1 to about 40 amino acids. In embodiments, the third peptidyl linker is an amino acid sequence of about 1 to about 30 amino acids. In embodiments, the third peptidyl linker is an amino acid sequence of about 1 to about 20 amino acids. In embodiments, the third peptidyl linker is an amino acid sequence of about 1 to about 10 amino acids. In embodiments, the third peptidyl linker is an amino acid sequence of about 1 to about 9 amino acids. In embodiments, the third peptidyl linker is an amino acid sequence of about 1 to about 8 amino acids. In embodiments, the third peptidyl linker is an amino acid sequence of about 1 to about 7 amino acids. In embodiments, the third peptidyl linker is an amino acid sequence of about 1 to about 6 amino acids. In embodiments, the third peptidyl linker is an amino acid sequence of about 1 to about 5 amino acids. In embodiments, the third peptidyl linker is an amino acid sequence of about 1 to about 4 amino acids. In embodiments, the third peptidyl linker is an amino acid sequence of about 1 to about 3 amino acids. In embodiments, the third peptidyl linker is an amino acid sequence of about 1 to about 2 amino acids.

In embodiments, the third peptidyl linker is an amino acid sequence of 1 to 1000 amino acids. In embodiments, the third peptidyl linker is an amino acid sequence of 2 to 1000 amino acids. In embodiments, the third peptidyl linker is an amino acid sequence of 3 to 1000 amino acids. In embodiments, the third peptidyl linker is an amino acid sequence of 4 to 1000 amino acids. In embodiments, the third peptidyl linker is an amino acid sequence of 5 to 1000 amino acids. In embodiments, the third peptidyl linker is an amino acid sequence of 6 to 1000 amino acids. In embodiments, the third peptidyl linker is an amino acid sequence of 7 to 1000 amino acids. In embodiments, the third peptidyl linker is an amino acid sequence of 8 to 1000 amino acids. In embodiments, the third peptidyl linker is an amino acid sequence of 9 to 1000 amino acids. In embodiments, the third peptidyl linker is an amino acid sequence of 10 to 1000 amino acids. In embodiments, the third peptidyl linker is an amino acid sequence of 20 to 1000 amino acids. In embodiments, the third peptidyl linker is an amino acid sequence of 30 to 1000 amino acids. In embodiments, the third peptidyl linker is an amino acid sequence of 40 to 1000 amino acids. In embodiments, the third peptidyl linker is an amino acid sequence of 50 to 1000 amino acids. In embodiments, the third peptidyl linker is an amino acid sequence of 100 to 1000 amino acids. In embodiments, the third peptidyl linker is an amino acid sequence of 200 to 1000 amino acids. In embodiments, the third peptidyl linker is an amino acid sequence of 300 to 1000 amino acids. In embodiments, the third peptidyl linker is an amino acid sequence of 400 to 1000 amino acids. In embodiments, the third peptidyl linker is an amino acid sequence of 500 to 1000 amino acids. In embodiments, the third peptidyl linker is an amino acid sequence of 600 to 1000 amino acids. In embodiments, the third peptidyl linker is an amino acid sequence of 700 to 1000 amino acids. In embodiments, the third peptidyl linker is an amino acid sequence of 800 to 1000 amino acids. In embodiments, the third peptidyl linker is an amino acid sequence of 900 to 1000 amino acids.

In embodiments, the third peptidyl linker is an amino acid sequence of 1 to 900 amino acids. In embodiments, the third peptidyl linker is an amino acid sequence of 1 to 800 amino acids. In embodiments, the third peptidyl linker is an amino acid sequence of 1 to 700 amino acids. In embodiments, the third peptidyl linker is an amino acid sequence of 1 to 600 amino acids. In embodiments, the third peptidyl linker is an amino acid sequence of 1 to 500 amino acids. In embodiments, the third peptidyl linker is an amino acid sequence of 1 to 400 amino acids. In embodiments, the third peptidyl linker is an amino acid sequence of 1 to 300 amino acids. In embodiments, the third peptidyl linker is an amino acid sequence of 1 to 200 amino acids. In embodiments, the third peptidyl linker is an amino acid sequence of 1 to 100 amino acids. In embodiments, the third peptidyl linker is an amino acid sequence of 1 to 50 amino acids. In embodiments, the third peptidyl linker is an amino acid sequence of 1 to 40 amino acids. In embodiments, the third peptidyl linker is an amino acid sequence of 1 to 30 amino acids. In embodiments, the third peptidyl linker is an amino acid sequence of 1 to 20 amino acids. In embodiments, the third peptidyl linker is an amino acid sequence of 1 to 10 amino acids. In embodiments, the third peptidyl linker is an amino acid sequence of 1 to 9 amino acids. In embodiments, the third peptidyl linker is an amino acid sequence of 1 to 8 amino acids. In embodiments, the third peptidyl linker is an amino acid sequence of 1 to 7 amino acids. In embodiments, the third peptidyl linker is an amino acid sequence of 1 to 6 amino acids. In embodiments, the third peptidyl linker is an amino acid sequence of 1 to 5 amino acids. In embodiments, the third peptidyl linker is an amino acid sequence of 1 to 4 amino acids. In embodiments, the third peptidyl linker is an amino acid sequence of 1 to 3 amino acids. In embodiments, the third peptidyl linker is an amino acid sequence of 1 to 2 amino acids.

In embodiments, the fourth peptidyl linker is an amino acid sequence of about 1 to about 1000 amino acids. In embodiments, the fourth peptidyl linker is an amino acid sequence of about 2 to about 1000 amino acids. In embodiments, the fourth peptidyl linker is an amino acid sequence of about 3 to about 1000 amino acids. In embodiments, the fourth peptidyl linker is an amino acid sequence of about 4 to about 1000 amino acids. In embodiments, the fourth peptidyl linker is an amino acid sequence of about 5 to about 1000 amino acids. In embodiments, the fourth peptidyl linker is an amino acid sequence of about 6 to about 1000 amino acids. In embodiments, the fourth peptidyl linker is an amino acid sequence of about 7 to about 1000 amino acids. In embodiments, the fourth peptidyl linker is an amino acid sequence of about 8 to about 1000 amino acids. In embodiments, the fourth peptidyl linker is an amino acid sequence of about 9 to about 1000 amino acids. In embodiments, the fourth peptidyl linker is an amino acid sequence of about 10 to about 1000 amino acids. In embodiments, the fourth peptidyl linker is an amino acid sequence of about 20 to about 1000 amino acids. In embodiments, the fourth peptidyl linker is an amino acid sequence of about 30 to about 1000 amino acids. In embodiments, the fourth peptidyl linker is an amino acid sequence of about 40 to about 1000 amino acids. In embodiments, the fourth peptidyl linker is an amino acid sequence of about 50 to about 1000 amino acids. In embodiments, the fourth peptidyl linker is an amino acid sequence of about 100 to about 1000 amino acids. In embodiments, the fourth peptidyl linker is an amino acid sequence of about 200 to about 1000 amino acids. In embodiments, the fourth peptidyl linker is an amino acid sequence of about 300 to about 1000 amino acids. In embodiments, the fourth peptidyl linker is an amino acid sequence of about 400 to about 1000 amino acids. In embodiments, the fourth peptidyl linker is an amino acid sequence of about 500 to about 1000 amino acids. In embodiments, the fourth peptidyl linker is an amino acid sequence of about 600 to about 1000 amino acids. In embodiments, the fourth peptidyl linker is an amino acid sequence of about 700 to about 1000 amino acids. In embodiments, the fourth peptidyl linker is an amino acid sequence of about 800 to about 1000 amino acids. In embodiments, the fourth peptidyl linker is an amino acid sequence of about 900 to about 1000 amino acids.

In embodiments, the fourth peptidyl linker is an amino acid sequence of about 1 to about 900 amino acids. In embodiments, the fourth peptidyl linker is an amino acid sequence of about 1 to about 800 amino acids. In embodiments, the fourth peptidyl linker is an amino acid sequence of about 1 to about 700 amino acids. In embodiments, the fourth peptidyl linker is an amino acid sequence of about 1 to about 600 amino acids. In embodiments, the fourth peptidyl linker is an amino acid sequence of about 1 to about 500 amino acids. In embodiments, the fourth peptidyl linker is an amino acid sequence of about 1 to about 400 amino acids. In embodiments, the fourth peptidyl linker is an amino acid sequence of about 1 to about 300 amino acids. In embodiments, the fourth peptidyl linker is an amino acid sequence of about 1 to about 200 amino acids. In embodiments, the fourth peptidyl linker is an amino acid sequence of about 1 to about 100 amino acids. In embodiments, the fourth peptidyl linker is an amino acid sequence of about 1 to about 50 amino acids. In embodiments, the fourth peptidyl linker is an amino acid sequence of about 1 to about 40 amino acids. In embodiments, the fourth peptidyl linker is an amino acid sequence of about 1 to about 30 amino acids. In embodiments, the fourth peptidyl linker is an amino acid sequence of about 1 to about 20 amino acids. In embodiments, the fourth peptidyl linker is an amino acid sequence of about 1 to about 10 amino acids. In embodiments, the fourth peptidyl linker is an amino acid sequence of about 1 to about 9 amino acids. In embodiments, the fourth peptidyl linker is an amino acid sequence of about 1 to about 8 amino acids. In embodiments, the fourth peptidyl linker is an amino acid sequence of about 1 to about 7 amino acids. In embodiments, the fourth peptidyl linker is an amino acid sequence of about 1 to about 6 amino acids. In embodiments, the fourth peptidyl linker is an amino acid sequence of about 1 to about 5 amino acids. In embodiments, the fourth peptidyl linker is an amino acid sequence of about 1 to about 4 amino acids. In embodiments, the fourth peptidyl linker is an amino acid sequence of about 1 to about 3 amino acids. In embodiments, the fourth peptidyl linker is an amino acid sequence of about 1 to about 2 amino acids.

In embodiments, the fourth peptidyl linker is an amino acid sequence of 1 to 1000 amino acids. In embodiments, the fourth peptidyl linker is an amino acid sequence of 2 to 1000 amino acids. In embodiments, the fourth peptidyl linker is an amino acid sequence of 3 to 1000 amino acids. In embodiments, the fourth peptidyl linker is an amino acid sequence of 4 to 1000 amino acids. In embodiments, the fourth peptidyl linker is an amino acid sequence of 5 to 1000 amino acids. In embodiments, the fourth peptidyl linker is an amino acid sequence of 6 to 1000 amino acids. In embodiments, the fourth peptidyl linker is an amino acid sequence of 7 to 1000 amino acids. In embodiments, the fourth peptidyl linker is an amino acid sequence of 8 to 1000 amino acids. In embodiments, the fourth peptidyl linker is an amino acid sequence of 9 to 1000 amino acids. In embodiments, the fourth peptidyl linker is an amino acid sequence of 10 to 1000 amino acids. In embodiments, the fourth peptidyl linker is an amino acid sequence of 20 to 1000 amino acids. In embodiments, the fourth peptidyl linker is an amino acid sequence of 30 to 1000 amino acids. In embodiments, the fourth peptidyl linker is an amino acid sequence of 40 to 1000 amino acids. In embodiments, the fourth peptidyl linker is an amino acid sequence of 50 to 1000 amino acids. In embodiments, the fourth peptidyl linker is an amino acid sequence of 100 to 1000 amino acids. In embodiments, the fourth peptidyl linker is an amino acid sequence of 200 to 1000 amino acids. In embodiments, the fourth peptidyl linker is an amino acid sequence of 300 to 1000 amino acids. In embodiments, the fourth peptidyl linker is an amino acid sequence of 400 to 1000 amino acids. In embodiments, the fourth peptidyl linker is an amino acid sequence of 500 to 1000 amino acids. In embodiments, the fourth peptidyl linker is an amino acid sequence of 600 to 1000 amino acids. In embodiments, the fourth peptidyl linker is an amino acid sequence of 700 to 1000 amino acids. In embodiments, the fourth peptidyl linker is an amino acid sequence of 800 to 1000 amino acids. In embodiments, the fourth peptidyl linker is an amino acid sequence of 900 to 1000 amino acids.

In embodiments, the fourth peptidyl linker is an amino acid sequence of 1 to 900 amino acids. In embodiments, the fourth peptidyl linker is an amino acid sequence of 1 to 800 amino acids. In embodiments, the fourth peptidyl linker is an amino acid sequence of 1 to 700 amino acids. In embodiments, the fourth peptidyl linker is an amino acid sequence of 1 to 600 amino acids. In embodiments, the fourth peptidyl linker is an amino acid sequence of 1 to 500 amino acids. In embodiments, the fourth peptidyl linker is an amino acid sequence of 1 to 400 amino acids. In embodiments, the fourth peptidyl linker is an amino acid sequence of 1 to 300 amino acids. In embodiments, the fourth peptidyl linker is an amino acid sequence of 1 to 200 amino acids. In embodiments, the fourth peptidyl linker is an amino acid sequence of 1 to 100 amino acids. In embodiments, the fourth peptidyl linker is an amino acid sequence of 1 to 50 amino acids. In embodiments, the fourth peptidyl linker is an amino acid sequence of 1 to 40 amino acids. In embodiments, the fourth peptidyl linker is an amino acid sequence of 1 to 30 amino acids. In embodiments, the fourth peptidyl linker is an amino acid sequence of 1 to 20 amino acids. In embodiments, the fourth peptidyl linker is an amino acid sequence of 1 to 10 amino acids. In embodiments, the fourth peptidyl linker is an amino acid sequence of 1 to 9 amino acids. In embodiments, the fourth peptidyl linker is an amino acid sequence of 1 to 8 amino acids. In embodiments, the fourth peptidyl linker is an amino acid sequence of 1 to 7 amino acids. In embodiments, the fourth peptidyl linker is an amino acid sequence of 1 to 6 amino acids. In embodiments, the fourth peptidyl linker is an amino acid sequence of 1 to 5 amino acids. In embodiments, the fourth peptidyl linker is an amino acid sequence of 1 to 4 amino acids. In embodiments, the fourth peptidyl linker is an amino acid sequence of 1 to 3 amino acids. In embodiments, the fourth peptidyl linker is an amino acid sequence of 1 to 2 amino acids.

In embodiments, the first peptidyl linker, the second peptidyl linker, the third peptidyl linker, and/or the fourth peptidyl linker independently include at least 1 glycine, serine, or threonine. In embodiments, the first peptidyl linker, the second peptidyl linker, the third peptidyl linker, and/or the fourth peptidyl linker independently include at least 2 glycines, serines, or threonines. In embodiments, the first peptidyl linker, the second peptidyl linker, the third peptidyl linker, and/or the fourth peptidyl linker independently include at least 3 glycines, serines, or threonines. In embodiments, the first peptidyl linker, the second peptidyl linker, the third peptidyl linker, and/or the fourth peptidyl linker independently include at least 4 glycines, serines, or threonines. In embodiments, the first peptidyl linker, the second peptidyl linker, the third peptidyl linker, and/or the fourth peptidyl linker independently include at least 5 glycines, serines, or threonines. In embodiments, the first peptidyl linker, the second peptidyl linker, the third peptidyl linker, and/or the fourth peptidyl linker independently include at least 6 glycines, serines, or threonines. In embodiments, the first peptidyl linker, the second peptidyl linker, the third peptidyl linker, and/or the fourth peptidyl linker independently include at least 7 glycines, serines, or threonines. In embodiments, the first peptidyl linker, the second peptidyl linker, the third peptidyl linker, and/or the fourth peptidyl linker independently include at least 8 glycines, serines, or threonines. In embodiments, the first peptidyl linker, the second peptidyl linker, the third peptidyl linker, and/or the fourth peptidyl linker independently include at least 9 glycines, serines, or threonines. In embodiments, the first peptidyl linker, the second peptidyl linker, the third peptidyl linker, and/or the fourth peptidyl linker independently include at least 10 glycines, serines, or threonines. In embodiments, the first peptidyl linker, the second peptidyl linker, the third peptidyl linker, and/or the fourth peptidyl linker independently include at least 11 glycines, serines, or threonines. In embodiments, the first peptidyl linker, the second peptidyl linker, the third peptidyl linker, and/or the fourth peptidyl linker independently include at least 12 glycines, serines, or threonines. In embodiments, the first peptidyl linker, the second peptidyl linker, the third peptidyl linker, and/or the fourth peptidyl linker independently include at least 13 glycines, serines, or threonines. In embodiments, the first peptidyl linker, the second peptidyl linker, the third peptidyl linker, and/or the fourth peptidyl linker independently include at least 14 glycines, serines, or threonines. In embodiments, the first peptidyl linker, the second peptidyl linker, the third peptidyl linker, and/or the fourth peptidyl linker independently include at least 15 glycines, serines, or threonines.

In embodiments, the first peptidyl linker, the second peptidyl linker, the third peptidyl linker, and/or the fourth peptidyl linker independently include at least 1 glycine. In embodiments, the first peptidyl linker, the second peptidyl linker, the third peptidyl linker, and/or the fourth peptidyl linker independently include at least 2 glycines. In embodiments, the first peptidyl linker, the second peptidyl linker, the third peptidyl linker, and/or the fourth peptidyl linker independently include at least 3 glycines. In embodiments, the first peptidyl linker, the second peptidyl linker, the third peptidyl linker, and/or the fourth peptidyl linker independently include at least 4 glycines. In embodiments, the first peptidyl linker, the second peptidyl linker, the third peptidyl linker, and/or the fourth peptidyl linker independently include at least 5 glycines. In embodiments, the first peptidyl linker, the second peptidyl linker, the third peptidyl linker, and/or the fourth peptidyl linker independently include at least 6 glycines. In embodiments, the first peptidyl linker, the second peptidyl linker, the third peptidyl linker, and/or the fourth peptidyl linker independently include at least 7 glycines. In embodiments, the first peptidyl linker, the second peptidyl linker, the third peptidyl linker, and/or the fourth peptidyl linker independently include at least 8 glycines. In embodiments, the first peptidyl linker, the second peptidyl linker, the third peptidyl linker, and/or the fourth peptidyl linker independently include at least 9 glycines. In embodiments, the first peptidyl linker, the second peptidyl linker, the third peptidyl linker, and/or the fourth peptidyl linker independently include at least 10 glycines. In embodiments, the first peptidyl linker, the second peptidyl linker, the third peptidyl linker, and/or the fourth peptidyl linker independently include at least 11 glycines. In embodiments, the first peptidyl linker, the second peptidyl linker, the third peptidyl linker, and/or the fourth peptidyl linker independently include at least 12 glycines. In embodiments, the first peptidyl linker, the second peptidyl linker, the third peptidyl linker, and/or the fourth peptidyl linker independently include at least 13 glycines. In embodiments, the first peptidyl linker, the second peptidyl linker, the third peptidyl linker, and/or the fourth peptidyl linker independently include at least 14 glycines. In embodiments, the first peptidyl linker, the second peptidyl linker, the third peptidyl linker, and/or the fourth peptidyl linker independently include at least 15 glycines.

In embodiments, the first peptidyl linker, the second peptidyl linker, the third peptidyl linker, and/or the fourth peptidyl linker independently include at least 1 serine. In embodiments, the first peptidyl linker, the second peptidyl linker, the third peptidyl linker, and/or the fourth peptidyl linker independently include at least 2 serines. In embodiments, the first peptidyl linker, the second peptidyl linker, the third peptidyl linker, and/or the fourth peptidyl linker independently include at least 3 serines. In embodiments, the first peptidyl linker, the second peptidyl linker, the third peptidyl linker, and/or the fourth peptidyl linker independently include at least 4 serines. In embodiments, the first peptidyl linker, the second peptidyl linker, the third peptidyl linker, and/or the fourth peptidyl linker independently include at least 5 serines. In embodiments, the first peptidyl linker, the second peptidyl linker, the third peptidyl linker, and/or the fourth peptidyl linker independently include at least 6 serines. In embodiments, the first peptidyl linker, the second peptidyl linker, the third peptidyl linker, and/or the fourth peptidyl linker independently include at least 7 serines. In embodiments, the first peptidyl linker, the second peptidyl linker, the third peptidyl linker, and/or the fourth peptidyl linker independently include at least 8 serines. In embodiments, the first peptidyl linker, the second peptidyl linker, the third peptidyl linker, and/or the fourth peptidyl linker independently include at least 9 serines. In embodiments, the first peptidyl linker, the second peptidyl linker, the third peptidyl linker, and/or the fourth peptidyl linker independently include at least 10 serines. In embodiments, the first peptidyl linker, the second peptidyl linker, the third peptidyl linker, and/or the fourth peptidyl linker independently include at least 11 serines. In embodiments, the first peptidyl linker, the second peptidyl linker, the third peptidyl linker, and/or the fourth peptidyl linker independently include at least 12 serines. In embodiments, the first peptidyl linker, the second peptidyl linker, the third peptidyl linker, and/or the fourth peptidyl linker independently include at least 13 serines. In embodiments, the first peptidyl linker, the second peptidyl linker, the third peptidyl linker, and/or the fourth peptidyl linker independently include at least 14 serines. In embodiments, the first peptidyl linker, the second peptidyl linker, the third peptidyl linker, and/or the fourth peptidyl linker independently include at least 15 serines.

In embodiments, the first peptidyl linker, the second peptidyl linker, the third peptidyl linker, and/or the fourth peptidyl linker independently include at least 1 threonine. In embodiments, the first peptidyl linker, the second peptidyl linker, the third peptidyl linker, and/or the fourth peptidyl linker independently include at least 2 threonines. In embodiments, the first peptidyl linker, the second peptidyl linker, the third peptidyl linker, and/or the fourth peptidyl linker independently include at least 3 threonines. In embodiments, the first peptidyl linker, the second peptidyl linker, the third peptidyl linker, and/or the fourth peptidyl linker independently include at least 4 threonines. In embodiments, the first peptidyl linker, the second peptidyl linker, the third peptidyl linker, and/or the fourth peptidyl linker independently include at least 5 threonines. In embodiments, the first peptidyl linker, the second peptidyl linker, the third peptidyl linker, and/or the fourth peptidyl linker independently include at least 6 threonines. In embodiments, the first peptidyl linker, the second peptidyl linker, the third peptidyl linker, and/or the fourth peptidyl linker independently include at least 7 threonines. In embodiments, the first peptidyl linker, the second peptidyl linker, the third peptidyl linker, and/or the fourth peptidyl linker independently include at least 8 threonines. In embodiments, the first peptidyl linker, the second peptidyl linker, the third peptidyl linker, and/or the fourth peptidyl linker independently include at least 9 threonines. In embodiments, the first peptidyl linker, the second peptidyl linker, the third peptidyl linker, and/or the fourth peptidyl linker independently include at least 10 threonines. In embodiments, the first peptidyl linker, the second peptidyl linker, the third peptidyl linker, and/or the fourth peptidyl linker independently include at least 11 threonines. In embodiments, the first peptidyl linker, the second peptidyl linker, the third peptidyl linker, and/or the fourth peptidyl linker independently include at least 12 threonines. In embodiments, the first peptidyl linker, the second peptidyl linker, the third peptidyl linker, and/or the fourth peptidyl linker independently include at least 13 threonines. In embodiments, the first peptidyl linker, the second peptidyl linker, the third peptidyl linker, and/or the fourth peptidyl linker independently include at least 14 threonines. In embodiments, the first peptidyl linker, the second peptidyl linker, the third peptidyl linker, and/or the fourth peptidyl linker independently include at least 15 threonines.

In embodiments, the first peptidyl linker, the second peptidyl linker, the third peptidyl linker, or the fourth peptidyl linker independently includes at least 1 glycine, serine, or threonine. In embodiments, the first peptidyl linker, the second peptidyl linker, the third peptidyl linker, and the fourth peptidyl linker independently include at least 1 glycine, serine, or threonine. In embodiments, the first peptidyl linker, the second peptidyl linker, the third peptidyl linker, and the fourth peptidyl linker independently include at least 1 glycine. In embodiments, the first peptidyl linker, the second peptidyl linker, the third peptidyl linker, and the fourth peptidyl linker independently include at least 1 serine. In embodiments, the first peptidyl linker, the second peptidyl linker, the third peptidyl linker, and the fourth peptidyl linker independently include at least 1 threonine.

In embodiments, the first peptidyl linker includes at least 1 glycine, serine, or threonine. In embodiments, the first peptidyl linker includes at least 2 glycines, serines, or threonines. In embodiments, the first peptidyl linker includes at least 3 glycines, serines, or threonines. In embodiments, the first peptidyl linker includes at least 4 glycines, serines, or threonines. In embodiments, the first peptidyl linker includes at least 5 glycines, serines, or threonines. In embodiments, the first peptidyl linker includes at least 6 glycines, serines, or threonines. In embodiments, the first peptidyl linker includes at least 7 glycines, serines, or threonines. In embodiments, the first peptidyl linker includes at least 8 glycines, serines, or threonines. In embodiments, the first peptidyl linker includes at least 9 glycines, serines, or threonines. In embodiments, the first peptidyl linker includes at least 10 glycines, serines, or threonines. In embodiments, the first peptidyl linker includes at least 11 glycines, serines, or threonines. In embodiments, the first peptidyl linker includes at least 12 glycines, serines, or threonines. In embodiments, the first peptidyl linker includes at least 13 glycines, serines, or threonines. In embodiments, the first peptidyl linker includes at least 14 glycines, serines, or threonines. In embodiments, the first peptidyl linker includes at least 15 glycines, serines, or threonines.

In embodiments, the first peptidyl linker includes at least 1 glycine. In embodiments, the first peptidyl linker includes at least 2 glycines. In embodiments, the first peptidyl linker includes at least 3 glycines. In embodiments, the first peptidyl linker includes at least 4 glycines. In embodiments, the first peptidyl linker includes at least 5 glycines. In embodiments, the first peptidyl linker includes at least 6 glycines. In embodiments, the first peptidyl linker includes at least 7 glycines. In embodiments, the first peptidyl linker includes at least 8 glycines, serines, or threonines. In embodiments, the first peptidyl linker includes at least 9 glycines. In embodiments, the first peptidyl linker includes at least 10 glycines. In embodiments, the first peptidyl linker includes at least 11 glycines. In embodiments, the first peptidyl linker includes at least 12 glycines. In embodiments, the first peptidyl linker includes at least 13 glycines. In embodiments, the first peptidyl linker includes at least 14 glycines. In embodiments, the first peptidyl linker includes at least 15 glycines.

In embodiments, the first peptidyl linker includes at least 1 serine. In embodiments, the first peptidyl linker includes at least 2 serines. In embodiments, the first peptidyl linker includes at least 3 serines. In embodiments, the first peptidyl linker includes at least 4 serines. In embodiments, the first peptidyl linker includes at least 5 serines. In embodiments, the first peptidyl linker includes at least 6 serines. In embodiments, the first peptidyl linker includes at least 7 serines. In embodiments, the first peptidyl linker includes at least 8 serines, serines, or threonines. In embodiments, the first peptidyl linker includes at least 9 serines. In embodiments, the first peptidyl linker includes at least 10 serines. In embodiments, the first peptidyl linker includes at least 11 serines. In embodiments, the first peptidyl linker includes at least 12 serines.

In embodiments, the first peptidyl linker includes at least 13 serines. In embodiments, the first peptidyl linker includes at least 14 serines. In embodiments, the first peptidyl linker includes at least 15 serines. In embodiments, the first peptidyl linker includes at least 1 threonine. In embodiments, the first peptidyl linker includes at least 2 threonines. In embodiments, the first peptidyl linker includes at least 3 threonines. In embodiments, the first peptidyl linker includes at least 4 threonines. In embodiments, the first peptidyl linker includes at least 5 threonines. In embodiments, the first peptidyl linker includes at least 6 threonines. In embodiments, the first peptidyl linker includes at least 7 threonines. In embodiments, the first peptidyl linker includes at least 8 threonines, serines, or threonines. In embodiments, the first peptidyl linker includes at least 9 threonines. In embodiments, the first peptidyl linker includes at least 10 threonines. In embodiments, the first peptidyl linker includes at least 11 threonines. In embodiments, the first peptidyl linker includes at least 12 threonines. In embodiments, the first peptidyl linker includes at least 13 threonines. In embodiments, the first peptidyl linker includes at least 14 threonines. In embodiments, the first peptidyl linker includes at least 15 threonines.

In embodiments, the second peptidyl linker includes at least 1 glycine, serine, or threonine. In embodiments, the second peptidyl linker includes at least 2 glycines, serines, or threonines. In embodiments, the second peptidyl linker includes at least 3 glycines, serines, or threonines. In embodiments, the second peptidyl linker includes at least 4 glycines, serines, or threonines. In embodiments, the second peptidyl linker includes at least 5 glycines, serines, or threonines. In embodiments, the second peptidyl linker includes at least 6 glycines, serines, or threonines. In embodiments, the second peptidyl linker includes at least 7 glycines, serines, or threonines. In embodiments, the second peptidyl linker includes at least 8 glycines, serines, or threonines. In embodiments, the second peptidyl linker includes at least 9 glycines, serines, or threonines. In embodiments, the second peptidyl linker includes at least 10 glycines, serines, or threonines. In embodiments, the second peptidyl linker includes at least 11 glycines, serines, or threonines. In embodiments, the second peptidyl linker includes at least 12 glycines, serines, or threonines. In embodiments, the second peptidyl linker includes at least 13 glycines, serines, or threonines. In embodiments, the second peptidyl linker includes at least 14 glycines, serines, or threonines. In embodiments, the second peptidyl linker includes at least 15 glycines, serines, or threonines.

In embodiments, the second peptidyl linker includes at least 1 glycine. In embodiments, the second peptidyl linker includes at least 2 glycines. In embodiments, the second peptidyl linker includes at least 3 glycines. In embodiments, the second peptidyl linker includes at least 4 glycines. In embodiments, the second peptidyl linker includes at least 5 glycines. In embodiments, the second peptidyl linker includes at least 6 glycines. In embodiments, the second peptidyl linker includes at least 7 glycines. In embodiments, the second peptidyl linker includes at least 8 glycines, serines, or threonines. In embodiments, the second peptidyl linker includes at least 9 glycines. In embodiments, the second peptidyl linker includes at least 10 glycines. In embodiments, the second peptidyl linker includes at least 11 glycines. In embodiments, the second peptidyl linker includes at least 12 glycines. In embodiments, the second peptidyl linker includes at least 13 glycines. In embodiments, the second peptidyl linker includes at least 14 glycines. In embodiments, the second peptidyl linker includes at least 15 glycines.

In embodiments, the second peptidyl linker includes at least 1 serine. In embodiments, the second peptidyl linker includes at least 2 serines. In embodiments, the second peptidyl linker includes at least 3 serines. In embodiments, the second peptidyl linker includes at least 4 serines. In embodiments, the second peptidyl linker includes at least 5 serines. In embodiments, the second peptidyl linker includes at least 6 serines. In embodiments, the second peptidyl linker includes at least 7 serines. In embodiments, the second peptidyl linker includes at least 8 serines, serines, or threonines. In embodiments, the second peptidyl linker includes at least 9 serines. In embodiments, the second peptidyl linker includes at least 10 serines. In embodiments, the second peptidyl linker includes at least 11 serines. In embodiments, the second peptidyl linker includes at least 12 serines. In embodiments, the second peptidyl linker includes at least 13 serines. In embodiments, the second peptidyl linker includes at least 14 serines. In embodiments, the second peptidyl linker includes at least 15 serines.

In embodiments, the second peptidyl linker includes at least 1 threonine. In embodiments, the second peptidyl linker includes at least 2 threonines. In embodiments, the second peptidyl linker includes at least 3 threonines. In embodiments, the second peptidyl linker includes at least 4 threonines. In embodiments, the second peptidyl linker includes at least 5 threonines. In embodiments, the second peptidyl linker includes at least 6 threonines. In embodiments, the second peptidyl linker includes at least 7 threonines. In embodiments, the second peptidyl linker includes at least 8 threonines, serines, or threonines. In embodiments, the second peptidyl linker includes at least 9 threonines. In embodiments, the second peptidyl linker includes at least 10 threonines. In embodiments, the second peptidyl linker includes at least 11 threonines. In embodiments, the second peptidyl linker includes at least 12 threonines. In embodiments, the second peptidyl linker includes at least 13 threonines. In embodiments, the second peptidyl linker includes at least 14 threonines. In embodiments, the second peptidyl linker includes at least 15 threonines.

In embodiments, the third peptidyl linker includes at least 1 glycine, serine, or threonine. In embodiments, the third peptidyl linker includes at least 2 glycines, serines, or threonines. In embodiments, the third peptidyl linker includes at least 3 glycines, serines, or threonines. In embodiments, the third peptidyl linker includes at least 4 glycines, serines, or threonines. In embodiments, the third peptidyl linker includes at least 5 glycines, serines, or threonines. In embodiments, the third peptidyl linker includes at least 6 glycines, serines, or threonines. In embodiments, the third peptidyl linker includes at least 7 glycines, serines, or threonines. In embodiments, the third peptidyl linker includes at least 8 glycines, serines, or threonines. In embodiments, the third peptidyl linker includes at least 9 glycines, serines, or threonines. In embodiments, the third peptidyl linker includes at least 10 glycines, serines, or threonines. In embodiments, the third peptidyl linker includes at least 11 glycines, serines, or threonines. In embodiments, the third peptidyl linker includes at least 12 glycines, serines, or threonines. In embodiments, the third peptidyl linker includes at least 13 glycines, serines, or threonines. In embodiments, the third peptidyl linker includes at least 14 glycines, serines, or threonines. In embodiments, the third peptidyl linker includes at least 15 glycines, serines, or threonines.

In embodiments, the third peptidyl linker includes at least 1 glycine. In embodiments, the third peptidyl linker includes at least 2 glycines. In embodiments, the third peptidyl linker includes at least 3 glycines. In embodiments, the third peptidyl linker includes at least 4 glycines. In embodiments, the third peptidyl linker includes at least 5 glycines. In embodiments, the third peptidyl linker includes at least 6 glycines. In embodiments, the third peptidyl linker includes at least 7 glycines. In embodiments, the third peptidyl linker includes at least 8 glycines, serines, or threonines. In embodiments, the third peptidyl linker includes at least 9 glycines. In embodiments, the third peptidyl linker includes at least 10 glycines. In embodiments, the third peptidyl linker includes at least 11 glycines. In embodiments, the third peptidyl linker includes at least 12 glycines. In embodiments, the third peptidyl linker includes at least 13 glycines. In embodiments, the third peptidyl linker includes at least 14 glycines. In embodiments, the third peptidyl linker includes at least 15 glycines.

In embodiments, the third peptidyl linker includes at least 1 serine. In embodiments, the third peptidyl linker includes at least 2 serines. In embodiments, the third peptidyl linker includes at least 3 serines. In embodiments, the third peptidyl linker includes at least 4 serines. In embodiments, the third peptidyl linker includes at least 5 serines. In embodiments, the third peptidyl linker includes at least 6 serines. In embodiments, the third peptidyl linker includes at least 7 serines. In embodiments, the third peptidyl linker includes at least 8 serines, serines, or threonines. In embodiments, the third peptidyl linker includes at least 9 serines. In embodiments, the third peptidyl linker includes at least 10 serines. In embodiments, the third peptidyl linker includes at least 11 serines. In embodiments, the third peptidyl linker includes at least 12 serines. In embodiments, the third peptidyl linker includes at least 13 serines. In embodiments, the third peptidyl linker includes at least 14 serines. In embodiments, the third peptidyl linker includes at least 15 serines.

In embodiments, the third peptidyl linker includes at least 1 threonine. In embodiments, the third peptidyl linker includes at least 2 threonines. In embodiments, the third peptidyl linker includes at least 3 threonines. In embodiments, the third peptidyl linker includes at least 4 threonines. In embodiments, the third peptidyl linker includes at least 5 threonines. In embodiments, the third peptidyl linker includes at least 6 threonines. In embodiments, the third peptidyl linker includes at least 7 threonines. In embodiments, the third peptidyl linker includes at least 8 threonines, serines, or threonines. In embodiments, the third peptidyl linker includes at least 9 threonines. In embodiments, the third peptidyl linker includes at least 10 threonines. In embodiments, the third peptidyl linker includes at least 11 threonines. In embodiments, the third peptidyl linker includes at least 12 threonines. In embodiments, the third peptidyl linker includes at least 13 threonines. In embodiments, the third peptidyl linker includes at least 14 threonines. In embodiments, the third peptidyl linker includes at least 15 threonines.

In embodiments, the fourth peptidyl linker includes at least 1 glycine, serine, or threonine. In embodiments, the fourth peptidyl linker includes at least 2 glycines, serines, or threonines. In embodiments, the fourth peptidyl linker includes at least 3 glycines, serines, or threonines. In embodiments, the fourth peptidyl linker includes at least 4 glycines, serines, or threonines. In embodiments, the fourth peptidyl linker includes at least 5 glycines, serines, or threonines. In embodiments, the fourth peptidyl linker includes at least 6 glycines, serines, or threonines. In embodiments, the fourth peptidyl linker includes at least 7 glycines, serines, or threonines. In embodiments, the fourth peptidyl linker includes at least 8 glycines, serines, or threonines. In embodiments, the fourth peptidyl linker includes at least 9 glycines, serines, or threonines. In embodiments, the fourth peptidyl linker includes at least 10 glycines, serines, or threonines. In embodiments, the fourth peptidyl linker includes at least 11 glycines, serines, or threonines. In embodiments, the fourth peptidyl linker includes at least 12 glycines, serines, or threonines. In embodiments, the fourth peptidyl linker includes at least 13 glycines, serines, or threonines. In embodiments, the fourth peptidyl linker includes at least 14 glycines, serines, or threonines. In embodiments, the fourth peptidyl linker includes at least 15 glycines, serines, or threonines.

In embodiments, the fourth peptidyl linker includes at least 1 glycine. In embodiments, the fourth peptidyl linker includes at least 2 glycines. In embodiments, the fourth peptidyl linker includes at least 3 glycines. In embodiments, the fourth peptidyl linker includes at least 4 glycines. In embodiments, the fourth peptidyl linker includes at least 5 glycines. In embodiments, the fourth peptidyl linker includes at least 6 glycines. In embodiments, the fourth peptidyl linker includes at least 7 glycines. In embodiments, the fourth peptidyl linker includes at least 8 glycines, serines, or threonines. In embodiments, the fourth peptidyl linker includes at least 9 glycines. In embodiments, the fourth peptidyl linker includes at least 10 glycines. In embodiments, the fourth peptidyl linker includes at least 11 glycines. In embodiments, the fourth peptidyl linker includes at least 12 glycines. In embodiments, the fourth peptidyl linker includes at least 13 glycines. In embodiments, the fourth peptidyl linker includes at least 14 glycines. In embodiments, the fourth peptidyl linker includes at least 15 glycines.

In embodiments, the fourth peptidyl linker includes at least 1 serine. In embodiments, the fourth peptidyl linker includes at least 2 serines. In embodiments, the fourth peptidyl linker includes at least 3 serines. In embodiments, the fourth peptidyl linker includes at least 4 serines. In embodiments, the fourth peptidyl linker includes at least 5 serines. In embodiments, the fourth peptidyl linker includes at least 6 serines. In embodiments, the fourth peptidyl linker includes at least 7 serines. In embodiments, the fourth peptidyl linker includes at least 8 serines, serines, or threonines. In embodiments, the fourth peptidyl linker includes at least 9 serines. In embodiments, the fourth peptidyl linker includes at least 10 serines. In embodiments, the fourth peptidyl linker includes at least 11 serines. In embodiments, the fourth peptidyl linker includes at least 12 serines. In embodiments, the fourth peptidyl linker includes at least 13 serines. In embodiments, the fourth peptidyl linker includes at least 14 serines. In embodiments, the fourth peptidyl linker includes at least 15 serines.

In embodiments, the fourth peptidyl linker includes at least 1 threonine. In embodiments, the fourth peptidyl linker includes at least 2 threonines. In embodiments, the fourth peptidyl linker includes at least 3 threonines. In embodiments, the fourth peptidyl linker includes at least 4 threonines. In embodiments, the fourth peptidyl linker includes at least 5 threonines. In embodiments, the fourth peptidyl linker includes at least 6 threonines. In embodiments, the fourth peptidyl linker includes at least 7 threonines. In embodiments, the fourth peptidyl linker includes at least 8 threonines, serines, or threonines. In embodiments, the fourth peptidyl linker includes at least 9 threonines. In embodiments, the fourth peptidyl linker includes at least 10 threonines. In embodiments, the fourth peptidyl linker includes at least 11 threonines. In embodiments, the fourth peptidyl linker includes at least 12 threonines. In embodiments, the fourth peptidyl linker includes at least 13 threonines. In embodiments, the fourth peptidyl linker includes at least 14 threonines. In embodiments, the fourth peptidyl linker includes at least 15 threonines.

In embodiments, at least 60% of the amino acids of the first peptidyl linker, the second peptidyl linker, the third peptidyl linker, and/or the fourth peptidyl linker include glycine. In embodiments, at least 65% of the amino acids of the first peptidyl linker, the second peptidyl linker, the third peptidyl linker, and/or the fourth peptidyl linker include glycine. In embodiments, at least 70% of the amino acids of the first peptidyl linker, the second peptidyl linker, the third peptidyl linker, and/or the fourth peptidyl linker include glycine. In embodiments, at least 75% of the amino acids of the first peptidyl linker, the second peptidyl linker, the third peptidyl linker, and/or the fourth peptidyl linker include glycine. In embodiments, at least 80% of the amino acids of the first peptidyl linker, the second peptidyl linker, the third peptidyl linker, and/or the fourth peptidyl linker include glycine. In embodiments, at least 85% of the amino acids of the first peptidyl linker, the second peptidyl linker, the third peptidyl linker, and/or the fourth peptidyl linker include glycine. In embodiments, at least 90% of the amino acids of the first peptidyl linker, the second peptidyl linker, the third peptidyl linker, and/or the fourth peptidyl linker include glycine. In embodiments, at least 95% of the amino acids of the first peptidyl linker, the second peptidyl linker, the third peptidyl linker, and/or the fourth peptidyl linker include glycine. In embodiments, at least 96% of the amino acids of the first peptidyl linker, the second peptidyl linker, the third peptidyl linker, and/or the fourth peptidyl linker include glycine. In embodiments, at least 97% of the amino acids of the first peptidyl linker, the second peptidyl linker, the third peptidyl linker, and/or the fourth peptidyl linker include glycine. In embodiments, at least 98% of the amino acids of the first peptidyl linker, the second peptidyl linker, the third peptidyl linker, and/or the fourth peptidyl linker include glycine. In embodiments, at least 99% of the amino acids of the first peptidyl linker, the second peptidyl linker, the third peptidyl linker, and/or the fourth peptidyl linker include glycine. In embodiments, at least 100% of the amino acids of the first peptidyl linker, the second peptidyl linker, the third peptidyl linker, and/or the fourth peptidyl linker include glycine.

In embodiments, at least 60% of the amino acids of the first peptidyl linker, the second peptidyl linker, the third peptidyl linker, or the fourth peptidyl linker include glycine. In embodiments, at least 65% of the amino acids of the first peptidyl linker, the second peptidyl linker, the third peptidyl linker, or the fourth peptidyl linker include glycine. In embodiments, at least 70% of the amino acids of the first peptidyl linker, the second peptidyl linker, the third peptidyl linker, or the fourth peptidyl linker include glycine. In embodiments, at least 75% of the amino acids of the first peptidyl linker, the second peptidyl linker, the third peptidyl linker, or the fourth peptidyl linker include glycine. In embodiments, at least 80% of the amino acids of the first peptidyl linker, the second peptidyl linker, the third peptidyl linker, or the fourth peptidyl linker include glycine. In embodiments, at least 85% of the amino acids of the first peptidyl linker, the second peptidyl linker, the third peptidyl linker, or the fourth peptidyl linker include glycine. In embodiments, at least 90% of the amino acids of the first peptidyl linker, the second peptidyl linker, the third peptidyl linker, or the fourth peptidyl linker include glycine. In embodiments, at least 95% of the amino acids of the first peptidyl linker, the second peptidyl linker, the third peptidyl linker, or the fourth peptidyl linker include glycine. In embodiments, at least 96% of the amino acids of the first peptidyl linker, the second peptidyl linker, the third peptidyl linker, or the fourth peptidyl linker include glycine. In embodiments, at least 97% of the amino acids of the first peptidyl linker, the second peptidyl linker, the third peptidyl linker, or the fourth peptidyl linker include glycine. In embodiments, at least 98% of the amino acids of the first peptidyl linker, the second peptidyl linker, the third peptidyl linker, or the fourth peptidyl linker include glycine. In embodiments, at least 99% of the amino acids of the first peptidyl linker, the second peptidyl linker, the third peptidyl linker, or the fourth peptidyl linker include glycine. In embodiments, at least 100% of the amino acids of the first peptidyl linker, the second peptidyl linker, the third peptidyl linker, or the fourth peptidyl linker include glycine.

In embodiments, at least 60% of the amino acids of the first peptidyl linker, the second peptidyl linker, the third peptidyl linker, and the fourth peptidyl linker include glycine. In embodiments, at least 65% of the amino acids of the first peptidyl linker, the second peptidyl linker, the third peptidyl linker, and the fourth peptidyl linker include glycine. In embodiments, at least 70% of the amino acids of the first peptidyl linker, the second peptidyl linker, the third peptidyl linker, and the fourth peptidyl linker include glycine. In embodiments, at least 75% of the amino acids of the first peptidyl linker, the second peptidyl linker, the third peptidyl linker, and the fourth peptidyl linker include glycine. In embodiments, at least 80% of the amino acids of the first peptidyl linker, the second peptidyl linker, the third peptidyl linker, and the fourth peptidyl linker include glycine. In embodiments, at least 85% of the amino acids of the first peptidyl linker, the second peptidyl linker, the third peptidyl linker, and the fourth peptidyl linker include glycine. In embodiments, at least 90% of the amino acids of the first peptidyl linker, the second peptidyl linker, the third peptidyl linker, and the fourth peptidyl linker include glycine. In embodiments, at least 95% of the amino acids of the first peptidyl linker, the second peptidyl linker, the third peptidyl linker, and the fourth peptidyl linker include glycine. In embodiments, at least 96% of the amino acids of the first peptidyl linker, the second peptidyl linker, the third peptidyl linker, and the fourth peptidyl linker include glycine. In embodiments, at least 97% of the amino acids of the first peptidyl linker, the second peptidyl linker, the third peptidyl linker, and the fourth peptidyl linker include glycine. In embodiments, at least 98% of the amino acids of the first peptidyl linker, the second peptidyl linker, the third peptidyl linker, and the fourth peptidyl linker include glycine. In embodiments, at least 99% of the amino acids of the first peptidyl linker, the second peptidyl linker, the third peptidyl linker, and the fourth peptidyl linker include glycine. In embodiments, at least 100% of the amino acids of the first peptidyl linker, the second peptidyl linker, the third peptidyl linker, and the fourth peptidyl linker include glycine.

In embodiments, at least 60% of the amino acids of the first peptidyl linker include glycine. In embodiments, at least 65% of the amino acids of the first peptidyl linker include glycine. In embodiments, at least 70% of the amino acids of the first peptidyl linker include glycine. In embodiments, at least 75% of the amino acids of the first peptidyl linker include glycine. In embodiments, at least 80% of the amino acids of the first peptidyl linker include glycine. In embodiments, at least 85% of the amino acids of the first peptidyl linker include glycine. In embodiments, at least 90% of the amino acids of the first peptidyl linker include glycine. In embodiments, at least 95% of the amino acids of the first peptidyl linker include glycine. In embodiments, at least 96% of the amino acids of the first peptidyl linker include glycine. In embodiments, at least 97% of the amino acids of the first peptidyl linker include glycine. In embodiments, at least 98% of the amino acids of the first peptidyl linker include glycine. In embodiments, at least 99% of the amino acids of the first peptidyl linker include glycine. In embodiments, at least 100% of the amino acids of the first peptidyl linker include glycine.

In embodiments, at least 60% of the amino acids of the second peptidyl linker include glycine. In embodiments, at least 65% of the amino acids of the second peptidyl linker include glycine. In embodiments, at least 70% of the amino acids of the second peptidyl linker include glycine. In embodiments, at least 75% of the amino acids of the second peptidyl linker include glycine. In embodiments, at least 80% of the amino acids of the second peptidyl linker include glycine. In embodiments, at least 85% of the amino acids of the second peptidyl linker include glycine. In embodiments, at least 90% of the amino acids of the second peptidyl linker include glycine. In embodiments, at least 95% of the amino acids of the second peptidyl linker include glycine. In embodiments, at least 96% of the amino acids of the second peptidyl linker include glycine. In embodiments, at least 97% of the amino acids of the second peptidyl linker include glycine. In embodiments, at least 98% of the amino acids of the second peptidyl linker include glycine. In embodiments, at least 99% of the amino acids of the second peptidyl linker include glycine. In embodiments, at least 100% of the amino acids of the second peptidyl linker include glycine.

In embodiments, at least 60% of the amino acids of the third peptidyl linker include glycine. In embodiments, at least 65% of the amino acids of the third peptidyl linker include glycine. In embodiments, at least 70% of the amino acids of the third peptidyl linker include glycine. In embodiments, at least 75% of the amino acids of the third peptidyl linker include glycine. In embodiments, at least 80% of the amino acids of the third peptidyl linker include glycine. In embodiments, at least 85% of the amino acids of the third peptidyl linker include glycine. In embodiments, at least 90% of the amino acids of the third peptidyl linker include glycine. In embodiments, at least 95% of the amino acids of the third peptidyl linker include glycine. In embodiments, at least 96% of the amino acids of the third peptidyl linker include glycine. In embodiments, at least 97% of the amino acids of the third peptidyl linker include glycine. In embodiments, at least 98% of the amino acids of the third peptidyl linker include glycine. In embodiments, at least 99% of the amino acids of the third peptidyl linker include glycine. In embodiments, at least 100% of the amino acids of the third peptidyl linker include glycine.

In embodiments, at least 60% of the amino acids of the fourth peptidyl linker include glycine. In embodiments, at least 65% of the amino acids of the fourth peptidyl linker include glycine. In embodiments, at least 70% of the amino acids of the fourth peptidyl linker include glycine. In embodiments, at least 75% of the amino acids of the fourth peptidyl linker include glycine. In embodiments, at least 80% of the amino acids of the fourth peptidyl linker include glycine. In embodiments, at least 85% of the amino acids of the fourth peptidyl linker include glycine. In embodiments, at least 90% of the amino acids of the fourth peptidyl linker include glycine. In embodiments, at least 95% of the amino acids of the fourth peptidyl linker include glycine. In embodiments, at least 96% of the amino acids of the fourth peptidyl linker include glycine. In embodiments, at least 97% of the amino acids of the fourth peptidyl linker include glycine. In embodiments, at least 98% of the amino acids of the fourth peptidyl linker include glycine. In embodiments, at least 99% of the amino acids of the fourth peptidyl linker include glycine. In embodiments, at least 100% of the amino acids of the fourth peptidyl linker include glycine.

In embodiments, at least 20% of the amino acids of the first peptidyl linker, the second peptidyl linker, the third peptidyl linker, and/or the fourth peptidyl linker include serine. In embodiments, at least 30% of the amino acids of the first peptidyl linker, the second peptidyl linker, the third peptidyl linker, and/or the fourth peptidyl linker include serine. In embodiments, at least 40% of the amino acids of the first peptidyl linker, the second peptidyl linker, the third peptidyl linker, and/or the fourth peptidyl linker include serine. In embodiments, at least 50% of the amino acids of the first peptidyl linker, the second peptidyl linker, the third peptidyl linker, and/or the fourth peptidyl linker include serine. In embodiments, at least 60% of the amino acids of the first peptidyl linker, the second peptidyl linker, the third peptidyl linker, and/or the fourth peptidyl linker include serine. In embodiments, at least 70% of the amino acids of the first peptidyl linker, the second peptidyl linker, the third peptidyl linker, and/or the fourth peptidyl linker include serine. In embodiments, at least 80% of the amino acids of the first peptidyl linker, the second peptidyl linker, the third peptidyl linker, and/or the fourth peptidyl linker include serine. In embodiments, at least 90% of the amino acids of the first peptidyl linker, the second peptidyl linker, the third peptidyl linker, and/or the fourth peptidyl linker include serine. In embodiments, at least 95% of the amino acids of the first peptidyl linker, the second peptidyl linker, the third peptidyl linker, and/or the fourth peptidyl linker include serine. In embodiments, at least 96% of the amino acids of the first peptidyl linker, the second peptidyl linker, the third peptidyl linker, and/or the fourth peptidyl linker include serine. In embodiments, at least 97% of the amino acids of the first peptidyl linker, the second peptidyl linker, the third peptidyl linker, and/or the fourth peptidyl linker include serine. In embodiments, at least 98% of the amino acids of the first peptidyl linker, the second peptidyl linker, the third peptidyl linker, and/or the fourth peptidyl linker include serine. In embodiments, at least 99% of the amino acids of the first peptidyl linker, the second peptidyl linker, the third peptidyl linker, and/or the fourth peptidyl linker include serine. In embodiments, at least 100% of the amino acids of the first peptidyl linker, the second peptidyl linker, the third peptidyl linker, and/or the fourth peptidyl linker include serine.

In embodiments, at least 20% of the amino acids of the first peptidyl linker, the second peptidyl linker, the third peptidyl linker, or the fourth peptidyl linker include serine. In embodiments, at least 30% of the amino acids of the first peptidyl linker, the second peptidyl linker, the third peptidyl linker, or the fourth peptidyl linker include serine. In embodiments, at least 40% of the amino acids of the first peptidyl linker, the second peptidyl linker, the third peptidyl linker, or the fourth peptidyl linker include serine. In embodiments, at least 50% of the amino acids of the first peptidyl linker, the second peptidyl linker, the third peptidyl linker, or the fourth peptidyl linker include serine. In embodiments, at least 60% of the amino acids of the first peptidyl linker, the second peptidyl linker, the third peptidyl linker, or the fourth peptidyl linker include serine. In embodiments, at least 70% of the amino acids of the first peptidyl linker, the second peptidyl linker, the third peptidyl linker, or the fourth peptidyl linker include serine. In embodiments, at least 80% of the amino acids of the first peptidyl linker, the second peptidyl linker, the third peptidyl linker, or the fourth peptidyl linker include serine. In embodiments, at least 90% of the amino acids of the first peptidyl linker, the second peptidyl linker, the third peptidyl linker, or the fourth peptidyl linker include serine. In embodiments, at least 95% of the amino acids of the first peptidyl linker, the second peptidyl linker, the third peptidyl linker, or the fourth peptidyl linker include serine. In embodiments, at least 96% of the amino acids of the first peptidyl linker, the second peptidyl linker, the third peptidyl linker, or the fourth peptidyl linker include serine. In embodiments, at least 97% of the amino acids of the first peptidyl linker, the second peptidyl linker, the third peptidyl linker, or the fourth peptidyl linker include serine. In embodiments, at least 98% of the amino acids of the first peptidyl linker, the second peptidyl linker, the third peptidyl linker, or the fourth peptidyl linker include serine. In embodiments, at least 99% of the amino acids of the first peptidyl linker, the second peptidyl linker, the third peptidyl linker, or the fourth peptidyl linker include serine. In embodiments, at least 100% of the amino acids of the first peptidyl linker, the second peptidyl linker, the third peptidyl linker, or the fourth peptidyl linker include serine.

In embodiments, at least 20% of the amino acids of the first peptidyl linker, the second peptidyl linker, the third peptidyl linker, and the fourth peptidyl linker include serine. In embodiments, at least 30% of the amino acids of the first peptidyl linker, the second peptidyl linker, the third peptidyl linker, and the fourth peptidyl linker include serine. In embodiments, at least 40% of the amino acids of the first peptidyl linker, the second peptidyl linker, the third peptidyl linker, and the fourth peptidyl linker include serine. In embodiments, at least 50% of the amino acids of the first peptidyl linker, the second peptidyl linker, the third peptidyl linker, and the fourth peptidyl linker include serine. In embodiments, at least 60% of the amino acids of the first peptidyl linker, the second peptidyl linker, the third peptidyl linker, and the fourth peptidyl linker include serine. In embodiments, at least 70% of the amino acids of the first peptidyl linker, the second peptidyl linker, the third peptidyl linker, and the fourth peptidyl linker include serine. In embodiments, at least 80% of the amino acids of the first peptidyl linker, the second peptidyl linker, the third peptidyl linker, and the fourth peptidyl linker include serine. In embodiments, at least 90% of the amino acids of the first peptidyl linker, the second peptidyl linker, the third peptidyl linker, and the fourth peptidyl linker include serine. In embodiments, at least 95% of the amino acids of the first peptidyl linker, the second peptidyl linker, the third peptidyl linker, and the fourth peptidyl linker include serine. In embodiments, at least 96% of the amino acids of the first peptidyl linker, the second peptidyl linker, the third peptidyl linker, and the fourth peptidyl linker include serine. In embodiments, at least 97% of the amino acids of the first peptidyl linker, the second peptidyl linker, the third peptidyl linker, and the fourth peptidyl linker include serine. In embodiments, at least 98% of the amino acids of the first peptidyl linker, the second peptidyl linker, the third peptidyl linker, and the fourth peptidyl linker include serine. In embodiments, at least 99% of the amino acids of the first peptidyl linker, the second peptidyl linker, the third peptidyl linker, and the fourth peptidyl linker include serine. In embodiments, at least 100% of the amino acids of the first peptidyl linker, the second peptidyl linker, the third peptidyl linker, and the fourth peptidyl linker include serine.

In embodiments, at least 20% of the amino acids of the first peptidyl linker include serine. In embodiments, at least 30% of the amino acids of the first peptidyl linker include serine. In embodiments, at least 40% of the amino acids of the first peptidyl linker include serine. In embodiments, at least 50% of the amino acids of the first peptidyl linker include serine. In embodiments, at least 60% of the amino acids of the first peptidyl linker include serine. In embodiments, at least 70% of the amino acids of the first peptidyl linker include serine. In embodiments, at least 80% of the amino acids of the first peptidyl linker include serine. In embodiments, at least 90% of the amino acids of the first peptidyl linker include serine. In embodiments, at least 95% of the amino acids of the first peptidyl linker include serine. In embodiments, at least 96% of the amino acids of the first peptidyl linker include serine. In embodiments, at least 97% of the amino acids of the first peptidyl linker include serine. In embodiments, at least 98% of the amino acids of the first peptidyl linker include serine. In embodiments, at least 99% of the amino acids of the first peptidyl linker include serine. In embodiments, at least 100% of the amino acids of the first peptidyl linker include serine.

In embodiments, at least 20% of the amino acids of the second peptidyl linker include serine. In embodiments, at least 30% of the amino acids of the second peptidyl linker include serine. In embodiments, at least 40% of the amino acids of the second peptidyl linker include serine. In embodiments, at least 50% of the amino acids of the second peptidyl linker include serine. In embodiments, at least 60% of the amino acids of the second peptidyl linker include serine. In embodiments, at least 70% of the amino acids of the second peptidyl linker include serine. In embodiments, at least 80% of the amino acids of the second peptidyl linker include serine. In embodiments, at least 90% of the amino acids of the second peptidyl linker include serine. In embodiments, at least 95% of the amino acids of the second peptidyl linker include serine. In embodiments, at least 96% of the amino acids of the second peptidyl linker include serine. In embodiments, at least 97% of the amino acids of the second peptidyl linker include serine. In embodiments, at least 98% of the amino acids of the second peptidyl linker include serine. In embodiments, at least 99% of the amino acids of the second peptidyl linker include serine. In embodiments, at least 100% of the amino acids of the second peptidyl linker include serine.

In embodiments, at least 20% of the amino acids of the third peptidyl linker include serine. In embodiments, at least 30% of the amino acids of the third peptidyl linker include serine. In embodiments, at least 40% of the amino acids of the third peptidyl linker include serine. In embodiments, at least 50% of the amino acids of the third peptidyl linker include serine. In embodiments, at least 60% of the amino acids of the third peptidyl linker include serine. In embodiments, at least 70% of the amino acids of the third peptidyl linker include serine. In embodiments, at least 80% of the amino acids of the third peptidyl linker include serine. In embodiments, at least 90% of the amino acids of the third peptidyl linker include serine. In embodiments, at least 95% of the amino acids of the third peptidyl linker include serine. In embodiments, at least 96% of the amino acids of the third peptidyl linker include serine. In embodiments, at least 97% of the amino acids of the third peptidyl linker include serine. In embodiments, at least 98% of the amino acids of the third peptidyl linker include serine. In embodiments, at least 99% of the amino acids of the third peptidyl linker include serine. In embodiments, at least 100% of the amino acids of the third peptidyl linker include serine.

In embodiments, at least 20% of the amino acids of the fourth peptidyl linker include serine. In embodiments, at least 30% of the amino acids of the fourth peptidyl linker include serine. In embodiments, at least 40% of the amino acids of the fourth peptidyl linker include serine. In embodiments, at least 50% of the amino acids of the fourth peptidyl linker include serine. In embodiments, at least 60% of the amino acids of the fourth peptidyl linker include serine. In embodiments, at least 70% of the amino acids of the fourth peptidyl linker include serine. In embodiments, at least 80% of the amino acids of the fourth peptidyl linker include serine. In embodiments, at least 90% of the amino acids of the fourth peptidyl linker include serine. In embodiments, at least 95% of the amino acids of the fourth peptidyl linker include serine. In embodiments, at least 96% of the amino acids of the fourth peptidyl linker include serine. In embodiments, at least 97% of the amino acids of the fourth peptidyl linker include serine. In embodiments, at least 98% of the amino acids of the fourth peptidyl linker include serine. In embodiments, at least 99% of the amino acids of the fourth peptidyl linker include serine. In embodiments, at least 100% of the amino acids of the fourth peptidyl linker include serine.

In embodiments, at least 10% of the amino acids of the first peptidyl linker, the second peptidyl linker, the third peptidyl linker, and/or the fourth peptidyl linker include threonine. In embodiments, at least 20% of the amino acids of the first peptidyl linker, the second peptidyl linker, the third peptidyl linker, and/or the fourth peptidyl linker include threonine. In embodiments, at least 30% of the amino acids of the first peptidyl linker, the second peptidyl linker, the third peptidyl linker, and/or the fourth peptidyl linker include threonine. In embodiments, at least 40% of the amino acids of the first peptidyl linker, the second peptidyl linker, the third peptidyl linker, and/or the fourth peptidyl linker include threonine. In embodiments, at least 50% of the amino acids of the first peptidyl linker, the second peptidyl linker, the third peptidyl linker, and/or the fourth peptidyl linker include threonine. In embodiments, at least 60% of the amino acids of the first peptidyl linker, the second peptidyl linker, the third peptidyl linker, and/or the fourth peptidyl linker include threonine. In embodiments, at least 70% of the amino acids of the first peptidyl linker, the second peptidyl linker, the third peptidyl linker, and/or the fourth peptidyl linker include threonine. In embodiments, at least 80% of the amino acids of the first peptidyl linker, the second peptidyl linker, the third peptidyl linker, and/or the fourth peptidyl linker include threonine. In embodiments, at least 90% of the amino acids of the first peptidyl linker, the second peptidyl linker, the third peptidyl linker, and/or the fourth peptidyl linker include threonine. In embodiments, at least 95% of the amino acids of the first peptidyl linker, the second peptidyl linker, the third peptidyl linker, and/or the fourth peptidyl linker include threonine. In embodiments, at least 96% of the amino acids of the first peptidyl linker, the second peptidyl linker, the third peptidyl linker, and/or the fourth peptidyl linker include threonine. In embodiments, at least 97% of the amino acids of the first peptidyl linker, the second peptidyl linker, the third peptidyl linker, and/or the fourth peptidyl linker include threonine. In embodiments, at least 98% of the amino acids of the first peptidyl linker, the second peptidyl linker, the third peptidyl linker, and/or the fourth peptidyl linker include threonine. In embodiments, at least 99% of the amino acids of the first peptidyl linker, the second peptidyl linker, the third peptidyl linker, and/or the fourth peptidyl linker include threonine. In embodiments, at least 100% of the amino acids of the first peptidyl linker, the second peptidyl linker, the third peptidyl linker, and/or the fourth peptidyl linker include threonine.

In embodiments, at least 10% of the amino acids of the first peptidyl linker, the second peptidyl linker, the third peptidyl linker, or the fourth peptidyl linker include threonine. In embodiments, at least 20% of the amino acids of the first peptidyl linker, the second peptidyl linker, the third peptidyl linker, or the fourth peptidyl linker include threonine. In embodiments, at least 30% of the amino acids of the first peptidyl linker, the second peptidyl linker, the third peptidyl linker, or the fourth peptidyl linker include threonine. In embodiments, at least 40% of the amino acids of the first peptidyl linker, the second peptidyl linker, the third peptidyl linker, or the fourth peptidyl linker include threonine. In embodiments, at least 50% of the amino acids of the first peptidyl linker, the second peptidyl linker, the third peptidyl linker, or the fourth peptidyl linker include threonine. In embodiments, at least 60% of the amino acids of the first peptidyl linker, the second peptidyl linker, the third peptidyl linker, or the fourth peptidyl linker include threonine. In embodiments, at least 70% of the amino acids of the first peptidyl linker, the second peptidyl linker, the third peptidyl linker, or the fourth peptidyl linker include threonine. In embodiments, at least 80% of the amino acids of the first peptidyl linker, the second peptidyl linker, the third peptidyl linker, or the fourth peptidyl linker include threonine. In embodiments, at least 90% of the amino acids of the first peptidyl linker, the second peptidyl linker, the third peptidyl linker, or the fourth peptidyl linker include threonine. In embodiments, at least 95% of the amino acids of the first peptidyl linker, the second peptidyl linker, the third peptidyl linker, or the fourth peptidyl linker include threonine. In embodiments, at least 96% of the amino acids of the first peptidyl linker, the second peptidyl linker, the third peptidyl linker, or the fourth peptidyl linker include threonine. In embodiments, at least 97% of the amino acids of the first peptidyl linker, the second peptidyl linker, the third peptidyl linker, or the fourth peptidyl linker include threonine. In embodiments, at least 98% of the amino acids of the first peptidyl linker, the second peptidyl linker, the third peptidyl linker, or the fourth peptidyl linker include threonine. In embodiments, at least 99% of the amino acids of the first peptidyl linker, the second peptidyl linker, the third peptidyl linker, or the fourth peptidyl linker include threonine. In embodiments, at least 100% of the amino acids of the first peptidyl linker, the second peptidyl linker, the third peptidyl linker, or the fourth peptidyl linker include threonine.

In embodiments, at least 10% of the amino acids of the first peptidyl linker, the second peptidyl linker, the third peptidyl linker, and the fourth peptidyl linker include threonine. In embodiments, at least 20% of the amino acids of the first peptidyl linker, the second peptidyl linker, the third peptidyl linker, and the fourth peptidyl linker include threonine. In embodiments, at least 30% of the amino acids of the first peptidyl linker, the second peptidyl linker, the third peptidyl linker, and the fourth peptidyl linker include threonine. In embodiments, at least 40% of the amino acids of the first peptidyl linker, the second peptidyl linker, the third peptidyl linker, and the fourth peptidyl linker include threonine. In embodiments, at least 50% of the amino acids of the first peptidyl linker, the second peptidyl linker, the third peptidyl linker, and the fourth peptidyl linker include threonine. In embodiments, at least 60% of the amino acids of the first peptidyl linker, the second peptidyl linker, the third peptidyl linker, and the fourth peptidyl linker include threonine. In embodiments, at least 70% of the amino acids of the first peptidyl linker, the second peptidyl linker, the third peptidyl linker, and the fourth peptidyl linker include threonine. In embodiments, at least 80% of the amino acids of the first peptidyl linker, the second peptidyl linker, the third peptidyl linker, and the fourth peptidyl linker include threonine. In embodiments, at least 90% of the amino acids of the first peptidyl linker, the second peptidyl linker, the third peptidyl linker, and the fourth peptidyl linker include threonine. In embodiments, at least 95% of the amino acids of the first peptidyl linker, the second peptidyl linker, the third peptidyl linker, and the fourth peptidyl linker include threonine. In embodiments, at least 96% of the amino acids of the first peptidyl linker, the second peptidyl linker, the third peptidyl linker, and the fourth peptidyl linker include threonine. In embodiments, at least 97% of the amino acids of the first peptidyl linker, the second peptidyl linker, the third peptidyl linker, and the fourth peptidyl linker include threonine. In embodiments, at least 98% of the amino acids of the first peptidyl linker, the second peptidyl linker, the third peptidyl linker, and the fourth peptidyl linker include threonine. In embodiments, at least 99% of the amino acids of the first peptidyl linker, the second peptidyl linker, the third peptidyl linker, and the fourth peptidyl linker include threonine. In embodiments, at least 100% of the amino acids of the first peptidyl linker, the second peptidyl linker, the third peptidyl linker, and the fourth peptidyl linker include threonine.

In embodiments, at least 10% of the amino acids of the first peptidyl linker include threonine. In embodiments, at least 20% of the amino acids of the first peptidyl linker include threonine. In embodiments, at least 30% of the amino acids of the first peptidyl linker include threonine. In embodiments, at least 40% of the amino acids of the first peptidyl linker include threonine. In embodiments, at least 50% of the amino acids of the first peptidyl linker include threonine. In embodiments, at least 60% of the amino acids of the first peptidyl linker include threonine. In embodiments, at least 70% of the amino acids of the first peptidyl linker include threonine. In embodiments, at least 80% of the amino acids of the first peptidyl linker include threonine. In embodiments, at least 90% of the amino acids of the first peptidyl linker include threonine. In embodiments, at least 95% of the amino acids of the first peptidyl linker include threonine. In embodiments, at least 96% of the amino acids of the first peptidyl linker include threonine. In embodiments, at least 97% of the amino acids of the first peptidyl linker include threonine. In embodiments, at least 98% of the amino acids of the first peptidyl linker include threonine. In embodiments, at least 99% of the amino acids of the first peptidyl linker include threonine. In embodiments, at least 100% of the amino acids of the first peptidyl linker include threonine.

In embodiments, at least 10% of the amino acids of the second peptidyl linker include threonine. In embodiments, at least 20% of the amino acids of the second peptidyl linker include threonine. In embodiments, at least 30% of the amino acids of the second peptidyl linker include threonine. In embodiments, at least 40% of the amino acids of the second peptidyl linker include threonine. In embodiments, at least 50% of the amino acids of the second peptidyl linker include threonine. In embodiments, at least 60% of the amino acids of the second peptidyl linker include threonine. In embodiments, at least 70% of the amino acids of the second peptidyl linker include threonine. In embodiments, at least 80% of the amino acids of the second peptidyl linker include threonine. In embodiments, at least 90% of the amino acids of the second peptidyl linker include threonine. In embodiments, at least 95% of the amino acids of the second peptidyl linker include threonine. In embodiments, at least 96% of the amino acids of the second peptidyl linker include threonine. In embodiments, at least 97% of the amino acids of the second peptidyl linker include threonine. In embodiments, at least 98% of the amino acids of the second peptidyl linker include threonine. In embodiments, at least 99% of the amino acids of the second peptidyl linker include threonine. In embodiments, at least 100% of the amino acids of the second peptidyl linker include threonine.

In embodiments, at least 10% of the amino acids of the third peptidyl linker include threonine. In embodiments, at least 20% of the amino acids of the third peptidyl linker include threonine. In embodiments, at least 30% of the amino acids of the third peptidyl linker include threonine. In embodiments, at least 40% of the amino acids of the third peptidyl linker include threonine. In embodiments, at least 50% of the amino acids of the third peptidyl linker include threonine. In embodiments, at least 60% of the amino acids of the third peptidyl linker include threonine. In embodiments, at least 70% of the amino acids of the third peptidyl linker include threonine. In embodiments, at least 80% of the amino acids of the third peptidyl linker include threonine. In embodiments, at least 90% of the amino acids of the third peptidyl linker include threonine. In embodiments, at least 95% of the amino acids of the third peptidyl linker include threonine. In embodiments, at least 96% of the amino acids of the third peptidyl linker include threonine. In embodiments, at least 97% of the amino acids of the third peptidyl linker include threonine. In embodiments, at least 98% of the amino acids of the third peptidyl linker include threonine. In embodiments, at least 99% of the amino acids of the third peptidyl linker include threonine. In embodiments, at least 100% of the amino acids of the third peptidyl linker include threonine.

In embodiments, at least 10% of the amino acids of the fourth peptidyl linker include threonine. In embodiments, at least 20% of the amino acids of the fourth peptidyl linker include threonine. In embodiments, at least 30% of the amino acids of the fourth peptidyl linker include threonine. In embodiments, at least 40% of the amino acids of the fourth peptidyl linker include threonine. In embodiments, at least 50% of the amino acids of the fourth peptidyl linker include threonine. In embodiments, at least 60% of the amino acids of the fourth peptidyl linker include threonine. In embodiments, at least 70% of the amino acids of the fourth peptidyl linker include threonine. In embodiments, at least 80% of the amino acids of the fourth peptidyl linker include threonine. In embodiments, at least 90% of the amino acids of the fourth peptidyl linker include threonine. In embodiments, at least 95% of the amino acids of the fourth peptidyl linker include threonine. In embodiments, at least 96% of the amino acids of the fourth peptidyl linker include threonine. In embodiments, at least 97% of the amino acids of the fourth peptidyl linker include threonine. In embodiments, at least 98% of the amino acids of the fourth peptidyl linker include threonine. In embodiments, at least 99% of the amino acids of the fourth peptidyl linker include threonine. In embodiments, at least 100% of the amino acids of the fourth peptidyl linker include threonine.

In embodiments, the first peptidyl linker, the second peptidyl linker, the third peptidyl linker, and/or the fourth peptidyl linker independently include the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, and/or the fourth peptidyl linker independently include the amino acid sequence of SEQ ID NO:7. In embodiments, the first peptidyl linker, the second peptidyl linker, the third peptidyl linker, and/or the fourth peptidyl linker independently include the amino acid sequence of SEQ ID NO:8. In embodiments, the first peptidyl linker, the second peptidyl linker, the third peptidyl linker, and/or the fourth peptidyl linker independently include the amino acid sequence of SEQ ID NO:9. In embodiments, the first peptidyl linker, the second peptidyl linker, the third peptidyl linker, and/or the fourth peptidyl linker independently include the amino acid sequence of SEQ ID NO:10.

In embodiments, the first peptidyl linker, the second peptidyl linker, the third peptidyl linker, and/or the fourth peptidyl linker independently are the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, and/or the fourth peptidyl linker independently are the amino acid sequence of SEQ ID NO: 7. In embodiments, the first peptidyl linker, the second peptidyl linker, the third peptidyl linker, and/or the fourth peptidyl linker independently are the amino acid sequence of SEQ ID NO:8. In embodiments, the first peptidyl linker, the second peptidyl linker, the third peptidyl linker, and/or the fourth peptidyl linker independently are the amino acid sequence of SEQ ID NO: 9. In embodiments, the first peptidyl linker, the second peptidyl linker, the third peptidyl linker, and/or the fourth peptidyl linker independently are the amino acid sequence of SEQ ID NO:10.

In embodiments, the first peptidyl linker, the second peptidyl linker, the third peptidyl linker, and the fourth peptidyl linker independently include the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, or the fourth peptidyl linker independently include the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, and the fourth peptidyl linker independently are the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, or the fourth peptidyl linker independently are the amino acid sequence of SEQ ID NO:7, SEQ ID NO:8, SEQ ID NO:9, or SEQ ID NO:10.

In embodiments, the first peptidyl linker includes the amino acid sequence of SEQ ID NO:7, SEQ ID NO:8, SEQ ID NO:9, or SEQ ID NO:10. In embodiments, the first peptidyl linker includes the amino acid sequence of SEQ ID NO:7. In embodiments, the first peptidyl linker includes the amino acid sequence of SEQ ID NO:8. In embodiments, the first peptidyl linker includes the amino acid sequence of SEQ ID NO:9. In embodiments, the first peptidyl linker includes the amino acid sequence of SEQ ID NO: 10. In embodiments, the first peptidyl linker is the amino acid sequence of SEQ ID NO:7, SEQ ID NO:8, SEQ ID NO:9, or SEQ ID NO:10. In embodiments, the first peptidyl linker is the amino acid sequence of SEQ ID NO:7. In embodiments, the first peptidyl linker is the amino acid sequence of SEQ ID NO:8. In embodiments, the first peptidyl linker is the amino acid sequence of SEQ ID NO:9. In embodiments, the first peptidyl linker is the amino acid sequence of SEQ ID NO:10.

In embodiments, the second peptidyl linker includes the amino acid sequence of SEQ ID NO:7, SEQ ID NO:8, SEQ ID NO:9, or SEQ ID NO: 10. In embodiments, the second peptidyl linker includes the amino acid sequence of SEQ ID NO:7. In embodiments, the second peptidyl linker includes the amino acid sequence of SEQ ID NO:8 In embodiments, the second peptidyl linker includes the amino acid sequence of SEQ ID NO:9. In embodiments, the second peptidyl linker includes the amino acid sequence of SEQ ID NO: 10. In embodiments, the second peptidyl linker is the amino acid sequence of SEQ ID NO: 7, SEQ ID NO:8, SEQ ID NO:9, or SEQ ID NO:10. In embodiments, the second peptidyl linker is the amino acid sequence of SEQ ID NO:7. In embodiments, the second peptidyl linker is the amino acid sequence of SEQ ID NO: 8. In embodiments, the second peptidyl linker is the amino acid sequence of SEQ ID NO: 9. In embodiments, the second peptidyl linker is the amino acid sequence of SEQ ID NO: 10.

In embodiments, the third peptidyl linker includes the amino acid sequence of SEQ ID NO:7, SEQ ID NO:8, SEQ ID NO:9, or SEQ ID NO: 10. In embodiments, the third peptidyl linker includes the amino acid sequence of SEQ ID NO:7. In embodiments, the third peptidyl linker includes the amino acid sequence of SEQ ID NO:8 In embodiments, the third peptidyl linker includes the amino acid sequence of SEQ ID NO:9. In embodiments, the third peptidyl linker includes the amino acid sequence of SEQ ID NO: 10. In embodiments, the third peptidyl linker is the amino acid sequence of SEQ ID NO: 7, SEQ ID NO:8, SEQ ID NO:9, or SEQ ID NO:10. In embodiments, the third peptidyl linker is the amino acid sequence of SEQ ID NO:7. In embodiments, the third peptidyl linker is the amino acid sequence of SEQ ID NO:8. In embodiments, the third peptidyl linker is the amino acid sequence of SEQ ID NO: 9. In embodiments, the third peptidyl linker is the amino acid sequence of SEQ ID NO: 10.

In embodiments, the fourth peptidyl linker includes the amino acid sequence of SEQ ID NO:7, SEQ ID NO:8, SEQ ID NO:9, or SEQ ID NO: 10. In embodiments, the fourth peptidyl linker includes the amino acid sequence of SEQ ID NO:7. In embodiments, the fourth peptidyl linker includes the amino acid sequence of SEQ ID NO:8 In embodiments, the fourth peptidyl linker includes the amino acid sequence of SEQ ID NO:9. In embodiments, the fourth peptidyl linker includes the amino acid sequence of SEQ ID NO: 10. In embodiments, the fourth peptidyl linker is the amino acid sequence of SEQ ID NO:7, SEQ ID NO:8, SEQ ID NO:9, or SEQ ID NO:10. In embodiments, the fourth peptidyl linker is the amino acid sequence of SEQ ID NO: 7. In embodiments, the fourth peptidyl linker is the amino acid sequence of SEQ ID NO: 8. In embodiments, the fourth peptidyl linker is the amino acid sequence of SEQ ID NO: 9. In embodiments, the fourth peptidyl linker is the amino acid sequence of SEQ ID NO: 10.

In embodiments, the ligand binding domain is non-covalently bound to the pseudoligand domain.

In embodiments, the first fluorophore and the second fluorophore are a FRET pair. In embodiments, the FRET pair is a cyan-yellow or a red-green FRET pair. In embodiments, the FRET pair is a cyan-yellow FRET pair. In embodiments, the FRET pair is a red-green FRET pair. In embodiments, the cyan-yellow FRET pair includes a mCerulean3 fluorophore and a YPet fluorophore.

In embodiments, the ligand binding domain binds a Ras protein with higher affinity than the pseudoligand domain. In embodiments, the Ras protein is an active Ras protein. In embodiments, the Ras protein is bound to guanosine triphosphate (GTP).

### Biosensor Complexes

The compositions provided herein include complexes including biosensor recombinant proteins (e.g., RasAR) or portions thereof provided herein including embodiments thereof bound to a protein (i.e. Ras). The biosensor recombinant proteins (e.g., RasAR) provided herein are described in detail throughout this application (including the description above and in the examples section). Thus, in an aspect, a complex including the recombinant protein provided herein including embodiments thereof non-covalently bound to a Ras protein.

In embodiments, the ligand binding domain is non-covalently bound to the Ras protein. In embodiments, the the Ras protein is an active Ras protein. In embodiments, the Ras protein is bound to guanosine triphosphate (GTP).

### Nucleic Acid Compositions

The compositions provided herein include nucleic acid molecules encoding the biosensor recombinant proteins (e.g., RasAR) or portions thereof provided herein including embodiments thereof. The biosensor recombinant proteins (e.g., RasAR) encoded by the isolated nucleic acid provided herein are described in detail throughout this application (including the description above and in the examples section). Thus, in an aspect, an isolated nucleic acid encoding the recombinant protein as provided herein including embodiments thereof is provided.

### Expression Vector Compositions

The compositions provided herein include expression vectors including an isolated nucleic acid encoding biosensor recombinant proteins (e.g., RasAR) or portions thereof provided herein including embodiments thereof. The biosensor recombinant proteins (e.g., RasAR) encoded by the isolated nucleic acid provided herein are described in detail throughout this application (including the description above and in the examples section). Thus, in an aspect, an expression vector including the isolated nucleic acid provided herein including embodiments thereof encoding the recombinant protein as provided herein including embodiments thereof is provided.

In embodiments, the expression vector is a viral vector. In embodiments, the viral vector is an Adeno-associated viral vector, an Adenovirus vector, or a lentiviral vector. In embodiments, the viral vector is an Adeno-associated viral vector. In embodiments, the viral vector is an Adenovirus vector. In embodiments, the viral vector is a lentiviral vector.

### Methods of Use

The biosensor recombinant proteins (e.g. RasAR) provided herein including embodiments thereof may be used for detecting a protein (e.g. Ras) or the activity of a protein (e.g. Ras) in a cell. Any of the biosensor recombinant proteins provided herein including embodiments thereof may be used for methods of detecting a Ras protein or the activity of a Ras protein in a cell. Thus, in an aspect is provided a method for detecting a Ras protein in a cell, the method including: (a) contacting a cell with an expression vector including an isolated nucleic acid, wherein the isolated nucleic acid encodes a recombinant protein including: a first fluorophore, a ligand binding domain, a pseudoligand domain, and a second fluorophore; (b) transducing the cell with the expression vector; (c) allowing the cell to express the recombinant protein and bind to the Ras protein within the cell; and (d) detecting a fluorescent signal form the recombinant protein thereby detecting the Ras protein in the cell.

In embodiments, the first fluorophore is bound to the ligand binding domain through a first peptidyl linker, the pseudoligand domain is bound to the ligand binding domain through a second peptidyl linker, and the second fluorophore is bound to the pseudoligand domain through a third peptidyl linker.

In embodiments, the cell is imaged at a first time point to generate a first image. In embodiments, the cell is imaged at a second time point to generate a second image. In embodiments, step (d) includes comparing the first image to the second image to determine activity of the Ras protein at the first time point compared to the second time point.

In embodiments, the method further includes contacting the cell with an inhibitor at a third time point, wherein the third time point is between the first time point and the second time point. In embodiments, the inhibitor targets the Ras protein or the Ras-Extracellular signal-regulated kinase (ERK) signaling pathway. In embodiments, the inhibitor is a Ras inhibitor, a SHP2 inhibitor, a Ras guanine-nucleotide exchange factor (GEF) inhibitor, or a tyrosine kinase inhibitor. In embodiments, the inhibitor is a Ras inhibitor. In embodiments, the inhibitor is a SHP2 inhibitor. In embodiments, the inhibitor is a Ras guanine-nucleotide exchange factor (GEF) inhibitor. In embodiments, the inhibitor is a tyrosine kinase inhibitor. In embodiments, the inhibitor is a KRasG12C inhibitor.

In embodiments, the cell is a living cell. In embodiments, the cell is imaged using a fluorescence microscope.

In embodiments, the first fluorophore and the second fluorophore are a Forster Resonance Energy Transfer (FRET) pair. In embodiments, the FRET pair is a cyan-yellow or a red-green FRET pair. In embodiments, the FRET pair is a cyan-yellow FRET pair. In embodiments, the FRET pair is a red-green FRET pair. In embodiments, the first fluorophore or the second fluorophore is a mCerulean3 fluorophore or a YPet fluorophore. In embodiments, the first fluorophore or the second fluorophore is a mCerulean3 fluorophore. In embodiments, the first fluorophore or the second fluorophore is a YPet fluorophore. In embodiments, the first fluorophore is a mCerulean3 fluorophore or a YPet fluorophore. In embodiments, the the second fluorophore is a mCerulean3 fluorophore or a YPet fluorophore. In embodiments, the first fluorophore is a mCerulean3 fluorophore and the second fluorophore is a YPet fluorophore. In embodiments, the the first fluorophore is a YPet fluorophore and the second fluorophore is a mCerulean3 fluorophore. In embodiments, the first fluorophore is a mCerulean3 fluorophore. In embodiments, the first fluorophore is a YPet fluorophore. In embodiments, the the second fluorophore is a mCerulean3 fluorophore. In embodiments, the the second fluorophore is a YPet fluorophore.

In embodiments, the pseudoligand domain includes the amino acid sequence of SEQ ID NO:1, SEQ ID NO:2, SEQ ID NO:3, SEQ ID NO:4, SEQ ID NO:5, or SEQ ID NO:6 In embodiments, the pseudoligand domain includes the amino acid sequence of SEQ ID NO: 1. In embodiments, the pseudoligand domain includes the amino acid sequence of SEQ ID NO:2. In embodiments, the pseudoligand domain includes the amino acid sequence of SEQ ID NO:3. In embodiments, the pseudoligand domain includes the amino acid sequence of SEQ ID NO:4. In embodiments, the pseudoligand domain includes the amino acid sequence of SEQ ID NO:5. In embodiments, the pseudoligand domain includes the amino acid sequence of SEQ ID NO:6. In embodiments, the pseudoligand domain is the amino acid sequence of SEQ ID NO: 1, SEQ ID NO:2, SEQ ID NO:3, SEQ ID NO:4, SEQ ID NO:5, or SEQ ID NO:6. In embodiments, the pseudoligand domain is the amino acid sequence of SEQ ID NO: 1. In embodiments, the pseudoligand domain is the amino acid sequence of SEQ ID NO: 2. In embodiments, the pseudoligand domain is the amino acid sequence of SEQ ID NO:3. In embodiments, the pseudoligand domain is the amino acid sequence of SEQ ID NO:4. In embodiments, the pseudoligand domain is the amino acid sequence of SEQ ID NO:5. In embodiments, the pseudoligand domain is the amino acid sequence of SEQ ID NO: 6.

In embodiments, the pseudoligand domain includes a sequence having 70%, 75%, 80%, 85%, 90%, 95%, 96%, 97%, 98%, 99% or 100% sequence identity to 5, 6, 7, 8, 9, 10, 11, or 12 contiguous amino acids of the sequence of SEQ ID NO:1. In embodiments, the pseudoligand domain includes a sequence having 70% sequence identity to 5, 6, 7, 8, 9, 10, 11, or 12 contiguous amino acids of the sequence of SEQ ID NO:1. In embodiments, the pseudoligand domain includes a sequence having 75% sequence identity to 5, 6, 7, 8, 9, 10, 11, or 12 contiguous amino acids of the sequence of SEQ ID NO:1. In embodiments, the pseudoligand domain includes a sequence having 80% sequence identity to 5, 6, 7, 8, 9, 10, 11, or 12 contiguous amino acids of the sequence of SEQ ID NO:1. In embodiments, the pseudoligand domain includes a sequence having 85% sequence identity to 5, 6, 7, 8, 9, 10, 11, or 12 contiguous amino acids of the sequence of SEQ ID NO:1. In embodiments, the pseudoligand domain includes a sequence having 90% sequence identity to 5, 6, 7, 8, 9, 10, 11, or 12 contiguous amino acids of the sequence of SEQ ID NO:1. In embodiments, the pseudoligand domain includes a sequence having 95% sequence identity to 5, 6, 7, 8, 9, 10, 11, or 12 contiguous amino acids of the sequence of SEQ ID NO:1. In embodiments, the pseudoligand domain includes a sequence having 96% sequence identity to 5, 6, 7, 8, 9, 10, 11, or 12 contiguous amino acids of the sequence of SEQ ID NO:1. In embodiments, the pseudoligand domain includes a sequence having 97% sequence identity to 5, 6, 7, 8, 9, 10, 11, or 12 contiguous amino acids of the sequence of SEQ ID NO:1. In embodiments, the pseudoligand domain includes a sequence having 98% sequence identity to 5, 6, 7, 8, 9, 10, 11, or 12 contiguous amino acids of the sequence of SEQ ID NO:1. In embodiments, the pseudoligand domain includes a sequence having 99% sequence identity to 5, 6, 7, 8, 9, 10, 11, or 12 contiguous amino acids of the sequence of SEQ ID NO:1. In embodiments, the pseudoligand domain includes a sequence having 100% sequence identity to 5, 6, 7, 8, 9, 10, 11, or 12 contiguous amino acids of the sequence of SEQ ID NO:1.

In embodiments, the pseudoligand domain includes a sequence having 70%, 75%, 80%, 85%, 90%, 95%, 96%, 97%, 98%, 99% or 100% sequence identity to 5, 6, 7, 8, 9, or 10 contiguous amino acids of the sequence of SEQ ID NO:2. In embodiments, the pseudoligand domain includes a sequence having 70% sequence identity to 5, 6, 7, 8, 9, or 10 contiguous amino acids of the sequence of SEQ ID NO:2. In embodiments, the pseudoligand domain includes a sequence having 75% sequence identity to 5, 6, 7, 8, 9, or 10 contiguous amino acids of the sequence of SEQ ID NO:2. In embodiments, the pseudoligand domain includes a sequence having 80% sequence identity to 5, 6, 7, 8, 9, or 10 contiguous amino acids of the sequence of SEQ ID NO:2. In embodiments, the pseudoligand domain includes a sequence having 85% sequence identity to 5, 6, 7, 8, 9, or 10 contiguous amino acids of the sequence of SEQ ID NO:2. In embodiments, the pseudoligand domain includes a sequence having 90% sequence identity to 5, 6, 7, 8, 9, or 10 contiguous amino acids of the sequence of SEQ ID NO:2. In embodiments, the pseudoligand domain includes a sequence having 95% sequence identity to 5, 6, 7, 8, 9, or 10 contiguous amino acids of the sequence of SEQ ID NO:2. In embodiments, the pseudoligand domain includes a sequence having 96% sequence identity to 5, 6, 7, 8, 9, or 10 contiguous amino acids of the sequence of SEQ ID NO:2. In embodiments, the pseudoligand domain includes a sequence having 97% sequence identity to 5, 6, 7, 8, 9, or 10 contiguous amino acids of the sequence of SEQ ID NO:2. In embodiments, the pseudoligand domain includes a sequence having 98% sequence identity to 5, 6, 7, 8, 9, or 10 contiguous amino acids of the sequence of SEQ ID NO:2. In embodiments, the pseudoligand domain includes a sequence having 99% sequence identity to 5, 6, 7, 8, 9, or 10 contiguous amino acids of the sequence of SEQ ID NO:2. In embodiments, the pseudoligand domain includes a sequence having 100% sequence identity to 5, 6, 7, 8, 9, or 10 contiguous amino acids of the sequence of SEQ ID NO:2.

In embodiments, the pseudoligand domain includes a sequence having 70%, 75%, 80%, 85%, 90%, 95%, 96%, 97%, 98%, 99% or 100% sequence identity to 5, 6, 7, 8, 9, 10, 11, 12, or 13 contiguous amino acids of the sequence of SEQ ID NO:3. In embodiments, the pseudoligand domain includes a sequence having 70% sequence identity to 5, 6, 7, 8, 9, 10, 11, 12, or 13 contiguous amino acids of the sequence of SEQ ID NO:3. In embodiments, the pseudoligand domain includes a sequence having 75% sequence identity to 5, 6, 7, 8, 9, 10, 11, 12, or 13 contiguous amino acids of the sequence of SEQ ID NO:3. In embodiments, the pseudoligand domain includes a sequence having 80% sequence identity to 5, 6, 7, 8, 9, 10, 11, 12, or 13 contiguous amino acids of the sequence of SEQ ID NO:3. In embodiments, the pseudoligand domain includes a sequence having 85% sequence identity to 5, 6, 7, 8, 9, 10, 11, 12, or 13 contiguous amino acids of the sequence of SEQ ID NO:3. In embodiments, the pseudoligand domain includes a sequence having 90% sequence identity to 5, 6, 7, 8, 9, 10, 11, 12, or 13 contiguous amino acids of the sequence of SEQ ID NO:3. In embodiments, the pseudoligand domain includes a sequence having 95% sequence identity to 5, 6, 7, 8, 9, 10, 11, 12, or 13 contiguous amino acids of the sequence of SEQ ID NO:3. In embodiments, the pseudoligand domain includes a sequence having 96% sequence identity to 5, 6, 7, 8, 9, 10, 11, 12, or 13 contiguous amino acids of the sequence of SEQ ID NO:3. In embodiments, the pseudoligand domain includes a sequence having 97% sequence identity to 5, 6, 7, 8, 9, 10, 11, 12, or 13 contiguous amino acids of the sequence of SEQ ID NO:3. In embodiments, the pseudoligand domain includes a sequence having 98% sequence identity to 5, 6, 7, 8, 9, 10, 11, 12, or 13 contiguous amino acids of the sequence of SEQ ID NO:3. In embodiments, the pseudoligand domain includes a sequence having 99% sequence identity to 5, 6, 7, 8, 9, 10, 11, 12, or 13 contiguous amino acids of the sequence of SEQ ID NO:3. In embodiments, the pseudoligand domain includes a sequence having 100% sequence identity to 5, 6, 7, 8, 9, 10, 11, 12, or 13 contiguous amino acids of the sequence of SEQ ID NO:3.

In embodiments, the pseudoligand domain includes a sequence having 70%, 75%, 80%, 85%, 90%, 95%, 96%, 97%, 98%, 99% or 100% sequence identity to 5, 6, 7, 8, or 9 contiguous amino acids of the sequence of SEQ ID NO:4. In embodiments, the pseudoligand domain includes a sequence having 70% sequence identity to 5, 6, 7, 8, or 9 contiguous amino acids of the sequence of SEQ ID NO:4. In embodiments, the pseudoligand domain includes a sequence having 75% sequence identity to 5, 6, 7, 8, or 9 contiguous amino acids of the sequence of SEQ ID NO:4. In embodiments, the pseudoligand domain includes a sequence having 80% sequence identity to 5, 6, 7, 8, or 9 contiguous amino acids of the sequence of SEQ ID NO:4. In embodiments, the pseudoligand domain includes a sequence having 85% sequence identity to 5, 6, 7, 8, or 9 contiguous amino acids of the sequence of SEQ ID NO:4. In embodiments, the pseudoligand domain includes a sequence having 90% sequence identity to 5, 6, 7, 8, or 9 contiguous amino acids of the sequence of SEQ ID NO:4. In embodiments, the pseudoligand domain includes a sequence having 95% sequence identity to 5, 6, 7, 8, or 9 contiguous amino acids of the sequence of SEQ ID NO:4. In embodiments, the pseudoligand domain includes a sequence having 96% sequence identity to 5, 6, 7, 8, or 9 contiguous amino acids of the sequence of SEQ ID NO:4. In embodiments, the pseudoligand domain includes a sequence having 97% sequence identity to 5, 6, 7, 8, or 9 contiguous amino acids of the sequence of SEQ ID NO:4. In embodiments, the pseudoligand domain includes a sequence having 98% sequence identity to 5, 6, 7, 8, or 9 contiguous amino acids of the sequence of SEQ ID NO:4. In embodiments, the pseudoligand domain includes a sequence having 99% sequence identity to 5, 6, 7, 8, or 9 contiguous amino acids of the sequence of SEQ ID NO:4. In embodiments, the pseudoligand domain includes a sequence having 100% sequence identity to 5, 6, 7, 8, or 9 contiguous amino acids of the sequence of SEQ ID NO:4.

In embodiments, the pseudoligand domain includes a sequence having 70%, 75%, 80%, 85%, 90%, 95%, 96%, 97%, 98%, 99% or 100% sequence identity to 5, 10, 11, 12, 13, 14, 15, or 16 contiguous amino acids of the sequence of SEQ ID NO:5. In embodiments, the pseudoligand domain includes a sequence having 70% sequence identity to 5, 10, 11, 12, 13, 14, 15, or 16 contiguous amino acids of the sequence of SEQ ID NO:5. In embodiments, the pseudoligand domain includes a sequence having 75% sequence identity to 5, 10, 11, 12, 13, 14, 15, or 16 contiguous amino acids of the sequence of SEQ ID NO:5. In embodiments, the pseudoligand domain includes a sequence having 80% sequence identity to 5, 10, 11, 12, 13, 14, 15, or 16 contiguous amino acids of the sequence of SEQ ID NO:5. In embodiments, the pseudoligand domain includes a sequence having 85% sequence identity to 5, 10, 11, 12, 13, 14, 15, or 16 contiguous amino acids of the sequence of SEQ ID NO:5. In embodiments, the pseudoligand domain includes a sequence having 90% sequence identity to 5, 10, 11, 12, 13, 14, 15, or 16 contiguous amino acids of the sequence of SEQ ID NO:5. In embodiments, the pseudoligand domain includes a sequence having 95% sequence identity to 5, 10, 11, 12, 13, 14, 15, or 16 contiguous amino acids of the sequence of SEQ ID NO:5. In embodiments, the pseudoligand domain includes a sequence having 96% sequence identity to 5, 10, 11, 12, 13, 14, 15, or 16 contiguous amino acids of the sequence of SEQ ID NO:5. In embodiments, the pseudoligand domain includes a sequence having 97% sequence identity to 5, 10, 11, 12, 13, 14, 15, or 16 contiguous amino acids of the sequence of SEQ ID NO:5. In embodiments, the pseudoligand domain includes a sequence having 98% sequence identity to 5, 10, 11, 12, 13, 14, 15, or 16 contiguous amino acids of the sequence of SEQ ID NO:5. In embodiments, the pseudoligand domain includes a sequence having 99% sequence identity to 5, 10, 11, 12, 13, 14, 15, or 16 contiguous amino acids of the sequence of SEQ ID NO:5. In embodiments, the pseudoligand domain includes a sequence having 100% sequence identity to 5, 10, 11, 12, 13, 14, 15, or 16 contiguous amino acids of the sequence of SEQ ID NO:5.

In embodiments, the pseudoligand domain includes a sequence having 70%, 75%, 80%, 85%, 90%, 95%, 96%, 97%, 98%, 99% or 100% sequence identity to 5, 10, 11, 12, 13, 14, 15, 16, or 17 contiguous amino acids of the sequence of SEQ ID NO:6. In embodiments, the pseudoligand domain includes a sequence having 70% sequence identity to 5, 10, 11, 12, 13, 14, 15, 16, or 17 contiguous amino acids of the sequence of SEQ ID NO:6. In embodiments, the pseudoligand domain includes a sequence having 75% sequence identity to 5, 10, 11, 12, 13, 14, 15, 16, or 17 contiguous amino acids of the sequence of SEQ ID NO:6. In embodiments, the pseudoligand domain includes a sequence having 80% sequence identity to 5, 10, 11, 12, 13, 14, 15, 16, or 17 contiguous amino acids of the sequence of SEQ ID NO:6. In embodiments, the pseudoligand domain includes a sequence having 85% sequence identity to 5, 10, 11, 12, 13, 14, 15, 16, or 17 contiguous amino acids of the sequence of SEQ ID NO:6. In embodiments, the pseudoligand domain includes a sequence having 90% sequence identity to 5, 10, 11, 12, 13, 14, 15, 16, or 17 contiguous amino acids of the sequence of SEQ ID NO:6. In embodiments, the pseudoligand domain includes a sequence having 95% sequence identity to 5, 10, 11, 12, 13, 14, 15, 16, or 17 contiguous amino acids of the sequence of SEQ ID NO:6. In embodiments, the pseudoligand domain includes a sequence having 96% sequence identity to 5, 10, 11, 12, 13, 14, 15, 16, or 17 contiguous amino acids of the sequence of SEQ ID NO:6. In embodiments, the pseudoligand domain includes a sequence having 97% sequence identity to 5, 10, 11, 12, 13, 14, 15, 16, or 17 contiguous amino acids of the sequence of SEQ ID NO:6. In embodiments, the pseudoligand domain includes a sequence having 98% sequence identity to 5, 10, 11, 12, 13, 14, 15, 16, or 17 contiguous amino acids of the sequence of SEQ ID NO:6. In embodiments, the pseudoligand domain includes a sequence having 99% sequence identity to 5, 10, 11, 12, 13, 14, 15, 16, or 17 contiguous amino acids of the sequence of SEQ ID NO:6. In embodiments, the pseudoligand domain includes a sequence having 100% sequence identity to 5, 10, 11, 12, 13, 14, 15, 16, or 17 contiguous amino acids of the sequence of SEQ ID NO:6.

In embodiments, the pseudoligand domain includes a sequence having 70%, 75%, 80%, 85%, 90%, 95%, 96%, 97%, 98%, 99% or 100% sequence identity to 5, 10, 15, 20, 30, 40, 50, 60, or 70 contiguous amino acids of the sequence of SEQ ID NO:11. In embodiments, the pseudoligand domain includes a sequence having 70% sequence identity to 5, 10, 15, 20, 30, 40, 50, 60, or 70 contiguous amino acids of the sequence of SEQ ID NO:11. In embodiments, the pseudoligand domain includes a sequence having 75% sequence identity to 5, 10, 15, 20, 30, 40, 50, 60, or 70 contiguous amino acids of the sequence of SEQ ID NO: 11. In embodiments, the pseudoligand domain includes a sequence having 80% sequence identity to 5, 10, 15, 20, 30, 40, 50, 60, or 70 contiguous amino acids of the sequence of SEQ ID NO:11. In embodiments, the pseudoligand domain includes a sequence having 85% sequence identity to 5, 10, 15, 20, 30, 40, 50, 60, or 70 contiguous amino acids of the sequence of SEQ ID NO:11. In embodiments, the pseudoligand domain includes a sequence having 90% sequence identity to 5, 10, 15, 20, 30, 40, 50, 60, or 70 contiguous amino acids of the sequence of SEQ ID NO:11. In embodiments, the pseudoligand domain includes a sequence having 95% sequence identity to 5, 10, 15, 20, 30, 40, 50, 60, or 70 contiguous amino acids of the sequence of SEQ ID NO:11. In embodiments, the pseudoligand domain includes a sequence having 96% sequence identity to 5, 10, 15, 20, 30, 40, 50, 60, or 70 contiguous amino acids of the sequence of SEQ ID NO:11. In embodiments, the pseudoligand domain includes a sequence having 97% sequence identity to 5, 10, 15, 20, 30, 40, 50, 60, or 70 contiguous amino acids of the sequence of SEQ ID NO:11. In embodiments, the pseudoligand domain includes a sequence having 98% sequence identity to 55, 10, 15, 20, 30, 40, 50, 60, or 70 contiguous amino acids of the sequence of SEQ ID NO:11. In embodiments, the pseudoligand domain includes a sequence having 99% sequence identity to 5, 10, 15, 20, 30, 40, 50, 60, or 70 contiguous amino acids of the sequence of SEQ ID NO:11. In embodiments, the pseudoligand domain includes a sequence having 100% sequence identity to 5, 10, 15, 20, 30, 40, 50, 60, or 70 contiguous amino acids of the sequence of SEQ ID NO:11.

In embodiments, the ligand binding domain includes a Ras binding domain (RBD). In embodiments, the ligand binding domain includes the amino acid sequence of SEQ ID NO:17, SEQ ID NO:18, SEQ ID NO:19, SEQ ID NO:20, or SEQ ID NO:21. In embodiments, the ligand binding domain includes the amino acid sequence of SEQ ID NO: 17. In embodiments, the ligand binding domain includes the amino acid sequence of SEQ ID NO: 18. In embodiments, the ligand binding domain includes the amino acid sequence of SEQ ID NO: 19. In embodiments, the ligand binding domain includes the amino acid sequence of SEQ ID NO:20. In embodiments, the ligand binding domain includes the amino acid sequence of SEQ ID NO:21. In embodiments, the ligand binding domain is the amino acid sequence of SEQ ID NO:17, SEQ ID NO:18, SEQ ID NO:19, SEQ ID NO:20, or SEQ ID NO:21. In embodiments, the ligand binding domain is the amino acid sequence of SEQ ID NO: 17. In embodiments, the ligand binding domain is the amino acid sequence of SEQ ID NO: 18. In embodiments, the ligand binding domain is the amino acid sequence of SEQ ID NO: 19. In embodiments, the ligand binding domain is the amino acid sequence of SEQ ID NO:20. In embodiments, the ligand binding domain is the amino acid sequence of SEQ ID NO:21.

In embodiments, the C-terminus of the first fluorophore is attached to the N-terminus of the ligand binding domain through the first peptidyl linker. In embodiments, the C-terminus of the ligand binding domain is attached to the N-terminus of the pseudoligand domain through the second peptidyl linker. In embodiments, the C-terminus of the pseudoligand binding domain is attached to the N-terminus of the second fluorophore through the third peptidyl linker.

In embodiments, the recombinant protein further includes a targeting moiety. In embodiments, the targeting moiety is a cytosolic targeting moiety, a plasma membrane targeting moiety, a Golgi apparatus targeting moiety. In embodiments, the targeting moiety is a cytosolic targeting moiety. In embodiments, the targeting moiety is a plasma membrane targeting moiety. In embodiments, the targeting moiety is a Golgi apparatus targeting moiety. In embodiments, the targeting moiety is a nuclear export signal (NES), a Giantin tag, or a Lyn-targeting motif. In embodiments, the targeting moiety is a nuclear export signal (NES). In embodiments, the targeting moiety is a Giantin tag. In embodiments, the targeting moiety is a Lyn-targeting motif.

In embodiments, the targeting moiety includes the amino acid sequence of SEQ ID NO: 14, SEQ ID NO:15, or SEQ ID NO: 16. In embodiments, the targeting moiety includes the amino acid sequence of SEQ ID NO: 14. In embodiments, the targeting moiety includes the amino acid sequence of SEQ ID NO:15. In embodiments, the targeting moiety includes the amino acid sequence of SEQ ID NO: 16. In embodiments, the targeting moiety is the amino acid sequence of SEQ ID NO: 14. In embodiments, the targeting moiety is the amino acid sequence of SEQ ID NO:15. In embodiments, the targeting moiety is the amino acid sequence of SEQ ID NO:16.

In embodiments, the C-terminus of the targeting moiety is attached to the N-terminus of the first fluorophore through a fourth peptidyl linker. In embodiments, the C-terminus of the second fluorophore is attached to the N-terminus of the targeting moiety through a fourth peptidyl linker.

In embodiments, the first peptidyl linker, the second peptidyl linker, the third peptidyl linker, and/or the fourth peptidyl linker are independently an amino acid sequence of about 1 to about 1000 amino acids. In embodiments, the first peptidyl linker, the second peptidyl linker, the third peptidyl linker, and/or the fourth peptidyl linker are independently an amino acid sequence of about 2 to about 1000 amino acids. In embodiments, the first peptidyl linker, the second peptidyl linker, the third peptidyl linker, and/or the fourth peptidyl linker are independently an amino acid sequence of about 3 to about 1000 amino acids. In embodiments, the first peptidyl linker, the second peptidyl linker, the third peptidyl linker, and/or the fourth peptidyl linker are independently an amino acid sequence of about 4 to about 1000 amino acids. In embodiments, the first peptidyl linker, the second peptidyl linker, the third peptidyl linker, and/or the fourth peptidyl linker are independently an amino acid sequence of about 5 to about 1000 amino acids. In embodiments, the first peptidyl linker, the second peptidyl linker, the third peptidyl linker, and/or the fourth peptidyl linker are independently an amino acid sequence of about 6 to about 1000 amino acids. In embodiments, the first peptidyl linker, the second peptidyl linker, the third peptidyl linker, and/or the fourth peptidyl linker are independently an amino acid sequence of about 7 to about 1000 amino acids. In embodiments, the first peptidyl linker, the second peptidyl linker, the third peptidyl linker, and/or the fourth peptidyl linker are independently an amino acid sequence of about 8 to about 1000 amino acids. In embodiments, the first peptidyl linker, the second peptidyl linker, the third peptidyl linker, and/or the fourth peptidyl linker are independently an amino acid sequence of about 9 to about 1000 amino acids. In embodiments, the first peptidyl linker, the second peptidyl linker, the third peptidyl linker, and/or the fourth peptidyl linker are independently an amino acid sequence of about 10 to about 1000 amino acids. In embodiments, the first peptidyl linker, the second peptidyl linker, the third peptidyl linker, and/or the fourth peptidyl linker are independently an amino acid sequence of about 20 to about 1000 amino acids. In embodiments, the first peptidyl linker, the second peptidyl linker, the third peptidyl linker, and/or the fourth peptidyl linker are independently an amino acid sequence of about 30 to about 1000 amino acids. In embodiments, the first peptidyl linker, the second peptidyl linker, the third peptidyl linker, and/or the fourth peptidyl linker are independently an amino acid sequence of about 40 to about 1000 amino acids. In embodiments, the first peptidyl linker, the second peptidyl linker, the third peptidyl linker, and/or the fourth peptidyl linker are independently an amino acid sequence of about 50 to about 1000 amino acids. In embodiments, the first peptidyl linker, the second peptidyl linker, the third peptidyl linker, and/or the fourth peptidyl linker are independently an amino acid sequence of about 100 to about 1000 amino acids. In embodiments, the first peptidyl linker, the second peptidyl linker, the third peptidyl linker, and/or the fourth peptidyl linker are independently an amino acid sequence of about 200 to about 1000 amino acids. In embodiments, the first peptidyl linker, the second peptidyl linker, the third peptidyl linker, and/or the fourth peptidyl linker are independently an amino acid sequence of about 300 to about 1000 amino acids. In embodiments, the first peptidyl linker, the second peptidyl linker, the third peptidyl linker, and/or the fourth peptidyl linker are independently an amino acid sequence of about 400 to about 1000 amino acids. In embodiments, the first peptidyl linker, the second peptidyl linker, the third peptidyl linker, and/or the fourth peptidyl linker are independently an amino acid sequence of about 500 to about 1000 amino acids. In embodiments, the first peptidyl linker, the second peptidyl linker, the third peptidyl linker, and/or the fourth peptidyl linker are independently an amino acid sequence of about 600 to about 1000 amino acids. In embodiments, the first peptidyl linker, the second peptidyl linker, the third peptidyl linker, and/or the fourth peptidyl linker are independently an amino acid sequence of about 700 to about 1000 amino acids. In embodiments, the first peptidyl linker, the second peptidyl linker, the third peptidyl linker, and/or the fourth peptidyl linker are independently an amino acid sequence of about 800 to about 1000 amino acids. In embodiments, the first peptidyl linker, the second peptidyl linker, the third peptidyl linker, and/or the fourth peptidyl linker are independently an amino acid sequence of about 900 to about 1000 amino acids.

In embodiments, the first peptidyl linker, the second peptidyl linker, the third peptidyl linker, and/or the fourth peptidyl linker are independently an amino acid sequence of about 1 to about 900 amino acids. In embodiments, the first peptidyl linker, the second peptidyl linker, the third peptidyl linker, and/or the fourth peptidyl linker are independently an amino acid sequence of about 1 to about 800 amino acids. In embodiments, the first peptidyl linker, the second peptidyl linker, the third peptidyl linker, and/or the fourth peptidyl linker are independently an amino acid sequence of about 1 to about 700 amino acids. In embodiments, the first peptidyl linker, the second peptidyl linker, the third peptidyl linker, and/or the fourth peptidyl linker are independently an amino acid sequence of about 1 to about 600 amino acids. In embodiments, the first peptidyl linker, the second peptidyl linker, the third peptidyl linker, and/or the fourth peptidyl linker are independently an amino acid sequence of about 1 to about 500 amino acids. In embodiments, the first peptidyl linker, the second peptidyl linker, the third peptidyl linker, and/or the fourth peptidyl linker are independently an amino acid sequence of about 1 to about 400 amino acids. In embodiments, the first peptidyl linker, the second peptidyl linker, the third peptidyl linker, and/or the fourth peptidyl linker are independently an amino acid sequence of about 1 to about 300 amino acids. In embodiments, the first peptidyl linker, the second peptidyl linker, the third peptidyl linker, and/or the fourth peptidyl linker are independently an amino acid sequence of about 1 to about 200 amino acids. In embodiments, the first peptidyl linker, the second peptidyl linker, the third peptidyl linker, and/or the fourth peptidyl linker are independently an amino acid sequence of about 1 to about 100 amino acids. In embodiments, the first peptidyl linker, the second peptidyl linker, the third peptidyl linker, and/or the fourth peptidyl linker are independently an amino acid sequence of about 1 to about 50 amino acids. In embodiments, the first peptidyl linker, the second peptidyl linker, the third peptidyl linker, and/or the fourth peptidyl linker are independently an amino acid sequence of about 1 to about 40 amino acids. In embodiments, the first peptidyl linker, the second peptidyl linker, the third peptidyl linker, and/or the fourth peptidyl linker are independently an amino acid sequence of about 1 to about 30 amino acids. In embodiments, the first peptidyl linker, the second peptidyl linker, the third peptidyl linker, and/or the fourth peptidyl linker are independently an amino acid sequence of about 1 to about 20 amino acids. In embodiments, the first peptidyl linker, the second peptidyl linker, the third peptidyl linker, and/or the fourth peptidyl linker are independently an amino acid sequence of about 1 to about 10 amino acids. In embodiments, the first peptidyl linker, the second peptidyl linker, the third peptidyl linker, and/or the fourth peptidyl linker are independently an amino acid sequence of about 1 to about 9 amino acids. In embodiments, the first peptidyl linker, the second peptidyl linker, the third peptidyl linker, and/or the fourth peptidyl linker are independently an amino acid sequence of about 1 to about 8 amino acids. In embodiments, the first peptidyl linker, the second peptidyl linker, the third peptidyl linker, and/or the fourth peptidyl linker are independently an amino acid sequence of about 1 to about 7 amino acids. In embodiments, the first peptidyl linker, the second peptidyl linker, the third peptidyl linker, and/or the fourth peptidyl linker are independently an amino acid sequence of about 1 to about 6 amino acids. In embodiments, the first peptidyl linker, the second peptidyl linker, the third peptidyl linker, and/or the fourth peptidyl linker are independently an amino acid sequence of about 1 to about 5 amino acids. In embodiments, the first peptidyl linker, the second peptidyl linker, the third peptidyl linker, and/or the fourth peptidyl linker are independently an amino acid sequence of about 1 to about 4 amino acids. In embodiments, the first peptidyl linker, the second peptidyl linker, the third peptidyl linker, and/or the fourth peptidyl linker are independently an amino acid sequence of about 1 to about 3 amino acids. In embodiments, the first peptidyl linker, the second peptidyl linker, the third peptidyl linker, and/or the fourth peptidyl linker are independently an amino acid sequence of about 1 to about 2 amino acids.

In embodiments, the first peptidyl linker, the second peptidyl linker, the third peptidyl linker, and/or the fourth peptidyl linker are independently an amino acid sequence of 1 to 1000 amino acids. In embodiments, the first peptidyl linker, the second peptidyl linker, the third peptidyl linker, and/or the fourth peptidyl linker are independently an amino acid sequence of 2 to 1000 amino acids. In embodiments, the first peptidyl linker, the second peptidyl linker, the third peptidyl linker, and/or the fourth peptidyl linker are independently an amino acid sequence of 3 to 1000 amino acids. In embodiments, the first peptidyl linker, the second peptidyl linker, the third peptidyl linker, and/or the fourth peptidyl linker are independently an amino acid sequence of 4 to 1000 amino acids. In embodiments, the first peptidyl linker, the second peptidyl linker, the third peptidyl linker, and/or the fourth peptidyl linker are independently an amino acid sequence of 5 to 1000 amino acids. In embodiments, the first peptidyl linker, the second peptidyl linker, the third peptidyl linker, and/or the fourth peptidyl linker are independently an amino acid sequence of 6 to 1000 amino acids. In embodiments, the first peptidyl linker, the second peptidyl linker, the third peptidyl linker, and/or the fourth peptidyl linker are independently an amino acid sequence of 7 to 1000 amino acids. In embodiments, the first peptidyl linker, the second peptidyl linker, the third peptidyl linker, and/or the fourth peptidyl linker are independently an amino acid sequence of 8 to 1000 amino acids. In embodiments, the first peptidyl linker, the second peptidyl linker, the third peptidyl linker, and/or the fourth peptidyl linker are independently an amino acid sequence of 9 to 1000 amino acids. In embodiments, the first peptidyl linker, the second peptidyl linker, the third peptidyl linker, and/or the fourth peptidyl linker are independently an amino acid sequence of 10 to 1000 amino acids. In embodiments, the first peptidyl linker, the second peptidyl linker, the third peptidyl linker, and/or the fourth peptidyl linker are independently an amino acid sequence of 20 to 1000 amino acids. In embodiments, the first peptidyl linker, the second peptidyl linker, the third peptidyl linker, and/or the fourth peptidyl linker are independently an amino acid sequence of 30 to 1000 amino acids. In embodiments, the first peptidyl linker, the second peptidyl linker, the third peptidyl linker, and/or the fourth peptidyl linker are independently an amino acid sequence of 40 to 1000 amino acids. In embodiments, the first peptidyl linker, the second peptidyl linker, the third peptidyl linker, and/or the fourth peptidyl linker are independently an amino acid sequence of 50 to 1000 amino acids. In embodiments, the first peptidyl linker, the second peptidyl linker, the third peptidyl linker, and/or the fourth peptidyl linker are independently an amino acid sequence of 100 to 1000 amino acids. In embodiments, the first peptidyl linker, the second peptidyl linker, the third peptidyl linker, and/or the fourth peptidyl linker are independently an amino acid sequence of 200 to 1000 amino acids. In embodiments, the first peptidyl linker, the second peptidyl linker, the third peptidyl linker, and/or the fourth peptidyl linker are independently an amino acid sequence of 300 to 1000 amino acids. In embodiments, the first peptidyl linker, the second peptidyl linker, the third peptidyl linker, and/or the fourth peptidyl linker are independently an amino acid sequence of 400 to 1000 amino acids. In embodiments, the first peptidyl linker, the second peptidyl linker, the third peptidyl linker, and/or the fourth peptidyl linker are independently an amino acid sequence of 500 to 1000 amino acids. In embodiments, the first peptidyl linker, the second peptidyl linker, the third peptidyl linker, and/or the fourth peptidyl linker are independently an amino acid sequence of 600 to 1000 amino acids. In embodiments, the first peptidyl linker, the second peptidyl linker, the third peptidyl linker, and/or the fourth peptidyl linker are independently an amino acid sequence of 700 to 1000 amino acids. In embodiments, the first peptidyl linker, the second peptidyl linker, the third peptidyl linker, and/or the fourth peptidyl linker are independently an amino acid sequence of 800 to 1000 amino acids. In embodiments, the first peptidyl linker, the second peptidyl linker, the third peptidyl linker, and/or the fourth peptidyl linker are independently an amino acid sequence of 900 to 1000 amino acids.

In embodiments, the first peptidyl linker, the second peptidyl linker, the third peptidyl linker, and/or the fourth peptidyl linker are independently an amino acid sequence of 1 to 900 amino acids. In embodiments, the first peptidyl linker, the second peptidyl linker, the third peptidyl linker, and/or the fourth peptidyl linker are independently an amino acid sequence of 1 to 800 amino acids. In embodiments, the first peptidyl linker, the second peptidyl linker, the third peptidyl linker, and/or the fourth peptidyl linker are independently an amino acid sequence of 1 to 700 amino acids. In embodiments, the first peptidyl linker, the second peptidyl linker, the third peptidyl linker, and/or the fourth peptidyl linker are independently an amino acid sequence of 1 to 600 amino acids. In embodiments, the first peptidyl linker, the second peptidyl linker, the third peptidyl linker, and/or the fourth peptidyl linker are independently an amino acid sequence of 1 to 500 amino acids. In embodiments, the first peptidyl linker, the second peptidyl linker, the third peptidyl linker, and/or the fourth peptidyl linker are independently an amino acid sequence of 1 to 400 amino acids. In embodiments, the first peptidyl linker, the second peptidyl linker, the third peptidyl linker, and/or the fourth peptidyl linker are independently an amino acid sequence of 1 to 300 amino acids. In embodiments, the first peptidyl linker, the second peptidyl linker, the third peptidyl linker, and/or the fourth peptidyl linker are independently an amino acid sequence of 1 to 200 amino acids. In embodiments, the first peptidyl linker, the second peptidyl linker, the third peptidyl linker, and/or the fourth peptidyl linker are independently an amino acid sequence of 1 to 100 amino acids. In embodiments, the first peptidyl linker, the second peptidyl linker, the third peptidyl linker, and/or the fourth peptidyl linker are independently an amino acid sequence of 1 to 50 amino acids. In embodiments, the first peptidyl linker, the second peptidyl linker, the third peptidyl linker, and/or the fourth peptidyl linker are independently an amino acid sequence of 1 to 40 amino acids. In embodiments, the first peptidyl linker, the second peptidyl linker, the third peptidyl linker, and/or the fourth peptidyl linker are independently an amino acid sequence of 1 to 30 amino acids. In embodiments, the first peptidyl linker, the second peptidyl linker, the third peptidyl linker, and/or the fourth peptidyl linker are independently an amino acid sequence of 1 to 20 amino acids. In embodiments, the first peptidyl linker, the second peptidyl linker, the third peptidyl linker, and/or the fourth peptidyl linker are independently an amino acid sequence of 1 to 10 amino acids. In embodiments, the first peptidyl linker, the second peptidyl linker, the third peptidyl linker, and/or the fourth peptidyl linker are independently an amino acid sequence of 1 to 9 amino acids. In embodiments, the first peptidyl linker, the second peptidyl linker, the third peptidyl linker, and/or the fourth peptidyl linker are independently an amino acid sequence of 1 to 8 amino acids. In embodiments, the first peptidyl linker, the second peptidyl linker, the third peptidyl linker, and/or the fourth peptidyl linker are independently an amino acid sequence of 1 to 7 amino acids. In embodiments, the first peptidyl linker, the second peptidyl linker, the third peptidyl linker, and/or the fourth peptidyl linker are independently an amino acid sequence of 1 to 6 amino acids. In embodiments, the first peptidyl linker, the second peptidyl linker, the third peptidyl linker, and/or the fourth peptidyl linker are independently an amino acid sequence of 1 to 5 amino acids. In embodiments, the first peptidyl linker, the second peptidyl linker, the third peptidyl linker, and/or the fourth peptidyl linker are independently an amino acid sequence of 1 to 4 amino acids. In embodiments, the first peptidyl linker, the second peptidyl linker, the third peptidyl linker, and/or the fourth peptidyl linker are independently an amino acid sequence of 1 to 3 amino acids. In embodiments, the first peptidyl linker, the second peptidyl linker, the third peptidyl linker, and/or the fourth peptidyl linker are independently an amino acid sequence of 1 to 2 amino acids.

In embodiments, the first peptidyl linker, the second peptidyl linker, the third peptidyl linker, or the fourth peptidyl linker is an amino acid sequence of about 1 to about 1000 amino acids. In embodiments, the first peptidyl linker, the second peptidyl linker, the third peptidyl linker, and the fourth peptidyl linker are an amino acid sequence of about 1 to about 1000 amino acids.

In embodiments, the first peptidyl linker is an amino acid sequence of about 1 to about 1000 amino acids. In embodiments, the first peptidyl linker is an amino acid sequence of about 2 to about 1000 amino acids. In embodiments, the first peptidyl linker is an amino acid sequence of about 3 to about 1000 amino acids. In embodiments, the first peptidyl linker is an amino acid sequence of about 4 to about 1000 amino acids. In embodiments, the first peptidyl linker is an amino acid sequence of about 5 to about 1000 amino acids. In embodiments, the first peptidyl linker is an amino acid sequence of about 6 to about 1000 amino acids. In embodiments, the first peptidyl linker is an amino acid sequence of about 7 to about 1000 amino acids. In embodiments, the first peptidyl linker is an amino acid sequence of about 8 to about 1000 amino acids. In embodiments, the first peptidyl linker is an amino acid sequence of about 9 to about 1000 amino acids. In embodiments, the first peptidyl linker is an amino acid sequence of about 10 to about 1000 amino acids. In embodiments, the first peptidyl linker is an amino acid sequence of about 20 to about 1000 amino acids. In embodiments, the first peptidyl linker is an amino acid sequence of about 30 to about 1000 amino acids. In embodiments, the first peptidyl linker is an amino acid sequence of about 40 to about 1000 amino acids. In embodiments, the first peptidyl linker is an amino acid sequence of about 50 to about 1000 amino acids. In embodiments, the first peptidyl linker is an amino acid sequence of about 100 to about 1000 amino acids. In embodiments, the first peptidyl linker is an amino acid sequence of about 200 to about 1000 amino acids. In embodiments, the first peptidyl linker is an amino acid sequence of about 300 to about 1000 amino acids. In embodiments, the first peptidyl linker is an amino acid sequence of about 400 to about 1000 amino acids. In embodiments, the first peptidyl linker is an amino acid sequence of about 500 to about 1000 amino acids. In embodiments, the first peptidyl linker is an amino acid sequence of about 600 to about 1000 amino acids. In embodiments, the first peptidyl linker is an amino acid sequence of about 700 to about 1000 amino acids. In embodiments, the first peptidyl linker is an amino acid sequence of about 800 to about 1000 amino acids. In embodiments, the first peptidyl linker is an amino acid sequence of about 900 to about 1000 amino acids.

In embodiments, the first peptidyl linker is an amino acid sequence of about 1 to about 900 amino acids. In embodiments, the first peptidyl linker is an amino acid sequence of about 1 to about 800 amino acids. In embodiments, the first peptidyl linker is an amino acid sequence of about 1 to about 700 amino acids. In embodiments, the first peptidyl linker is an amino acid sequence of about 1 to about 600 amino acids. In embodiments, the first peptidyl linker is an amino acid sequence of about 1 to about 500 amino acids. In embodiments, the first peptidyl linker is an amino acid sequence of about 1 to about 400 amino acids. In embodiments, the first peptidyl linker is an amino acid sequence of about 1 to about 300 amino acids. In embodiments, the first peptidyl linker is an amino acid sequence of about 1 to about 200 amino acids. In embodiments, the first peptidyl linker is an amino acid sequence of about 1 to about 100 amino acids. In embodiments, the first peptidyl linker is an amino acid sequence of about 1 to about 50 amino acids. In embodiments, the first peptidyl linker is an amino acid sequence of about 1 to about 40 amino acids. In embodiments, the first peptidyl linker is an amino acid sequence of about 1 to about 30 amino acids. In embodiments, the first peptidyl linker is an amino acid sequence of about 1 to about 20 amino acids. In embodiments, the first peptidyl linker is an amino acid sequence of about 1 to about 10 amino acids. In embodiments, the first peptidyl linker is an amino acid sequence of about 1 to about 9 amino acids. In embodiments, the first peptidyl linker is an amino acid sequence of about 1 to about 8 amino acids. In embodiments, the first peptidyl linker is an amino acid sequence of about 1 to about 7 amino acids. In embodiments, the first peptidyl linker is an amino acid sequence of about 1 to about 6 amino acids. In embodiments, the first peptidyl linker is an amino acid sequence of about 1 to about 5 amino acids. In embodiments, the first peptidyl linker is an amino acid sequence of about 1 to about 4 amino acids. In embodiments, the first peptidyl linker is an amino acid sequence of about 1 to about 3 amino acids. In embodiments, the first peptidyl linker is an amino acid sequence of about 1 to about 2 amino acids.

In embodiments, the first peptidyl linker is an amino acid sequence of 1 to 1000 amino acids. In embodiments, the first peptidyl linker is an amino acid sequence of 2 to 1000 amino acids. In embodiments, the first peptidyl linker is an amino acid sequence of 3 to 1000 amino acids. In embodiments, the first peptidyl linker is an amino acid sequence of 4 to 1000 amino acids. In embodiments, the first peptidyl linker is an amino acid sequence of 5 to 1000 amino acids. In embodiments, the first peptidyl linker is an amino acid sequence of 6 to 1000 amino acids. In embodiments, the first peptidyl linker is an amino acid sequence of 7 to 1000 amino acids. In embodiments, the first peptidyl linker is an amino acid sequence of 8 to 1000 amino acids. In embodiments, the first peptidyl linker is an amino acid sequence of 9 to 1000 amino acids. In embodiments, the first peptidyl linker is an amino acid sequence of 10 to 1000 amino acids. In embodiments, the first peptidyl linker is an amino acid sequence of 20 to 1000 amino acids. In embodiments, the first peptidyl linker is an amino acid sequence of 30 to 1000 amino acids. In embodiments, the first peptidyl linker is an amino acid sequence of 40 to 1000 amino acids. In embodiments, the first peptidyl linker is an amino acid sequence of 50 to 1000 amino acids. In embodiments, the first peptidyl linker is an amino acid sequence of 100 to 1000 amino acids. In embodiments, the first peptidyl linker is an amino acid sequence of 200 to 1000 amino acids. In embodiments, the first peptidyl linker is an amino acid sequence of 300 to 1000 amino acids. In embodiments, the first peptidyl linker is an amino acid sequence of 400 to 1000 amino acids. In embodiments, the first peptidyl linker is an amino acid sequence of 500 to 1000 amino acids. In embodiments, the first peptidyl linker is an amino acid sequence of 600 to 1000 amino acids. In embodiments, the first peptidyl linker is an amino acid sequence of 700 to 1000 amino acids. In embodiments, the first peptidyl linker is an amino acid sequence of 800 to 1000 amino acids. In embodiments, the first peptidyl linker is an amino acid sequence of 900 to 1000 amino acids.

In embodiments, the first peptidyl linker is an amino acid sequence of 1 to 900 amino acids. In embodiments, the first peptidyl linker is an amino acid sequence of 1 to 800 amino acids. In embodiments, the first peptidyl linker is an amino acid sequence of 1 to 700 amino acids. In embodiments, the first peptidyl linker is an amino acid sequence of 1 to 600 amino acids. In embodiments, the first peptidyl linker is an amino acid sequence of 1 to 500 amino acids. In embodiments, the first peptidyl linker is an amino acid sequence of 1 to 400 amino acids. In embodiments, the first peptidyl linker is an amino acid sequence of 1 to 300 amino acids. In embodiments, the first peptidyl linker is an amino acid sequence of 1 to 200 amino acids. In embodiments, the first peptidyl linker is an amino acid sequence of 1 to 100 amino acids. In embodiments, the first peptidyl linker is an amino acid sequence of 1 to 50 amino acids. In embodiments, the first peptidyl linker is an amino acid sequence of 1 to 40 amino acids. In embodiments, the first peptidyl linker is an amino acid sequence of 1 to 30 amino acids. In embodiments, the first peptidyl linker is an amino acid sequence of 1 to 20 amino acids. In embodiments, the first peptidyl linker is an amino acid sequence of 1 to 10 amino acids. In embodiments, the first peptidyl linker is an amino acid sequence of 1 to 9 amino acids. In embodiments, the first peptidyl linker is an amino acid sequence of 1 to 8 amino acids. In embodiments, the first peptidyl linker is an amino acid sequence of 1 to 7 amino acids. In embodiments, the first peptidyl linker is an amino acid sequence of 1 to 6 amino acids. In embodiments, the first peptidyl linker is an amino acid sequence of 1 to 5 amino acids. In embodiments, the first peptidyl linker is an amino acid sequence of 1 to 4 amino acids. In embodiments, the first peptidyl linker is an amino acid sequence of 1 to 3 amino acids. In embodiments, the first peptidyl linker is an amino acid sequence of 1 to 2 amino acids.

In embodiments, the second peptidyl linker is an amino acid sequence of about 1 to about 1000 amino acids. In embodiments, the second peptidyl linker is an amino acid sequence of about 2 to about 1000 amino acids. In embodiments, the second peptidyl linker is an amino acid sequence of about 3 to about 1000 amino acids. In embodiments, the second peptidyl linker is an amino acid sequence of about 4 to about 1000 amino acids. In embodiments, the second peptidyl linker is an amino acid sequence of about 5 to about 1000 amino acids. In embodiments, the second peptidyl linker is an amino acid sequence of about 6 to about 1000 amino acids. In embodiments, the second peptidyl linker is an amino acid sequence of about 7 to about 1000 amino acids. In embodiments, the second peptidyl linker is an amino acid sequence of about 8 to about 1000 amino acids. In embodiments, the second peptidyl linker is an amino acid sequence of about 9 to about 1000 amino acids. In embodiments, the second peptidyl linker is an amino acid sequence of about 10 to about 1000 amino acids. In embodiments, the second peptidyl linker is an amino acid sequence of about 20 to about 1000 amino acids. In embodiments, the second peptidyl linker is an amino acid sequence of about 30 to about 1000 amino acids. In embodiments, the second peptidyl linker is an amino acid sequence of about 40 to about 1000 amino acids. In embodiments, the second peptidyl linker is an amino acid sequence of about 50 to about 1000 amino acids. In embodiments, the second peptidyl linker is an amino acid sequence of about 100 to about 1000 amino acids. In embodiments, the second peptidyl linker is an amino acid sequence of about 200 to about 1000 amino acids. In embodiments, the second peptidyl linker is an amino acid sequence of about 300 to about 1000 amino acids. In embodiments, the second peptidyl linker is an amino acid sequence of about 400 to about 1000 amino acids. In embodiments, the second peptidyl linker is an amino acid sequence of about 500 to about 1000 amino acids. In embodiments, the second peptidyl linker is an amino acid sequence of about 600 to about 1000 amino acids. In embodiments, the second peptidyl linker is an amino acid sequence of about 700 to about 1000 amino acids. In embodiments, the second peptidyl linker is an amino acid sequence of about 800 to about 1000 amino acids. In embodiments, the second peptidyl linker is an amino acid sequence of about 900 to about 1000 amino acids.

In embodiments, the second peptidyl linker is an amino acid sequence of about 1 to about 900 amino acids. In embodiments, the second peptidyl linker is an amino acid sequence of about 1 to about 800 amino acids. In embodiments, the second peptidyl linker is an amino acid sequence of about 1 to about 700 amino acids. In embodiments, the second peptidyl linker is an amino acid sequence of about 1 to about 600 amino acids. In embodiments, the second peptidyl linker is an amino acid sequence of about 1 to about 500 amino acids. In embodiments, the second peptidyl linker is an amino acid sequence of about 1 to about 400 amino acids. In embodiments, the second peptidyl linker is an amino acid sequence of about 1 to about 300 amino acids. In embodiments, the second peptidyl linker is an amino acid sequence of about 1 to about 200 amino acids. In embodiments, the second peptidyl linker is an amino acid sequence of about 1 to about 100 amino acids. In embodiments, the second peptidyl linker is an amino acid sequence of about 1 to about 50 amino acids. In embodiments, the second peptidyl linker is an amino acid sequence of about 1 to about 40 amino acids. In embodiments, the second peptidyl linker is an amino acid sequence of about 1 to about 30 amino acids. In embodiments, the second peptidyl linker is an amino acid sequence of about 1 to about 20 amino acids. In embodiments, the second peptidyl linker is an amino acid sequence of about 1 to about 10 amino acids. In embodiments, the second peptidyl linker is an amino acid sequence of about 1 to about 9 amino acids. In embodiments, the second peptidyl linker is an amino acid sequence of about 1 to about 8 amino acids. In embodiments, the second peptidyl linker is an amino acid sequence of about 1 to about 7 amino acids. In embodiments, the second peptidyl linker is an amino acid sequence of about 1 to about 6 amino acids. In embodiments, the second peptidyl linker is an amino acid sequence of about 1 to about 5 amino acids. In embodiments, the second peptidyl linker is an amino acid sequence of about 1 to about 4 amino acids. In embodiments, the second peptidyl linker is an amino acid sequence of about 1 to about 3 amino acids. In embodiments, the second peptidyl linker is an amino acid sequence of about 1 to about 2 amino acids.

In embodiments, the second peptidyl linker is an amino acid sequence of 1 to 1000 amino acids. In embodiments, the second peptidyl linker is an amino acid sequence of 2 to 1000 amino acids. In embodiments, the second peptidyl linker is an amino acid sequence of 3 to 1000 amino acids. In embodiments, the second peptidyl linker is an amino acid sequence of 4 to 1000 amino acids. In embodiments, the second peptidyl linker is an amino acid sequence of 5 to 1000 amino acids. In embodiments, the second peptidyl linker is an amino acid sequence of 6 to 1000 amino acids. In embodiments, the second peptidyl linker is an amino acid sequence of 7 to 1000 amino acids. In embodiments, the second peptidyl linker is an amino acid sequence of 8 to 1000 amino acids. In embodiments, the second peptidyl linker is an amino acid sequence of 9 to 1000 amino acids. In embodiments, the second peptidyl linker is an amino acid sequence of 10 to 1000 amino acids. In embodiments, the second peptidyl linker is an amino acid sequence of 20 to 1000 amino acids. In embodiments, the second peptidyl linker is an amino acid sequence of 30 to 1000 amino acids. In embodiments, the second peptidyl linker is an amino acid sequence of 40 to 1000 amino acids. In embodiments, the second peptidyl linker is an amino acid sequence of 50 to 1000 amino acids. In embodiments, the second peptidyl linker is an amino acid sequence of 100 to 1000 amino acids. In embodiments, the second peptidyl linker is an amino acid sequence of 200 to 1000 amino acids. In embodiments, the second peptidyl linker is an amino acid sequence of 300 to 1000 amino acids. In embodiments, the second peptidyl linker is an amino acid sequence of 400 to 1000 amino acids. In embodiments, the second peptidyl linker is an amino acid sequence of 500 to 1000 amino acids. In embodiments, the second peptidyl linker is an amino acid sequence of 600 to 1000 amino acids. In embodiments, the second peptidyl linker is an amino acid sequence of 700 to 1000 amino acids. In embodiments, the second peptidyl linker is an amino acid sequence of 800 to 1000 amino acids. In embodiments, the second peptidyl linker is an amino acid sequence of 900 to 1000 amino acids.

In embodiments, the second peptidyl linker is an amino acid sequence of 1 to 900 amino acids. In embodiments, the second peptidyl linker is an amino acid sequence of 1 to 800 amino acids. In embodiments, the second peptidyl linker is an amino acid sequence of 1 to 700 amino acids. In embodiments, the second peptidyl linker is an amino acid sequence of 1 to 600 amino acids. In embodiments, the second peptidyl linker is an amino acid sequence of 1 to 500 amino acids. In embodiments, the second peptidyl linker is an amino acid sequence of 1 to 400 amino acids. In embodiments, the second peptidyl linker is an amino acid sequence of 1 to 300 amino acids. In embodiments, the second peptidyl linker is an amino acid sequence of 1 to 200 amino acids. In embodiments, the second peptidyl linker is an amino acid sequence of 1 to 100 amino acids. In embodiments, the second peptidyl linker is an amino acid sequence of 1 to 50 amino acids. In embodiments, the second peptidyl linker is an amino acid sequence of 1 to 40 amino acids. In embodiments, the second peptidyl linker is an amino acid sequence of 1 to 30 amino acids. In embodiments, the second peptidyl linker is an amino acid sequence of 1 to 20 amino acids. In embodiments, the second peptidyl linker is an amino acid sequence of 1 to 10 amino acids. In embodiments, the second peptidyl linker is an amino acid sequence of 1 to 9 amino acids. In embodiments, the second peptidyl linker is an amino acid sequence of 1 to 8 amino acids. In embodiments, the second peptidyl linker is an amino acid sequence of 1 to 7 amino acids. In embodiments, the second peptidyl linker is an amino acid sequence of 1 to 6 amino acids. In embodiments, the second peptidyl linker is an amino acid sequence of 1 to 5 amino acids. In embodiments, the second peptidyl linker is an amino acid sequence of 1 to 4 amino acids. In embodiments, the second peptidyl linker is an amino acid sequence of 1 to 3 amino acids. In embodiments, the second peptidyl linker is an amino acid sequence of 1 to 2 amino acids.

In embodiments, the third peptidyl linker is an amino acid sequence of about 1 to about 1000 amino acids. In embodiments, the third peptidyl linker is an amino acid sequence of about 2 to about 1000 amino acids. In embodiments, the third peptidyl linker is an amino acid sequence of about 3 to about 1000 amino acids. In embodiments, the third peptidyl linker is an amino acid sequence of about 4 to about 1000 amino acids. In embodiments, the third peptidyl linker is an amino acid sequence of about 5 to about 1000 amino acids. In embodiments, the third peptidyl linker is an amino acid sequence of about 6 to about 1000 amino acids. In embodiments, the third peptidyl linker is an amino acid sequence of about 7 to about 1000 amino acids. In embodiments, the third peptidyl linker is an amino acid sequence of about 8 to about 1000 amino acids. In embodiments, the third peptidyl linker is an amino acid sequence of about 9 to about 1000 amino acids. In embodiments, the third peptidyl linker is an amino acid sequence of about 10 to about 1000 amino acids. In embodiments, the third peptidyl linker is an amino acid sequence of about 20 to about 1000 amino acids. In embodiments, the third peptidyl linker is an amino acid sequence of about 30 to about 1000 amino acids. In embodiments, the third peptidyl linker is an amino acid sequence of about 40 to about 1000 amino acids. In embodiments, the third peptidyl linker is an amino acid sequence of about 50 to about 1000 amino acids. In embodiments, the third peptidyl linker is an amino acid sequence of about 100 to about 1000 amino acids. In embodiments, the third peptidyl linker is an amino acid sequence of about 200 to about 1000 amino acids. In embodiments, the third peptidyl linker is an amino acid sequence of about 300 to about 1000 amino acids. In embodiments, the third peptidyl linker is an amino acid sequence of about 400 to about 1000 amino acids. In embodiments, the third peptidyl linker is an amino acid sequence of about 500 to about 1000 amino acids. In embodiments, the third peptidyl linker is an amino acid sequence of about 600 to about 1000 amino acids. In embodiments, the third peptidyl linker is an amino acid sequence of about 700 to about 1000 amino acids. In embodiments, the third peptidyl linker is an amino acid sequence of about 800 to about 1000 amino acids. In embodiments, the third peptidyl linker is an amino acid sequence of about 900 to about 1000 amino acids.

In embodiments, the third peptidyl linker is an amino acid sequence of about 1 to about 900 amino acids. In embodiments, the third peptidyl linker is an amino acid sequence of about 1 to about 800 amino acids. In embodiments, the third peptidyl linker is an amino acid sequence of about 1 to about 700 amino acids. In embodiments, the third peptidyl linker is an amino acid sequence of about 1 to about 600 amino acids. In embodiments, the third peptidyl linker is an amino acid sequence of about 1 to about 500 amino acids. In embodiments, the third peptidyl linker is an amino acid sequence of about 1 to about 400 amino acids. In embodiments, the third peptidyl linker is an amino acid sequence of about 1 to about 300 amino acids. In embodiments, the third peptidyl linker is an amino acid sequence of about 1 to about 200 amino acids. In embodiments, the third peptidyl linker is an amino acid sequence of about 1 to about 100 amino acids. In embodiments, the third peptidyl linker is an amino acid sequence of about 1 to about 50 amino acids. In embodiments, the third peptidyl linker is an amino acid sequence of about 1 to about 40 amino acids. In embodiments, the third peptidyl linker is an amino acid sequence of about 1 to about 30 amino acids. In embodiments, the third peptidyl linker is an amino acid sequence of about 1 to about 20 amino acids. In embodiments, the third peptidyl linker is an amino acid sequence of about 1 to about 10 amino acids. In embodiments, the third peptidyl linker is an amino acid sequence of about 1 to about 9 amino acids. In embodiments, the third peptidyl linker is an amino acid sequence of about 1 to about 8 amino acids. In embodiments, the third peptidyl linker is an amino acid sequence of about 1 to about 7 amino acids. In embodiments, the third peptidyl linker is an amino acid sequence of about 1 to about 6 amino acids. In embodiments, the third peptidyl linker is an amino acid sequence of about 1 to about 5 amino acids. In embodiments, the third peptidyl linker is an amino acid sequence of about 1 to about 4 amino acids. In embodiments, the third peptidyl linker is an amino acid sequence of about 1 to about 3 amino acids. In embodiments, the third peptidyl linker is an amino acid sequence of about 1 to about 2 amino acids.

In embodiments, the third peptidyl linker is an amino acid sequence of 1 to 1000 amino acids. In embodiments, the third peptidyl linker is an amino acid sequence of 2 to 1000 amino acids. In embodiments, the third peptidyl linker is an amino acid sequence of 3 to 1000 amino acids. In embodiments, the third peptidyl linker is an amino acid sequence of 4 to 1000 amino acids. In embodiments, the third peptidyl linker is an amino acid sequence of 5 to 1000 amino acids. In embodiments, the third peptidyl linker is an amino acid sequence of 6 to 1000 amino acids. In embodiments, the third peptidyl linker is an amino acid sequence of 7 to 1000 amino acids. In embodiments, the third peptidyl linker is an amino acid sequence of 8 to 1000 amino acids. In embodiments, the third peptidyl linker is an amino acid sequence of 9 to 1000 amino acids. In embodiments, the third peptidyl linker is an amino acid sequence of 10 to 1000 amino acids. In embodiments, the third peptidyl linker is an amino acid sequence of 20 to 1000 amino acids. In embodiments, the third peptidyl linker is an amino acid sequence of 30 to 1000 amino acids. In embodiments, the third peptidyl linker is an amino acid sequence of 40 to 1000 amino acids. In embodiments, the third peptidyl linker is an amino acid sequence of 50 to 1000 amino acids. In embodiments, the third peptidyl linker is an amino acid sequence of 100 to 1000 amino acids. In embodiments, the third peptidyl linker is an amino acid sequence of 200 to 1000 amino acids. In embodiments, the third peptidyl linker is an amino acid sequence of 300 to 1000 amino acids. In embodiments, the third peptidyl linker is an amino acid sequence of 400 to 1000 amino acids. In embodiments, the third peptidyl linker is an amino acid sequence of 500 to 1000 amino acids. In embodiments, the third peptidyl linker is an amino acid sequence of 600 to 1000 amino acids. In embodiments, the third peptidyl linker is an amino acid sequence of 700 to 1000 amino acids. In embodiments, the third peptidyl linker is an amino acid sequence of 800 to 1000 amino acids. In embodiments, the third peptidyl linker is an amino acid sequence of 900 to 1000 amino acids.

In embodiments, the third peptidyl linker is an amino acid sequence of 1 to 900 amino acids. In embodiments, the third peptidyl linker is an amino acid sequence of 1 to 800 amino acids. In embodiments, the third peptidyl linker is an amino acid sequence of 1 to 700 amino acids. In embodiments, the third peptidyl linker is an amino acid sequence of 1 to 600 amino acids. In embodiments, the third peptidyl linker is an amino acid sequence of 1 to 500 amino acids. In embodiments, the third peptidyl linker is an amino acid sequence of 1 to 400 amino acids. In embodiments, the third peptidyl linker is an amino acid sequence of 1 to 300 amino acids. In embodiments, the third peptidyl linker is an amino acid sequence of 1 to 200 amino acids. In embodiments, the third peptidyl linker is an amino acid sequence of 1 to 100 amino acids. In embodiments, the third peptidyl linker is an amino acid sequence of 1 to 50 amino acids. In embodiments, the third peptidyl linker is an amino acid sequence of 1 to 40 amino acids. In embodiments, the third peptidyl linker is an amino acid sequence of 1 to 30 amino acids. In embodiments, the third peptidyl linker is an amino acid sequence of 1 to 20 amino acids. In embodiments, the third peptidyl linker is an amino acid sequence of 1 to 10 amino acids. In embodiments, the third peptidyl linker is an amino acid sequence of 1 to 9 amino acids. In embodiments, the third peptidyl linker is an amino acid sequence of 1 to 8 amino acids. In embodiments, the third peptidyl linker is an amino acid sequence of 1 to 7 amino acids. In embodiments, the third peptidyl linker is an amino acid sequence of 1 to 6 amino acids. In embodiments, the third peptidyl linker is an amino acid sequence of 1 to 5 amino acids. In embodiments, the third peptidyl linker is an amino acid sequence of 1 to 4 amino acids. In embodiments, the third peptidyl linker is an amino acid sequence of 1 to 3 amino acids. In embodiments, the third peptidyl linker is an amino acid sequence of 1 to 2 amino acids.

In embodiments, the fourth peptidyl linker is an amino acid sequence of about 1 to about 1000 amino acids. In embodiments, the fourth peptidyl linker is an amino acid sequence of about 2 to about 1000 amino acids. In embodiments, the fourth peptidyl linker is an amino acid sequence of about 3 to about 1000 amino acids. In embodiments, the fourth peptidyl linker is an amino acid sequence of about 4 to about 1000 amino acids. In embodiments, the fourth peptidyl linker is an amino acid sequence of about 5 to about 1000 amino acids. In embodiments, the fourth peptidyl linker is an amino acid sequence of about 6 to about 1000 amino acids. In embodiments, the fourth peptidyl linker is an amino acid sequence of about 7 to about 1000 amino acids. In embodiments, the fourth peptidyl linker is an amino acid sequence of about 8 to about 1000 amino acids. In embodiments, the fourth peptidyl linker is an amino acid sequence of about 9 to about 1000 amino acids. In embodiments, the fourth peptidyl linker is an amino acid sequence of about 10 to about 1000 amino acids. In embodiments, the fourth peptidyl linker is an amino acid sequence of about 20 to about 1000 amino acids. In embodiments, the fourth peptidyl linker is an amino acid sequence of about 30 to about 1000 amino acids. In embodiments, the fourth peptidyl linker is an amino acid sequence of about 40 to about 1000 amino acids. In embodiments, the fourth peptidyl linker is an amino acid sequence of about 50 to about 1000 amino acids. In embodiments, the fourth peptidyl linker is an amino acid sequence of about 100 to about 1000 amino acids. In embodiments, the fourth peptidyl linker is an amino acid sequence of about 200 to about 1000 amino acids. In embodiments, the fourth peptidyl linker is an amino acid sequence of about 300 to about 1000 amino acids. In embodiments, the fourth peptidyl linker is an amino acid sequence of about 400 to about 1000 amino acids. In embodiments, the fourth peptidyl linker is an amino acid sequence of about 500 to about 1000 amino acids. In embodiments, the fourth peptidyl linker is an amino acid sequence of about 600 to about 1000 amino acids. In embodiments, the fourth peptidyl linker is an amino acid sequence of about 700 to about 1000 amino acids. In embodiments, the fourth peptidyl linker is an amino acid sequence of about 800 to about 1000 amino acids. In embodiments, the fourth peptidyl linker is an amino acid sequence of about 900 to about 1000 amino acids.

In embodiments, the fourth peptidyl linker is an amino acid sequence of about 1 to about 900 amino acids. In embodiments, the fourth peptidyl linker is an amino acid sequence of about 1 to about 800 amino acids. In embodiments, the fourth peptidyl linker is an amino acid sequence of about 1 to about 700 amino acids. In embodiments, the fourth peptidyl linker is an amino acid sequence of about 1 to about 600 amino acids. In embodiments, the fourth peptidyl linker is an amino acid sequence of about 1 to about 500 amino acids. In embodiments, the fourth peptidyl linker is an amino acid sequence of about 1 to about 400 amino acids. In embodiments, the fourth peptidyl linker is an amino acid sequence of about 1 to about 300 amino acids. In embodiments, the fourth peptidyl linker is an amino acid sequence of about 1 to about 200 amino acids. In embodiments, the fourth peptidyl linker is an amino acid sequence of about 1 to about 100 amino acids. In embodiments, the fourth peptidyl linker is an amino acid sequence of about 1 to about 50 amino acids. In embodiments, the fourth peptidyl linker is an amino acid sequence of about 1 to about 40 amino acids. In embodiments, the fourth peptidyl linker is an amino acid sequence of about 1 to about 30 amino acids. In embodiments, the fourth peptidyl linker is an amino acid sequence of about 1 to about 20 amino acids. In embodiments, the fourth peptidyl linker is an amino acid sequence of about 1 to about 10 amino acids. In embodiments, the fourth peptidyl linker is an amino acid sequence of about 1 to about 9 amino acids. In embodiments, the fourth peptidyl linker is an amino acid sequence of about 1 to about 8 amino acids. In embodiments, the fourth peptidyl linker is an amino acid sequence of about 1 to about 7 amino acids. In embodiments, the fourth peptidyl linker is an amino acid sequence of about 1 to about 6 amino acids. In embodiments, the fourth peptidyl linker is an amino acid sequence of about 1 to about 5 amino acids. In embodiments, the fourth peptidyl linker is an amino acid sequence of about 1 to about 4 amino acids. In embodiments, the fourth peptidyl linker is an amino acid sequence of about 1 to about 3 amino acids. In embodiments, the fourth peptidyl linker is an amino acid sequence of about 1 to about 2 amino acids.

In embodiments, the fourth peptidyl linker is an amino acid sequence of 1 to 1000 amino acids. In embodiments, the fourth peptidyl linker is an amino acid sequence of 2 to 1000 amino acids. In embodiments, the fourth peptidyl linker is an amino acid sequence of 3 to 1000 amino acids. In embodiments, the fourth peptidyl linker is an amino acid sequence of 4 to 1000 amino acids. In embodiments, the fourth peptidyl linker is an amino acid sequence of 5 to 1000 amino acids. In embodiments, the fourth peptidyl linker is an amino acid sequence of 6 to 1000 amino acids. In embodiments, the fourth peptidyl linker is an amino acid sequence of 7 to 1000 amino acids. In embodiments, the fourth peptidyl linker is an amino acid sequence of 8 to 1000 amino acids. In embodiments, the fourth peptidyl linker is an amino acid sequence of 9 to 1000 amino acids. In embodiments, the fourth peptidyl linker is an amino acid sequence of 10 to 1000 amino acids. In embodiments, the fourth peptidyl linker is an amino acid sequence of 20 to 1000 amino acids. In embodiments, the fourth peptidyl linker is an amino acid sequence of 30 to 1000 amino acids. In embodiments, the fourth peptidyl linker is an amino acid sequence of 40 to 1000 amino acids. In embodiments, the fourth peptidyl linker is an amino acid sequence of 50 to 1000 amino acids. In embodiments, the fourth peptidyl linker is an amino acid sequence of 100 to 1000 amino acids. In embodiments, the fourth peptidyl linker is an amino acid sequence of 200 to 1000 amino acids. In embodiments, the fourth peptidyl linker is an amino acid sequence of 300 to 1000 amino acids. In embodiments, the fourth peptidyl linker is an amino acid sequence of 400 to 1000 amino acids. In embodiments, the fourth peptidyl linker is an amino acid sequence of 500 to 1000 amino acids. In embodiments, the fourth peptidyl linker is an amino acid sequence of 600 to 1000 amino acids. In embodiments, the fourth peptidyl linker is an amino acid sequence of 700 to 1000 amino acids. In embodiments, the fourth peptidyl linker is an amino acid sequence of 800 to 1000 amino acids. In embodiments, the fourth peptidyl linker is an amino acid sequence of 900 to 1000 amino acids.

In embodiments, the fourth peptidyl linker is an amino acid sequence of 1 to 900 amino acids. In embodiments, the fourth peptidyl linker is an amino acid sequence of 1 to 800 amino acids. In embodiments, the fourth peptidyl linker is an amino acid sequence of 1 to 700 amino acids. In embodiments, the fourth peptidyl linker is an amino acid sequence of 1 to 600 amino acids. In embodiments, the fourth peptidyl linker is an amino acid sequence of 1 to 500 amino acids. In embodiments, the fourth peptidyl linker is an amino acid sequence of 1 to 400 amino acids. In embodiments, the fourth peptidyl linker is an amino acid sequence of 1 to 300 amino acids. In embodiments, the fourth peptidyl linker is an amino acid sequence of 1 to 200 amino acids. In embodiments, the fourth peptidyl linker is an amino acid sequence of 1 to 100 amino acids. In embodiments, the fourth peptidyl linker is an amino acid sequence of 1 to 50 amino acids. In embodiments, the fourth peptidyl linker is an amino acid sequence of 1 to 40 amino acids. In embodiments, the fourth peptidyl linker is an amino acid sequence of 1 to 30 amino acids. In embodiments, the fourth peptidyl linker is an amino acid sequence of 1 to 20 amino acids. In embodiments, the fourth peptidyl linker is an amino acid sequence of 1 to 10 amino acids. In embodiments, the fourth peptidyl linker is an amino acid sequence of 1 to 9 amino acids. In embodiments, the fourth peptidyl linker is an amino acid sequence of 1 to 8 amino acids. In embodiments, the fourth peptidyl linker is an amino acid sequence of 1 to 7 amino acids. In embodiments, the fourth peptidyl linker is an amino acid sequence of 1 to 6 amino acids. In embodiments, the fourth peptidyl linker is an amino acid sequence of 1 to 5 amino acids. In embodiments, the fourth peptidyl linker is an amino acid sequence of 1 to 4 amino acids. In embodiments, the fourth peptidyl linker is an amino acid sequence of 1 to 3 amino acids. In embodiments, the fourth peptidyl linker is an amino acid sequence of 1 to 2 amino acids.

In embodiments, the first peptidyl linker, the second peptidyl linker, the third peptidyl linker, and/or the fourth peptidyl linker independently include at least 1 glycine, serine, or threonine. In embodiments, the first peptidyl linker, the second peptidyl linker, the third peptidyl linker, and/or the fourth peptidyl linker independently include at least 2 glycines, serines, or threonines. In embodiments, the first peptidyl linker, the second peptidyl linker, the third peptidyl linker, and/or the fourth peptidyl linker independently include at least 3 glycines, serines, or threonines. In embodiments, the first peptidyl linker, the second peptidyl linker, the third peptidyl linker, and/or the fourth peptidyl linker independently include at least 4 glycines, serines, or threonines. In embodiments, the first peptidyl linker, the second peptidyl linker, the third peptidyl linker, and/or the fourth peptidyl linker independently include at least 5 glycines, serines, or threonines. In embodiments, the first peptidyl linker, the second peptidyl linker, the third peptidyl linker, and/or the fourth peptidyl linker independently include at least 6 glycines, serines, or threonines. In embodiments, the first peptidyl linker, the second peptidyl linker, the third peptidyl linker, and/or the fourth peptidyl linker independently include at least 7 glycines, serines, or threonines. In embodiments, the first peptidyl linker, the second peptidyl linker, the third peptidyl linker, and/or the fourth peptidyl linker independently include at least 8 glycines, serines, or threonines. In embodiments, the first peptidyl linker, the second peptidyl linker, the third peptidyl linker, and/or the fourth peptidyl linker independently include at least 9 glycines, serines, or threonines. In embodiments, the first peptidyl linker, the second peptidyl linker, the third peptidyl linker, and/or the fourth peptidyl linker independently include at least 10 glycines, serines, or threonines. In embodiments, the first peptidyl linker, the second peptidyl linker, the third peptidyl linker, and/or the fourth peptidyl linker independently include at least 11 glycines, serines, or threonines. In embodiments, the first peptidyl linker, the second peptidyl linker, the third peptidyl linker, and/or the fourth peptidyl linker independently include at least 12 glycines, serines, or threonines. In embodiments, the first peptidyl linker, the second peptidyl linker, the third peptidyl linker, and/or the fourth peptidyl linker independently include at least 13 glycines, serines, or threonines. In embodiments, the first peptidyl linker, the second peptidyl linker, the third peptidyl linker, and/or the fourth peptidyl linker independently include at least 14 glycines, serines, or threonines. In embodiments, the first peptidyl linker, the second peptidyl linker, the third peptidyl linker, and/or the fourth peptidyl linker independently include at least 15 glycines, serines, or threonines.

In embodiments, the first peptidyl linker, the second peptidyl linker, the third peptidyl linker, and/or the fourth peptidyl linker independently include at least 1 glycine. In embodiments, the first peptidyl linker, the second peptidyl linker, the third peptidyl linker, and/or the fourth peptidyl linker independently include at least 2 glycines. In embodiments, the first peptidyl linker, the second peptidyl linker, the third peptidyl linker, and/or the fourth peptidyl linker independently include at least 3 glycines. In embodiments, the first peptidyl linker, the second peptidyl linker, the third peptidyl linker, and/or the fourth peptidyl linker independently include at least 4 glycines. In embodiments, the first peptidyl linker, the second peptidyl linker, the third peptidyl linker, and/or the fourth peptidyl linker independently include at least 5 glycines. In embodiments, the first peptidyl linker, the second peptidyl linker, the third peptidyl linker, and/or the fourth peptidyl linker independently include at least 6 glycines. In embodiments, the first peptidyl linker, the second peptidyl linker, the third peptidyl linker, and/or the fourth peptidyl linker independently include at least 7 glycines. In embodiments, the first peptidyl linker, the second peptidyl linker, the third peptidyl linker, and/or the fourth peptidyl linker independently include at least 8 glycines. In embodiments, the first peptidyl linker, the second peptidyl linker, the third peptidyl linker, and/or the fourth peptidyl linker independently include at least 9 glycines. In embodiments, the first peptidyl linker, the second peptidyl linker, the third peptidyl linker, and/or the fourth peptidyl linker independently include at least 10 glycines. In embodiments, the first peptidyl linker, the second peptidyl linker, the third peptidyl linker, and/or the fourth peptidyl linker independently include at least 11 glycines. In embodiments, the first peptidyl linker, the second peptidyl linker, the third peptidyl linker, and/or the fourth peptidyl linker independently include at least 12 glycines. In embodiments, the first peptidyl linker, the second peptidyl linker, the third peptidyl linker, and/or the fourth peptidyl linker independently include at least 13 glycines. In embodiments, the first peptidyl linker, the second peptidyl linker, the third peptidyl linker, and/or the fourth peptidyl linker independently include at least 14 glycines. In embodiments, the first peptidyl linker, the second peptidyl linker, the third peptidyl linker, and/or the fourth peptidyl linker independently include at least 15 glycines.

In embodiments, the first peptidyl linker, the second peptidyl linker, the third peptidyl linker, and/or the fourth peptidyl linker independently include at least 1 serine. In embodiments, the first peptidyl linker, the second peptidyl linker, the third peptidyl linker, and/or the fourth peptidyl linker independently include at least 2 serines. In embodiments, the first peptidyl linker, the second peptidyl linker, the third peptidyl linker, and/or the fourth peptidyl linker independently include at least 3 serines. In embodiments, the first peptidyl linker, the second peptidyl linker, the third peptidyl linker, and/or the fourth peptidyl linker independently include at least 4 serines. In embodiments, the first peptidyl linker, the second peptidyl linker, the third peptidyl linker, and/or the fourth peptidyl linker independently include at least 5 serines. In embodiments, the first peptidyl linker, the second peptidyl linker, the third peptidyl linker, and/or the fourth peptidyl linker independently include at least 6 serines. In embodiments, the first peptidyl linker, the second peptidyl linker, the third peptidyl linker, and/or the fourth peptidyl linker independently include at least 7 serines. In embodiments, the first peptidyl linker, the second peptidyl linker, the third peptidyl linker, and/or the fourth peptidyl linker independently include at least 8 serines. In embodiments, the first peptidyl linker, the second peptidyl linker, the third peptidyl linker, and/or the fourth peptidyl linker independently include at least 9 serines. In embodiments, the first peptidyl linker, the second peptidyl linker, the third peptidyl linker, and/or the fourth peptidyl linker independently include at least 10 serines. In embodiments, the first peptidyl linker, the second peptidyl linker, the third peptidyl linker, and/or the fourth peptidyl linker independently include at least 11 serines. In embodiments, the first peptidyl linker, the second peptidyl linker, the third peptidyl linker, and/or the fourth peptidyl linker independently include at least 12 serines. In embodiments, the first peptidyl linker, the second peptidyl linker, the third peptidyl linker, and/or the fourth peptidyl linker independently include at least 13 serines. In embodiments, the first peptidyl linker, the second peptidyl linker, the third peptidyl linker, and/or the fourth peptidyl linker independently include at least 14 serines. In embodiments, the first peptidyl linker, the second peptidyl linker, the third peptidyl linker, and/or the fourth peptidyl linker independently include at least 15 serines.

In embodiments, the first peptidyl linker, the second peptidyl linker, the third peptidyl linker, and/or the fourth peptidyl linker independently include at least 1 threonine. In embodiments, the first peptidyl linker, the second peptidyl linker, the third peptidyl linker, and/or the fourth peptidyl linker independently include at least 2 threonines. In embodiments, the first peptidyl linker, the second peptidyl linker, the third peptidyl linker, and/or the fourth peptidyl linker independently include at least 3 threonines. In embodiments, the first peptidyl linker, the second peptidyl linker, the third peptidyl linker, and/or the fourth peptidyl linker independently include at least 4 threonines. In embodiments, the first peptidyl linker, the second peptidyl linker, the third peptidyl linker, and/or the fourth peptidyl linker independently include at least 5 threonines. In embodiments, the first peptidyl linker, the second peptidyl linker, the third peptidyl linker, and/or the fourth peptidyl linker independently include at least 6 threonines. In embodiments, the first peptidyl linker, the second peptidyl linker, the third peptidyl linker, and/or the fourth peptidyl linker independently include at least 7 threonines. In embodiments, the first peptidyl linker, the second peptidyl linker, the third peptidyl linker, and/or the fourth peptidyl linker independently include at least 8 threonines. In embodiments, the first peptidyl linker, the second peptidyl linker, the third peptidyl linker, and/or the fourth peptidyl linker independently include at least 9 threonines. In embodiments, the first peptidyl linker, the second peptidyl linker, the third peptidyl linker, and/or the fourth peptidyl linker independently include at least 10 threonines. In embodiments, the first peptidyl linker, the second peptidyl linker, the third peptidyl linker, and/or the fourth peptidyl linker independently include at least 11 threonines. In embodiments, the first peptidyl linker, the second peptidyl linker, the third peptidyl linker, and/or the fourth peptidyl linker independently include at least 12 threonines. In embodiments, the first peptidyl linker, the second peptidyl linker, the third peptidyl linker, and/or the fourth peptidyl linker independently include at least 13 threonines. In embodiments, the first peptidyl linker, the second peptidyl linker, the third peptidyl linker, and/or the fourth peptidyl linker independently include at least 14 threonines. In embodiments, the first peptidyl linker, the second peptidyl linker, the third peptidyl linker, and/or the fourth peptidyl linker independently include at least 15 threonines.

In embodiments, the first peptidyl linker, the second peptidyl linker, the third peptidyl linker, or the fourth peptidyl linker independently includes at least 1 glycine, serine, or threonine. In embodiments, the first peptidyl linker, the second peptidyl linker, the third peptidyl linker, and the fourth peptidyl linker independently include at least 1 glycine, serine, or threonine. In embodiments, the first peptidyl linker, the second peptidyl linker, the third peptidyl linker, and the fourth peptidyl linker independently include at least 1 glycine. In embodiments, the first peptidyl linker, the second peptidyl linker, the third peptidyl linker, and the fourth peptidyl linker independently include at least 1 serine. In embodiments, the first peptidyl linker, the second peptidyl linker, the third peptidyl linker, and the fourth peptidyl linker independently include at least 1 threonine.

In embodiments, the first peptidyl linker includes at least 1 glycine, serine, or threonine. In embodiments, the first peptidyl linker includes at least 2 glycines, serines, or threonines. In embodiments, the first peptidyl linker includes at least 3 glycines, serines, or threonines. In embodiments, the first peptidyl linker includes at least 4 glycines, serines, or threonines. In embodiments, the first peptidyl linker includes at least 5 glycines, serines, or threonines. In embodiments, the first peptidyl linker includes at least 6 glycines, serines, or threonines. In embodiments, the first peptidyl linker includes at least 7 glycines, serines, or threonines. In embodiments, the first peptidyl linker includes at least 8 glycines, serines, or threonines. In embodiments, the first peptidyl linker includes at least 9 glycines, serines, or threonines. In embodiments, the first peptidyl linker includes at least 10 glycines, serines, or threonines. In embodiments, the first peptidyl linker includes at least 11 glycines, serines, or threonines. In embodiments, the first peptidyl linker includes at least 12 glycines, serines, or threonines. In embodiments, the first peptidyl linker includes at least 13 glycines, serines, or threonines. In embodiments, the first peptidyl linker includes at least 14 glycines, serines, or threonines. In embodiments, the first peptidyl linker includes at least 15 glycines, serines, or threonines.

In embodiments, the first peptidyl linker includes at least 1 glycine. In embodiments, the first peptidyl linker includes at least 2 glycines. In embodiments, the first peptidyl linker includes at least 3 glycines. In embodiments, the first peptidyl linker includes at least 4 glycines. In embodiments, the first peptidyl linker includes at least 5 glycines. In embodiments, the first peptidyl linker includes at least 6 glycines. In embodiments, the first peptidyl linker includes at least 7 glycines. In embodiments, the first peptidyl linker includes at least 8 glycines, serines, or threonines. In embodiments, the first peptidyl linker includes at least 9 glycines. In embodiments, the first peptidyl linker includes at least 10 glycines. In embodiments, the first peptidyl linker includes at least 11 glycines. In embodiments, the first peptidyl linker includes at least 12 glycines. In embodiments, the first peptidyl linker includes at least 13 glycines. In embodiments, the first peptidyl linker includes at least 14 glycines. In embodiments, the first peptidyl linker includes at least 15 glycines.

In embodiments, the first peptidyl linker includes at least 1 serine. In embodiments, the first peptidyl linker includes at least 2 serines. In embodiments, the first peptidyl linker includes at least 3 serines. In embodiments, the first peptidyl linker includes at least 4 serines. In embodiments, the first peptidyl linker includes at least 5 serines. In embodiments, the first peptidyl linker includes at least 6 serines. In embodiments, the first peptidyl linker includes at least 7 serines. In embodiments, the first peptidyl linker includes at least 8 serines, serines, or threonines. In embodiments, the first peptidyl linker includes at least 9 serines. In embodiments, the first peptidyl linker includes at least 10 serines. In embodiments, the first peptidyl linker includes at least 11 serines. In embodiments, the first peptidyl linker includes at least 12 serines. In embodiments, the first peptidyl linker includes at least 13 serines. In embodiments, the first peptidyl linker includes at least 14 serines. In embodiments, the first peptidyl linker includes at least 15 serines.

In embodiments, the first peptidyl linker includes at least 1 threonine. In embodiments, the first peptidyl linker includes at least 2 threonines. In embodiments, the first peptidyl linker includes at least 3 threonines. In embodiments, the first peptidyl linker includes at least 4 threonines. In embodiments, the first peptidyl linker includes at least 5 threonines. In embodiments, the first peptidyl linker includes at least 6 threonines. In embodiments, the first peptidyl linker includes at least 7 threonines. In embodiments, the first peptidyl linker includes at least 8 threonines, serines, or threonines. In embodiments, the first peptidyl linker includes at least 9 threonines. In embodiments, the first peptidyl linker includes at least 10 threonines. In embodiments, the first peptidyl linker includes at least 11 threonines. In embodiments, the first peptidyl linker includes at least 12 threonines. In embodiments, the first peptidyl linker includes at least 13 threonines. In embodiments, the first peptidyl linker includes at least 14 threonines. In embodiments, the first peptidyl linker includes at least 15 threonines.

In embodiments, the second peptidyl linker includes at least 1 glycine, serine, or threonine. In embodiments, the second peptidyl linker includes at least 2 glycines, serines, or threonines. In embodiments, the second peptidyl linker includes at least 3 glycines, serines, or threonines. In embodiments, the second peptidyl linker includes at least 4 glycines, serines, or threonines. In embodiments, the second peptidyl linker includes at least 5 glycines, serines, or threonines. In embodiments, the second peptidyl linker includes at least 6 glycines, serines, or threonines. In embodiments, the second peptidyl linker includes at least 7 glycines, serines, or threonines. In embodiments, the second peptidyl linker includes at least 8 glycines, serines, or threonines. In embodiments, the second peptidyl linker includes at least 9 glycines, serines, or threonines. In embodiments, the second peptidyl linker includes at least 10 glycines, serines, or threonines. In embodiments, the second peptidyl linker includes at least 11 glycines, serines, or threonines. In embodiments, the second peptidyl linker includes at least 12 glycines, serines, or threonines. In embodiments, the second peptidyl linker includes at least 13 glycines, serines, or threonines. In embodiments, the second peptidyl linker includes at least 14 glycines, serines, or threonines. In embodiments, the second peptidyl linker includes at least 15 glycines, serines, or threonines.

In embodiments, the second peptidyl linker includes at least 1 glycine. In embodiments, the second peptidyl linker includes at least 2 glycines. In embodiments, the second peptidyl linker includes at least 3 glycines. In embodiments, the second peptidyl linker includes at least 4 glycines. In embodiments, the second peptidyl linker includes at least 5 glycines. In embodiments, the second peptidyl linker includes at least 6 glycines. In embodiments, the second peptidyl linker includes at least 7 glycines. In embodiments, the second peptidyl linker includes at least 8 glycines, serines, or threonines. In embodiments, the second peptidyl linker includes at least 9 glycines. In embodiments, the second peptidyl linker includes at least 10 glycines. In embodiments, the second peptidyl linker includes at least 11 glycines. In embodiments, the second peptidyl linker includes at least 12 glycines. In embodiments, the second peptidyl linker includes at least 13 glycines. In embodiments, the second peptidyl linker includes at least 14 glycines. In embodiments, the second peptidyl linker includes at least 15 glycines.

In embodiments, the second peptidyl linker includes at least 1 serine. In embodiments, the second peptidyl linker includes at least 2 serines. In embodiments, the second peptidyl linker includes at least 3 serines. In embodiments, the second peptidyl linker includes at least 4 serines. In embodiments, the second peptidyl linker includes at least 5 serines. In embodiments, the second peptidyl linker includes at least 6 serines. In embodiments, the second peptidyl linker includes at least 7 serines. In embodiments, the second peptidyl linker includes at least 8 serines, serines, or threonines. In embodiments, the second peptidyl linker includes at least 9 serines. In embodiments, the second peptidyl linker includes at least 10 serines. In embodiments, the second peptidyl linker includes at least 11 serines. In embodiments, the second peptidyl linker includes at least 12 serines. In embodiments, the second peptidyl linker includes at least 13 serines. In embodiments, the second peptidyl linker includes at least 14 serines. In embodiments, the second peptidyl linker includes at least 15 serines.

In embodiments, the second peptidyl linker includes at least 1 threonine. In embodiments, the second peptidyl linker includes at least 2 threonines. In embodiments, the second peptidyl linker includes at least 3 threonines. In embodiments, the second peptidyl linker includes at least 4 threonines. In embodiments, the second peptidyl linker includes at least 5 threonines. In embodiments, the second peptidyl linker includes at least 6 threonines. In embodiments, the second peptidyl linker includes at least 7 threonines. In embodiments, the second peptidyl linker includes at least 8 threonines, serines, or threonines. In embodiments, the second peptidyl linker includes at least 9 threonines. In embodiments, the second peptidyl linker includes at least 10 threonines. In embodiments, the second peptidyl linker includes at least 11 threonines. In embodiments, the second peptidyl linker includes at least 12 threonines. In embodiments, the second peptidyl linker includes at least 13 threonines. In embodiments, the second peptidyl linker includes at least 14 threonines. In embodiments, the second peptidyl linker includes at least 15 threonines.

In embodiments, the third peptidyl linker includes at least 1 glycine, serine, or threonine. In embodiments, the third peptidyl linker includes at least 2 glycines, serines, or threonines. In embodiments, the third peptidyl linker includes at least 3 glycines, serines, or threonines. In embodiments, the third peptidyl linker includes at least 4 glycines, serines, or threonines. In embodiments, the third peptidyl linker includes at least 5 glycines, serines, or threonines. In embodiments, the third peptidyl linker includes at least 6 glycines, serines, or threonines. In embodiments, the third peptidyl linker includes at least 7 glycines, serines, or threonines. In embodiments, the third peptidyl linker includes at least 8 glycines, serines, or threonines. In embodiments, the third peptidyl linker includes at least 9 glycines, serines, or threonines. In embodiments, the third peptidyl linker includes at least 10 glycines, serines, or threonines. In embodiments, the third peptidyl linker includes at least 11 glycines, serines, or threonines. In embodiments, the third peptidyl linker includes at least 12 glycines, serines, or threonines. In embodiments, the third peptidyl linker includes at least 13 glycines, serines, or threonines. In embodiments, the third peptidyl linker includes at least 14 glycines, serines, or threonines. In embodiments, the third peptidyl linker includes at least 15 glycines, serines, or threonines.

In embodiments, the third peptidyl linker includes at least 1 glycine. In embodiments, the third peptidyl linker includes at least 2 glycines. In embodiments, the third peptidyl linker includes at least 3 glycines. In embodiments, the third peptidyl linker includes at least 4 glycines. In embodiments, the third peptidyl linker includes at least 5 glycines. In embodiments, the third peptidyl linker includes at least 6 glycines. In embodiments, the third peptidyl linker includes at least 7 glycines. In embodiments, the third peptidyl linker includes at least 8 glycines, serines, or threonines. In embodiments, the third peptidyl linker includes at least 9 glycines. In embodiments, the third peptidyl linker includes at least 10 glycines. In embodiments, the third peptidyl linker includes at least 11 glycines. In embodiments, the third peptidyl linker includes at least 12 glycines. In embodiments, the third peptidyl linker includes at least 13 glycines. In embodiments, the third peptidyl linker includes at least 14 glycines. In embodiments, the third peptidyl linker includes at least 15 glycines.

In embodiments, the third peptidyl linker includes at least 1 serine. In embodiments, the third peptidyl linker includes at least 2 serines. In embodiments, the third peptidyl linker includes at least 3 serines. In embodiments, the third peptidyl linker includes at least 4 serines. In embodiments, the third peptidyl linker includes at least 5 serines. In embodiments, the third peptidyl linker includes at least 6 serines. In embodiments, the third peptidyl linker includes at least 7 serines. In embodiments, the third peptidyl linker includes at least 8 serines, serines, or threonines. In embodiments, the third peptidyl linker includes at least 9 serines. In embodiments, the third peptidyl linker includes at least 10 serines. In embodiments, the third peptidyl linker includes at least 11 serines. In embodiments, the third peptidyl linker includes at least 12 serines. In embodiments, the third peptidyl linker includes at least 13 serines. In embodiments, the third peptidyl linker includes at least 14 serines. In embodiments, the third peptidyl linker includes at least 15 serines.

In embodiments, the third peptidyl linker includes at least 1 threonine. In embodiments, the third peptidyl linker includes at least 2 threonines. In embodiments, the third peptidyl linker includes at least 3 threonines. In embodiments, the third peptidyl linker includes at least 4 threonines. In embodiments, the third peptidyl linker includes at least 5 threonines. In embodiments, the third peptidyl linker includes at least 6 threonines. In embodiments, the third peptidyl linker includes at least 7 threonines. In embodiments, the third peptidyl linker includes at least 8 threonines, serines, or threonines. In embodiments, the third peptidyl linker includes at least 9 threonines. In embodiments, the third peptidyl linker includes at least 10 threonines. In embodiments, the third peptidyl linker includes at least 11 threonines. In embodiments, the third peptidyl linker includes at least 12 threonines. In embodiments, the third peptidyl linker includes at least 13 threonines. In embodiments, the third peptidyl linker includes at least 14 threonines. In embodiments, the third peptidyl linker includes at least 15 threonines.

In embodiments, the fourth peptidyl linker includes at least 1 glycine, serine, or threonine. In embodiments, the fourth peptidyl linker includes at least 2 glycines, serines, or threonines. In embodiments, the fourth peptidyl linker includes at least 3 glycines, serines, or threonines. In embodiments, the fourth peptidyl linker includes at least 4 glycines, serines, or threonines. In embodiments, the fourth peptidyl linker includes at least 5 glycines, serines, or threonines. In embodiments, the fourth peptidyl linker includes at least 6 glycines, serines, or threonines. In embodiments, the fourth peptidyl linker includes at least 7 glycines, serines, or threonines. In embodiments, the fourth peptidyl linker includes at least 8 glycines, serines, or threonines. In embodiments, the fourth peptidyl linker includes at least 9 glycines, serines, or threonines. In embodiments, the fourth peptidyl linker includes at least 10 glycines, serines, or threonines. In embodiments, the fourth peptidyl linker includes at least 11 glycines, serines, or threonines. In embodiments, the fourth peptidyl linker includes at least 12 glycines, serines, or threonines. In embodiments, the fourth peptidyl linker includes at least 13 glycines, serines, or threonines. In embodiments, the fourth peptidyl linker includes at least 14 glycines, serines, or threonines. In embodiments, the fourth peptidyl linker includes at least 15 glycines, serines, or threonines.

In embodiments, the fourth peptidyl linker includes at least 1 glycine. In embodiments, the fourth peptidyl linker includes at least 2 glycines. In embodiments, the fourth peptidyl linker includes at least 3 glycines. In embodiments, the fourth peptidyl linker includes at least 4 glycines. In embodiments, the fourth peptidyl linker includes at least 5 glycines. In embodiments, the fourth peptidyl linker includes at least 6 glycines. In embodiments, the fourth peptidyl linker includes at least 7 glycines. In embodiments, the fourth peptidyl linker includes at least 8 glycines, serines, or threonines. In embodiments, the fourth peptidyl linker includes at least 9 glycines. In embodiments, the fourth peptidyl linker includes at least 10 glycines. In embodiments, the fourth peptidyl linker includes at least 11 glycines. In embodiments, the fourth peptidyl linker includes at least 12 glycines. In embodiments, the fourth peptidyl linker includes at least 13 glycines. In embodiments, the fourth peptidyl linker includes at least 14 glycines. In embodiments, the fourth peptidyl linker includes at least 15 glycines.

In embodiments, the fourth peptidyl linker includes at least 1 serine. In embodiments, the fourth peptidyl linker includes at least 2 serines. In embodiments, the fourth peptidyl linker includes at least 3 serines. In embodiments, the fourth peptidyl linker includes at least 4 serines. In embodiments, the fourth peptidyl linker includes at least 5 serines. In embodiments, the fourth peptidyl linker includes at least 6 serines. In embodiments, the fourth peptidyl linker includes at least 7 serines. In embodiments, the fourth peptidyl linker includes at least 8 serines, serines, or threonines. In embodiments, the fourth peptidyl linker includes at least 9 serines. In embodiments, the fourth peptidyl linker includes at least 10 serines. In embodiments, the fourth peptidyl linker includes at least 11 serines. In embodiments, the fourth peptidyl linker includes at least 12 serines. In embodiments, the fourth peptidyl linker includes at least 13 serines. In embodiments, the fourth peptidyl linker includes at least 14 serines. In embodiments, the fourth peptidyl linker includes at least 15 serines.

In embodiments, the fourth peptidyl linker includes at least 1 threonine. In embodiments, the fourth peptidyl linker includes at least 2 threonines. In embodiments, the fourth peptidyl linker includes at least 3 threonines. In embodiments, the fourth peptidyl linker includes at least 4 threonines. In embodiments, the fourth peptidyl linker includes at least 5 threonines. In embodiments, the fourth peptidyl linker includes at least 6 threonines. In embodiments, the fourth peptidyl linker includes at least 7 threonines. In embodiments, the fourth peptidyl linker includes at least 8 threonines, serines, or threonines. In embodiments, the fourth peptidyl linker includes at least 9 threonines. In embodiments, the fourth peptidyl linker includes at least 10 threonines. In embodiments, the fourth peptidyl linker includes at least 11 threonines. In embodiments, the fourth peptidyl linker includes at least 12 threonines. In embodiments, the fourth peptidyl linker includes at least 13 threonines. In embodiments, the fourth peptidyl linker includes at least 14 threonines. In embodiments, the fourth peptidyl linker includes at least 15 threonines.

In embodiments, at least 60% of the amino acids of the first peptidyl linker, the second peptidyl linker, the third peptidyl linker, and/or the fourth peptidyl linker include glycine. In embodiments, at least 65% of the amino acids of the first peptidyl linker, the second peptidyl linker, the third peptidyl linker, and/or the fourth peptidyl linker include glycine. In embodiments, at least 70% of the amino acids of the first peptidyl linker, the second peptidyl linker, the third peptidyl linker, and/or the fourth peptidyl linker include glycine. In embodiments, at least 75% of the amino acids of the first peptidyl linker, the second peptidyl linker, the third peptidyl linker, and/or the fourth peptidyl linker include glycine. In embodiments, at least 80% of the amino acids of the first peptidyl linker, the second peptidyl linker, the third peptidyl linker, and/or the fourth peptidyl linker include glycine. In embodiments, at least 85% of the amino acids of the first peptidyl linker, the second peptidyl linker, the third peptidyl linker, and/or the fourth peptidyl linker include glycine. In embodiments, at least 90% of the amino acids of the first peptidyl linker, the second peptidyl linker, the third peptidyl linker, and/or the fourth peptidyl linker include glycine. In embodiments, at least 95% of the amino acids of the first peptidyl linker, the second peptidyl linker, the third peptidyl linker, and/or the fourth peptidyl linker include glycine. In embodiments, at least 96% of the amino acids of the first peptidyl linker, the second peptidyl linker, the third peptidyl linker, and/or the fourth peptidyl linker include glycine. In embodiments, at least 97% of the amino acids of the first peptidyl linker, the second peptidyl linker, the third peptidyl linker, and/or the fourth peptidyl linker include glycine. In embodiments, at least 98% of the amino acids of the first peptidyl linker, the second peptidyl linker, the third peptidyl linker, and/or the fourth peptidyl linker include glycine. In embodiments, at least 99% of the amino acids of the first peptidyl linker, the second peptidyl linker, the third peptidyl linker, and/or the fourth peptidyl linker include glycine. In embodiments, at least 100% of the amino acids of the first peptidyl linker, the second peptidyl linker, the third peptidyl linker, and/or the fourth peptidyl linker include glycine.

In embodiments, at least 60% of the amino acids of the first peptidyl linker, the second peptidyl linker, the third peptidyl linker, or the fourth peptidyl linker include glycine. In embodiments, at least 65% of the amino acids of the first peptidyl linker, the second peptidyl linker, the third peptidyl linker, or the fourth peptidyl linker include glycine. In embodiments, at least 70% of the amino acids of the first peptidyl linker, the second peptidyl linker, the third peptidyl linker, or the fourth peptidyl linker include glycine. In embodiments, at least 75% of the amino acids of the first peptidyl linker, the second peptidyl linker, the third peptidyl linker, or the fourth peptidyl linker include glycine. In embodiments, at least 80% of the amino acids of the first peptidyl linker, the second peptidyl linker, the third peptidyl linker, or the fourth peptidyl linker include glycine. In embodiments, at least 85% of the amino acids of the first peptidyl linker, the second peptidyl linker, the third peptidyl linker, or the fourth peptidyl linker include glycine. In embodiments, at least 90% of the amino acids of the first peptidyl linker, the second peptidyl linker, the third peptidyl linker, or the fourth peptidyl linker include glycine. In embodiments, at least 95% of the amino acids of the first peptidyl linker, the second peptidyl linker, the third peptidyl linker, or the fourth peptidyl linker include glycine. In embodiments, at least 96% of the amino acids of the first peptidyl linker, the second peptidyl linker, the third peptidyl linker, or the fourth peptidyl linker include glycine. In embodiments, at least 97% of the amino acids of the first peptidyl linker, the second peptidyl linker, the third peptidyl linker, or the fourth peptidyl linker include glycine. In embodiments, at least 98% of the amino acids of the first peptidyl linker, the second peptidyl linker, the third peptidyl linker, or the fourth peptidyl linker include glycine. In embodiments, at least 99% of the amino acids of the first peptidyl linker, the second peptidyl linker, the third peptidyl linker, or the fourth peptidyl linker include glycine. In embodiments, at least 100% of the amino acids of the first peptidyl linker, the second peptidyl linker, the third peptidyl linker, or the fourth peptidyl linker include glycine.

In embodiments, at least 60% of the amino acids of the first peptidyl linker, the second peptidyl linker, the third peptidyl linker, and the fourth peptidyl linker include glycine. In embodiments, at least 65% of the amino acids of the first peptidyl linker, the second peptidyl linker, the third peptidyl linker, and the fourth peptidyl linker include glycine. In embodiments, at least 70% of the amino acids of the first peptidyl linker, the second peptidyl linker, the third peptidyl linker, and the fourth peptidyl linker include glycine. In embodiments, at least 75% of the amino acids of the first peptidyl linker, the second peptidyl linker, the third peptidyl linker, and the fourth peptidyl linker include glycine. In embodiments, at least 80% of the amino acids of the first peptidyl linker, the second peptidyl linker, the third peptidyl linker, and the fourth peptidyl linker include glycine. In embodiments, at least 85% of the amino acids of the first peptidyl linker, the second peptidyl linker, the third peptidyl linker, and the fourth peptidyl linker include glycine. In embodiments, at least 90% of the amino acids of the first peptidyl linker, the second peptidyl linker, the third peptidyl linker, and the fourth peptidyl linker include glycine. In embodiments, at least 95% of the amino acids of the first peptidyl linker, the second peptidyl linker, the third peptidyl linker, and the fourth peptidyl linker include glycine. In embodiments, at least 96% of the amino acids of the first peptidyl linker, the second peptidyl linker, the third peptidyl linker, and the fourth peptidyl linker include glycine. In embodiments, at least 97% of the amino acids of the first peptidyl linker, the second peptidyl linker, the third peptidyl linker, and the fourth peptidyl linker include glycine. In embodiments, at least 98% of the amino acids of the first peptidyl linker, the second peptidyl linker, the third peptidyl linker, and the fourth peptidyl linker include glycine. In embodiments, at least 99% of the amino acids of the first peptidyl linker, the second peptidyl linker, the third peptidyl linker, and the fourth peptidyl linker include glycine. In embodiments, at least 100% of the amino acids of the first peptidyl linker, the second peptidyl linker, the third peptidyl linker, and the fourth peptidyl linker include glycine.

In embodiments, at least 60% of the amino acids of the first peptidyl linker include glycine. In embodiments, at least 65% of the amino acids of the first peptidyl linker include glycine. In embodiments, at least 70% of the amino acids of the first peptidyl linker include glycine. In embodiments, at least 75% of the amino acids of the first peptidyl linker include glycine. In embodiments, at least 80% of the amino acids of the first peptidyl linker include glycine. In embodiments, at least 85% of the amino acids of the first peptidyl linker include glycine. In embodiments, at least 90% of the amino acids of the first peptidyl linker include glycine. In embodiments, at least 95% of the amino acids of the first peptidyl linker include glycine. In embodiments, at least 96% of the amino acids of the first peptidyl linker include glycine. In embodiments, at least 97% of the amino acids of the first peptidyl linker include glycine. In embodiments, at least 98% of the amino acids of the first peptidyl linker include glycine. In embodiments, at least 99% of the amino acids of the first peptidyl linker include glycine. In embodiments, at least 100% of the amino acids of the first peptidyl linker include glycine.

In embodiments, at least 60% of the amino acids of the second peptidyl linker include glycine. In embodiments, at least 65% of the amino acids of the second peptidyl linker include glycine. In embodiments, at least 70% of the amino acids of the second peptidyl linker include glycine. In embodiments, at least 75% of the amino acids of the second peptidyl linker include glycine. In embodiments, at least 80% of the amino acids of the second peptidyl linker include glycine. In embodiments, at least 85% of the amino acids of the second peptidyl linker include glycine. In embodiments, at least 90% of the amino acids of the second peptidyl linker include glycine. In embodiments, at least 95% of the amino acids of the second peptidyl linker include glycine. In embodiments, at least 96% of the amino acids of the second peptidyl linker include glycine. In embodiments, at least 97% of the amino acids of the second peptidyl linker include glycine. In embodiments, at least 98% of the amino acids of the second peptidyl linker include glycine. In embodiments, at least 99% of the amino acids of the second peptidyl linker include glycine. In embodiments, at least 100% of the amino acids of the second peptidyl linker include glycine.

In embodiments, at least 60% of the amino acids of the third peptidyl linker include glycine. In embodiments, at least 65% of the amino acids of the third peptidyl linker include glycine. In embodiments, at least 70% of the amino acids of the third peptidyl linker include glycine. In embodiments, at least 75% of the amino acids of the third peptidyl linker include glycine. In embodiments, at least 80% of the amino acids of the third peptidyl linker include glycine. In embodiments, at least 85% of the amino acids of the third peptidyl linker include glycine. In embodiments, at least 90% of the amino acids of the third peptidyl linker include glycine. In embodiments, at least 95% of the amino acids of the third peptidyl linker include glycine. In embodiments, at least 96% of the amino acids of the third peptidyl linker include glycine. In embodiments, at least 97% of the amino acids of the third peptidyl linker include glycine. In embodiments, at least 98% of the amino acids of the third peptidyl linker include glycine. In embodiments, at least 99% of the amino acids of the third peptidyl linker include glycine. In embodiments, at least 100% of the amino acids of the third peptidyl linker include glycine.

In embodiments, at least 60% of the amino acids of the fourth peptidyl linker include glycine. In embodiments, at least 65% of the amino acids of the fourth peptidyl linker include glycine. In embodiments, at least 70% of the amino acids of the fourth peptidyl linker include glycine. In embodiments, at least 75% of the amino acids of the fourth peptidyl linker include glycine. In embodiments, at least 80% of the amino acids of the fourth peptidyl linker include glycine. In embodiments, at least 85% of the amino acids of the fourth peptidyl linker include glycine. In embodiments, at least 90% of the amino acids of the fourth peptidyl linker include glycine. In embodiments, at least 95% of the amino acids of the fourth peptidyl linker include glycine. In embodiments, at least 96% of the amino acids of the fourth peptidyl linker include glycine. In embodiments, at least 97% of the amino acids of the fourth peptidyl linker include glycine. In embodiments, at least 98% of the amino acids of the fourth peptidyl linker include glycine. In embodiments, at least 99% of the amino acids of the fourth peptidyl linker include glycine. In embodiments, at least 100% of the amino acids of the fourth peptidyl linker include glycine.

In embodiments, at least 20% of the amino acids of the first peptidyl linker, the second peptidyl linker, the third peptidyl linker, and/or the fourth peptidyl linker include serine. In embodiments, at least 30% of the amino acids of the first peptidyl linker, the second peptidyl linker, the third peptidyl linker, and/or the fourth peptidyl linker include serine. In embodiments, at least 40% of the amino acids of the first peptidyl linker, the second peptidyl linker, the third peptidyl linker, and/or the fourth peptidyl linker include serine. In embodiments, at least 50% of the amino acids of the first peptidyl linker, the second peptidyl linker, the third peptidyl linker, and/or the fourth peptidyl linker include serine. In embodiments, at least 60% of the amino acids of the first peptidyl linker, the second peptidyl linker, the third peptidyl linker, and/or the fourth peptidyl linker include serine. In embodiments, at least 70% of the amino acids of the first peptidyl linker, the second peptidyl linker, the third peptidyl linker, and/or the fourth peptidyl linker include serine. In embodiments, at least 80% of the amino acids of the first peptidyl linker, the second peptidyl linker, the third peptidyl linker, and/or the fourth peptidyl linker include serine. In embodiments, at least 90% of the amino acids of the first peptidyl linker, the second peptidyl linker, the third peptidyl linker, and/or the fourth peptidyl linker include serine. In embodiments, at least 95% of the amino acids of the first peptidyl linker, the second peptidyl linker, the third peptidyl linker, and/or the fourth peptidyl linker include serine. In embodiments, at least 96% of the amino acids of the first peptidyl linker, the second peptidyl linker, the third peptidyl linker, and/or the fourth peptidyl linker include serine. In embodiments, at least 97% of the amino acids of the first peptidyl linker, the second peptidyl linker, the third peptidyl linker, and/or the fourth peptidyl linker include serine. In embodiments, at least 98% of the amino acids of the first peptidyl linker, the second peptidyl linker, the third peptidyl linker, and/or the fourth peptidyl linker include serine. In embodiments, at least 99% of the amino acids of the first peptidyl linker, the second peptidyl linker, the third peptidyl linker, and/or the fourth peptidyl linker include serine. In embodiments, at least 100% of the amino acids of the first peptidyl linker, the second peptidyl linker, the third peptidyl linker, and/or the fourth peptidyl linker include serine.

In embodiments, at least 20% of the amino acids of the first peptidyl linker, the second peptidyl linker, the third peptidyl linker, or the fourth peptidyl linker include serine. In embodiments, at least 30% of the amino acids of the first peptidyl linker, the second peptidyl linker, the third peptidyl linker, or the fourth peptidyl linker include serine. In embodiments, at least 40% of the amino acids of the first peptidyl linker, the second peptidyl linker, the third peptidyl linker, or the fourth peptidyl linker include serine. In embodiments, at least 50% of the amino acids of the first peptidyl linker, the second peptidyl linker, the third peptidyl linker, or the fourth peptidyl linker include serine. In embodiments, at least 60% of the amino acids of the first peptidyl linker, the second peptidyl linker, the third peptidyl linker, or the fourth peptidyl linker include serine. In embodiments, at least 70% of the amino acids of the first peptidyl linker, the second peptidyl linker, the third peptidyl linker, or the fourth peptidyl linker include serine. In embodiments, at least 80% of the amino acids of the first peptidyl linker, the second peptidyl linker, the third peptidyl linker, or the fourth peptidyl linker include serine. In embodiments, at least 90% of the amino acids of the first peptidyl linker, the second peptidyl linker, the third peptidyl linker, or the fourth peptidyl linker include serine. In embodiments, at least 95% of the amino acids of the first peptidyl linker, the second peptidyl linker, the third peptidyl linker, or the fourth peptidyl linker include serine. In embodiments, at least 96% of the amino acids of the first peptidyl linker, the second peptidyl linker, the third peptidyl linker, or the fourth peptidyl linker include serine. In embodiments, at least 97% of the amino acids of the first peptidyl linker, the second peptidyl linker, the third peptidyl linker, or the fourth peptidyl linker include serine. In embodiments, at least 98% of the amino acids of the first peptidyl linker, the second peptidyl linker, the third peptidyl linker, or the fourth peptidyl linker include serine. In embodiments, at least 99% of the amino acids of the first peptidyl linker, the second peptidyl linker, the third peptidyl linker, or the fourth peptidyl linker include serine. In embodiments, at least 100% of the amino acids of the first peptidyl linker, the second peptidyl linker, the third peptidyl linker, or the fourth peptidyl linker include serine.

In embodiments, at least 20% of the amino acids of the first peptidyl linker, the second peptidyl linker, the third peptidyl linker, and the fourth peptidyl linker include serine. In embodiments, at least 30% of the amino acids of the first peptidyl linker, the second peptidyl linker, the third peptidyl linker, and the fourth peptidyl linker include serine. In embodiments, at least 40% of the amino acids of the first peptidyl linker, the second peptidyl linker, the third peptidyl linker, and the fourth peptidyl linker include serine. In embodiments, at least 50% of the amino acids of the first peptidyl linker, the second peptidyl linker, the third peptidyl linker, and the fourth peptidyl linker include serine. In embodiments, at least 60% of the amino acids of the first peptidyl linker, the second peptidyl linker, the third peptidyl linker, and the fourth peptidyl linker include serine. In embodiments, at least 70% of the amino acids of the first peptidyl linker, the second peptidyl linker, the third peptidyl linker, and the fourth peptidyl linker include serine. In embodiments, at least 80% of the amino acids of the first peptidyl linker, the second peptidyl linker, the third peptidyl linker, and the fourth peptidyl linker include serine. In embodiments, at least 90% of the amino acids of the first peptidyl linker, the second peptidyl linker, the third peptidyl linker, and the fourth peptidyl linker include serine. In embodiments, at least 95% of the amino acids of the first peptidyl linker, the second peptidyl linker, the third peptidyl linker, and the fourth peptidyl linker include serine. In embodiments, at least 96% of the amino acids of the first peptidyl linker, the second peptidyl linker, the third peptidyl linker, and the fourth peptidyl linker include serine. In embodiments, at least 97% of the amino acids of the first peptidyl linker, the second peptidyl linker, the third peptidyl linker, and the fourth peptidyl linker include serine. In embodiments, at least 98% of the amino acids of the first peptidyl linker, the second peptidyl linker, the third peptidyl linker, and the fourth peptidyl linker include serine. In embodiments, at least 99% of the amino acids of the first peptidyl linker, the second peptidyl linker, the third peptidyl linker, and the fourth peptidyl linker include serine. In embodiments, at least 100% of the amino acids of the first peptidyl linker, the second peptidyl linker, the third peptidyl linker, and the fourth peptidyl linker include serine.

In embodiments, at least 20% of the amino acids of the first peptidyl linker include serine. In embodiments, at least 30% of the amino acids of the first peptidyl linker include serine. In embodiments, at least 40% of the amino acids of the first peptidyl linker include serine. In embodiments, at least 50% of the amino acids of the first peptidyl linker include serine. In embodiments, at least 60% of the amino acids of the first peptidyl linker include serine. In embodiments, at least 70% of the amino acids of the first peptidyl linker include serine. In embodiments, at least 80% of the amino acids of the first peptidyl linker include serine. In embodiments, at least 90% of the amino acids of the first peptidyl linker include serine. In embodiments, at least 95% of the amino acids of the first peptidyl linker include serine. In embodiments, at least 96% of the amino acids of the first peptidyl linker include serine. In embodiments, at least 97% of the amino acids of the first peptidyl linker include serine. In embodiments, at least 98% of the amino acids of the first peptidyl linker include serine. In embodiments, at least 99% of the amino acids of the first peptidyl linker include serine. In embodiments, at least 100% of the amino acids of the first peptidyl linker include serine.

In embodiments, at least 20% of the amino acids of the second peptidyl linker include serine. In embodiments, at least 30% of the amino acids of the second peptidyl linker include serine. In embodiments, at least 40% of the amino acids of the second peptidyl linker include serine. In embodiments, at least 50% of the amino acids of the second peptidyl linker include serine. In embodiments, at least 60% of the amino acids of the second peptidyl linker include serine. In embodiments, at least 70% of the amino acids of the second peptidyl linker include serine. In embodiments, at least 80% of the amino acids of the second peptidyl linker include serine. In embodiments, at least 90% of the amino acids of the second peptidyl linker include serine. In embodiments, at least 95% of the amino acids of the second peptidyl linker include serine. In embodiments, at least 96% of the amino acids of the second peptidyl linker include serine. In embodiments, at least 97% of the amino acids of the second peptidyl linker include serine. In embodiments, at least 98% of the amino acids of the second peptidyl linker include serine. In embodiments, at least 99% of the amino acids of the second peptidyl linker include serine. In embodiments, at least 100% of the amino acids of the second peptidyl linker include serine.

In embodiments, at least 20% of the amino acids of the third peptidyl linker include serine. In embodiments, at least 30% of the amino acids of the third peptidyl linker include serine. In embodiments, at least 40% of the amino acids of the third peptidyl linker include serine. In embodiments, at least 50% of the amino acids of the third peptidyl linker include serine. In embodiments, at least 60% of the amino acids of the third peptidyl linker include serine. In embodiments, at least 70% of the amino acids of the third peptidyl linker include serine. In embodiments, at least 80% of the amino acids of the third peptidyl linker include serine. In embodiments, at least 90% of the amino acids of the third peptidyl linker include serine. In embodiments, at least 95% of the amino acids of the third peptidyl linker include serine. In embodiments, at least 96% of the amino acids of the third peptidyl linker include serine. In embodiments, at least 97% of the amino acids of the third peptidyl linker include serine. In embodiments, at least 98% of the amino acids of the third peptidyl linker include serine. In embodiments, at least 99% of the amino acids of the third peptidyl linker include serine. In embodiments, at least 100% of the amino acids of the third peptidyl linker include serine.

In embodiments, at least 20% of the amino acids of the fourth peptidyl linker include serine. In embodiments, at least 30% of the amino acids of the fourth peptidyl linker include serine. In embodiments, at least 40% of the amino acids of the fourth peptidyl linker include serine. In embodiments, at least 50% of the amino acids of the fourth peptidyl linker include serine. In embodiments, at least 60% of the amino acids of the fourth peptidyl linker include serine. In embodiments, at least 70% of the amino acids of the fourth peptidyl linker include serine. In embodiments, at least 80% of the amino acids of the fourth peptidyl linker include serine. In embodiments, at least 90% of the amino acids of the fourth peptidyl linker include serine. In embodiments, at least 95% of the amino acids of the fourth peptidyl linker include serine. In embodiments, at least 96% of the amino acids of the fourth peptidyl linker include serine. In embodiments, at least 97% of the amino acids of the fourth peptidyl linker include serine. In embodiments, at least 98% of the amino acids of the fourth peptidyl linker include serine. In embodiments, at least 99% of the amino acids of the fourth peptidyl linker include serine. In embodiments, at least 100% of the amino acids of the fourth peptidyl linker include serine.

In embodiments, at least 10% of the amino acids of the first peptidyl linker, the second peptidyl linker, the third peptidyl linker, and/or the fourth peptidyl linker include threonine. In embodiments, at least 20% of the amino acids of the first peptidyl linker, the second peptidyl linker, the third peptidyl linker, and/or the fourth peptidyl linker include threonine. In embodiments, at least 30% of the amino acids of the first peptidyl linker, the second peptidyl linker, the third peptidyl linker, and/or the fourth peptidyl linker include threonine. In embodiments, at least 40% of the amino acids of the first peptidyl linker, the second peptidyl linker, the third peptidyl linker, and/or the fourth peptidyl linker include threonine. In embodiments, at least 50% of the amino acids of the first peptidyl linker, the second peptidyl linker, the third peptidyl linker, and/or the fourth peptidyl linker include threonine. In embodiments, at least 60% of the amino acids of the first peptidyl linker, the second peptidyl linker, the third peptidyl linker, and/or the fourth peptidyl linker include threonine. In embodiments, at least 70% of the amino acids of the first peptidyl linker, the second peptidyl linker, the third peptidyl linker, and/or the fourth peptidyl linker include threonine. In embodiments, at least 80% of the amino acids of the first peptidyl linker, the second peptidyl linker, the third peptidyl linker, and/or the fourth peptidyl linker include threonine. In embodiments, at least 90% of the amino acids of the first peptidyl linker, the second peptidyl linker, the third peptidyl linker, and/or the fourth peptidyl linker include threonine. In embodiments, at least 95% of the amino acids of the first peptidyl linker, the second peptidyl linker, the third peptidyl linker, and/or the fourth peptidyl linker include threonine. In embodiments, at least 96% of the amino acids of the first peptidyl linker, the second peptidyl linker, the third peptidyl linker, and/or the fourth peptidyl linker include threonine. In embodiments, at least 97% of the amino acids of the first peptidyl linker, the second peptidyl linker, the third peptidyl linker, and/or the fourth peptidyl linker include threonine. In embodiments, at least 98% of the amino acids of the first peptidyl linker, the second peptidyl linker, the third peptidyl linker, and/or the fourth peptidyl linker include threonine. In embodiments, at least 99% of the amino acids of the first peptidyl linker, the second peptidyl linker, the third peptidyl linker, and/or the fourth peptidyl linker include threonine. In embodiments, at least 100% of the amino acids of the first peptidyl linker, the second peptidyl linker, the third peptidyl linker, and/or the fourth peptidyl linker include threonine.

In embodiments, at least 10% of the amino acids of the first peptidyl linker, the second peptidyl linker, the third peptidyl linker, or the fourth peptidyl linker include threonine. In embodiments, at least 20% of the amino acids of the first peptidyl linker, the second peptidyl linker, the third peptidyl linker, or the fourth peptidyl linker include threonine. In embodiments, at least 30% of the amino acids of the first peptidyl linker, the second peptidyl linker, the third peptidyl linker, or the fourth peptidyl linker include threonine. In embodiments, at least 40% of the amino acids of the first peptidyl linker, the second peptidyl linker, the third peptidyl linker, or the fourth peptidyl linker include threonine. In embodiments, at least 50% of the amino acids of the first peptidyl linker, the second peptidyl linker, the third peptidyl linker, or the fourth peptidyl linker include threonine. In embodiments, at least 60% of the amino acids of the first peptidyl linker, the second peptidyl linker, the third peptidyl linker, or the fourth peptidyl linker include threonine. In embodiments, at least 70% of the amino acids of the first peptidyl linker, the second peptidyl linker, the third peptidyl linker, or the fourth peptidyl linker include threonine. In embodiments, at least 80% of the amino acids of the first peptidyl linker, the second peptidyl linker, the third peptidyl linker, or the fourth peptidyl linker include threonine. In embodiments, at least 90% of the amino acids of the first peptidyl linker, the second peptidyl linker, the third peptidyl linker, or the fourth peptidyl linker include threonine. In embodiments, at least 95% of the amino acids of the first peptidyl linker, the second peptidyl linker, the third peptidyl linker, or the fourth peptidyl linker include threonine. In embodiments, at least 96% of the amino acids of the first peptidyl linker, the second peptidyl linker, the third peptidyl linker, or the fourth peptidyl linker include threonine. In embodiments, at least 97% of the amino acids of the first peptidyl linker, the second peptidyl linker, the third peptidyl linker, or the fourth peptidyl linker include threonine. In embodiments, at least 98% of the amino acids of the first peptidyl linker, the second peptidyl linker, the third peptidyl linker, or the fourth peptidyl linker include threonine. In embodiments, at least 99% of the amino acids of the first peptidyl linker, the second peptidyl linker, the third peptidyl linker, or the fourth peptidyl linker include threonine. In embodiments, at least 100% of the amino acids of the first peptidyl linker, the second peptidyl linker, the third peptidyl linker, or the fourth peptidyl linker include threonine.

In embodiments, at least 10% of the amino acids of the first peptidyl linker, the second peptidyl linker, the third peptidyl linker, and the fourth peptidyl linker include threonine. In embodiments, at least 20% of the amino acids of the first peptidyl linker, the second peptidyl linker, the third peptidyl linker, and the fourth peptidyl linker include threonine. In embodiments, at least 30% of the amino acids of the first peptidyl linker, the second peptidyl linker, the third peptidyl linker, and the fourth peptidyl linker include threonine. In embodiments, at least 40% of the amino acids of the first peptidyl linker, the second peptidyl linker, the third peptidyl linker, and the fourth peptidyl linker include threonine. In embodiments, at least 50% of the amino acids of the first peptidyl linker, the second peptidyl linker, the third peptidyl linker, and the fourth peptidyl linker include threonine. In embodiments, at least 60% of the amino acids of the first peptidyl linker, the second peptidyl linker, the third peptidyl linker, and the fourth peptidyl linker include threonine. In embodiments, at least 70% of the amino acids of the first peptidyl linker, the second peptidyl linker, the third peptidyl linker, and the fourth peptidyl linker include threonine. In embodiments, at least 80% of the amino acids of the first peptidyl linker, the second peptidyl linker, the third peptidyl linker, and the fourth peptidyl linker include threonine. In embodiments, at least 90% of the amino acids of the first peptidyl linker, the second peptidyl linker, the third peptidyl linker, and the fourth peptidyl linker include threonine. In embodiments, at least 95% of the amino acids of the first peptidyl linker, the second peptidyl linker, the third peptidyl linker, and the fourth peptidyl linker include threonine. In embodiments, at least 96% of the amino acids of the first peptidyl linker, the second peptidyl linker, the third peptidyl linker, and the fourth peptidyl linker include threonine. In embodiments, at least 97% of the amino acids of the first peptidyl linker, the second peptidyl linker, the third peptidyl linker, and the fourth peptidyl linker include threonine. In embodiments, at least 98% of the amino acids of the first peptidyl linker, the second peptidyl linker, the third peptidyl linker, and the fourth peptidyl linker include threonine. In embodiments, at least 99% of the amino acids of the first peptidyl linker, the second peptidyl linker, the third peptidyl linker, and the fourth peptidyl linker include threonine. In embodiments, at least 100% of the amino acids of the first peptidyl linker, the second peptidyl linker, the third peptidyl linker, and the fourth peptidyl linker include threonine.

In embodiments, at least 10% of the amino acids of the first peptidyl linker include threonine. In embodiments, at least 20% of the amino acids of the first peptidyl linker include threonine. In embodiments, at least 30% of the amino acids of the first peptidyl linker include threonine. In embodiments, at least 40% of the amino acids of the first peptidyl linker include threonine. In embodiments, at least 50% of the amino acids of the first peptidyl linker include threonine. In embodiments, at least 60% of the amino acids of the first peptidyl linker include threonine. In embodiments, at least 70% of the amino acids of the first peptidyl linker include threonine. In embodiments, at least 80% of the amino acids of the first peptidyl linker include threonine. In embodiments, at least 90% of the amino acids of the first peptidyl linker include threonine. In embodiments, at least 95% of the amino acids of the first peptidyl linker include threonine. In embodiments, at least 96% of the amino acids of the first peptidyl linker include threonine. In embodiments, at least 97% of the amino acids of the first peptidyl linker include threonine. In embodiments, at least 98% of the amino acids of the first peptidyl linker include threonine. In embodiments, at least 99% of the amino acids of the first peptidyl linker include threonine. In embodiments, at least 100% of the amino acids of the first peptidyl linker include threonine.

In embodiments, at least 10% of the amino acids of the second peptidyl linker include threonine. In embodiments, at least 20% of the amino acids of the second peptidyl linker include threonine. In embodiments, at least 30% of the amino acids of the second peptidyl linker include threonine. In embodiments, at least 40% of the amino acids of the second peptidyl linker include threonine. In embodiments, at least 50% of the amino acids of the second peptidyl linker include threonine. In embodiments, at least 60% of the amino acids of the second peptidyl linker include threonine. In embodiments, at least 70% of the amino acids of the second peptidyl linker include threonine. In embodiments, at least 80% of the amino acids of the second peptidyl linker include threonine. In embodiments, at least 90% of the amino acids of the second peptidyl linker include threonine. In embodiments, at least 95% of the amino acids of the second peptidyl linker include threonine. In embodiments, at least 96% of the amino acids of the second peptidyl linker include threonine. In embodiments, at least 97% of the amino acids of the second peptidyl linker include threonine. In embodiments, at least 98% of the amino acids of the second peptidyl linker include threonine. In embodiments, at least 99% of the amino acids of the second peptidyl linker include threonine. In embodiments, at least 100% of the amino acids of the second peptidyl linker include threonine.

In embodiments, at least 10% of the amino acids of the third peptidyl linker include threonine. In embodiments, at least 20% of the amino acids of the third peptidyl linker include threonine. In embodiments, at least 30% of the amino acids of the third peptidyl linker include threonine. In embodiments, at least 40% of the amino acids of the third peptidyl linker include threonine. In embodiments, at least 50% of the amino acids of the third peptidyl linker include threonine. In embodiments, at least 60% of the amino acids of the third peptidyl linker include threonine. In embodiments, at least 70% of the amino acids of the third peptidyl linker include threonine. In embodiments, at least 80% of the amino acids of the third peptidyl linker include threonine. In embodiments, at least 90% of the amino acids of the third peptidyl linker include threonine. In embodiments, at least 95% of the amino acids of the third peptidyl linker include threonine. In embodiments, at least 96% of the amino acids of the third peptidyl linker include threonine. In embodiments, at least 97% of the amino acids of the third peptidyl linker include threonine. In embodiments, at least 98% of the amino acids of the third peptidyl linker include threonine. In embodiments, at least 99% of the amino acids of the third peptidyl linker include threonine. In embodiments, at least 100% of the amino acids of the third peptidyl linker include threonine.

In embodiments, at least 10% of the amino acids of the fourth peptidyl linker include threonine. In embodiments, at least 20% of the amino acids of the fourth peptidyl linker include threonine. In embodiments, at least 30% of the amino acids of the fourth peptidyl linker include threonine. In embodiments, at least 40% of the amino acids of the fourth peptidyl linker include threonine. In embodiments, at least 50% of the amino acids of the fourth peptidyl linker include threonine. In embodiments, at least 60% of the amino acids of the fourth peptidyl linker include threonine. In embodiments, at least 70% of the amino acids of the fourth peptidyl linker include threonine. In embodiments, at least 80% of the amino acids of the fourth peptidyl linker include threonine. In embodiments, at least 90% of the amino acids of the fourth peptidyl linker include threonine. In embodiments, at least 95% of the amino acids of the fourth peptidyl linker include threonine. In embodiments, at least 96% of the amino acids of the fourth peptidyl linker include threonine. In embodiments, at least 97% of the amino acids of the fourth peptidyl linker include threonine. In embodiments, at least 98% of the amino acids of the fourth peptidyl linker include threonine. In embodiments, at least 99% of the amino acids of the fourth peptidyl linker include threonine. In embodiments, at least 100% of the amino acids of the fourth peptidyl linker include threonine.

In embodiments, the first peptidyl linker, the second peptidyl linker, the third peptidyl linker, and/or the fourth peptidyl linker independently include the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, and/or the fourth peptidyl linker independently include the amino acid sequence of SEQ ID NO:7. In embodiments, the first peptidyl linker, the second peptidyl linker, the third peptidyl linker, and/or the fourth peptidyl linker independently include the amino acid sequence of SEQ ID NO:8. In embodiments, the first peptidyl linker, the second peptidyl linker, the third peptidyl linker, and/or the fourth peptidyl linker independently include the amino acid sequence of SEQ ID NO:9. In embodiments, the first peptidyl linker, the second peptidyl linker, the third peptidyl linker, and/or the fourth peptidyl linker independently include the amino acid sequence of SEQ ID NO:10.

In embodiments, the first peptidyl linker, the second peptidyl linker, the third peptidyl linker, and/or the fourth peptidyl linker independently are the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, and/or the fourth peptidyl linker independently are the amino acid sequence of SEQ ID NO:7. In embodiments, the first peptidyl linker, the second peptidyl linker, the third peptidyl linker, and/or the fourth peptidyl linker independently are the amino acid sequence of SEQ ID NO:8. In embodiments, the first peptidyl linker, the second peptidyl linker, the third peptidyl linker, and/or the fourth peptidyl linker independently are the amino acid sequence of SEQ ID NO:9. In embodiments, the first peptidyl linker, the second peptidyl linker, the third peptidyl linker, and/or the fourth peptidyl linker independently are the amino acid sequence of SEQ ID NO:10.

In embodiments, the first peptidyl linker, the second peptidyl linker, the third peptidyl linker, and the fourth peptidyl linker independently include the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, or the fourth peptidyl linker independently include the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, and the fourth peptidyl linker independently are the amino acid sequence of SEQ ID NO:7, SEQ ID NO:8, SEQ ID NO:9, or SEQ ID NO:10. In embodiments, the first peptidyl linker, the second peptidyl linker, the third peptidyl linker, or the fourth peptidyl linker independently are the amino acid sequence of SEQ ID NO:7, SEQ ID NO:8, SEQ ID NO:9, or SEQ ID NO:10.

In embodiments, the first peptidyl linker includes the amino acid sequence of SEQ ID NO:7, SEQ ID NO:8, SEQ ID NO:9, or SEQ ID NO:10. In embodiments, the first peptidyl linker includes the amino acid sequence of SEQ ID NO:7. In embodiments, the first peptidyl linker includes the amino acid sequence of SEQ ID NO:8. In embodiments, the first peptidyl linker includes the amino acid sequence of SEQ ID NO:9. In embodiments, the first peptidyl linker includes the amino acid sequence of SEQ ID NO: 10. In embodiments, the first peptidyl linker is the amino acid sequence of SEQ ID NO:7, SEQ ID NO:8, SEQ ID NO:9, or SEQ ID NO:10. In embodiments, the first peptidyl linker is the amino acid sequence of SEQ ID NO:7. In embodiments, the first peptidyl linker is the amino acid sequence of SEQ ID NO: 8. In embodiments, the first peptidyl linker is the amino acid sequence of SEQ ID NO:9. In embodiments, the first peptidyl linker is the amino acid sequence of SEQ ID NO: 10.

In embodiments, the second peptidyl linker includes the amino acid sequence of SEQ ID NO:7, SEQ ID NO:8, SEQ ID NO:9, or SEQ ID NO: 10. In embodiments, the second peptidyl linker includes the amino acid sequence of SEQ ID NO:7. In embodiments, the second peptidyl linker includes the amino acid sequence of SEQ ID NO:8 In embodiments, the second peptidyl linker includes the amino acid sequence of SEQ ID NO:9. In embodiments, the second peptidyl linker includes the amino acid sequence of SEQ ID NO: 10. In embodiments, the second peptidyl linker is the amino acid sequence of SEQ ID NO: 7, SEQ ID NO:8, SEQ ID NO:9, or SEQ ID NO:10. In embodiments, the second peptidyl linker is the amino acid sequence of SEQ ID NO:7. In embodiments, the second peptidyl linker is the amino acid sequence of SEQ ID NO:8. In embodiments, the second peptidyl linker is the amino acid sequence of SEQ ID NO: 9. In embodiments, the second peptidyl linker is the amino acid sequence of SEQ ID NO: 10.

In embodiments, the third peptidyl linker includes the amino acid sequence of SEQ ID NO:7, SEQ ID NO:8, SEQ ID NO:9, or SEQ ID NO: 10. In embodiments, the third peptidyl linker includes the amino acid sequence of SEQ ID NO:7. In embodiments, the third peptidyl linker includes the amino acid sequence of SEQ ID NO:8 In embodiments, the third peptidyl linker includes the amino acid sequence of SEQ ID NO:9. In embodiments, the third peptidyl linker includes the amino acid sequence of SEQ ID NO: 10. In embodiments, the third peptidyl linker is the amino acid sequence of SEQ ID NO:7, SEQ ID NO:8, SEQ ID NO:9, or SEQ ID NO:10. In embodiments, the third peptidyl linker is the amino acid sequence of SEQ ID NO:7. In embodiments, the third peptidyl linker is the amino acid sequence of SEQ ID NO: 8. In embodiments, the third peptidyl linker is the amino acid sequence of SEQ ID NO: 9. In embodiments, the third peptidyl linker is the amino acid sequence of SEQ ID NO: 10.

In embodiments, the fourth peptidyl linker includes the amino acid sequence of SEQ ID NO:7, SEQ ID NO:8, SEQ ID NO:9, or SEQ ID NO: 10. In embodiments, the fourth peptidyl linker includes the amino acid sequence of SEQ ID NO:7. In embodiments, the fourth peptidyl linker includes the amino acid sequence of SEQ ID NO:8. In embodiments, the fourth peptidyl linker includes the amino acid sequence of SEQ ID NO:9. In embodiments, the fourth peptidyl linker includes the amino acid sequence of SEQ ID NO: 10. In embodiments, the fourth peptidyl linker is the amino acid sequence of SEQ ID NO: 7, SEQ ID NO:8, SEQ ID NO:9, or SEQ ID NO:10. In embodiments, the fourth peptidyl linker is the amino acid sequence of SEQ ID NO:7. In embodiments, the fourth peptidyl linker is the amino acid sequence of SEQ ID NO: 8. In embodiments, the fourth peptidyl linker is the amino acid sequence of SEQ ID NO: 9. In embodiments, the fourth peptidyl linker is the amino acid sequence of SEQ ID NO: 10.

In embodiments, the ligand binding domain is non-covalently bound to the pseudoligand domain.

In embodiments, the ligand binding domain binds a Ras protein with higher affinity than the pseudoligand domain. In embodiments, the Ras protein is an active Ras protein. In

### EXAMPLES

### Example 1: A fluorescent biosensor for Measuring Ras Activity in Living Cells

### Abstract

Here we developed a ratiometric Ras activity reporter (RasAR) that provides quantitative measurement of Ras activity in living cells with high spatiotemporal resolution. We demonstrated that RasAR can probe live-cell activities of all the primary isoforms of Ras. Given that the functional roles of different isoforms of Ras are intimately linked to their subcellular distribution and regulation, we interrogated the spatiotemporal regulation of Ras utilizing subcellularly targeted RasAR and uncovered the role of Src kinase as an upstream regulator to inhibit HRas. Furthermore, we showed that RasAR enables capture of KRasG12C inhibition dynamics in living cells upon treatment with KRasG12C covalent inhibitors, including ARS1620, Sotorasib, and Adagrasib. We found in living cells a residual Ras activity lingers for hours in the presence of these inhibitors. Together, RasAR represents a powerful molecular tool to enable live-cell interrogation of Ras activity and facilitate the development of Ras inhibitors.

### Introduction

As a small GTPase, Ras regulates cell growth and proliferation by acting as a molecular switch downstream of growth factor receptors.¹ Like all GTPases, Ras is a guanine nucleotide binding protein and regulated by Guanine Exchange Factors (GEFs) and GTPase activating proteins (GAPs).² GTP-bound Ras has an effector binding region poised for high-affinity interactions with downstream effectors such as Raf,³ which result in propagation of signaling. While Ras signaling is canonically initiated from the plasma membrane, it has become increasingly clear that the spatiotemporal dynamics of Ras are complex, and that functional roles of different isoforms of Ras are tightly coupled with their subcellular distribution and regulation.^{4,5} Ras mutations are prevalent in cancers, and targeting mutant Ras has been a priority in cancer therapy for decades.^{6,7} Although it was once considered “undruggable”, pioneering work on K-RasG12C inhibitors⁸⁻¹¹ elicited a substantial amount of excitement and extensive efforts in the field of anti-Ras therapy and eventually led to the FDA-approved breakthrough therapy for the treatment of patients with locally advanced or metastatic non-small cell lung cancer with KrasG12C mutation.¹² Given that KRasG12C inhibitors target a mere fraction of potential oncogenic Ras targets, development of new anti-Ras therapy is still extensively pursued. Both understanding the spatiotemporal regulation of Ras and development of new anti-Ras therapy require probing Ras activity in its native context. To measure the activity of Ras, researchers have traditionally relied on Ras pulldown assays, which utilize the Ras binding domain (RBD) of Raf1 to enrich active Ras from cell lysates followed by Western Blot analysis, which suffers from poor spatiotemporal resolution, is difficult to scale to high throughput and essentially removes the target from its native context.¹³ Therefore, there is an urgent need to develop live-cell Ras activity assays.

Genetically encoded fluorescent biosensors have emerged to be powerful tools for investigating the spatiotemporal regulation of signaling molecules. In the context of drug development, live-cell assays can provide quantitative, real-time assessment of target engagement within native biological contexts,¹⁴ which better mimic the disease setting than in vitro assays and allow assessment of drug properties such as permeability and stability. Many Ras biosensors have been previously developed and can be classified into two types: translocation-based and ratiometric, Förster Resonance Energy Transfer (FRET)-based. Translocation-based biosensors track the spatiotemporal dynamics of Ras activity through translocation of a cytosolically localized RBD, fused to a fluorescent protein, to compartments of Ras activity.¹⁵ This approach was used in early studies to report the presence of Ras activity at the Golgi Apparatus.^{16,17} However, the subcellular localization of these biosensors could be influenced by multiple factors and signal quantitation is prone to artifacts from variations in cell thickness and probe expression levels, which may have contributed to some controversial observations.^{16,18,9} On the other hand, current FRET-based Raichu biosensors contain full-length Ras and the RBD of Raf1 coupled to fluorescent protein FRET pairs; activation of Ras within the sensor drives Ras-RBD interaction, leading to an increase in FRET.20 While Raichu and other biosensors with similar designs^{21,22} allow more reliable quantitation, they entail heterologous expression of modified Ras as part of the biosensor, and essentially serve as GEF/GAP biosensors, as the measurements rely on the activity of cellular GEFs and GAPs. To provide reliable, quantitative measurements of cellular Ras activity, new Ras biosensor designs are required. Here we report the development and characterization of a new biosensor enabling direct Ras activity measurements. Using this biosensor, we provide new mechanistic insights into the regulation of HRas by Src kinase and demonstrate the utility of this biosensor in a live-cell Ras inhibitor assay.

### Results

### Design and Validation of RasAR.

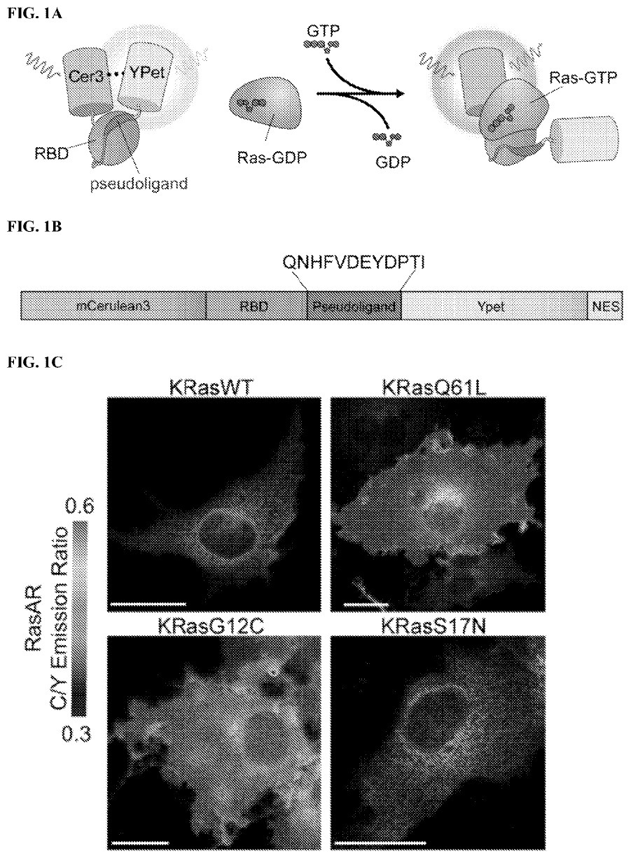

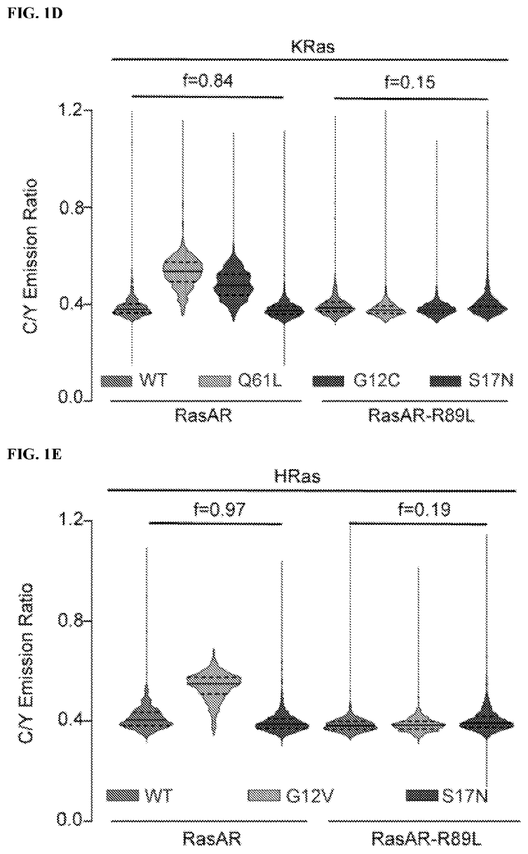

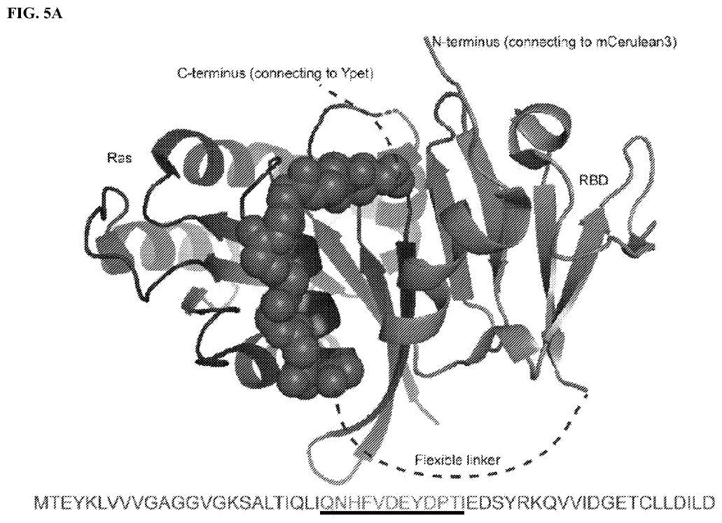

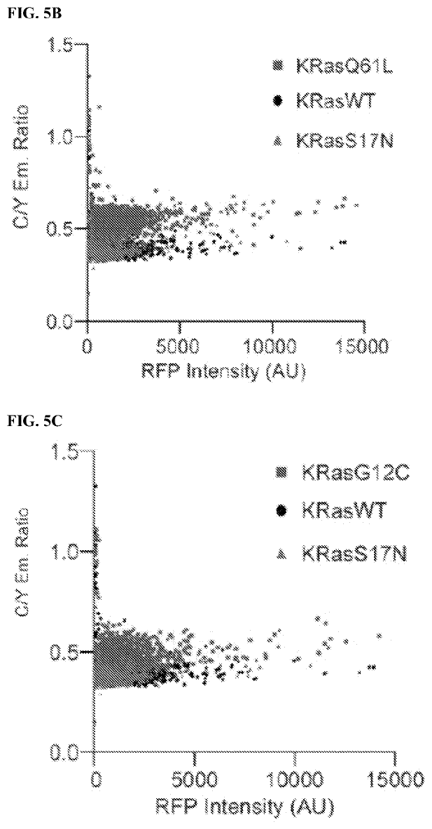

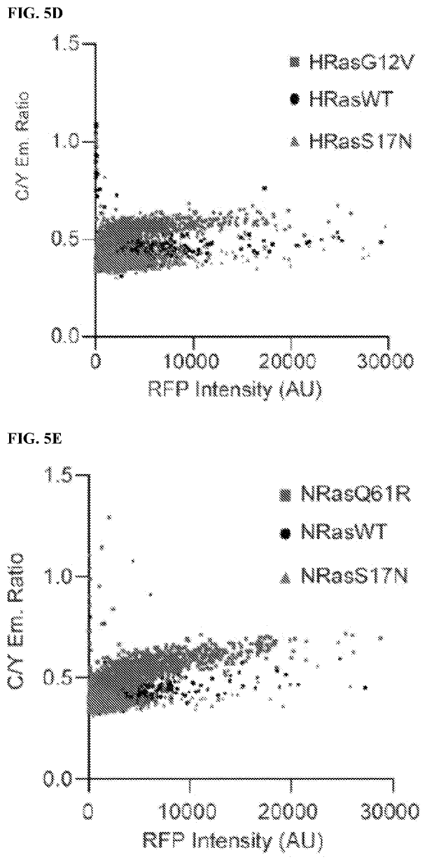

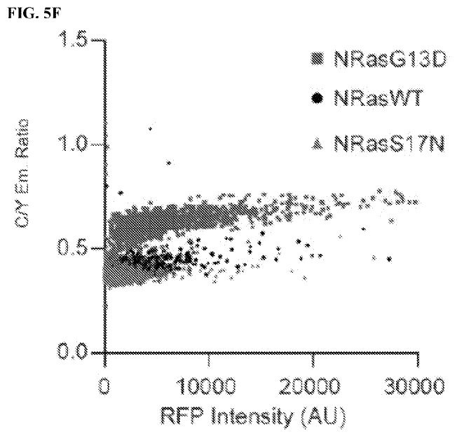

To develop a biosensor capable of quantitative measurement of cellular Ras activity, we utilized the RBD from Raf1 as the sensing component and sought to translate a binding event to a conformational change by employing a pseudoligand design.²² We derived a pseudoligand from the effector binding region of Ras, using a crystal structure of active Ras bound to Raf1 RBD23 as a blueprint. GTP binding to Ras triggers a conformational change that allows the effector binding region of Ras to bind to the effector RBD. Peptides derived from this region have been shown to bind RBD with low affinity.²⁴ We chose the pseudoligand sequence QNHFVDEYDPTI based on its ability to bind RBD in an appropriate orientation to poise a fluorescent protein pair that can undergo FRET (FIG. 5A). The Raf1 RBD and pseudoligand were joined by a linker and sandwiched between a pair of fluorescent proteins, mCerulean3 and YPet (FIGS. 1A-1B). A nuclear export sequence (NES) was also attached to the C-terminus of the construct to promote cytosolic localization (FIG. 1B). In the absence of Ras, the pseudoligand engages the RBD, bringing the fluorescent proteins into proximity to promote FRET. Binding of active Ras to the RBD displaces the pseudoligand, leading to a decrease in FRET (FIG. 1A).

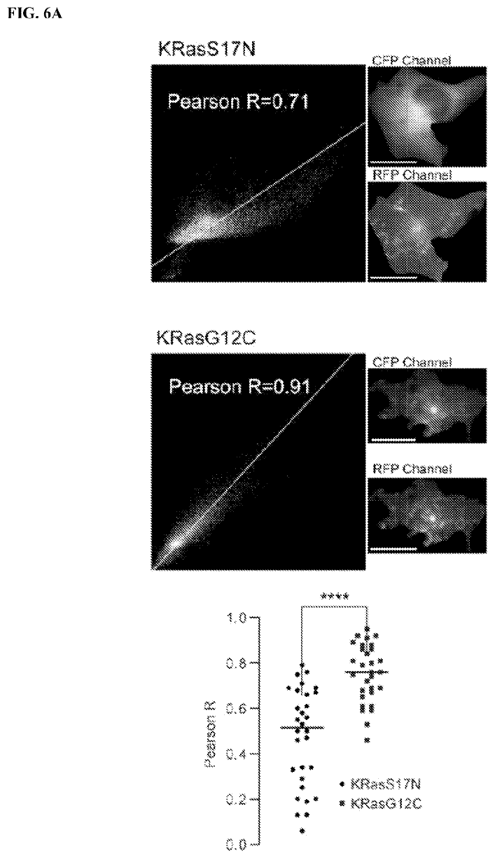

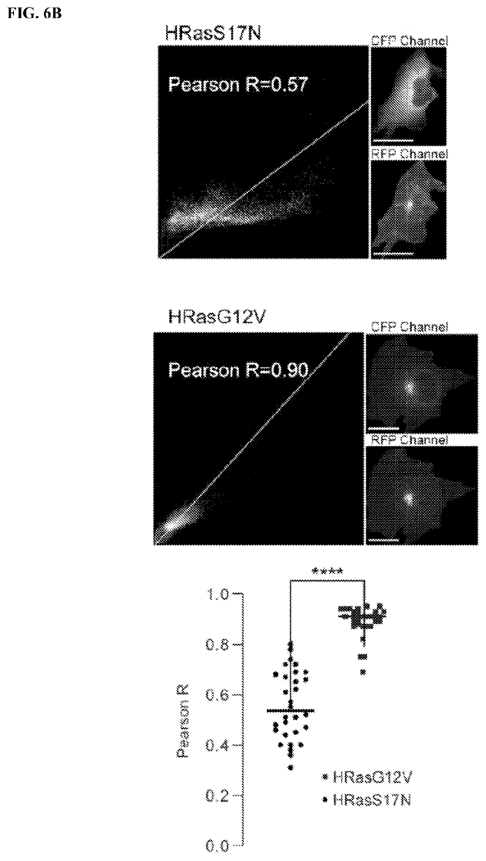

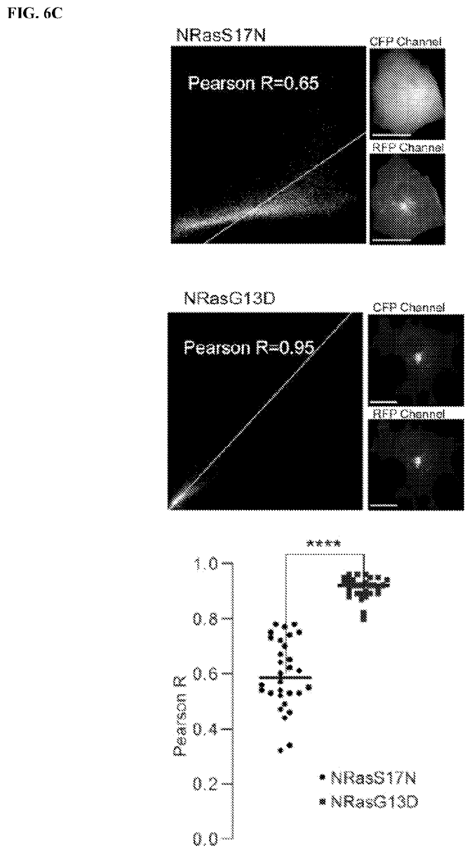

To test for proper functionality, we co-expressed the biosensor, which we named Ras Activity Reporter (RasAR), with KRasWT, constitutively active (CA) KRasQ61L or KRasG12C, or dominant negative (DN) KRasS17N, each N-terminally fused to mCherry, in COS-7 cells. As shown in FIGS. 1C-1D, cells expressing KRasQ61L or G12C exhibited significantly higher cyan-over-yellow (C/Y) emission ratios than cells expressing KRasWT or S17N, suggesting higher Ras activity in Q61L- or G12C-expressing cells. For instance, a higher emission ratio was observed for KRasQ61L compared with the DN mutant KRasS17N (n=1077 and 873 cells for mCherry-KRasQ61L and mCherry-KRasS17N, respectively, Cohen's d=2.19; FIG. 11). Incorporating an R89L mutation, which is known to substantially disrupt Ras binding,25 within the biosensor RBD substantially reduced the emission ratio differences (FIG. 1D, Cohen's f=0.15 for RasAR(R89L), f=0.84 for RasAR). The observed differences in biosensor C/Y emission ratio were not caused by variations in overexpression of Ras wildtype or mutants, as C/Y emission ratios were elevated across varying mCherry intensities (FIGS. 5B-5C). In addition to differences in C/Y emission ratio, the biosensor subcellular localization varied based on the activity of the co-expressed Ras. In cells expressing KRasS17N, biosensor fluorescence was distributed throughout the cytosol, whereas increased plasma membrane and perinuclear localization was observed upon co-expression with KRasG12C (FIG. 6A). Co-localization analysis showed that the biosensor colocalizes with KRasG12C, but not KRasS17N (FIG. 6A).

To examine whether RasAR detects the activities of Ras isoforms other than KRas, we assessed the functionality of RasAR in cells co-expressing RasAR and mCherry-tagged WT, CA or DN HRas and NRas in COS-7 cells. Similar to the results with KRas, co-expressing the biosensor with CA HRas or CA NRas led to enhanced co-localization between the biosensor and active Ras (FIGS. 6B-6C) and significantly elevated C/Y emission ratios compared with the corresponding WT or DN variants (FIGS. 1E-1F), independent of Ras over-expression levels (FIGS. 5D-5F). The most pronounced difference in C/Y emission ratio was observed between NRasS17N and NRasG13D populations, with a 66.9±0.6 [mean±SEM]% higher C/Y emission ratio (ΔR/R0) in the NRasG13D group, marking the dynamic range of the biosensor. Meanwhile, the R89L negative-control sensor displayed similar C/Y emission ratios regardless of the co-expressed Ras variant (FIGS. 1E-1F). These data suggest that RasAR successfully reports on active Ras through changes in its C/Y emission ratio.

### RasAR Captures Spatiotemporal Changes of Ras Activity in Living Cells

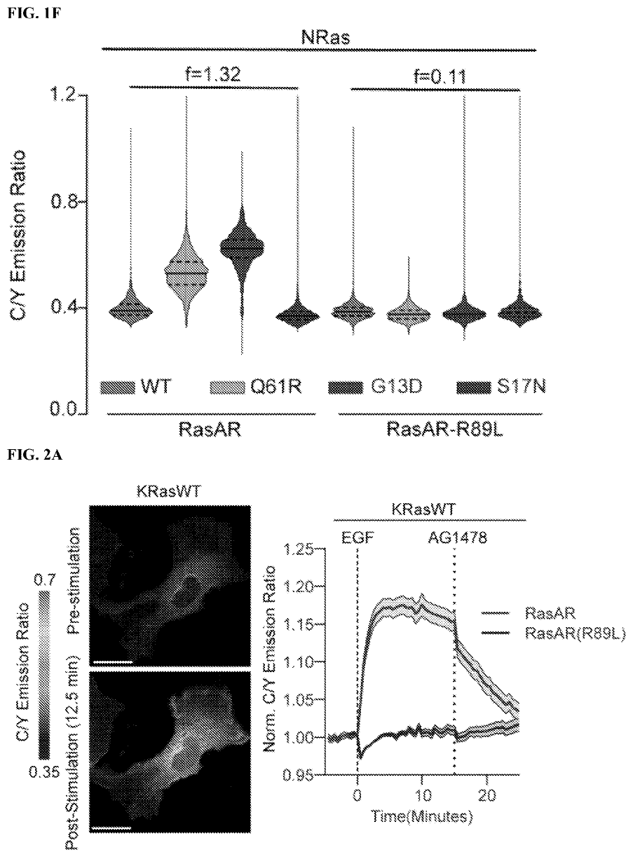

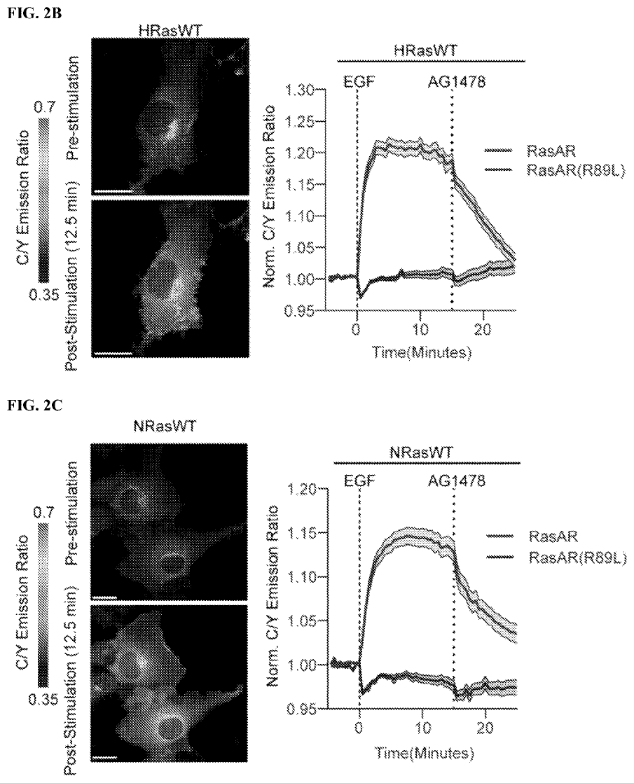

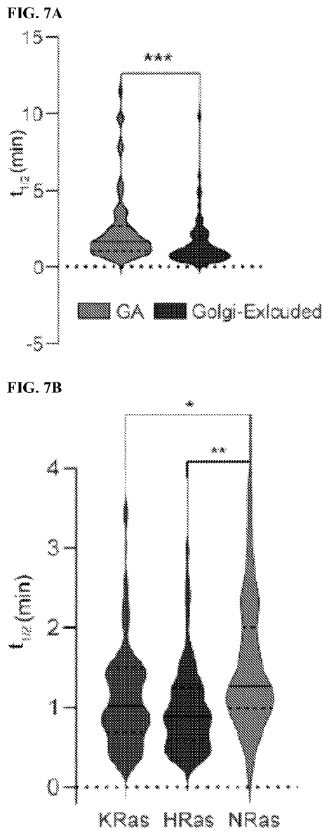

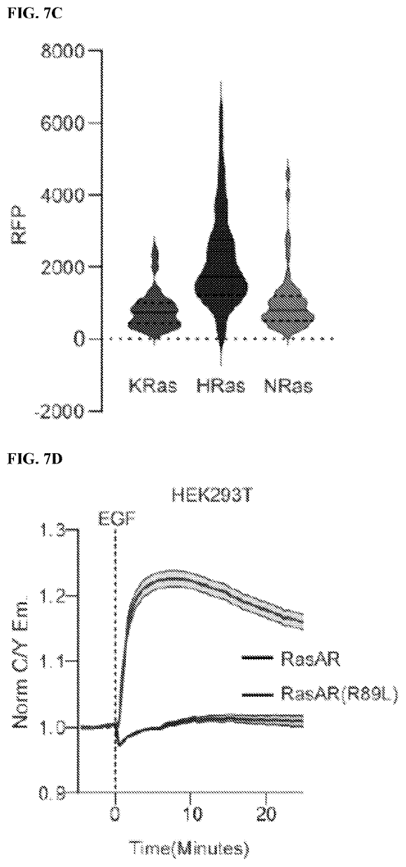

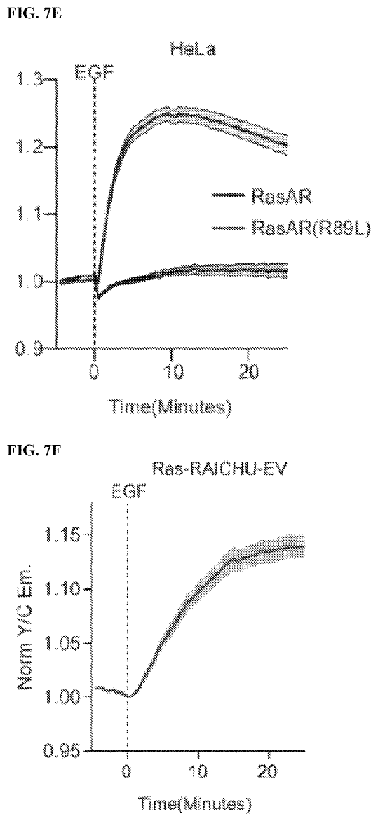

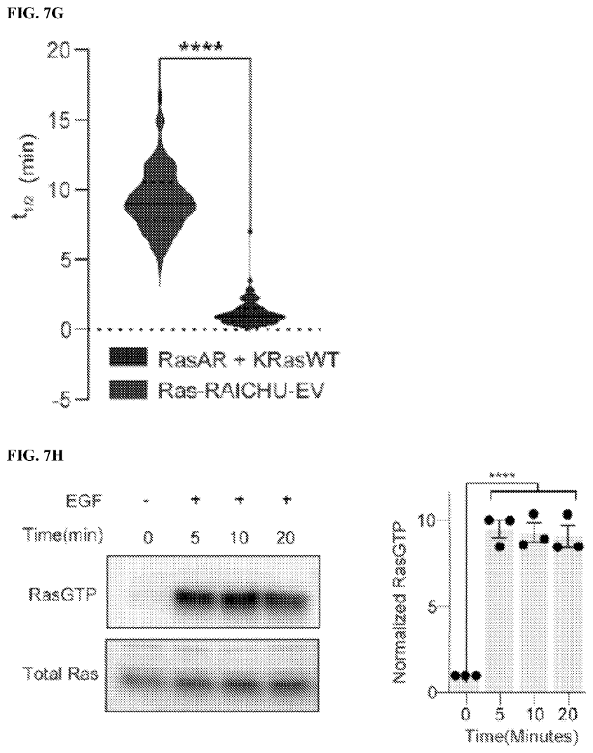

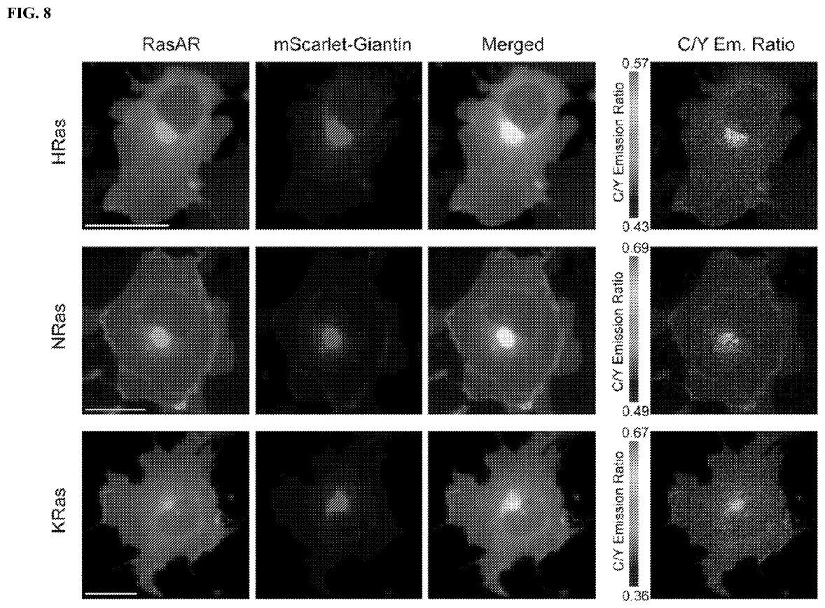

To determine whether RasAR can detect dynamic Ras activity, we co-expressed the sensor with mCherry-KRasWT in COS-7 cells and stimulated the cells with epidermal growth factor (EGF). Upon activation of the Ras pathway, we observed a rapid increase in the C/Y emission ratio in cells expressing RasAR (FIG. 2A; 17.0±1.2% [mean±s.e.m.], t½=1.4±0.15 min, n=90 cells, 3 experimental replicates). Consistent with previous studies,26 we observed higher C/Y emission ratios in membrane ruffles following EGF stimulation, indicating enhanced KRas activity in these regions. In addition, consistent with the Golgi localization of Ras,26 we observed an increased activity in the Golgi compartment (FIGS. 2A and 8). Addition of EGFR inhibitor AG1478 then led to a decrease in the C/Y emission ratio (FIG. 2A), showing the reversibility of RasAR. In contrast, cells co-expressing the RasAR (R89L) negative control sensor with mCherry-KRasWT showed no emission ratio change. In cells expressing mCherry-HRasWT or mCherry-NRasWT, EGF stimulation also led to increases in the RasAR C/Y emission ratio (FIGS. 2B-2C; HRas: 21.0±1.1%, t½=1.11±0.14 min; NRas: 14.1±0.9%, t½=1.5±0.08 min; n=90 cells from 3 experimental replicates, each), whereas no C/Y emission ratio increase was observed with the RasAR (R89L) negative control upon EGF stimulation. The EGF-stimulated increases in the emission ratio occurred in both the membrane ruffles as well as the Golgi compartment (FIGS. 2B-2C and 8), with slower kinetics observed at the Golgi (FIG. 7A). Notably, NRasWT showed significantly slower activation kinetics than KRasWT and HRasWT (FIG. 7B).

To assess the ability of RasAR to detect changes in Ras activity in multiple cell types, we co-expressed RasAR with mCherry-HRasWT in HeLa and HEK293T cells. In both cell types we observed an increase in C/Y emission ratio which was not observed in the negative control sensor RasAR (R89L) (FIGS. 7D-7E). We then compared the response of RasAR to the current Ras biosensor Ras-RAICHU-EV. EGF stimulation of COS-7 cells expressing Ras-RAICHU-EV led to a 13.5±1.0% increase in yellow-to-cyan emission ratio with a t½ of 9.31±0.28 min (FIGS. 7F-7G, n=65 cells from 3 experimental replicates), suggesting that the kinetics of Ras activity accumulation reported by Ras-RAICHU-EV were slower than those reported by RasAR. We then sought to validate RasAR responses with a pulldown assay measuring endogenous Ras activity. Notably, endogenous Ras showed fast kinetics reaching maximal activation within 5 min, similar to RasAR responses (FIG. 711). Together, these results suggest that RasAR is capable of reporting Ras activity dynamics in living cells.

### RasAR Probes the Regulation of Ras by Src

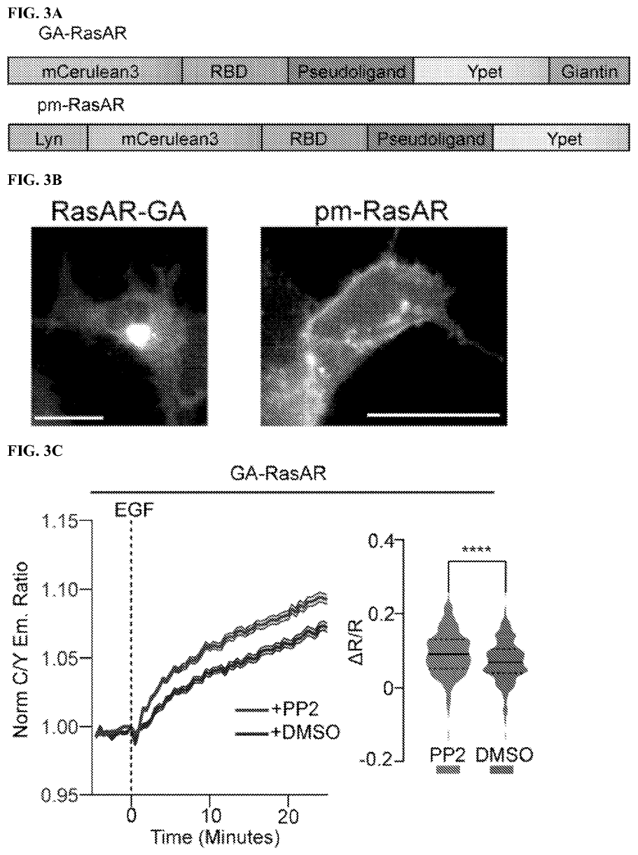

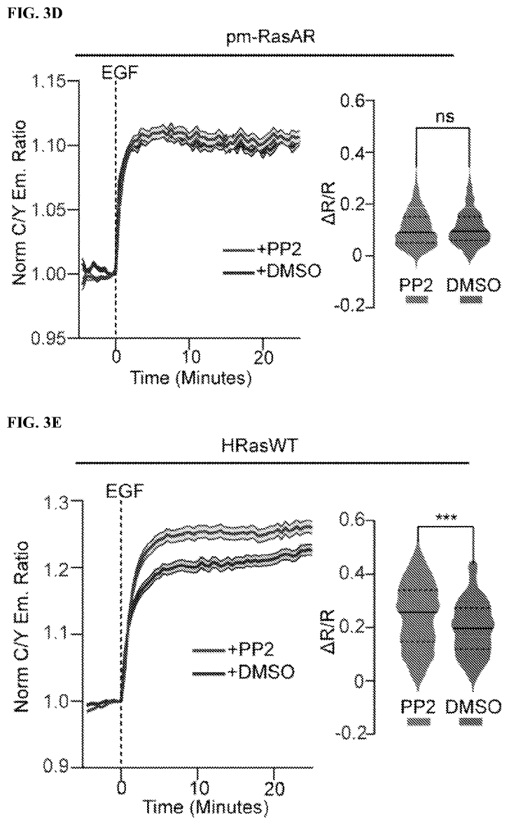

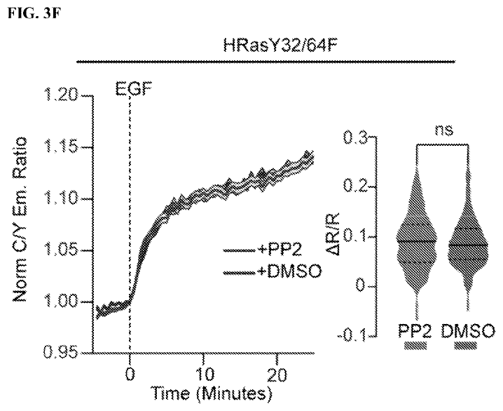

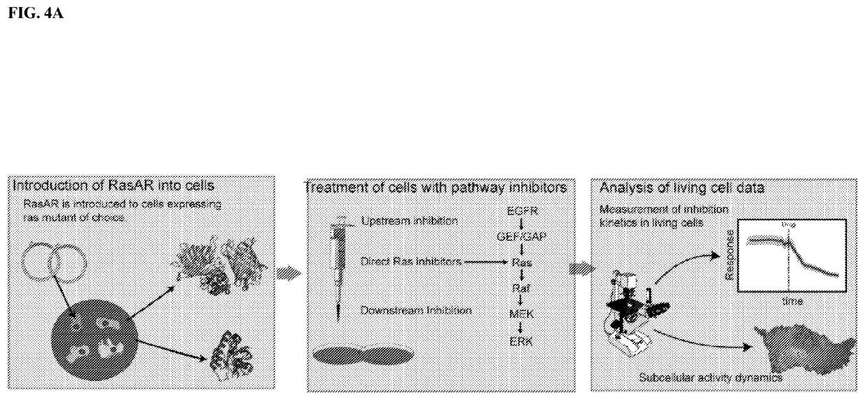

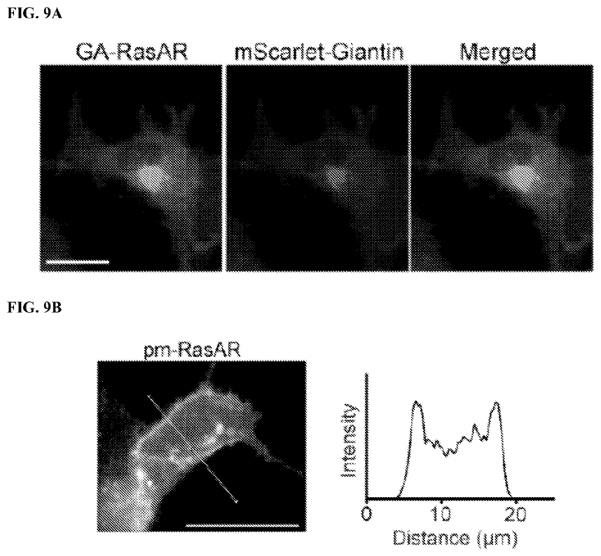

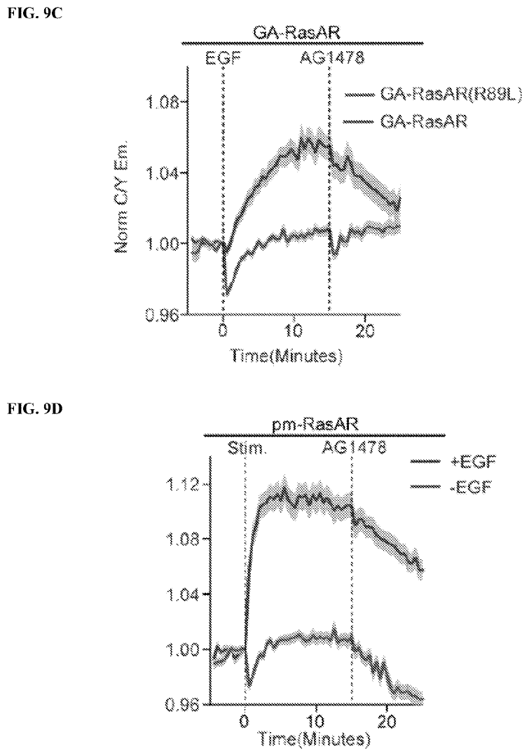

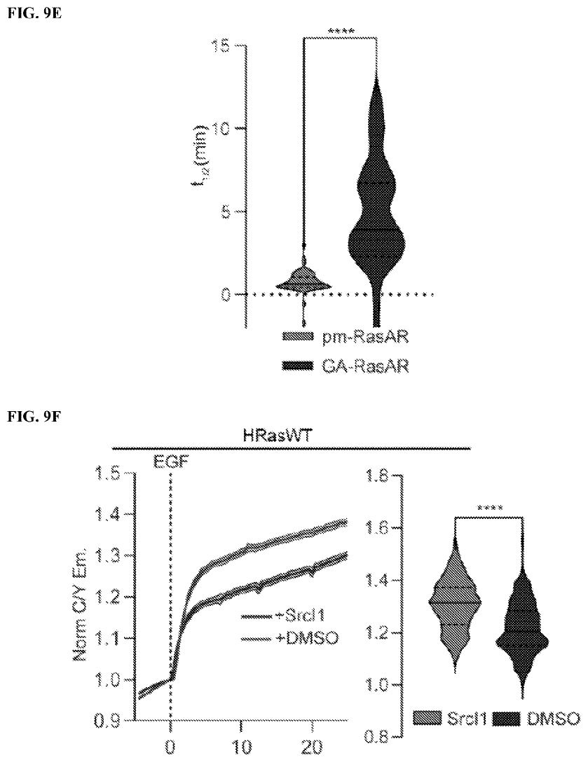

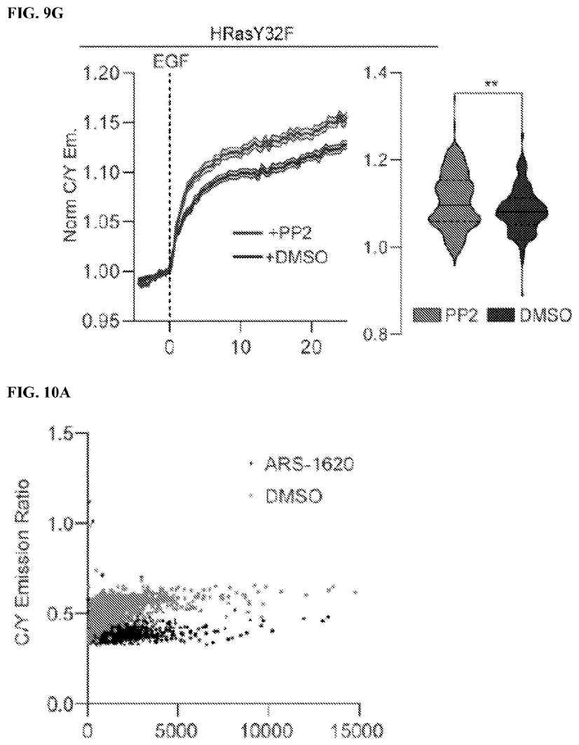

Early reports using translocation probes indicated that HRas may be activated at the Golgi in response to EGF stimulation.^{22,23} These initial studies proposed EGFR-mediated activation of Src as a prerequisite for Ras activity at the Golgi apparatus, as inhibition of Src with PP2 was shown to abrogate translocation of an RBD-GFP probe to the Golgi following EGF stimulation. However, other groups have shown an inhibitory role of Src via tyrosine phosphorylation at Y32 of HRas using a combination of pulldown assays and in vitro techniques, which preclude spatiotemporal analysis.²⁷ With its ratiometric readout and unimolecular design, RasAR is uniquely poised to measure cellular Ras activity within spatially defined compartments with the aid of subcellular targeting motifs. Noting this, we constructed two different RasAR variants, pm-RasAR, targeted to the plasma membrane with an N-terminal tag from Lyn kinase, and GA-RasAR, targeted to the Golgi apparatus via C-terminal fusion with a Giantin fragment (FIGS. 3A-3B and 9A-9B). An increase in C/Y emission ratio was observed in cells expressing GA-RasAR and mCherry-HRasWT stimulated with EGF but not in cells expressing a negative-control sensor, GA-RasAR (R89L) (FIG. 9C). Similarly, pm-RasAR showed an EGF-stimulated increase in C/Y emission when co-expressed with mCherry-HRasWT in COS-7 cells, which was not observed in cells treated with vehicle control (FIG. 9D). EGF-induced Ras activity increases showed distinct kinetics at the plasma membrane and Golgi compartment, with a longer t½ for GA-RasAR compared to pm-RasAR (FIG. 9E, t½=5.3±0.4 min for GA-RasAR, t½=0.86±0.07 min for pm-RasAR, p<0.0001), consistent with the trend observed using cytosolic RasAR (FIG. 7A). These results corroborate previous studies showing delayed and prolonged HRas activation at the Golgi apparatus in response to EGF stimulation.¹⁶

To clarify the regulatory role of Src on HRas at the Golgi, we monitored HRas activity at the Golgi with GA-RasAR in the presence of Src inhibitor PP2. As shown in FIG. 3C, in cells expressing GA-RasAR and mCherry-HRas, EGF-induced responses were enhanced by PP2 pretreatment (n=300 cells each for PP2 and DMSO-treated groups, p<0.0001), suggesting an inhibitory role for Src on Ras activity at the Golgi. Interestingly, PP2 pretreatment did not affect EGF-induced Ras activity at the plasma membrane (FIG. 3D). In sum, these results suggest that Src inhibition increases EGF-induced Ras activity specifically in the Golgi compartment, but not at the plasma membrane.

Cytosolic RasAR, when co-expressed with HRasWT, showed a larger EGF-induced response with PP2 pretreatment (FIG. 3E, n=180 cells, p<0.001). Additionally, treatment with the more specific Src inhibitor SrcI1 similarly enhanced EGF-induced Ras activity (FIG. 9F, n=150 cells for SrcI1, n=144 for DMSO, p<0.0001). Phosphorylation of residue Y32 has been implicated in the negative regulation of HRas by Src.31 To verify this mechanism of Src inhibition we co-expressed RasAR with HRasY32F and stimulated with EGF. However, mutating Y32 did not abolish the inhibitory effect of Src on HRas, as PP2-pretreated cells still showed an enhanced response to EGF in comparison with control cells (FIG. 9G). KRas was recently shown to be negatively regulated by Src via dual phosphorylation of Y32 and Y64,28 which negatively impacts multiple steps of the GTPase cycle and impairs binding to effectors. However, the role of Y64 in HRas has not been investigated. To test the hypothesis that phosphorylation of both Y32 and Y64 is involved in Src regulation of HRas, we expressed RasAR and an mCherry-labeled HRas mutant containing Y32F/Y64F mutations and monitored EGF-induced responses in the presence and absence of PP2. In this case, we observed no significant difference between PP2-pretreated cells and control cells (FIG. 3F, n=180 cells each for PP2- and DMSO-treated groups). These data indicate that Src plays an inhibitory role in EGF-induced HRas activity in a manner that specifically targets subcellular pools of Golgi-Ras and involves Y64.

### RasAR Detects Ras Inhibition in Living Cells

Ras is a prominent oncogene and an extensively pursued molecular target for cancer therapy. However, cellular assays for characterizing inhibitor effects require the use of Ras pulldown assays, which are cumbersome and difficult to extend to high throughput. Furthermore, these assays require cell lysis and do not allow characterization of spatiotemporal activities of the inhibitors. With a ratiometric biosensor capable of measuring Ras activity, we sought to measure live-cell Ras inhibition with RasAR. This assay should allow quantitative measurement of Ras inhibition in living cells with high spatiotemporal resolution. Temporal dynamics of Ras inhibition can be tracked and subcellular inhibitory effects can be compared to provide important information about target engagement in living cells (FIG. 4A). In addition, the intact cellular environment contains the entire Ras/Raf/Extracellular signal-regulated kinase (ERK) signaling pathway²⁹ and additional cross-regulatory networks, which should allow testing of inhibitory effects on the entire pathway.

To test the utility of RasAR in a live-cell Ras inhibitor assay, we first focused on ARS1620, a previously developed KRasG12C inhibitor, which binds to GDP-bound KRasG12C and prevents nucleotide exchange.¹⁰ Cells co-expressing KRasG12C and RasAR were incubated with 10 μM ARS1620 or DMSO for 24 hr. We observed a higher C/Y emission ratio in KRasG12C-expressing cells treated with DMSO compared with ARS1620 treatment, whereas KRasWT controls showed no effect of inhibitor treatment (FIG. 4B). Additionally, we tested different concentrations (10 μM, 3 μM, 1 μM, 0.3 μM, 0.1 μM) of ARS1620 in COS-7 cells expressing both RasAR and mCherry-KRasG12C. Decreasing dosages of ARS1620 corresponded to higher C/Y emission ratios, demonstrating the capability of RasAR to measure dose-dependent inhibition of KRasG12C in living cells (FIG. 4C). Assessing Ras expression by RFP intensity suggested that differences in C/Y emission ratio are related to changes in activity and not expression levels (FIGS. 10A-10B). To further demonstrate the capability of live-cell assays for characterizing Ras inhibitors, we next sought to test the effect of ARS1620 on the Ras signaling pathway by using our ERK activity reporter (EKAR4)³⁰ to measure the activity of the key downstream master regulator ERK. EKAR4 serves a surrogate substrate of ERK and reports ERK-mediated phosphorylation as an increase in FRET between CFP and YFP. We co-expressed mCherry-KRasG12C with EKAR4 and treated the cells with ARS1620 (FIG. 4D). As expected, we observed a larger effect of ARS1620 treatment on the EKAR4 Y/C emission ratio in mCherry-KRasG12C expressing cells, compared to mCherry-KRasWT expressing COS-7 cells (FIG. 4D, Cohen's d=0.02, 0.84 for KRasWT and KRasG12C, respectively), indicating that ARS1620 can effectively inhibit the Ras-ERK pathway in KRasG12C expressing cells.

We next assessed the kinetics of KRasG12C inhibition by treating RasAR and KRasG12C co-expressing COS-7 cells with 10 μM ARS1620 and measuring the C/Y emission ratio at different time points (FIG. 4E). Surprisingly, we observed a steady decline in the C/Y emission ratio over the first 5 hr of treatment (FIG. 11), suggesting relatively slow cellular kinetics, which may correspond to the slow intrinsic rate of GTP hydrolysis by KRasG12C. Given that KRasG12C is continually being synthesized during these experiments, we next tested whether ongoing protein synthesis contributes to the slow inhibition kinetics that we observed by treating cells with ARS1620 in the presence of cycloheximide. As expected, cycloheximide treatment reduced KRasG12C expression levels, confirming that KrasG12C is re-synthesized within the 24-hr timeframe of our assay (FIG. 10C). However, inhibiting this re-synthesis did not speed up the kinetics of Ras inactivation (FIGS. 10D and 11). We also considered the possibility that the slow kinetics of inhibition were related to Ras overexpression; however, dividing cells into low-, medium-, and high-expression groups according to RFP intensity revealed no relationship between the kinetics of inhibition and KRasG12C expression levels (FIGS. 10E-10H). To further investigate Ras inhibition kinetics, we treated cells with Sotorasib¹² or Adagrasib,³¹ two additional KRasG12C inhibitors. We observed similar inhibition kinetics with Sotorasib and Adagrasib, with a continuous C/Y emission ratio decrease for the first 5 hr post treatment (FIGS. 4F-4G and 11). In addition, the slow inhibition kinetics of Sotorasib was also observed at the ERK activity level shown by EKAR4 emission ratio changes (FIG. 10I). These data demonstrate the utility of RasAR in the context of Ras inhibition assays, making it possible to bring live-cell data to the field of Ras inhibitor development.

### Discussion

We developed anew biosensor, RasAR, featuring a pseudoligand-based design, to report dynamic changes in compartment-specific Ras activities in living cells. Compared with traditional biochemical assays, RasAR enables live-cell tracking of Ras activity dynamics. RasAR provides a ratiometric readout, allowing for the cancellation of cellular variations and quantitative measurement of Ras activity. Compared with current Ras biosensors, the Ras RAICHU series, the main advantage of RasAR is that the biosensor itself does not contain Ras and is therefore capable of measuring cellular activity of different Ras isoforms. We demonstrated the fast kinetics of RasAR, which correlate well with endogenous Ras activation, whereas Ras RAICHU showed slow kinetics, possibly due to steric hindrance of the embedded Ras within the unimolecular construct. Furthermore, RasAR allows for interrogation of subcellular Ras activity in living cells via a genetic targeting approach.^{14,32} The dynamic range of RasAR (66.9±0.6%) is on par or even compares favorably with many widely used FRET-based biosensors.^{30,33,34} While RasAR is currently capable of detecting the activity of co-expressed Ras in COS-7 cells, it does not show clear responses to EGF stimulation in COS-7 cells with only endogenous Ras expression. Ongoing experiments will test its capability of detecting endogenous Ras activity in a variety of cancer cell lines. Future development will also focus on further enhancing the sensitivity and dynamic range of RasAR to sensitively measure endogenous Ras activity. In particular, we hypothesize that enhancing the affinity of the RBD towards active Ras will be critical for detecting endogenous Ras activity, as previous research has demonstrated that wild-type RBD has insufficient affinity to effectively outcompete endogenous Ras effectors within the cell.³⁵

It was unclear whether the different Ras isoforms follow similar activation kinetics. From our findings, NRas has the slowest kinetics among the Ras isoforms, a property that may be related to the unique subcellular localization of this isoform.³⁶ NRas has a distinct hypervariable region, with post-translational modifications consisting of a single cysteine palmitoylation in addition to the C-terminal cysteine farnesylation, which is conserved among all Ras isoforms. This differs from the dual palmitoylation of HRas, and the polybasic stretch of KRas.^{4,37} While HRas and KRas both show prominent localization at the plasma membrane, with some Golgi localization,²⁶ consistent with the activity detected at these locations by RasAR (FIGS. 2A-2B and 8), a substantial portion of NRas has also been observed to accumulate at the Golgi apparatus and in the cytoplasm, where it forms a complex with VPS35.36 The unique post-translational modifications and distinct cellular distribution of NRas may contribute to its delayed activation, as EGF-stimulated rapid Ras activation is mediated by Sos-induced nucleotide exchange at the plasma membrane. Given that cytosolic NRas is protected by the carrier VPS35, this pool of NRas may not exhibit significant activity. Indeed, the majority of NRas activity that we detected with RasAR occurred at the plasma membrane and Golgi (FIGS. 2C and 8).

RasAR also revealed the nature of Src regulation of Ras activity at the Golgi compartment. In stark contrast to a previous model in which Src promotes Golgi Ras activity, we showed that Src negatively regulates Ras activity at the Golgi compartment, but not at the plasma membrane (FIGS. 3A-3F). This discrepancy may result from the use of different cell systems. For example, RasGRP1, a Ras GEF that has been suggested to act downstream of Src, is endogenously expressed in PC12 cells, but not in COS-7 cells.³⁸ Mechanistically, our data suggest that Src regulates Golgi HRas through phosphorylation of tyrosines 32 and 64, contrasting with previous studies²⁷ and identifying Y64 as an important mechanistic link in Src regulation of HRas. Residue Y64 is conserved among Ras isoforms. Thus, similar to previous reports with KRas28 and our results with HRas, Y64 likely plays a critical role in NRas function as well. How does Src achieve specific regulation of the Golgi pool of HRas?Further experiments will explore subcellular differences in Src activity.³⁹ Interestingly, it has been demonstrated that Ras signaling at the Golgi compartment acts through Ral GTPase, antagonizes ERK activity, and promotes apoptosis in MCF-7 cells.⁴⁰ On the other hand, it was shown that site-selective activation of Ras at the Golgi can produce Golgi-specific ERK activation.⁴¹ Future experiments will examine the downstream effects of Src regulation on the Ras pathway. The specific subcellular regulation identified here thus adds to an already very complex picture of subcellular Ras activity that is differentially regulated and coupled to downstream signaling.

While development of effective inhibitors to target oncogenic Ras mutants is an ongoing pursuit, RasAR offers an amenable strategy for fluorescence imaging of Ras activity dynamics in living cells treated with different candidate inhibitors. KRasG12C inhibitors such as ARS1620 and Sotorasib bind to the Switch II pocket found in GDP-bound KRasG12C. The binding of these inhibitors prevents Sos-induced nucleotide exchange and abrogates signaling activity.8,10 Using a RasAR-based live-cell assay, we found that the maximal effect of these KRasG12C inhibitors requires at least 5 hr, longer than the 2-hr window for achieving full target engagement suggested by previous mass spectrometry experiments,¹⁰ yet similar to previous studies of ARS853 utilizing Ras pulldown assays.⁸ One explanation for this discrepancy may lie within the details of the assays and models employed. In previous studies, cells were lysed prior to engagement or activity measurements in mouse or cell models expressing endogenous KRasG12C. In contrast, our data were collected in living cells where KRasG12C was co-expressed with our fluorescent biosensor. It is possible that different expression levels of KRasG12C play a role in producing different results, although we did not observe a dependence of the inhibitory effect on the expression levels of KRasG12C in different cells (FIGS. 10E-10H). In fact, the slow kinetics that we observed are consistent with the idea that KRasG12C inhibition by these compounds requires nucleotide cycling and cooperation from cellular factors. GDP-bound KRasG12C can be formed via intrinsic GTPase activity or specific GAPs but can also be removed by cellular GEFs. The kinetics of KRas inhibition are therefore intrinsically slower than for inhibition of a single target, as previously shown in the case of ARS853.8 Considering the role that GEFs and GAPs play in regulating the nucleotide state of KRasG12C mutants, cell-type specific differences in these regulators may also affect inhibition kinetics. The ability to detect this residual Ras activity showcases the sensitivity of our live-cell Ras inhibition assay, which can be used to investigate the influence of other cellular factors, test combination therapies and explore potential resistance mechanisms. RasAR is therefore a powerful tool not only for probing isoform-selective and location-specific Ras signaling but also for revealing live-cell properties and behaviors of Ras inhibitors.

### Methods

### Gene Construction

All constructs were generated using a one-step multi-fragment Gibson Assembly reaction (NEB 2× High Fidelity Master Mix). All fragments were PCR amplified with Q5 polymerase (NEB). For RasAR, mCerulean3 was generated via PCR amplification using primers 1 and 2 (see Supplementary Table 2). The RBD with 3′-pseudoligand was amplified using overlap extension from CRY2PHP-mCherry-Raf1, a gift from the Cui Lab,42 using primers 3 and 4. YPet with a 3′-NES (LPPLERLTL; SEQ ID NO:13) was amplified using primers 5 and 6. pcDNA3.1+ (Invitrogen) backbone, incorporating NES overlap sequence was synthesized in two fragments by Gibson assembly with primer pairs 7/8. For GA-RasAR, RasAR without any targeting motif was amplified using primers 9 and 10. pcDNA3.1+ with a 5′ Giantin tag and 8-residue linker was amplified from CIAR-Golgi, provided by the Maly lab,43 using primers 11 and 12. For pm-RasAR, the N-terminal Lyn-targeting motif was amplified with overlap extension using primers 13 and 14. Untargeted RasAR was amplified with primers 15 and 16. Ras constructs are gifts from Maly, Inoue and Devaraj labs, with mCherry tagged at the N-terminal. Codon-optimized KRasWT and NRasWT with overlap sequences, 5′-AAGCTGGCTAGCGTTTAAACTTAAGCTTGCCACC- and -TCTAGAGGGCCCGTTTAAACCCGCTGATCAGC-3′, were purchased from Genscript. These constructs were amplified with a primers 31 and 32. The fidelity of construct assembly was verified by Sanger sequencing.

### Cell Culture

COS-7 cells were cultured in Dulbecco modified Eagle medium (DMEM, Gibco) containing 4.5 g/L glucose and supplemented with 10% (v/v) fetal bovine serum (FBS; Sigma) and 1% (v/v) penicillin-streptomycin (Pen-Strep, Sigma-Aldrich). Cells were maintained in a humidity-controlled incubator at 37° C. with 5% CO2. Cells were seeded onto sterile 35-mm glass-bottomed dishes and grown to 50-70% confluency. Cells were transfected with PolyJet (SignaGen Laboratories) 24 hr prior to imaging. Prior to transfection the COS-7 cells were washed with Dulbecco Phosphate Buffered Saline (DPBS, Gibco), and medium was changed to a serum-free DMEM to serum starve the cells for the 24 hr during transfection prior to imaging.

### Ras-GTP Pulldown Assay

COS-7 cells were starved in serum-free medium for 24 hours, stimulated with 100 ng/ml EGF, and then collected at different time points following stimulation. Cells (2-3×10\hat{ }6 cells) were rinsed with ice-cold PBS and then lysed with 300 μl of lysis/binding/wash buffer (25 mM Tris-HCl, pH 7.2, 150 mM NaCl, 5 mM MgCl2, 5% glycerol, 1% NP40) from Active Ras Detection kit (Cell Signaling Technology, #8821) supplemented with complete, EDTA-free protease cocktail inhibitors (Millipore Sigma, 11697498001), 1 mM PMSF, and 80 μg of GST-Raf-RBD (Cell signaling Technology, #8821). Lysates were votexed briefly, incubated on ice for 5 min and subsequently pre-cleared at 16,000×g at 4° C. for 15 min. 90% of the pre-cleared lysates were subsequently added to prewashed glutathione agarose beads from Active Ras Detection kit (Cell signaling Technology, #8821) for 1 hour at 4° C. under constant rocking. 5-10% of the lysates were saved for total Ras analysis. The beads were then spun down and washed once with lysis/binding/wash buffer, and eluted with 50 μl of 2×SDS-PAGE sample buffer with 200 mM dithiothreitol (DTT). The eluted samples were heated for 5 min at 98° C. and used for Western blotting to determine the level of GTP-bound Ras. Protein was separated via 4-15% SDS-PAGE and transferred to PVDF membranes. The membranes were blocked with TBS containing 0.1% Tween-20 and 5% bovine serum albumin and then incubated with rabbit anti-Ras antibody (CST, #3965, 1:1000) overnight at 4° C. After incubation with the goat anti-Rabbit horseradish peroxidase-conjugated secondary antibody, the membranes were developed using horseradish peroxidase-based chemiluminescent substrates (34579 and 34076, Thermoscientific). The intensity of the bands was quantified with ImageJ 1.52s software. Active GTP-bound Ras was normalized to total Ras in cell lysate.

### Time-Lapse Widefield Epifluorescence Imaging

Cells were washed three times with Hanks's balanced salt solution (HBSS, Gibco) prior to imaging. Cells were imaged in HBSS in the dark at 37° C. Epidermal growth factor (EGF, Sigma-Aldrich), AG1478 (Selleckchem, S2728), MRTX-1257 (MedChemExpress, HY-114436), Sotorasib (MedChemExpress, HY-114277), and ARS1620 (gift of Kevan Shokat, University of California San Francisco) were added at the indicated concentrations. Cells were imaged on a Zeiss Axio Observer Z1 microscope equipped with a 20×/0.8 NA objective and a Hamamatsu Flash 4.0 sCMOS camera, controlled using MicroManager 2.0 software. For time series, images from multiple fields of view were acquired at each time point at 30 sec intervals. RFP was imaged using a 568DF55 excitation filter, a 600DRLP dichroic mirror, and a 653DF95 emission filter. CFP was imaged using a 420DF20 excitation filter, a 455DRLP dichroic mirror, and a 470DF40 emission filter. CYFRET was imaged using a 420DF20 excitation filter, a 455DRLP dichroic mirror, and a 535DF25 emission filter. YFP was imaged using a 495DF10 excitation filter, a 515DRLP dichroic mirror, and a 535DF25 emission filter. Exposure times for each channel were 250-500 ms. All filter sets were controlled by a Lambda 10-2 filter-changer (Sutter Instruments). All fluorescence images were background-correcting by rolling circle background subtraction in ImageJ using a rolling-ball circle radius of 350 pixels. Cyan/yellow or yellow/cyan emission ratios were then calculated by dividing the CFP channel values by the CYFRET channel values, or the CYFRET channel values by CFP channel values, respectively. For the time-series analysis, all values were normalized to the time point prior to drug addition by dividing each value by basal ratio value prior to drug addition. Co-expression experiments were processed using Cell Profiler on background subtracted images and segmented using CYFRET channel images. Individual t½ measurements were acquired using custom script within Matlab.

### Statistics and Reproducibility

All imaging experiments were performed with independent experimental replicates with all replicate attempts being successful. Experiments from FIGS. 1D-1F, 2A-2C, 3E-3F, 4B-4G, 5B-5F, 7A-7H, 9F-9G were performed with 3 independent replicates. 5 independent replicates were performed for FIGS. 3C-3D. Two experimental replicates were performed for FIGS. 9C, 9D, 9G, and 10A-10I. Statistical analyses were performed using GraphPad Prism 8 (GraphPad Software). In data sets with large sample sizes (FIGS. 1D-1F, 4B-4G, 10A-10I), small effects led to statistically significant changes in typical statistical comparisons. Due to this, we performed Cohen's f, effect size calculations based on one-way ANOVA, and Cohen's d, effect size calculations based on pairwise i-test. For smaller data sets (FIG. 3E-3F, 6A-C, 7A-7H, and 9C-9G) Mann-Whitney pairwise comparisons are performed.

Averaged live-cell imaging time-courses depict mean z s.e.m.; and violin plots depict the median and quartiles, as indicated in the figure legends.

### REFERENCES

1. Lowy, D. R. & Willumsen, B. M. Function and Regulation of Ras. Annu. Rev. Biochem. 62, 851-91 (1993).
2. Lu, S. et al. Ras Conformational Ensembles, Allostery, and Signaling. Chem. Rev. 116, 6607-6665 (2016).
3. Terrell, E. M. & Morrison, D. K. Ras-mediated activation of the Raf family kinases. Cold Spring Harb. Perspect. Med. 9, a033746 (2019).
4. Mo, S. P., Coulson, J. M. & Prior, I. A. RAS variant signalling. Biochem. Soc. Trans. 46, 1325-1332 (2018).
5. Prior, I. A. & Hancock, J. F. Ras trafficking, localization and compartmentalized signalling. Semin. Cell Dev. Biol. 23, 145-153 (2012).
6. Simanshu, D. K., Nissley, D. V. & McCormick, F. RAS Proteins and Their Regulators in Human Disease. Cell 170, 17-33 (2017).
7. Stalnecker, C. A. & Der, C. J. RAS, wanted dead or alive: Advances in targeting RAS mutant cancers. Sci. Signal. 13, 1-7 (2020).
8. Lito, P., Solomon, M., Li, L. S., Hansen, R. & Rosen, N. Cancer therapeutics: Allele-specific inhibitors inactivate mutant KRAS G12C by a trapping mechanism. Science 351, 604-608 (2016).
9. Patricelli, M. P. et al. Selective inhibition of oncogenic KRAS output with small molecules targeting the inactive state. Cancer Discov. 6, 316-329 (2016).
10. Janes, M. R. et al. Targeting KRAS Mutant Cancers with a Covalent G12C-Specific Inhibitor. Cell 172, 578-589.e17 (2018).
11. Ostrem, J. M., Peters, U., Sos, M. L., Wells, J. a. & Shokat, K. M. RASG (12C) inhibitors alloserically control GTP affinity and effector interactions Supplementary information. Nature 503, 1-27 (2013).
12. Blair, H. A. Sotorasib: First Approval. Drugs (2021) doi:10.1007/s40265-021-01574-2.
13. Baker, M. J. & Rubio, I. Active GTPase Pulldown Protocol. in Methods in Molecular Biology vol. 2262 117-135 (2021).
14. Lang, P., Yeow, K., Nichols, A. & Scheer, A. Cellular imaging in drug discovery. Nat. Rev. Drug Discov. 5, 343-356 (2006).
15. Bivona, T. G., Quatela, S. & Philips, M. R. Analysis of Ras Activation in Living Cells with GFP-RBD. Methods Enzymol. 407, 128-143 (2006).
16. Chiu, V. K. et al. Ras signalling on the endoplasmic reticulum and the Golgi. Nat. Cell Biol. 4, 343-350 (2002).
17. Bivona, T. G. et al. Phospholipase Cy activates Ras on the Golgi apparatus by means of RasGRP1. Nature 424, 694-698 (2003).
18. Rubio, I. et al. TCR-Induced Activation of Ras Proceeds at the Plasma Membrane and Requires Palmitoylation of N-Ras. J. Immunol. 185, 3536-3543 (2010).
19. Augsten, M. et al. Live-cell imaging of endogenous Ras-GTP illustrates predominant Ras activation at the plasma membrane. EMBO Rep. 7, 46-51 (2006).
20. Mochizuki, N. et al. Spatio-temporal images of growth-factor-induced activation of Ras and Rapt. Nature 411, 1065-1068 (2001).
21. Bery, N. et al. BRET-based RAS biosensors that show a novel small molecule is an inhibitor of RAS-effector protein-protein interactions. Elife 7, 1-28 (2018).
22. Greenwald, E. C., Mehta, S. & Zhang, J. Genetically encoded fluorescent biosensors illuminate the spatiotemporal regulation of signaling networks. Chem. Rev. 118, 11707-11794 (2018).
23. Fetics, S. K. et al. Allosteric effects of the oncogenic rasq61l mutant on raf-RBD. Structure 23, 505-516 (2015).
24. Ohnishi, M. et al. Selective inhibition of Ras interaction with its particular effector by synthetic peptides corresponding to the Ras effector region. J. Biol. Chem. 273, 10210-10215 (1998).
25. Kiel, C., Serrano, L. & Herrmann, C. A detailed thermodynamic analysis of Ras/effector complex interfaces. J. Mol. Biol. 340, 1039-1058 (2004).
26. Choy, E. et al. Endomembrane trafficking of ras: The CAAX motif targets proteins to the ER and Golgi. Cell 98, 69-80 (1999).
27. Bunda, S. et al. Src promotes GTPase activity of Ras via tyrosine 32 phosphorylation. Proc. Natl. Acad. Sci. U.S.A. 111, E3785-E3794 (2014).
28. Kano, Y. et al. Tyrosyl phosphorylation of KRAS stalls GTPase cycle via alteration of switch I and II conformation. Nat. Commun. 10, https://doi.org/10.1038/s41467-018-08115-8 (2019).
29. Lavoie, H., Gagnon, J. & Therrien, M. ERK signalling: a master regulator of cell behaviour, life and fate. Nat. Rev. Mol. Cell Biol. 21, 607-632 (2020).
30. Keyes, J. et al. Signaling diversity enabled by rap1-regulated plasma membrane ERK with distinct temporal dynamics. Elife 9, 1-18 (2020).
31. Hallin, J. et al. The KRASG12C inhibitor MRTX849 provides insight toward therapeutic susceptibility of KRAS-mutant cancers in mouse models and patients. Cancer Discov. 10, 54-71 (2020).
32. Zhou, X., Mehta, S. & Zhang, J. Genetically Encodable Fluorescent and Bioluminescent Biosensors Light Up Signaling Networks. Trends Biochem. Sci. 45, 889-905 (2020).
33. Allen, M. D. & Zhang, J. Subcellular dynamics of protein kinase A activity visualized by FRET-based reporters. Biochem. Biophys. Res. Commun. 348, 716-721 (2006).
34. Zhou, X. et al. Dynamic Visualization of mTORC1 Activity in Living Cells. Cell Rep. 10, 1767-1777 (2015).
35. Wiechmann, S. et al. Conformation-specific inhibitors of activated Ras GTPases reveal limited Ras dependency of patient-derived cancer organoids. J. Biol. Chem. 295, 4526-4540 (2020).
36. Zhou, M. et al. VPS35 binds farnesylated N-Ras in the cytosol to regulate N-Ras trafficking. J. Cell Biol. 214, 445-458 (2016).
37. Zhou, B., Der, C. J. & Cox, A. D. The role of wild type RAS isoforms in cancer. Semin. Cell Dev. Biol. 58, 60-69 (2016).
38. Rubio, I. et al. Ras activation in response to phorbol ester proceeds independently of the EGFR via an unconventional nucleotide-exchange factor system in COS-7 cells. Biochem. J. 398, 243-256 (2006).
39. Seong, J. et al. Visualization of Src Activity at Different Compartments of the Plasma Membrane by FRET Imaging. Chem. Biol. 16, 48-57 (2009).
40. Casar, B. et al. RAS at the Golgi antagonizes malignant transformation through PTPRκ-mediated inhibition of ERK activation. Nat. Commun. 9, 1-17 (2018).
41. Komatsu, T. et al. Organelle-specific, rapid induction of molecular activities and membrane tethering. Nat. Methods 7, 206-208 (2010).
42. Zhang, K. et al. Light-mediated kinetic control reveals the temporal effect of the Raf/MEK/ERK pathway in PC12 cell neurite outgrowth. PLoS One 9, e92917 (2014).
43. Rose, J. C. et al. A computationally engineered RAS rheostat reveals RAS-ERK signaling dynamics. Nat. Chem. Biol. 13, 119-126 (2017).

### Example 2: Designing RasAR Biosensor

### Pseudoligand is Designed Based on a Limited Region of the Effector Binding Region of Ras.

The pseudoligand was designed with regard to the crystal structure 4GON. Residues from Ras that were not considered for the pseudoligand design are omitted from the figure. The entire region of interest is amino acid sequence QNHFVDEYDPTIEDSYR (SEQ ID NO:6). Note: the pseudoligand used in the current version of RasAR is the 12 amino acid long QNHFVDEYDPTI (SEQ ID NO:1). The scope of tested variants of RasAR include, in addition to current RasAR, a 10 amino acid long sequence EYDPTIEDSY (SEQ ID NO:2), a 13 amino acid long sequence FVDEYDPTIEDSY (SEQ ID NO:3), a 9 amino acid long sequence QNHFVDEYD (SEQ ID NO:4), and a 16 amino acid long QNHFVDEYDPTIEDSY (SEQ ID NO:5). This pseudoligand design is similar to the PIP3 biosensor previously developed, but unique to Ras.¹

The Ras Binding Domain (RBD) is Derived from Ras Effector C-Raf

Ras is part of a family of proteins, known as the Ras family of proteins. While there is a large variety of RBD's from the large pool of Ras and Ras family effector proteins, the primary effector for Ras is C-Raf. Whose binding domain has high affinity for Ras, and specificity for Ras over Ras family protein Rap, due to a positively charged lysine at residue 84 of the RBD, which clashes with a similarly charged lysine within the effector binding interface of Rap but forms a charge-charge interaction with glutamate in Ras.² We have also made use a high affinity version of RBD, known as RBDv12. This variant was optimized using phage display to have high affinity binding to active Ras, in a manner that outcompetes endogenous effectors' Swapping the RBDwt with RBDv12, we have produced RasARv12, capable of binding endogenous Ras.

### Linker Screening of the RasARv12

To find a variant of RasARv12, a screening of linker variants between the mCerulean-RBDv12, and PL-Ypet was performed. To do so, truncated variants of the GGSGGSGGT (SEQ ID NO:7) linker of different lengths were assessed. 3-3 refers to original RasAR linker, with the left 3 referring to 3 amino acid linker GGS (SEQ ID NO:8) between mCerulean3 and RBDv12, and right 3 referring to the 3 amino acid linker GGS (SEQ ID NO:8) between PL and Ypet, whereas 9-9 refers to full GGSGGSGGT (SEQ ID NO:7) between these two positions. Variants with 5/7 amino acid linkers have truncations of 3′ end of the linker producing GGSGGSGG (SEQ ID NO:9) or GGSGG (SEQ ID NO:10). To test out the impact of these linker variants on the dynamic range of the sensor variants, cells were coexpressed with linker variants and HRas dominant negative (DN) mutant or HRas constitutively active mutant G12V. ΔR/R is defined by individual HRasG12V C/Y emission values normalized to the median C/Y emission value of the entire HRasDN population. Linker variant 3-5 has a GGS linker between mCer3-RBDv12 and a GGSGG (SEQ ID NO:10) linker between PL-Ypet. It's ΔR/R is measured as approximately ˜40% higher than the 10% response of original RasARv12.

### FRET-Pair Constitutes the Reporting Unit of the Sensor.

The RBD-pseudoligand pair forms what we refer to as a “sensing unit.” This unit is the portion of the biosensor that interacts with GTPase Ras. The Forster Resonance Energy Transfer (FRET)-pair constitutes the reporting unit, this translates the interaction of RBD-pseudoligand and Ras into a fluorescent signal. FRET pairs are constituted of a donor and acceptor fluorophore. To be compatible with resonant energy transfer, the wavelength of donor fluorophore emission requires sufficient overlap with the excitation wavelength of the acceptor fluorophore. Common fluorophore pairs compatible for FRET include cyan-yellow, or green-red, but many combinations are possible.⁴

### REFERENCES

1. Ni, Q., Fosbrink, M. & Zhang, J. Illuminating the phosphatidylinositol 3-kinase/Akt pathway. Small Anim. Whole-Body Opt. Imaging Based Genet. Eng. Probes 6868, 686809 (2008).
2. Wohlgemuth, S. et al. Recognizing and defining true ras binding domains I. Biochemical analysis. J. Mol. Biol. 348, 741-758 (2005).
3. Wiechmann, S. et al. Conformation-specific inhibitors of activated Ras GTPases reveal limited Ras dependency of patient-derived cancer organoids. J. Biol. Chem. 295, 4526-4540 (2020).
4. Bajar, B. T., Wang, E. S., Zhang, S., Lin, M. Z. & Chu, J. A guide to fluorescent protein FRET pairs. Sensors (Switzerland) 16, 1-24 (2016).

INFORMAL SEQUENCE LISTING

| SEQ ID NO: | Name of sequence | Sequence |
|---|---|---|
| 1. | Pseudoligand #1 | QNHFVDEYDPTI |
| 2. | Pscudoligand #2 | EYDPTIEDSY |
| 3. | Pseudoligand #3 | FVDEYDPTIEDSY |
| 4. | Pseudoligand #4 | QNHFVDEYD |
| 5. | Pseudoligand #5 | QNHFVDEYDPTIEDSY |
| 6. | Pseudoligand #6 | QNHFVDEYDPTIEDSYR |
| 7. | Linker #1 | GGSGGSGGT |
| 8. | Linker #2 | GGS |
| 9. | Linker #3 | GGSGGSGG |
| 10. | Linker #4 | GGSGG |
| 11. | Pseudoligand | YKLVVVGAGGVGKSALTIQLIQNHFVDEYDPTIEDSYRKQ |
| | domain region | VVIDGETCLLDILDTAGQEEYSAMRDQYMRTGE |
| 12. | KRas (Wildtype) | MTEYKLVVVGAGGVGKSALTIQLIQNHFVDEYDPTIEDSY |
| | | RKQVVIDGETCLLDILDTAGQEEYSAMRDQYMRTGEGFLC |
| | | VFAINNTKSFEDIHHYREQIKRVKDSEDVPMVLVGNKCDLP |
| | | SRTVDTKQAQDLARSYGIPFIETSAKTRQGVDDAFYTLVRE |
| | | IRKHKEKMSKDGKKKKKKSKTKCVIM |
| 13. | KRas fragment | MTEYKLVVVGAGGVGKSALTIQLIQNHFVDEYDPTIEDSY |
| | | RKQVVIDGETCLLDILD |
| 14. | Nuclear export | LPPLERLTL |
| | signal (NES) | |
| 15. | Giantin tag | EPQQSFSEAQQQLCNTRQEVNELRKLLEEERDQRVAA |
| | | ENALSVAEEQIRRLEHSEWDSSRTPIIGSCGTQEQALLI |
| | | DLTSNSCRRTRSGVGWKRVLRSLCHSRTRVPLLAAIY |
| | | FLMIHVLLILCFTGHL |
| 16. | Lyn-targeting motif | MGCIKSKRKDK |
| 17. | RBDwt (C-Raf) | PSKTSNTIRVFLPNKQRTVVNVRNGMSLHDCLMKAL |
| | | KVRGLQPECCAVFRLLHEHKGKKARLDWNTDAASLI |
| | | GEELQVDFL |
| 18. | RBDv12 | PSKTSNTIR VLLPNHERTVVKVRNGMSLHDSLMKALK |
| | | RHGLQPESSAVFRLLHEHKGKKARLDWNTDAASLIGE |
| | | ELHVDFL |
| 19. | RBDv1 | PSKTSNTIRVLLPNQEWTVVKVRNGMSLHDSLMKAL |
| | | KRHGLQPESSAVFRLLHEHKGKKARLDWNTDAASLI |
| | | GEELHVDFL |
| 20. | RBDv2 | PSKTSNTIRVFLPNKESTVVNVRNGMSLHDSLMKALK |
| | | RHGLQPESSAVFRLLHEHKGKKARLDWNTDAASLIGE |
| | | ELHVDFL |
| 21. | RBDv13 | PSKTSNTIRVLLPNNERTVVNVRNGMSLHDSLMKALK |
| | | RHGLQPESSAVFRLLHEHKGKKARLDWNTDAASLIGE |
| | | ELHVDFL |
| 22. | RasAR | GTTTAAACTTAAGCTTGCCACCATGGTGAGCAAGG |
| | mCerulean3 | GCGA |
| | fragment primer #1 | |
| 23. | RasAR | GCATGCGCTTGTACAGCTCGTCCATGCC |
| | mCerulean3 | |
| | fragment primer #2 | |
| 24. | RasAR Raf1-RBD | CTGTACAAGCGCATGCATCCTTCTAAGACAAGCAA |
| | fragment primer #1 | CACTATCC |
| 25. | RasAR Raf1-RBD | ACCTCCTATAGTGGGGTCGTATTCGTCCACAAAATG |
| | fragment primer #2 | ATTCTGACT |
| 26. | RasAR Raf1-RBD | CCTGTACCTCCACTACCTCCCAGGAAATCTACTTGA |
| | fragment primer #3 | AGTTCTTCTCCAAT |
| 27. | RasAR Raf1-RBD | TCCACAAAATGATTCTGACTACCTCCTGTACCTCCA |
| | fragment primer #4 | CTACCTCCCA |
| 28. | RasAR YPet | ACGACCCCACTATAGGAGGTACATCTAAAGGTGAA |
| | fragment primer #1 | GAATTATTCACTGG |
| 29. | RasAR YPet | GCAGACCACTTCCTTTGTACAATTCATTCATACCCT |
| | fragment primer #2 | CGG |
| 30. | RasAR YPet | GCTCCAGGGGGGGCAGACCACTTCCTTTGTACAATT |
| | fragment primer #3 | |
| 31. | RasAR pcDNA | GCCTGACCCTGTAaTCTAGAGGGCCCGTTTAAACC |
| | 3.1+ fragment | |
| | primer #1 | |
| 32. | RasAR pcDNA | CCCCTGGAGCGCCTGACCCTGTAaTCTAGAGGG |
| | 3.1+ fragment | |
| | primer #2 | |
| 33. | RasAR pcDNA | GTCTGCCCCCCCTGGAGCGCCTG |
| | 3.1+ fragment | |
| | primer #3 | |
| 34. | RasAR pcDNA | GCGAGCCCCTGATGCTCTTCGTCCAGATC |
| | 3.1+ fragment | |
| | primer #4 | |
| 35. | GA-RasAR RasAR | GTTTAAACTTAAGCTTGCCACCATGGTGAGCAAGG |
| | fragment primer #1 | GCGA |
| 36. | GA-RasAR RasAR | TCCCACTACCTTTGTACAATTCATTCATACCCTCGGT |
| | fragment primer #2 | AATACCAGCAGCAGTCAAAAATT |
| 37. | GA-RasAR | ATTACCGAGGGTATGAATGAATTGTACAAAGGTAG |
| | Giantin-pcDNA+ | TGGGACTGCCTCCGGAACGG |
| | fragment primer #1 | |
| 38. | GA-RasAR | GCGAGCCCCTGATGCTCTTCGTCCAGATC |
| | Giantin-pcDNA+ | |
| | fragment primer #2 | |
| 39. | pm-RasAR pcDNA | GATCTGGACGAAGAGCATCAGGGGCTCGC |
| | 3.1+-Lyn | |
| | fragment primer #1 | |
| 40. | pm-RasAR pcDNA | CATGGTGGCAAGCTTAAGTTTAAACGCTAGCCAGCT |
| | 3.1+-Lyn | T |
| | fragment primer #2 | |
| 41. | pm-RasAR pcDNA | CTTGCTCTTGATGCAGCCCATGGTGGCAAGCTTAA |
| | 3.1+-Lyn | |
| | fragment primer #3 | |
| 42. | pm-RasAR pcDNA | TTGTCCTTGCGCTTGCTCTTGATGCAGCCCA |
| | 3.1+-Lyn | |
| | fragment primer #4 | |
| 43. | pm-RasAR pcDNA | TTCCACCACTTCCACCCTTGTCCTTGCGCTTGCTCT |
| | 3.1+-Lyn | |
| | fragment primer #5 | |
| 44. | pm-RasAR RasAR | CAGGTGGTAGCGTGAGCAAGGGCGAGGA |
| | fragment primer #1 | |
| 45. | pm-RasAR RasAR | GGTGGAAGTGGTGGAACAGGTGGTAGCGTGAGC |
| | fragment primer #2 | |
| 46. | pm-RasAR RasAR | GCGAGCCCCTGATGCTCTTCGTCCAGATC |
| | fragment primer #3 | |
| 47. | mCherry fragment | GGCTAGCGTTTAAACTTAAGCTTGCCACCATGGTGA |
| | primer #1 | GCAAGGGCGAGGAGGATAACATGG |
| 48. | mCherry fragment | AGAGCCTCCTGTGCCACCAGAGCCACCCTTGTACAG |
| | primer #2 | CTCGTCCATGCCGCCG |
| 49. | mCherry | GATCTGGACGAAGAGCATCAGGGGCTCGC |
| | Optimized | |
| | KRas/NRas WT | |
| | fragment primer #1 | |
| 50. | mCherry | GATCTGGACGAAGAGCATCAGGGGCTCGC |
| | Optimized | |
| | KRas/NRas WT | |
| | fragment primer #2 | |
| 51. | Shared backbone | TTGATGAGTTTGGACAAACCACAACTAGAAT |
| | pcDNA 3.1+ | |
| | fragment primer #1 | |
| 52 | Shared backbone | TCTAGAGGGCCCGTTTAAACCCGCTGATCAG |
| | pcDNA 3.1+ | |
| | fragment primer #2 | |
| 53. | Shared backbone | GATCTGGACGAAGAGCATCAGGGGCTCGC |
| | pcDNA 3.1+ | |
| | fragment primer #3 | |
| 54. | Shared backbone | CATGGTGGCAAGCTTAAGTTTAAACGCTAGCCAGCT |
| | pcDNA 3.1+ | T |
| | fragment primer #4 | |

### P EMBODIMENTS

P Embodiment 1. Methods, systems, and articles of manufacture for biosensors as substantially described and shown herein.

### EMBODIMENTS

Embodiment 1. A recombinant protein comprising a first fluorophore, a ligand binding domain, a pseudoligand domain, and a second fluorophore.

Embodiment 2. The recombinant protein of Embodiment 1, wherein the first fluorophore is bound to the ligand binding domain through a first peptidyl linker, the pseudoligand domain is bound to the ligand binding domain through a second peptidyl linker, and the second fluorophore is bound to the pseudoligand domain through a third peptidyl linker.

Embodiment 3. The recombinant protein of Embodiment 1 or 2, wherein the first fluorophore and the second fluorophore are independently Forster Resonance Energy Transfer (FRET) fluorophores.

Embodiment 4. The recombinant protein of Embodiment 3, wherein the first FRET fluorophore or the second FRET fluorophore is a mCerulean3 fluorophore or a YPet fluorophore.

Embodiment 5. The recombinant protein of any one of Embodiments 1-4, wherein the pseudoligand domain comprises the amino acid sequence of SEQ ID NO:1, SEQ ID NO:2, SEQ ID NO:3, SEQ ID NO:4, SEQ ID NO:5, or SEQ ID NO:6.

Embodiment 6. The recombinant protein of any one of Embodiments 1-5, wherein the ligand binding domain comprises a Ras binding domain (RBD).

Embodiment 7. The recombinant protein of any one of Embodiments 1-6 wherein the ligand binding domain comprises the amino acid sequence of SEQ ID NO:17, SEQ ID NO:18, SEQ ID NO:19, SEQ ID NO:20, or SEQ ID NO:21

Embodiment 8. The recombinant protein of any one of Embodiments 2-7, wherein the C-terminus of the first fluorophore is attached to the N-terminus of the ligand binding domain through the first peptidyl linker.

Embodiment 9. The recombinant protein of any one of Embodiments 2-8, wherein the C-terminus of the ligand binding domain is attached to the N-terminus of the pseudoligand domain through the second peptidyl linker.

Embodiment 10. The recombinant protein of any one of Embodiments 2-9, wherein the C-terminus of the pseudoligand binding domain is attached to the N-terminus of the second fluorophore through the third peptidyl linker.

Embodiment 11. The recombinant protein of any one of Embodiments 1-10, further comprising a targeting moiety.

Embodiment 12. The recombinant protein of Embodiment 11, wherein the targeting moiety is a cytosolic targeting moiety, a plasma membrane targeting moiety, a Golgi apparatus targeting moiety.

Embodiment 13. The recombinant protein of Embodiment 11 or 12, wherein the targeting moiety is a nuclear export signal (NES), a Giantin tag, or a Lyn-targeting motif.

Embodiment 14. The recombinant protein of any one of Embodiments 11-13, wherein the targeting moiety comprises the amino acid sequence of SEQ ID NO:14, SEQ ID NO:15, or SEQ ID NO:16.

Embodiment 15. The recombinant protein of any one of Embodiments 11-14, wherein the C-terminus of the targeting moiety is attached to the N-terminus of the first fluorophore through a fourth peptidyl linker.

Embodiment 16. The recombinant protein of any one of Embodiments 11-14, wherein the C-terminus of the second fluorophore is attached to the N-terminus of the targeting moiety through a fourth peptidyl linker.

Embodiment 17. The recombinant protein of any one of Embodiments 2-16, wherein the first peptidyl linker, the second peptidyl linker, the third peptidyl linker, and/or the fourth peptidyl linker are independently an amino acid sequence of about 1 to about 1000 amino acids.

Embodiment 18. The recombinant protein of any one of Embodiments 2-17, wherein the first peptidyl linker, the second peptidyl linker, the third peptidyl linker, and/or the fourth peptidyl linker independently comprise at least 1 glycine, serine, or threonine.

Embodiment 19. The recombinant protein of any one of Embodiments 2-18, wherein at least 60% of the amino acids of the first peptidyl linker, the second peptidyl linker, the third peptidyl linker, and/or the fourth peptidyl linker comprise glycine.

Embodiment 20. The recombinant protein of any one of Embodiments 2-19, wherein at least 20% of the amino acids of the first peptidyl linker, the second peptidyl linker, the third peptidyl linker, and/or the fourth peptidyl linker independently comprise serine.

Embodiment 21. The recombinant protein of any one of Embodiments 2-20, wherein at least 10% of the amino acids of the first peptidyl linker, the second peptidyl linker, the third peptidyl linker, and/or the fourth peptidyl linker independently comprise threonine.

Embodiment 22. The recombinant protein of any one of Embodiments 2-21, wherein the first peptidyl linker, the second peptidyl linker, the third peptidyl linker, and/or the fourth peptidyl linker independently comprise the amino acid sequence of SEQ ID NO: 7, SEQ ID NO:8, SEQ ID NO:9, or SEQ ID NO:10.

Embodiment 23. The recombinant protein of any one of Embodiments 1-21, wherein the ligand binding domain is non-covalently bound to the pseudoligand domain.

Embodiment 24. The recombinant protein of any one of Embodiments 1-23, wherein the first fluorophore and the second fluorophore are a FRET pair.

Embodiment 25. The recombinant protein of any one of Embodiments 1-24, wherein the ligand binding domain binds a Ras protein with higher affinity than the pseudoligand domain.

Embodiment 26. The recombinant protein of Embodiment 25, wherein the Ras protein is an active Ras protein.

Embodiment 27. The recombinant protein of Embodiment 25 or 26, wherein the Ras protein is bound to guanosine triphosphate (GTP).

Embodiment 28. A complex comprising the recombinant protein of any one of Embodiments 1-27 non-covalently bound to a Ras protein.

Embodiment 29. The complex of Embodiment 28, wherein the ligand binding domain is non-covalently bound to the Ras protein.

Embodiment 30. The complex of Embodiment 29, wherein the Ras protein is an active Ras protein.

Embodiment 31. The complex of Embodiment 29 or 30, wherein the Ras protein is bound to guanosine triphosphate (GTP).

Embodiment 32. An isolated nucleic acid encoding the recombinant protein of any one of Embodiments 1-27.

Embodiment 33. An expression vector comprising the isolated nucleic acid of Embodiment 32.

Embodiment 34. The expression vector of Embodiment 33, wherein the expression vector is a viral vector.

Embodiment 35. The expression vector of Embodiment 34, wherein the viral vector is an Adeno-associated viral vector, an Adenovirus vector, or a lentiviral vector

Embodiment 36. A method for detecting a Ras protein in a cell, the method comprising: (a) contacting a cell with an expression vector comprising an isolated nucleic acid, wherein the isolated nucleic acid encodes a recombinant protein comprising: a first fluorophore, a ligand binding domain, a pseudoligand domain, and a second fluorophore; (b) transducing the cell with the expression vector; (c) allowing the cell to express the recombinant protein and bind to the Ras protein within the cell; and (d) detecting a fluorescent signal form the recombinant protein thereby detecting the Ras protein in the cell.

Embodiment 37. The method of Embodiment 36, wherein the first fluorophore is bound to the ligand binding domain through a first peptidyl linker, the pseudoligand domain is bound to the ligand binding domain through a second peptidyl linker, and the second fluorophore is bound to the pseudoligand domain through a third peptidyl linker.

Embodiment 38. The method of Embodiment 36 or 37, wherein the cell is imaged at a first time point to generate a first image.

Embodiment 39. The method of any one of Embodiments 36-38, wherein the cell is imaged at a second time point to generate a second image.

Embodiment 40. The method of any one of Embodiments 36-39, wherein step (d) comprises comparing the first image to the second image to determine activity of the Ras protein at the first time point compared to the second time point.

Embodiment 41. The method of any one of Embodiments 36-40, further comprising contacting the cell with an inhibitor at a third time point, wherein the third time point is between the first time point and the second time point.

Embodiment 42. The method of Embodiment 41, wherein the inhibitor targets the Ras protein or the Ras-Extracellular signal-regulated kinase (ERK) signaling pathway.

Embodiment 43. The method of Embodiment 41 or 42, wherein the inhibitor is a Ras inhibitor, a SHP2 inhibitor, a Ras guanine-nucleotide exchange factor (GEF) inhibitor, or a tyrosine kinase inhibitor.

Embodiment 44. The method of Embodiment 41-43, wherein the inhibitor is a KRasG12C inhibitor.

Embodiment 45. The method of any one of Embodiments 36-44, wherein the cell is a living cell.

Embodiment 46. The method of any one of Embodiments 36-45, wherein the cell is a mammalian cell.

Embodiment 47. The method of any one of Embodiments 38-41, wherein the cell is imaged using a fluorescence microscope.

Embodiment 48. The method of any one Embodiments 36-47, wherein the first fluorophore and the second fluorophore are a fluorescence resonance energy transfer (FRET) pair.

Embodiment 49. The method of any one of Embodiments 36-48, wherein the first fluorophore or the second fluorophore is a mCerulean3 fluorophore or a YPet fluorophore.

Embodiment 50. The method of any one of Embodiments 36-49, wherein the pseudoligand domain comprises the amino acid sequence of SEQ ID NO:1, SEQ ID NO:2, SEQ ID NO:3, SEQ ID NO:4, SEQ ID NO:5, or SEQ ID NO:6.

Embodiment 51. The method of any one of Embodiments 36-50, wherein the ligand binding domain comprises a Ras binding domain (RBD).

Embodiment 52. The recombinant protein of any one of Embodiments 36-51 wherein the ligand binding domain comprises the amino acid sequence of SEQ ID NO:17, SEQ ID NO:18, SEQ ID NO:19, SEQ ID NO:20, or SEQ ID NO:21

Embodiment 53. The method of any one of Embodiments 37-52, wherein the C-terminus of the first fluorophore is attached to the N-terminus of the ligand binding domain through the first peptidyl linker.

Embodiment 54. The method of any one of Embodiments 37-53, wherein the C-terminus of the ligand binding domain is attached to the N-terminus of the pseudoligand domain through the second peptidyl linker.

Embodiment 55. The method of any one of Embodiments 37-54, wherein the C-terminus of the pseudoligand binding domain is attached to the N-terminus of the second fluorophore through the third peptidyl linker.

Embodiment 56. The method of any one of Embodiments 36-55, further comprising a targeting moiety.

Embodiment 57. The method of Embodiment 56, wherein the targeting moiety is a cytosolic targeting moiety, a plasma membrane targeting moiety, a Golgi apparatus targeting moiety.

Embodiment 58. The method of Embodiment 56 or 57, wherein the targeting moiety is a nuclear export signal (NES), a Giantin tag, or a Lyn-targeting motif.

Embodiment 59. The recombinant protein of any one of Embodiments 56-58, wherein the targeting moiety comprises the amino acid sequence of SEQ ID NO:14, SEQ ID NO:15, or SEQ ID NO:16.

Embodiment 60. The method of any one of Embodiments 51-53, wherein the C-terminus of the targeting moiety is attached to the N-terminus of the first fluorophore through a fourth peptidyl linker.

Embodiment 61. The method of any one of Embodiments 56-59, wherein the C-terminus of the second fluorophore is attached to the N-terminus of the targeting moiety through a fourth peptidyl linker.

Embodiment 62. The method of any one of Embodiments 37-61, wherein the first peptidyl linker, the second peptidyl linker, the third peptidyl linker, and/or the fourth peptidyl linker are independently an amino acid sequence of about 1 to about 1000 amino acids.

Embodiment 63. The method of any one of Embodiments 37-62, wherein the first peptidyl linker, the second peptidyl linker, the third peptidyl linker, and/or the fourth peptidyl linker independently comprise at least 1 glycine, serine, or threonine.

Embodiment 64. The method of any one of Embodiments 37-63, wherein at least 60% of the amino acids of the first peptidyl linker, the second peptidyl linker, the third peptidyl linker, and/or the fourth peptidyl linker comprise glycine.

Embodiment 65. The method of any one of Embodiments 37-64, wherein at least 20% of the amino acids of the first peptidyl linker, the second peptidyl linker, the third peptidyl linker, and/or the fourth peptidyl linker independently comprise serine.

Embodiment 66. The method of any one of Embodiments 37-65, wherein at least 10% of the amino acids of the first peptidyl linker, the second peptidyl linker, the third peptidyl linker, and/or the fourth peptidyl linker independently comprise threonine.

Embodiment 67. The method of any one of Embodiments 36-66, wherein the ligand binding domain is non-covalently bound to the pseudoligand domain.

Embodiment 68. The method of any one of Embodiments 36-67, wherein the ligand binding domain binds the ligand with higher affinity than the pseudoligand domain.

Embodiment 69. The method of any one of Embodiments 36-68, wherein the Ras protein is an active Ras protein.

Embodiment 70. The method of any one of Embodiments 36-69, wherein the Ras protein is bound to guanosine triphosphate (GTP).

What is claimed is:

1. A recombinant protein comprising a first fluorophore, a ligand binding domain, a pseudoligand domain, and a second fluorophore.

2. The recombinant protein of claim 1, wherein the first fluorophore is bound to the ligand binding domain through a first peptidyl linker, the pseudoligand domain is bound to the ligand binding domain through a second peptidyl linker, and the second fluorophore is bound to the pseudoligand domain through a third peptidyl linker.

3. The recombinant protein of claim 1, wherein the first fluorophore and the second fluorophore are independently Forster Resonance Energy Transfer (FRET) fluorophores.

4. The recombinant protein of claim 3, wherein the first FRET fluorophore or the second FRET fluorophore is a mCerulean3 fluorophore or a YPet fluorophore.

5. The recombinant protein of claim 1, wherein the pseudoligand domain comprises the amino acid sequence of SEQ ID NO:1, SEQ ID NO:2, SEQ ID NO:3, SEQ ID NO:4, SEQ ID NO:5, or SEQ ID NO:6.

6. The recombinant protein of claim 1, wherein the ligand binding domain comprises a Ras binding domain (RBD).

7. The recombinant protein of claim 1, wherein the ligand binding domain comprises the amino acid sequence of SEQ ID NO:17, SEQ ID NO:18, SEQ ID NO:19, SEQ ID NO:20, or SEQ ID NO:21.

8. The recombinant protein of claim 2, wherein the C-terminus of the first fluorophore is attached to the N-terminus of the ligand binding domain through the first peptidyl linker.

9. The recombinant protein of claim 2, wherein the C-terminus of the ligand binding domain is attached to the N-terminus of the pseudoligand domain through the second peptidyl linker.

10. The recombinant protein of claim 2, wherein the C-terminus of the pseudoligand binding domain is attached to the N-terminus of the second fluorophore through the third peptidyl linker.

11. The recombinant protein of claim 1, further comprising a targeting moiety.

12. The recombinant protein of claim 11, wherein the targeting moiety is a cytosolic targeting moiety, a plasma membrane targeting moiety, a Golgi apparatus targeting moiety.

13. The recombinant protein of claim 11, wherein the targeting moiety is a nuclear export signal (NES), a Giantin tag, or a Lyn-targeting motif.

14. The recombinant protein of claim 11, wherein the targeting moiety comprises the amino acid sequence of SEQ ID NO:14, SEQ ID NO:15, or SEQ ID NO:16.

15. The recombinant protein of claim 11, wherein the C-terminus of the targeting moiety is attached to the N-terminus of the first fluorophore through a fourth peptidyl linker.

16. The recombinant protein of claim 11, wherein the C-terminus of the second fluorophore is attached to the N-terminus of the targeting moiety through a fourth peptidyl linker.

17. The recombinant protein of claim 2, wherein the first peptidyl linker, the second peptidyl linker, the third peptidyl linker, and/or the fourth peptidyl linker are independently an amino acid sequence of about 1 to about 1000 amino acids.

18. The recombinant protein of claim 2, wherein the first peptidyl linker, the second peptidyl linker, the third peptidyl linker, and/or the fourth peptidyl linker independently comprise at least 1 glycine, serine, or threonine.

19. The recombinant protein of claim 2, wherein at least 60% of the amino acids of the first peptidyl linker, the second peptidyl linker, the third peptidyl linker, and/or the fourth peptidyl linker comprise glycine.

20. The recombinant protein of claim 2, wherein at least 20% of the amino acids of the first peptidyl linker, the second peptidyl linker, the third peptidyl linker, and/or the fourth peptidyl linker independently comprise serine.

21. The recombinant protein of claim 2, wherein at least 10% of the amino acids of the first peptidyl linker, the second peptidyl linker, the third peptidyl linker, and/or the fourth peptidyl linker independently comprise threonine.

22. The recombinant protein of claim 2, wherein the first peptidyl linker, the second peptidyl linker, the third peptidyl linker, and/or the fourth peptidyl linker independently comprise the amino acid sequence of SEQ ID NO:7, SEQ ID NO:8, SEQ ID NO:9, or SEQ ID NO: 10.

23. The recombinant protein of claim 1, wherein the ligand binding domain is non-covalently bound to the pseudoligand domain.

24. The recombinant protein of claim 1, wherein the first fluorophore and the second fluorophore are a FRET pair.

25. The recombinant protein of claim 1, wherein the ligand binding domain binds a Ras protein with higher affinity than the pseudoligand domain.

26. The recombinant protein of claim 25, wherein the Ras protein is an active Ras protein.

27. The recombinant protein of claim 25, wherein the Ras protein is bound to guanosine triphosphate (GTP).

28. A complex comprising the recombinant protein of claim 1 non-covalently bound to a Ras protein.

29. The complex of claim 28, wherein the ligand binding domain is non-covalently bound to the Ras protein.

30. The complex of claim 29, wherein the Ras protein is an active Ras protein.

31. The complex of claim 29, wherein the Ras protein is bound to guanosine triphosphate (GTP).

32. An isolated nucleic acid encoding the recombinant protein of claim 1.

33. An expression vector comprising the isolated nucleic acid of claim 32.

34. The expression vector of claim 33, wherein the expression vector is a viral vector.

35. The expression vector of claim 34, wherein the viral vector is an Adeno-associated viral vector, an Adenovirus vector, or a lentiviral vector.

36. A method for detecting a Ras protein in a cell, the method comprising:
(a) contacting a cell with an expression vector comprising an isolated nucleic acid, wherein the isolated nucleic acid encodes a recombinant protein comprising: a first fluorophore, a ligand binding domain, a pseudoligand domain, and a second fluorophore;
(b) transducing the cell with the expression vector;
(c) allowing the cell to express the recombinant protein and bind to the Ras protein within the cell; and
(d) detecting a fluorescent signal form the recombinant protein thereby detecting the Ras protein in the cell.

37. The method of claim 36, wherein the first fluorophore is bound to the ligand binding domain through a first peptidyl linker, the pseudoligand domain is bound to the ligand binding domain through a second peptidyl linker, and the second fluorophore is bound to the pseudoligand domain through a third peptidyl linker.

38. The method of claim 36, wherein the cell is imaged at a first time point to generate a first image.

39. The method of claim 36, wherein the cell is imaged at a second time point to generate a second image.

40. The method of claim 36, wherein step (d) comprises comparing the first image to the second image to determine activity of the Ras protein at the first time point compared to the second time point.

41. The method of claim 36, further comprising contacting the cell with an inhibitor at a third time point, wherein the third time point is between the first time point and the second time point.

42. The method of claim 41, wherein the inhibitor targets the Ras protein or the Ras-Extracellular signal-regulated kinase (ERK) signaling pathway.

43. The method of claim 41, wherein the inhibitor is a Ras inhibitor, a SHP2 inhibitor, a Ras guanine-nucleotide exchange factor (GEF) inhibitor, or a tyrosine kinase inhibitor.

44. The method of claim 41, wherein the inhibitor is a KRasG12C inhibitor.

45. The method of claim 36, wherein the cell is a living cell.

46. The method of claim 36, wherein the cell is a mammalian cell.

47. The method of claim 38, wherein the cell is imaged using a fluorescence microscope.

48. The method of claim 36, wherein the first fluorophore and the second fluorophore are a fluorescence resonance energy transfer (FRET) pair.

49. The method of claim 36, wherein the first fluorophore or the second fluorophore is a mCerulean3 fluorophore or a YPet fluorophore.

50. The method of claim 36, wherein the pseudoligand domain comprises the amino acid sequence of SEQ ID NO:1, SEQ ID NO:2, SEQ ID NO:3, SEQ ID NO:4, SEQ ID NO:5, or SEQ ID NO:6.

51. The recombinant protein of claim 36, wherein the ligand binding domain comprises a Ras binding domain (RBD).

52. The recombinant protein of claim 36, wherein the ligand binding domain comprises the amino acid sequence of SEQ ID NO:17, SEQ ID NO:18, SEQ ID NO:19, SEQ ID NO:20, or SEQ ID NO:21.

53. The method of claim 37, wherein the C-terminus of the first fluorophore is attached to the N-terminus of the ligand binding domain through the first peptidyl linker.

54. The method of claim 37, wherein the C-terminus of the ligand binding domain is attached to the N-terminus of the pseudoligand domain through the second peptidyl linker.

55. The method of claim 37, wherein the C-terminus of the pseudoligand binding domain is attached to the N-terminus of the second fluorophore through the third peptidyl linker.

56. The method of claim 36, further comprising a targeting moiety.

57. The method of claim 56, wherein the targeting moiety is a cytosolic targeting moiety, a plasma membrane targeting moiety, a Golgi apparatus targeting moiety.

58. The method of claim 56, wherein the targeting moiety is a nuclear export signal (NES), a Giantin tag, or a Lyn-targeting motif.

59. The method of claim 56, wherein the targeting moiety comprises the amino acid sequence of SEQ ID NO: 14, SEQ ID NO:15, or SEQ ID NO:16.

60. The method of claim 56, wherein the C-terminus of the targeting moiety is attached to the N-terminus of the first fluorophore through a fourth peptidyl linker.

61. The method of claim 56, wherein the C-terminus of the second fluorophore is attached to the N-terminus of the targeting moiety through a fourth peptidyl linker.

62. The method of claim 37, wherein the first peptidyl linker, the second peptidyl linker, the third peptidyl linker, and/or the fourth peptidyl linker are independently an amino acid sequence of about 1 to about 1000 amino acids.

63. The method of claim 37, wherein the first peptidyl linker, the second peptidyl linker, the third peptidyl linker, and/or the fourth peptidyl linker independently comprise at least 1 glycine, serine, or threonine.

64. The method of claim 37, wherein at least 60% of the amino acids of the first peptidyl linker, the second peptidyl linker, the third peptidyl linker, and/or the fourth peptidyl linker comprise glycine.

65. The method of claim 37, wherein at least 20% of the amino acids of the first peptidyl linker, the second peptidyl linker, the third peptidyl linker, and/or the fourth peptidyl linker independently comprise serine.

66. The method of claim 37, wherein at least 10% of the amino acids of the first peptidyl linker, the second peptidyl linker, the third peptidyl linker, and/or the fourth peptidyl linker independently comprise threonine.

67. The method of claim 36, wherein the ligand binding domain is non-covalently bound to the pseudoligand domain.

68. The method of claim 36, wherein the ligand binding domain binds the ligand with higher affinity than the pseudoligand domain.

69. The method of claim 36, wherein the Ras protein is an active Ras protein.

70. The method of claim 36, wherein the Ras protein is bound to guanosine triphosphate (GTP).
